(19) United States
(12) Patent Application Publication
(10) Pub. No.: US 2024/0150277 A1
(43) Pub. Date: May 9, 2024

(54) Covalent PPARG inverse-agonists

(71) Applicants: Bayer Aktiengesellschaft, Leverkusen (DE); The Broad Institute, Inc., Cambridge, MA (US); Dana-Farber Cancer Institute, Inc., Boston, MA (US)

(72) Inventors: Stephan SIEGEL, Berlin (DE); Knut EIS, Newton, MA (US); Timo STELLFELD, Berlin (DE); Léa Aurelie BOUCHÉ, Basel (CH); Elisabeth POOK, Cambridge, MA (US); Joseph Peter BLUCK, Berlin (DE); Philip LIENAU, Berlin (DE); Constantia PANTELIDOU, Brookline, MA (US); Isabel Sophie JERCHEL-FURAU, Concord, MA (US); Ulf BRÜEGGEMEIER, Leichlingen (DE); Sven CHRISTIAN, Somerville, MA (US); Detlev SÜLZLE, Berlin (DE); Anders Roland FRIBERG, Berlin (DE); Simon HOLTON, Berlin (DE); Christian LECHNER, Berlin (DE); Stefan KAULFUSS, Berlin (DE); Hanna MEYER, Berlin (DE); Douglas ORSI, Cambridge, MA (US); Steven James FERRARA, Cambridge, MA (US); Jonathan GOLDSTEIN, Cambridge, MA (US); Matthew MEYERSON, Boston, MA (US); Christopher LEMKE, Cambridge, MA (US)

(73) Assignees: Bayer Aktiengesellschaft, Leverkusen (DE); The Broad Institute, Inc., Cambridge, MA (US); Dana-Farber Cancer Institute, Inc., Boston, MA (US)

(21) Appl. No.: 18/241,190

(22) Filed: Aug. 31, 2023

Related U.S. Application Data
(60) Provisional application No. 63/403,830, filed on Sep. 5, 2022.

(51) Int. Cl. C07C 237/42; A61P 35/00; C07D 211/22; C07D 213/40; C07D 241/20; C07D 263/57; C07D 271/06; C07D 271/10; C07D 305/06; C07D 405/04

(52) U.S. Cl. CPC C07C 237/42; A61P 35/00; C07D 211/22; C07D 213/40; C07D 241/20; C07D 263/57; C07D 271/06; C07D 271/10; C07D 305/06; C07D 405/04; C07C 2601/16; C07C 2602/10

(57) ABSTRACT

The present invention includes compounds of formula (I)

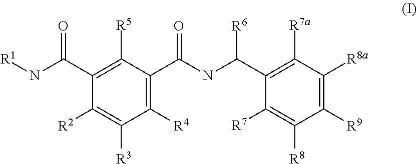

and methods for its preparation and treatment of disorders with activated peroxisome preoliferator-activated receptor gamma (PPARG), particularly cancer.

### CROSS-REFERENCE TO RELATED APPLICATION

This application claims priority to and the benefit U.S. Provisional Application No. 63/403,830, filed Sep. 5, 2022, the entire contents of which are incorporated by reference herein.

The present invention includes compositions and methods for treatment of disorders with activated peroxisome preoliferator-activated receptor gamma (PPARG). This invention more specifically includes novel covalent inverse agonists of PPARG that can be used to treat bladder cancer, pancreatic cancer, prostate cancer, colorectal cancer, esophagael cancer, gastric cancer, and breast cancer.

### BACKGROUND

Peroxisome Proliferator Activated Receptor Gamma (PPARG) is a ligand-activated transcription factor that regulates the expression of target genes. PPARG requires heterodimerization with a member of its obligate nuclear receptor partner retinoid X receptor (RXR); RXR-alpha (RXRA), RXR-beta (RXRB), or RXR-gamma (RXRG). Together they bind to DNA response elements in the promoter and enhancer regions of target genes. The PPARG/RXR complex interacts with nuclear receptor coactivators (NCOA1 and others) and nuclear receptor co-repressors NCOR1 and NCOR2, which recruit additional transcriptional regulatory proteins including histone acetyl-transferases and histone deacetylases, mediator complex (MED1) as well as the basal transcriptional machinery. These dynamic interactions lead to regulation of target gene expression.

Many canonical target genes of the PPARG transcription complex are involved in the regulation of lipid and glucose homeostasis. PPARG agonists improve glucose tolerance and insulin sensitivity, and the thiazolidinedione (glitazone) and glitazar anti-diabetic drug classes exploit this pharmacology for their therapeutic effect. The anti-diabetic thiazolidinedione drugs including rosiglitazone and pioglitazone are selective PPARG agonists, while members of the glitazar family (including saroglitazar and other failed drug candidates) are dual agonists of both PPARA and PPARG. Interestingly, high doses of the PPARG agonist pioglitazone is associated with an increased risk of bladder cancer in rodents and patients, which limited clinical use. Additionally, high incidence of bladder cancer in rodent toxicity studies during pre-clinical development of glitazars has been a challenge for clinical development of this class.

Peroxisome proliferator-activated receptor gamma (PPARG) activated cancers (e.g., breast cancer, esophageal cancer, pancreatic cancer, colorectal cancer, hepatocellular cancer, bladder cancer, and the like) represent a significant class of cancers. For example, bladder cancer is the fifth most commonly diagnosed cancer type in the United States. About half of all bladder cancer patients are diagnosed with non-invasive/superficial urothelial carcinoma of the bladder and respond well to existing chemotherapy regimens, with a 5-year survival rate of 96%. Patients diagnosed with invasive disease have a poorer prognosis, with a 5-year survival rate of 70% or less, depending upon extent of invasion beyond the bladder Currently, there are few therapeutic options available to these patients. While recent approvals of immune checkpoint inhibitors (ICI) have helped prognosis in a subset of patients, there is still a need for additional therapeutic options and potential therapeutics to enhance the activity of ICIs.

PPARG is an enriched genetic dependency in luminal bladder cancer. Hotspot mutations of the heterodimeric partner RXRA at S427 and focal gene ampli-fication of PPARG lead to ligand-independent activation of PPARG and appear to be oncogenic through multiple mechanisms. PPARG inverse agonists, exemplified by T0070907 and SR10221, which inhibit gene transactivation by promoting a repressive conformation show selective anti-proliferative activity in vitro against PPARG-dependent cell lines. Inverse agonists would have the opposite effect of an agonist and suppress PPARG transcriptional activity in cancers and other conditions including metabolism, bone biology, and inflammation.

PPARG is in the top ten most enriched genetic dependencies in pancreatic cancer and cancer of the urinary tract lineage within the Cancer Dependency Map genome-wide CRISPR loss-of-function screen across a panel of over 1000 cell lines (DepMap.org). Pancreatic cancer and cancer of the urinary tract lineage have amongst the highest average gene expression profiles for PPARG amongst all lineages indicating a potential patient population for consideration. There are very limited therapeutic options for patients with pancreatic cancer and prognosis remains grim.

Accordingly, there is an urgent need for compositions and methods for treating PPARG activated cancers such as bladder and pancreatic cancer.

### SUMMARY

The present disclosure relates, at least in part, to the discovery of peroxisome proliferator-activated receptor gamma (PPARG) inverse agonists as potential therapeutic for the treatment of various PPARG activated cancers such as, for example, breast cancer, bladder cancer, colorectal cancer, esophageal cancer, gastric cancer, hepatocellular cancer, pancreatic cancer, and prostate cancer. As described herein, the present disclosure provides PPARG modulators, more specifically inverse-agonists, that are able to down regulate PPARG signaling in PPARG activated cancers, thereby decreasing cellular proliferation associated with PPARG activated cancers. In particular, the present disclosure provides inverse-agonists that are able to reverse up-regulation of PPARG signaling in PPARG activated cancers, thereby providing a therapeutic modality capable of treating PPARG activated cancers such as, for example, cancer of the urinary tract, particularly bladder cancer or pancreatic cancer.

In an aspect, the present disclosure provides a method of treating a subject having a peroxisome proliferator-activated receptor gamma (PPARG) activated cancer that includes a step of administering a therapeutically effective amount of a PPARG signaling modulator, particularly an inverse-agonist to the subject.

In an embodiment, the PPARG inverse agonists are a compounds of formula (I)

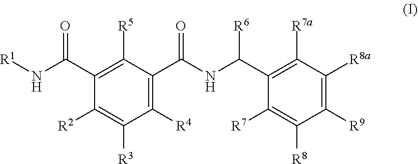

wherein, independently from each occurrence
R¹ is selected from

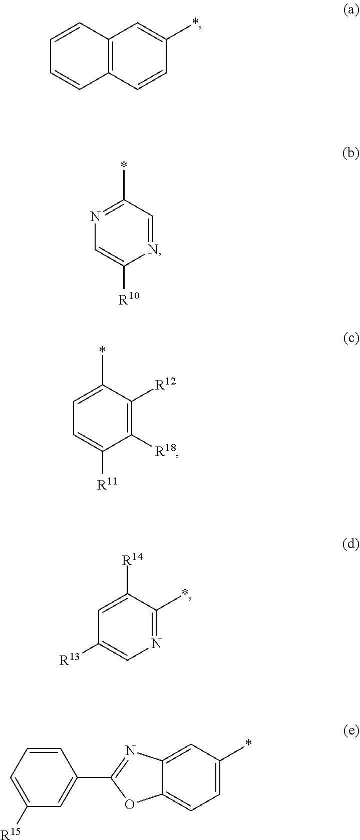

and * is the point of attachment to the nitrogen atom.
R² is selected from the group consisting of a halogen atom, and a —C═CH group;
R³ is selected from the group consisting of a hydrogen atom, an C₁-C₃-alkyl group, and a halogen atom;
R⁴ is selected from the group consisting of a hydrogen atom and a halogen atom;
R⁵ is selected from the group consisting of a hydrogen atom and a halogen atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, a (C₁-C₃)alkoxy group, and a —CH₂OCH₂CH₂R¹⁶ group;
R⁸, R^{8a} is independently selected from the group consisting of a hydrogen atom and a halogen atom;
R⁹ is selected from the group consisting of a hydrogen atom and a halogen atom,
R¹⁰ is selected from the group consisting of a hydrogen atom and a phenoxy group;
R¹¹ is selected from the group consisting of a hydrogen atom, an C₁-C₃-alkyl group, a C₁-C₃-hydroxyalkyl group, a 3-6 membered heterocycloalkyl group comprising 1 or 2 heteroatoms independently selected from oxygen and nitrogen which is optionally substituted with a hydroxy group, a C₁-C₃-haloalkyoxy group, a C₁-C₃-haloalkyl group, a 5-6-membered heteroaryl group which is optionally substituted with a C₁-C₃-alkyl group, a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a halogen atom;
R¹⁶ is selected from the group consisting of a C₁-C₃-alkyl group and a C₁-C₃-alkoxy group;
R¹⁷ is selected from the group consisting of a hydrogen atom and a C₁-C₃-alkyl group;
R¹⁸ is selected from the group consisting of a hydrogen atom and a halogen atom; or a stereoisomer, a tautomer, an N-oxide, a hydrate, a solvate, or a salt thereof, or a mixture of same as well as combinations thereof.

In an embodiment, the PPARG signaling modulator is an inverse-agonist of PPARG signaling.

### BRIEF DESCRIPTION OF THE DRAWINGS

FIG. 1 (FIG. 1): shows a graph for relative % proliferation vs compound concentration reflecting the effect of the compound of example 51 acting on the urinary bladder transitional cell carcinoma cell line SW780. Proliferation is reported as percent of DMSO control after 7 days treatment with compound with baseline normalization to reading on day 0. Data is reported as mean+/−standard deviation.

FIG. 2 (FIG. 2): shows a graph for relative % proliferation vs compound concentration reflecting the effect of the compound of example 51 acting on the urinary bladder transitional cell carcinoma cell line UMUC9. Proliferation reported as percent of DMSO control after 7 days treatment with compound with baseline normalization to reading on day 0. Data is reported as mean+/−standard deviation.

FIG. 3 (FIG. 3): shows a graph for relative % proliferation vs compound concentration reflecting the effect of the compound of example 51 acting on the primary urinary bladder carcinoma cell line RT112. Proliferation reported as percent of DMSO control after 7 days treatment with compound with baseline normalization to reading on day 0. Data is reported as mean+/−standard deviation.

FIG. 4 (FIG. 4): shows a graph for relative % proliferation vs compound concentration reflecting the effect of the compound of example 51 acting on the urinary bladder carcinoma cell line HT-1197. Proliferation reported as percent of DMSO control after 7 days treatment with compound with baseline normalization to reading on day 0. Data is reported as mean+/−standard deviation.

FIG. 5 (FIG. 5): shows a graph for relative % proliferation vs compound concentration reflecting the effect of the compound of example 51 acting on the grade II primary bladder carcinoma cell line 5637. Proliferation reported as percent of DMSO control after 7 days treatment with compound with baseline normalization to reading on day 0. Data is reported as mean+/−standard deviation.

FIG. 6 (FIG. 6): shows a graph for relative % proliferation vs compound concentration reflecting the effect of the compound of example 51 acting on the pancreatic ductal adenocarcinoma cell line PACADD188. Proliferation reported as percent of DMSO control after 7 days treatment with compound with baseline normalization to reading on day 0. Data is reported as mean+/−standard deviation.

FIG. 7 (FIG. 7): shows a graph for relative % proliferation vs compound concentration reflecting the effect of the compound of example 51 acting on the pancreatic ductal adenocarcinoma cell line HUPT4. Proliferation reported as percent of DMSO control after 7 days treatment with compound with baseline normalization to reading on day 0. Data is reported as mean+/−standard deviation.

FIG. 8 (FIG. 8): shows a graph for relative % proliferation vs compound concentration reflecting the effect of the compound of example 51 acting on the pancreatic ductal adenocarcinoma cell line PACADD161. Proliferation reported as percent of DMSO control after 7 days treatment with compound with baseline normalization to reading on day 0. Data is reported as mean+/−standard deviation.

FIG. 9 (FIG. 9): shows a graph for relative % proliferation vs compound concentration reflecting the effect of the compound of example 51 acting on the pancreatic ductal adenocarcinoma cell line PACADD119. Proliferation reported as percent of DMSO control after 7 days treatment with compound with baseline normalization to reading on day 0. Data is reported as mean+/−standard deviation.

FIG. 10 (FIG. 10): shows a graph for relative % proliferation vs compound concentration reflecting the effect of the compound of example 41 acting on the urinary bladder transitional cell carcinoma cell line SW780. Proliferation reported as percent of DMSO control after 7 days treatment with compound with baseline normalization to reading on day 0. Data is reported as mean+/−standard deviation.

FIG. 11 (FIG. 11): shows a graph for relative % proliferation vs compound concentration reflecting the effect of the compound of example 41 acting on the urinary bladder transitional cell carcinoma cell line UMUC9. Proliferation reported as percent of DMSO control after 7 days treatment with compound with baseline normalization to reading on day 0. Data is reported as mean+/−standard deviation.

FIG. 12 (FIG. 12): shows a graph for relative % proliferation vs compound concentration reflecting the effect of the compound of example 41 acting on the primary urinary bladder carcinoma cell line RT112. Proliferation reported as percent of DMSO control after 7 days treatment with compound with baseline normalization to reading on day 0. Data is reported as mean+/−standard deviation.

FIG. 13 (FIG. 13): shows a graph for relative % proliferation vs compound concentration reflecting the effect of the compound of example 41 acting on the urinary bladder carcinoma cell line HT-1197. Proliferation reported as percent of DMSO control after 7 days treatment with compound with baseline normalization to reading on day 0. Data is reported as mean+/−standard deviation.

FIG. 14 (FIG. 14): shows a graph for relative % proliferation vs compound concentration for the compound of example 41 acting on the grade II primary bladder carcinoma cell line 5637. Proliferation reported as percent of DMSO control after 7 days treatment with compound with baseline normalization to reading on day 0. Data is reported as mean+/−standard deviation.

FIG. 15 (FIG. 15): shows a graph for relative % proliferation vs compound concentration reflecting the effect of the compound of example 41 acting on the pancreatic ductal adenocarcinoma cell line PACADD188. Proliferation reported as percent of DMSO control after 7 days treatment with compound with baseline normalization to reading on day 0. Data is reported as mean+/−standard deviation.

FIG. 16 (FIG. 16): shows a graph for relative % proliferation vs compound concentration reflecting the effect of the compound of example 41 acting on the pancreatic ductal adenocarcinoma cell line HUPT4. Proliferation reported as percent of DMSO control after 7 days treatment with compound with baseline normalization to reading on day 0. Data is reported as mean+/−standard deviation.

FIG. 17 (FIG. 17): shows a graph for relative % proliferation vs compound concentration reflecting the effect of the compound of example 41 acting on pancreatic ductal adenocarcinoma cell line PACADD161. Proliferation reported as percent of DMSO control after 7 days treatment with compound with baseline normalization to reading on day 0. Data is reported as mean+/−standard deviation.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Structures drawn include all permissible rotations about bonds.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, in particular 1, or 2.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkyl)-O—(C₁-C₄-alkyl)-, a hyphen at the beginning or at the end of such a composite substituent indicates the point of attachment of said composite substituent to the rest of the molecule. Should the composite substituent be substituted said substitutent may be bound at any suitable carbon atom of the composite substitutent.

Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “alkylene” derives from the term “alkyl” as being a bivalent constituent named by addition of “ene” to the term “alkyl” e.g. “methyl” becomes “methylene” meaning a “—CH₂—” constituent whereby the open bonds of branched constituents are located at the respective ends of the longest chain.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl, more particularly trifluoromethyl or trifluoromethyl.

The term “hydroxyalkyl” means a linear or branched, saturated, monovalent hydrocarbon group in which the term “C₁-C₆-alkyl” is as defined supra, and in which one or more hydrogen atoms are replaced by a hydroxy group. Preferred is a monohydroxyalkyl group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₃-C₆-cycloalkyl” means a saturated, monovalent, mono- or bicyclic hydrocarbon ring which contains 3, 4, 5, or 6, carbon atoms. Said C₃-C₆-cycloalkyl group is for example, a monocyclic hydrocarbon ring, e.g. a cyclopropyl, cyclobutyl, cyclopentyl, or a cyclohexyl group.

The term “3- to 6-membered heterocycloalkyl” mean a saturated heterocycle with 3, 4, 5, or 6, ring atoms in total, which contains one or two identical or different ring heteroatoms independently selected from the series N, and O, said heterocycloalkyl group being attached to the rest of the molecule via any one of the carbon atoms or a nitrogen atom.

Said heterocycloalkyl group, without being limited thereto, can be a 3- to 4-membered ring, such as azacyclopropyl, oxacyclopropyl, azetidinyl, or oxetanyl for example; or a 5-membered ring, such as tetrahydrofuranyl, 1,3-dioxolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example.

Particularly, “5- or 6-membered heterocycloalkyl” means a monocyclic, saturated heterocycle with 5 or 6 ring atoms in total, containing one ring nitrogen atom and optionally one further ring heteroatom from the series: N, O. Said heterocycloalkyl group is being attached to the rest of the molecule via any carbon atom or where applicable via any nitrogen atom.

The term “heteroaryl” means a monovalent, monocyclic or bicyclic aromatic ring having 5, to 6 ring atoms (a “5- to 6-membered heteroaryl” group), containing at least one ring heteroatom and optionally one, two or three further ring heteroatoms from the series: N, O and/or S, and which is bound via a ring carbon atom to the rest of the molecule.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, or polypeptide, or fragments thereof.

“Alkylating agents” directly damage DNA to prevent the cancer cell from reproducing. As a class of drugs, these agents are not phase-specific; in other words, they work in all phases of the cell cycle. Alkylating agents are used to treat many different cancers. Examples of alkylating agents include, for example, nitrogen mustards (e.g., mechlorethamine, chlorambucil, cyclophosphamide (Cytoxan@), ifosfamide, and melphalan), alkyl sulfonates (e.g., busulfan), triazines (e.g., dacarbazine (DTIC), temozolomide (Temodar@)), Nitrosoureas (including streptozocin, carmustine (BCNU), and Iomustine), and ethylenimines (e.g., thiotepa and altretamine). In addition, platinum drugs (e.g., cisplatin, carboplatin, and oxalaplatin) are often considered alkylating agents because they kill cancer cells in a similar way. The disclosure contemplates all of these drugs, or combinations thereof.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Anti-metabolites” are a class of drugs that interfere with DNA and RNA growth by substituting for the normal building blocks of RNA and DNA. These agents damage cells during the S phase. They are commonly used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), Capecitabine (Xeloda@), Cladribine, Clofarabine, Cytarabine (Ara-C@), Floxuridine, Fludarabine, Gemcitabine (Gemzar@), Hydroxyurea, Methotrexate, Pemetrexed (Alimta@), Pentostatin, and Thioguanine.

“Anti-microtubule agents” interfere with the microtubule function, some of them directly binding to the soluble tubulin or to the tubulin in the mircrotubules. Microtubules are building the spindle apparatus during mitosis. Examples for these agents are the taxanes, like docetaxel, paclitaxel, and the vinca alkaloids like vincristine, vinblastine, vindesine.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disease that damages or interferes with the normal function of a cell, tissue, or organ.

By “effective amount” is meant the amount of a compound described herein required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount In still other embodiments, the PDE3A modulator is a compound of formula (I).

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

Unless specifically stated or obvious from context, as used herein, the term “or” is understood to be inclusive. Unless specifically stated or obvious from context, as used herein, the terms “a”, “an”, and “the” are understood to be singular or plural.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

PPARG modulators are compounds that bind to PPARG and can generally alter its function. Modulators can further be subdivided into agonists, antagonists, and inverse agonists. An agonist will lead to the canonical activation of PPARG resulting in the upregulation of canonical target genes. An inverse-agonist will have the opposite effect of an agonist, and lead to the repression of canonical target genes. While an antagonist can be neutral on its own, yet block the binding and activity of either agonist or antagonist. Inverse-agonists are preferred.

The term “prodrugs” or “prodrug” designates compounds which themselves can be biologically active or inactive, but are converted (for example metabolically or hydrolytically) into compounds according to the invention during their residence time in the body. Derivatives of the compound 6 and the salts thereof which are converted into compound 6 or a salt thereof in a biological system (bioprecursors or pro-drugs) are covered by the invention. Said biological system may be, for example, a mammalian organism, particularly a human subject. The bioprecursor is, for example, converted into a compound of formula (I) or a salt thereof by metabolic processes.

Unless specifically stated or obvious from context, as used herein, if a range is provided, the upper and lower limit are always meant to be included. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reference” is meant a standard or control condition.

“Topoisomerase inhibitors” interfere with enzymes called topoisomerases, which help separate the strands of DNA so they can be copied. They are used to treat certain leukemias, as well as lung-, ovarian-, gastrointestinal-, and other cancers. Examples of topoisomerase I inhibitors include topotecan and irinotecan (CPT-11). Examples of topoisomerase II inhibitors include etoposide (VP-16), teniposide and Mitoxantrone.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DETAILED DESCRIPTION

As a first aspect the invention provides compounds of formula (I)

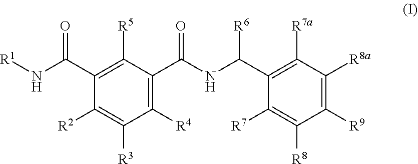

wherein, independently from each occurrence
R¹ is selected from

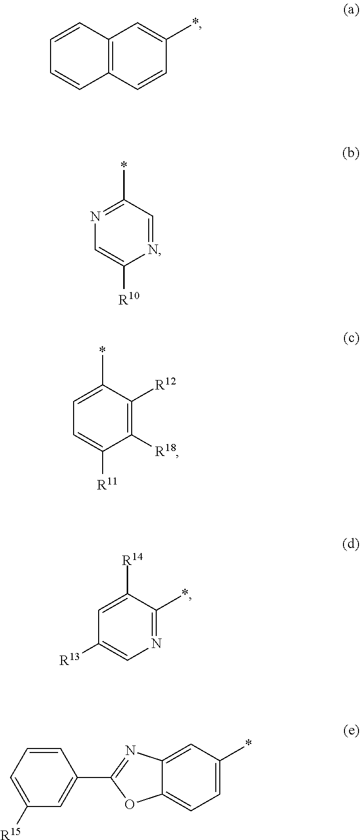

and * is the point of attachment to the nitrogen atom;
R² is selected from the group consisting of a halogen atom, and a —C═CH group;
R³ is selected from the group consisting of a hydrogen atom, an C₁-C₃-alkyl group, and a halogen atom;
R⁴ is selected from the group consisting of a hydrogen atom and a halogen atom;
R⁵ is selected from the group consisting of a hydrogen atom and a halogen atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, a (C₁-C₃)alkoxy group, and a —CH₂OCH₂CH₂R¹⁶ group;
R⁸, R^{8a} is independently selected from the group consisting of a hydrogen atom and a halogen atom;
R⁹ is selected from the group consisting of a hydrogen atom and a halogen atom,
R¹⁰ is selected from the group consisting of a hydrogen atom and a phenoxy group;
R¹¹ is selected from the group consisting of a hydrogen atom, an C₁-C₃-alkyl group, a C₁-C₃-hydroxyalkyl group, a 3-6 membered heterocycloalkyl group comprising 1 or 2 heteroatoms independently selected from oxygen and nitrogen which is optionally substituted with a hydroxy group, a C₁-C₃-haloalkyoxy group, a C₁-C₃-haloalkyl group, a 5-6-membered heteroaryl group which is optionally substituted with a C₁-C₃-alkyl group, a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a halogen atom;
R¹⁶ is selected from the group consisting of a C₁-C₃-alkyl group and a C₁-C₃-alkoxy group;
R¹⁷ is selected from the group consisting of a hydrogen atom and a C₁-C₃-alkyl group;
R¹⁸ is selected from the group consisting of a hydrogen atom and a halogen atom; or a stereoisomer, a tautomer, an N-oxide, a hydrate, a solvate, or a salt thereof, or a mixture of same as well as combinations thereof and methods of preparation.

In one embodiment the invention relates to a method of treatment of cancer, particularly a PPARG activated cancer, more particularly cancer of the urinary tract, particularly bladder cancer, breast cancer, colorectal cancer, esophagael cancer, gastric cancer, hepatocellular cancer, pancreatic cancer, and prostate cancer; and even more particularly bladder cancer or pancreatic cancer.

In an embodiment, the invention includes a method of treating a subject diagnosed with a peroxisome proliferator activated receptor gamma (PPARG) activated cancer, the method comprising a step of administering to the subject in need thereof a compound of formula (I) and in addition a step of administering one or more chemotherapeutic agents. In an embodiment, the one or more chemotherapeutic agents are selected from the group consisting of an alkylating agent, an anti-metabolite, an anti-microtubule agent, and a topoisomerase inhibitor.

In an embodiment, the method may further include a step of administering one or more immune checkpoint inhibitors or immune modulators. Immune checkpoint inhibitors are selected from the group consisting of agents targeting PD1, PD-L1, CTLA4, or immune modulators such as Bacillus Calmette-Guerin (BCG) vaccine, or other modulators.

In an embodiment, the method may further include a step of administering one or more receptor tyrosine kinase inhibitors (RTK). RTK inhibitors are selected from the group consisting of an agents targeting FGFR1, FGFR2, FGFR3, EGFR, ERBB2, or ERBB3.

In an embodiment, the method may further include a step of administering one or more Mitogen-activated protein kinase (MAPK) pathway inhibitors. MAPK inhibitors are selected from the group consisting of an agents targeting KRAS, NRAS, HRAS, BRAF, RAF1 (c-RAF), MAP2K1 (MEK1), MAP2K2 (MEK2), MAPK1 (ERK2), or MAPK3 (ERK1).

### Compound Forms and Salts

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as hydrochloric acid, hydrobromic acid, hydroiodic acid, sulfuric acid, bisulfuric acid, phosphoric acid, and nitric acid or with an organic acid, such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)-benzoic acid, camphoric acid, cinnamic acid, cyclopentanepropionic acid, digluconic acid, 3-hydroxy-2-naphthoic acid, nicotinic acid, pamoic acid, pectinic acid, persulfuric acid, 3-phenylpropionic acid, formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-*2-naphthoic, nicotinic, pamoic, pectinic, 3-phenylpropionic, picric acid, pivalic acid, 2-hydroxyethanesulfonate acid, itaconic acid, sulfamic acid, trifluoromethanesulfonic acid, dodecylsulfuric acid, ethansulfonic acid, benzenesulfonic acid, para-toluenesulfonic acid, methansulfonic acid, 2-naphthalenesulfonic acid, naphthalenedisulfonic acid, camphorsulfonic acid acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid, hemisulfuric acid and thiocyanic acid for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) or an aluminium salt or a zinc salt, or an ammonium salt derived from ammonia or from an organic primary, secondary or tertiary amine having 1 to 20 carbon atoms, such as by way of example ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol.

Additionally, the compounds according to the invention may form salts with a quaternary ammonium ion obtainable, e.g., by quaternisation of a basic nitrogen-containing group with agents such as lower alkylhalides, such as alkylchlorides, e.g. methylchloride, ethylchloride, propylchloride and butylchloride; such as alkylbromides, e.g. methylbromide, ethylbromide, propylbromide and butylbromide; and such as alkyliodides; e.g. methyliodide, ethyliodide, propyliodide and butyliodide; dialkylsulfates such as dimethylsulfate, diethylsulfate, dibutylsulfate and diamylsulfates, long chain halides such as e.g. decylchloride, laurylchloride, myristylchloride and stearylchloride, decylbromide, laurylbromide, myristylbromide and stearylbromide, decyliodide, lauryliodide, myristyliodide and stearyliodide, aralkylhalides such as benzylchloride, benzylbromide, benzyliodide and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF3000H”, “x Na+”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

The present invention also includes various hydrate and solvate forms of the compounds.

The compounds of the present invention optionally contain one or more asymmetric centres, depending upon the location and nature of the various substituents desired, which are e.g. carbon atoms having four different substituents. It is possible that one or more asymmetric carbon atoms are present in the (R) or (S) configuration, which can result in racemic mixtures in the case of a single asymmetric centre, and in diastereomeric mixtures in the case of multiple asymmetric centres. The term “(±)” is used to designate a racemic mixture where appropriate. In certain instances, it is possible that asymmetry also be present due to restricted rotation about a given bond, for example, the central bond adjoining two substituted aromatic rings of the specified compounds. When a compound is an enantiomer, the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art including chiral high pressure liquid chromatography (HPLC), the formation and crystallization of chiral salts, or prepared by asymmetric syntheses.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Preferred is the stereoisomer which shows the desired effect. For compounds of formula (I) wherein R⁴=methyl it is discovered that the compounds having said methyl group in the S-configuration do have a significantly better pharmacological effect.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as 2H (deuterium), 3H (tritium), 11C, 13C, 14C, 15N, 170, 180, 32P, 33P, 33S, 34S, 35S, 36S, 18F, 36Cl, 82Br, 123I, 124I, 125I, 129I and 131I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as 3H or 14C, are incorporated are useful e.g. in drug and/or substrate tissue distribution studies. These isotopes are particularly preferred for the ease of their incorporation and detectability. Positron emitting isotopes such as 18F or 11C may be incorporated into a compound of general formula (I). These isotopic variants of the compounds of general formula (I) are useful for in vivo imaging applications. Deuterium-containing and 13C-containing compounds of general formula (I) can be used in mass spectrometry analyses (H. J. Leis et al., Curr. Org. Chem., 1998, 2, 131) in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D20 can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron Letters, 2011, 52, 3865) is a rapid route for incorporation of deuterium. Metal catalysts (i.e. Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16(4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P450.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

One embodiment includes those compounds which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)- isomers, in any ratio. Isolation of a single stereoisomer, e.g. a single enantiomer or a single diastereomer, of a compound of the present invention is achieved by any suitable state of the art method, such as chromatography, especially chiral chromatography, for example.

The present invention also includes useful forms of the compounds of the present invention, such as metabolites, hydrates, solvates, prodrugs, salts, in particular pharmaceutically acceptable salts, and/or co-precipitates.

### DESCRIPTION

In some embodiments the invention includes compounds of formula (I) wherein, independently from each occurrence,
R¹ is selected from

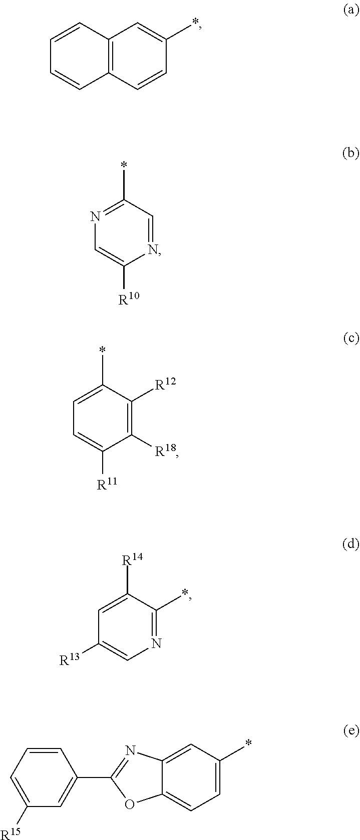

and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁ C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom, R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3-6-membered heterocycloalkyl group is optionally substituted with a hydroxy group, a C₁-C₃-hyloalkyoxy group, a C₁-C₃-haloalkyl group, a 5- to 6-membered heteroaryl group which is optionally substituted with a C₁-C₃-alkyl group, and a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a chlorine atom;
R¹⁷ is selected from the group consisting of a hydrogen atom and a C₁-C₃-alkyl group;
R¹⁸ is selected from the group consisting of a hydrogen atom and a fluorine atom; or a stereoisomer, a tautomer, an N-oxide, a hydrate, a solvate, or a salt thereof, or a mixture of same.

In some embodiments the invention includes compounds of formula (I) wherein, independently from each occurrence
R¹ is selected from

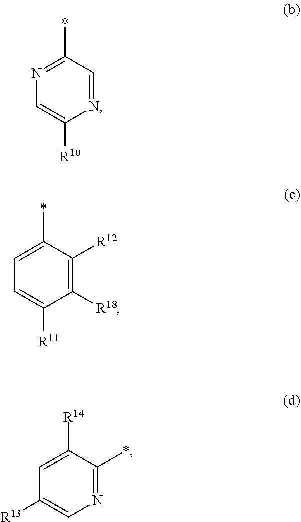

and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁ C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom, R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁸ is selected from the group consisting of a hydrogen atom and a fluorine atom; or a stereoisomer, a tautomer, an N-oxide, a hydrate, a solvate, or a salt thereof, or a mixture of same.

In further embodiments, the present invention includes compounds of formula (I), wherein independently from each occurrence
R¹ is

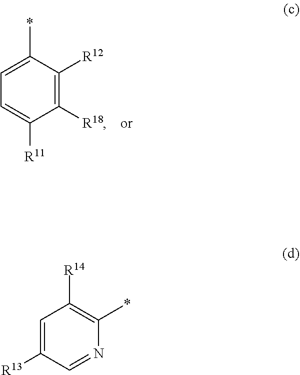

and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁ C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom, R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁸ is selected from the group consisting of a hydrogen atom and a fluorine atom;
or a stereoisomer, a tautomer, an N-oxide, a hydrate, a solvate, or a salt thereof, or a mixture of same.

In further embodiments, the present invention includes compounds of formula (I), wherein, independently from each occurrence,
R¹ is

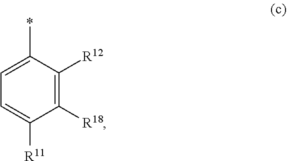

and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁ C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom, R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹⁸ is selected from the group consisting of a hydrogen atom and a fluorine atom; or a stereoisomer, a tautomer, an N-oxide, a hydrate, a solvate, or a salt thereof, or a mixture of same.

In other embodiments the invention includes compounds of formula (I), wherein, independently from each occurrence
R¹ is selected from

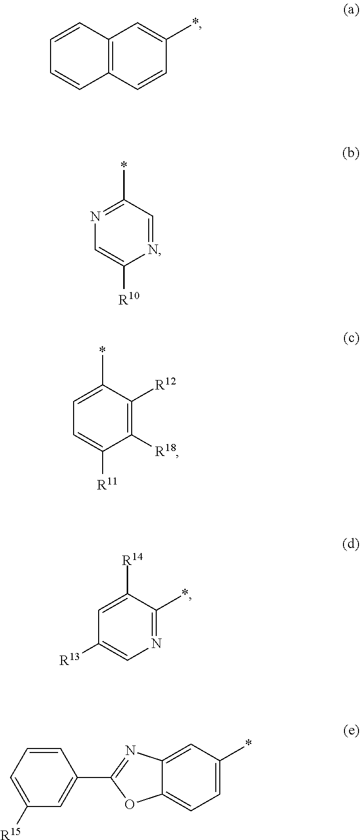

and * is the point of attachment to the nitrogen atom;
R² is selected from the group consisting of a chlorine atom, and a bromine atom,
R³ is selected from the group consisting of a hydrogen atom, a methyl group, and a chlorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxymethyl group;
R⁷, R^{7a} is independently selected from the group consisting of a hydrogen atom, a methyl group, a hydroxy group, a methoxy group, and a —CH₂OCH₂CH₂R¹⁶ group;
R⁸, R^{8a} is independently selected from the group consisting of a hydrogen atom and a fluorine atom;
R⁹ is selected from the group consisting of a hydrogen atom and a fluorine atom;
R¹¹ is selected from the group consisting of a hydrogen atom, a methyl group, a —C(CH₃)₂OH group, a trifluoromethyl group, a —OCHF₂ group, an oxetan-3-yl group, a morpholin-4-yl group, a 3-hydroxypiperidin-1-yl group, a 1,3,4-oxadiazol-2-yl group, a —OCH₂CH₂OR¹⁷ group, and a 3-methyl-1,2,4-oxadiazol-5-yl group;
R¹² is selected from the group consisting of a hydrogen atom and a methyl group;
R¹³ is selected from the group consisting of a hydrogen atom, a chlorine atom, a trifluoromethyl group, a cyclopropyl group, a oxetan-3-yl group, a morpholin-4-yl group, a —OCH₂C(CH₃)₂OH group, and a phenoxy group;
R¹⁴ is selected from the group consisting of a hydrogen atom and a methyl group;
R¹⁵ is selected from the group consisting of a methyl group and a chlorine atom;
R¹⁶ is selected from the group consisting of a methyl group and a methoxy group;
R¹⁷ is selected from the group consisting of a hydrogen atom and a methyl group;
R¹⁸ is selected from the group consisting of a hydrogen atom and a fluorine atom; or a stereoisomer, a tautomer, an N-oxide, a hydrate, a solvate, or a salt thereof, or a mixture of same.

In yet other embodiments the invention includes compounds of formula (I) selected from the group consisting of
4-chloro-N³-[4-(difluoromethoxy)-2-methylphenyl]-6-fluoro-N¹-[(2-hydroxyphenyl)methyl]benzene-1,3-dicarboxamide,
4-chloro-N³-[4-(difluoromethoxy)-2-methylphenyl]-6-fluoro-N¹-[1-(4-fluorophenyl)-2-hydroxyethyl]benzene-1,3-dicarboxamide,
4-chloro-N³-[4-(difluoromethoxy)phenyl]-N¹-[(3,4-difluorophenyl)methyl]-6-fluorobenzene-1,3-dicarboxamide,
4-chloro-6-fluoro-N¹-{[4-fluoro-2-(propoxymethyl)phenyl]methyl}-N³-[2-methyl-4-(trifluoromethyl)phenyl]benzene-1,3-dicarboxamide,
4-chloro-N¹-[(3,4-difluorophenyl)methyl]-6-fluoro-N³-{4-[3-hydroxypiperidin-1-yl]-2-methylphenyl}benzene-1,3-dicarboxamide (stereoisomer 1),
4-chloro-N¹-[(3,4-difluorophenyl)methyl]-6-fluoro-N³-{4-[3-hydroxypiperidin-1-yl]-2-methylphenyl}benzene-1,3-dicarboxamide (stereoisomer 2),
4,5-dichloro-N³-[4-(difluoromethoxy)-2-methylphenyl]-N¹-[(3,4-difluorophenyl)methyl]benzene-1,3-dicarboxamide,
4,5-dichloro-N¹-[(3,4-difluorophenyl)methyl]-N³-[4-(2-hydroxyethoxy)-2-methylphenyl]benzene-1,3-dicarboxamide,
4,5-dichloro-N¹-[(4-fluorophenyl)methyl]-N³-[2-(3-methylphenyl)-1,3-benzoxazol-5-yl]benzene-1,3-dicarboxamide,
4-chloro-N¹-[(3,4-difluorophenyl)methyl]-6-fluoro-N³-[2-methyl-4-(1,3,4-oxadiazol-2-yl)phenyl]benzene-1,3-dicarboxamide,
4-chloro-N¹-[(3,4-difluorophenyl)methyl]-6-fluoro-N³-{4-[3-hydroxypiperidin-1-yl]-2-methylphenyl}benzene-1,3-dicarboxamide,
4-chloro-N¹-[(3,4-difluorophenyl)methyl]-6-fluoro-N³-[4-(2-hydroxyethoxy)-2-methylphenyl]benzene-1,3-dicarboxamide,
4,5-dichloro-N¹-[(3,4-difluorophenyl)methyl]-N³-[5-(trifluoromethyl)pyridin-2-yl]benzene-1,3-dicarboxamide,
4,5-dichloro-N³-(5-cyclopropylpyridin-2-yl)-N¹-[(3,4-difluorophenyl)methyl]benzene-1,3-dicarboxamide,
4,5-dichloro-N¹-[(2-methoxyphenyl)methyl]-N³-phenylbenzene-1,3-dicarboxamide,
N¹-benzyl-4,5-dichloro-N³-phenylbenzene-1,3-dicarboxamide,
4,5-dichloro-N¹-[(2-methylphenyl)methyl]-N³-phenylbenzene-1,3-dicarboxamide,
4-chloro-N¹-[(3,4-difluorophenyl)methyl]-6-fluoro-5-methyl-N³-[3-methyl-5-(morpholin-4-yl)pyridin-2-yl]benzene-1,3-dicarboxamide,
4-chloro-N¹-[(3,4-difluorophenyl)methyl]-6-fluoro-N³-[4-(2-methoxyethoxy)-2-methylphenyl]-5-methylbenzene-1,3-dicarboxamide,
4-chloro-N¹-[(3,4-difluorophenyl)methyl]-6-fluoro-5-methyl-N³-[2-(3-methylphenyl)-1,3-benzoxazol-5-yl]benzene-1,3-dicarboxamide,
4-chloro-6-fluoro-N¹-[(4-fluorophenyl)methyl]-N³-(5-phenoxypyridin-2-yl)benzene-1,3-dicarboxamide,
4-chloro-N³-[2-(3-chlorophenyl)-1,3-benzoxazol-5-yl]-6-fluoro-N¹-[(4-fluorophenyl)methyl]benzene-1,3-dicarboxamide,
4-chloro-6-fluoro-N¹-[(4-fluorophenyl)methyl]-N³-[4-(3-methyl-1,2,4-oxadiazol-5-yl)phenyl]benzene-1,3-dicarboxamide,
N¹-benzyl-4-chloro-6-fluoro-N³-(4-methylphenyl)benzene-1,3-dicarboxamide,
N¹-benzyl-4-chloro-6-fluoro-N³-(2-methylphenyl)benzene-1,3-dicarboxamide,
4-chloro-6-fluoro-N¹-({4-fluoro-2-[(2-methoxyethoxy)methyl]phenyl}methyl)-N³-[2-methyl-4-(trifluoromethyl)phenyl]benzene-1,3-dicarboxamide,
4-chloro-6-fluoro-N¹-[(1R)-2-hydroxy-1-phenylethyl]-N³-[2-methyl-4-(trifluoromethyl)phenyl]benzene-1,3-dicarboxamide,
N¹-benzyl-4-chloro-6-fluoro-N³-phenylbenzene-1,3-dicarboxamide,
4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 1),
4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 2),
N¹-benzyl-4,5-dichloro-N³-[4-(difluoromethoxy)phenyl]-2-fluorobenzene-1,3-dicarboxamide,
N¹-benzyl-4,5-dichloro-N³-(5-chloropyridin-2-yl)-2-fluorobenzene-1,3-dicarboxamide,
N¹-benzyl-4,5-dichloro-2-fluoro-N3-[4-(oxetan-3-yl)phenyl]benzene-1,3-dicarboxamide,
4-chloro-N¹-(3,4-difluorobenzyl)-6-fluoro-N³-(pyridin-2-yl)isophthalamide,
4-chloro-N¹-(3,4-difluorobenzyl)-6-fluoro-N³-(4-morpholinophenyl)isophthalamide,
4-chloro-N¹-(3,4-difluorobenzyl)-6-fluoro-N³-(5-phenoxypyrazin-2-yl)isophthalamide,
4-chloro-N¹-(3,4-difluorobenzyl)-6-fluoro-N³-(3-methyl-5-(oxetan-3-yl)pyridin-2-yl)isophthalamide,
4,5-dichloro-N¹-(2-methoxybenzyl)-N³-(5-morpholinopyridin-2-yl)isophthalamide,
4-chloro-N¹-(3,4-difluorobenzyl)-6-fluoro-N³-(2-(m-tolyl)benzo[d]oxazol-5-yl)isophthalamide
N¹-(3,4-difluorobenzyl)-4-ethynyl-6-fluoro-N³-phenylisophthalamide,
4-chloro-N¹-(3,4-difluorobenzyl)-6-fluoro-N³-(3-methyl-5-morpholinopyridin-2-yl)isophthalamide,
,5-dichloro-N¹-(3,4-difluorobenzyl)-N³-(pyrazin-2-yl)isophthalamide,
N¹-benzyl-4-chloro-N³-phenylbenzene-1,3-dicarboxamide,
4-chloro-N¹-[(3,4-difluorophenyl)methyl]-N³-phenylbenzene-1,3-dicarboxamide,
4-chloro-N¹-[(4-fluorophenyl)methyl]-N³-phenylbenzene-1,3-dicarboxamide,
4-chloro-N¹-[(2-methoxyphenyl)methyl]-N³-phenylbenzene-1,3-dicarboxamide,
4-chloro-N¹-[(2-methylphenyl)methyl]-N³-phenylbenzene-1,3-dicarboxamide,
N¹-benzyl-4-bromo-N³-phenylbenzene-1,3-dicarboxamide
N¹-benzyl-4-bromo-5-chloro-N³-phenylbenzene-1,3-dicarboxamide,
N¹-benzyl-4-bromo-5-chloro-N³-(2-methylphenyl)benzene-1,3-dicarboxamide,
4-chloro-N¹-[(3,4-difluorophenyl)methyl]-6-fluoro-N³-[4-(2-hydroxypropan-2-yl)phenyl]benzene-1,3-dicarboxamide,
4-chloro-N¹-[(3,4-difluorophenyl)methyl]-6-fluoro-N³-[5-(2-hydroxy-2-methylpropoxy)pyridin-2-yl]-5-methylbenzene-1,3-dicarboxamide, and
4-chloro-N³-[4-(difluoromethoxy)-2-methylphenyl]-6-fluoro-N¹-[(4-fluorophenyl)methyl]benzene-1,3-dicarboxamide,
4-chloro-N¹-[(3,4-difluorophenyl)methyl]-6-fluoro-N³-(3-methyl-2-pyridyl)benzene-1,3-dicarboxamide
4-chloro-N¹-(3,4-difluorobenzyl)-6-fluoro-N³-(naphthalen-2-yl)isophthalamide
4,5-dichloro-N¹-[(2-methoxyphenyl)methyl]-N³-pyrazin-2-yl-benzene-1,3-dicarboxamide
4-chloro-N¹-[(3,4-difluorophenyl)methyl]-6-fluoro-N³-(5-phenoxy-2-pyridyl)benzene-1,3-dicarboxamide
4-chloro-N³-[4-(difluoromethoxy)-3-fluorophenyl]-6-fluoro-N¹-[(2-methoxyphenyl)methyl]benzene-1,3-dicarboxamide
4-chloro-6-fluoro-N³-[2-fluoro-4-(oxetan-3-yl) phenyl]-N¹-[(2-methoxyphenyl)methyl]benzene-1,3-dicarboxamide
4-chloro-6-fluoro-N¹-[(2-methoxyphenyl)methyl]-N³-5-(morpholin-4-yl)pyridin-2-yl]benzene-1,3-dicarboxamide
   or a stereoisomer, a tautomer, an N-oxide, a hydrate, a solvate, or a salt thereof, or a mixture of same.

In an embodiment, the inverse-agonist of PPARG signaling is selected from the group consisting of
4-chloro-N³-[4-(difluoromethoxy)-2-methylphenyl]-6-fluoro-N¹-[(4-fluorophenyl)methyl]benzene-1,3-dicarboxamide,
4-chloro-N¹-[(3,4-difluorophenyl)methyl]-6-fluoro-N³-[4-(2-hydroxypropan-2-yl)phenyl]benzene-1,3-dicarboxamide,
4-chloro-N¹-(3,4-difluorobenzyl)-6-fluoro-N³-(3-methyl-5-morpholinopyridin-2-yl)isophthalamide,
   or a stereoisomer, a tautomer, an N-oxide, a hydrate, a solvate, or a salt thereof, or a mixture of same.

In an embodiment, the inverse-agonist of PPARG signaling is selected from the group consisting of
4-chloro-N¹-[(3,4-difluorophenyl)methyl]-6-fluoro-N³-[4-(2-hydroxypropan-2-yl)phenyl]benzene-1,3-dicarboxamide,
4-chloro-N¹-(3,4-difluorobenzyl)-6-fluoro-N³-(3-methyl-5-morpholinopyridin-2-yl)isophthalamide,
   or a stereoisomer, a tautomer, an N-oxide, a hydrate, a solvate, or a salt thereof, or a mixture of same.

In an embodiment, the inverse-agonist of PPARG signaling is 4-chloro-N³-[4-(difluoromethoxy)-2-methylphenyl]-6-fluoro-N¹-[(4-fluorophenyl)methyl]benzene-1,3-dicarboxamide

or a stereoisomer, a tautomer, an N-oxide, a hydrate, a solvate, or a salt thereof, or a mixture of same.

In an embodiment, the inverse-agonist of PPARG signaling is 4-chloro-N¹-[(3,4-difluorophenyl)methyl]-6-fluoro-N³-[4-(2-hydroxypropan-2-yl)phenyl]benzene-1,3-dicarboxamide

or a stereoisomer, a tautomer, an N-oxide, a hydrate, a solvate, or a salt thereof, or a mixture of same.

In an embodiment, the inverse-agonist of PPARG signaling is 4-chloro-N¹-(3,4-difluorobenzyl)-6-fluoro-N³-(3-methyl-5-morpholinopyridin-2-yl)isophthalamide

or a stereoisomer, a tautomer, an N-oxide, a hydrate, a solvate, or a salt thereof, or a mixture of same.

In some embodiments the invention relates to compounds of formula (I), wherein R¹ is

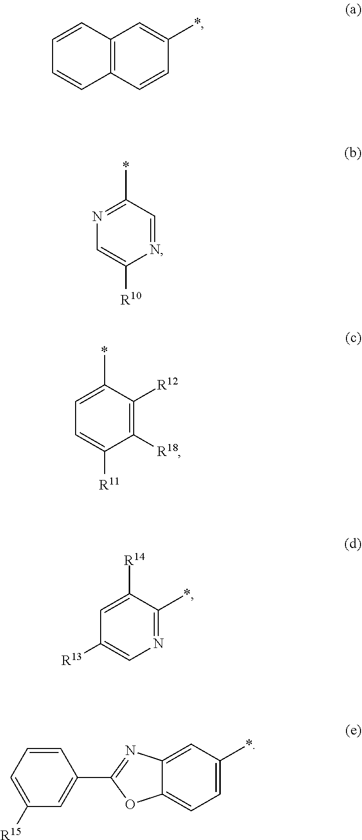

In some embodiments the invention relates to compounds of formula (I), wherein R¹ is

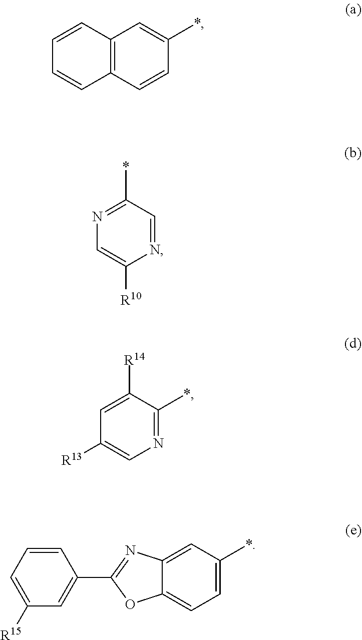

In some embodiments the invention relates to compounds of formula (I), wherein R¹ is

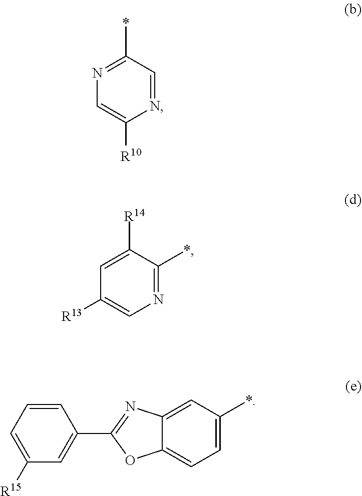

In some embodiments the invention relates to compounds of formula (I), wherein R¹ is

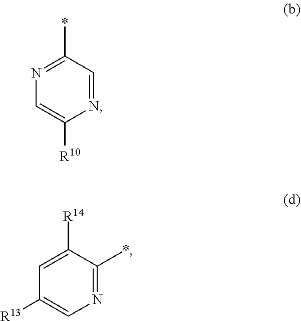

In some embodiments the invention relates to compounds of formula (I), wherein R¹ is

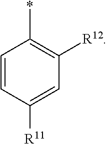

In some embodiments the invention relates to compounds of formula (I), wherein R¹ is

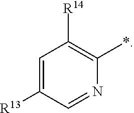

In yet other embodiments the invention relates to compounds of formula (I) wherein R¹ is

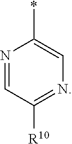

In other embodiments the invention relates to compounds of formula (I) wherein R¹ is

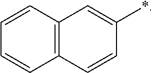

In other embodiments the invention relates to compounds wherein R¹ is

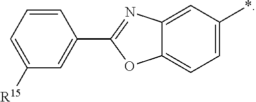

In further embodiments the invention relates to compounds of formula (I) wherein R² is a halogen atom or an acetylene group —C═CH.

In further embodiments the invention relates to compounds of formula (I) wherein R² is a halogen atom.

In further embodiments the invention relates to compounds of formula (I) wherein R² is a chlorine atom or a bromine atom or an acetylene group —C═CH.

In further embodiments the invention relates to compounds of formula (I) wherein R² is a chlorine or bromine atom.

In further embodiments the invention relates to compounds of formula (I) wherein R² is a chlorine atom.

In further embodiments the invention relates to compounds of formula (I) wherein R² is a bromine atom.

In further embodiments the invention relates to compounds of formula (I) wherein R² is an acetylene group —C═CH.

In other embodiments the invention relates to compounds of formula (I) wherein R³ is a hydrogen atom, a C₁-C₃-alkyl group or a halogen atom.

In other embodiments the invention relates to compounds of formula (I) wherein R³ is a hydrogen atom, a methyl group or a chlorine atom.

In other embodiments the invention relates to compounds of formula (I) wherein R³ is a hydrogen atom, or a chlorine atom.

In other embodiments the invention relates to compounds of formula (I) wherein R³ is a hydrogen atom, or a methyl group.

In other embodiments the invention relates to compounds of formula (I) wherein R³ is a methyl group or a chlorine atom.

In some embodiments the invention relates to compounds of formula (I) wherein R² is a chlorine atom and R³ is a chlorine atom.

In further embodiments the invention relates to compounds of formula (I) wherein R⁴ is a hydrogen atom or a halogen atom.

In further embodiments the invention relates to compounds of formula (I) wherein R⁴ is a hydrogen atom or a fluorine atom.

In further embodiments the invention relates to compounds of formula (I) wherein R⁴ is a hydrogen atom.

In further embodiments the invention relates to compounds of formula (I) wherein R⁴ is a fluorine atom.

In further embodiments the invention relates to compounds of formula (I) wherein R⁵ is a hydrogen atom or a fluorine atom.

In further embodiments the invention relates to compounds of formula (I) wherein R⁵ is a hydrogen atom.

In further embodiments the invention relates to compounds of formula (I) wherein R⁵ is a fluorine atom.

In other embodiments the invention relates compounds of formula (I) wherein R⁶ is a hydrogen atom or a C₁-C₃-hydroxyalkyl group.

In other embodiments the invention relates compounds of formula (I) wherein R⁶ is a hydrogen atom or a methyl group.

In other embodiments the invention relates compounds of formula (I) wherein R⁶ is a hydrogen atom or a methyl group.

In other embodiments the invention relates compounds of formula (I) wherein R⁶ is a hydrogen atom or a methyl group.

In further embodiments the invention relates compounds of formula (I) wherein R⁷/R^{7a} is independently selected from the group consisting of a hydrogen atom, C₁-C₃-alkyl group, a hydroxy group, a hydroxy group, a (C₁-C₃)alkoxy group, and a —CH₂OCH₂CH₂R¹⁶ group.

In further embodiments the invention relates compounds of formula (I) wherein R⁷/R^{7a} is independently selected from the group consisting of a hydrogen atom, C₁-C₃-alkyl group, a hydroxy group, a hydroxy group, and a (C₁-C₃)alkoxy group.

In further embodiments the invention relates compounds of formula (I) wherein R⁷/R^{7a} is independently selected from the group consisting of a hydrogen atom, methyl group, a hydroxy group, a methoxy group, and a —CH₂OCH₂CH₂R¹⁶ group.

In further embodiments the invention relates compounds of formula (I) wherein R⁷/R^{7a} is independently selected from the group consisting of a hydrogen atom, methyl group, a hydroxy group, and a methoxy group.

In further embodiments the invention relates compounds of formula (I) wherein R⁷/R^{7a} is a hydrogen atom,

In further embodiments the invention relates compounds of formula (I) wherein R⁷/R^{7a} is a methyl group.

In further embodiments the invention relates compounds of formula (I) wherein R⁷/R^{7a} is selected from the group consisting of a hydroxy group and a methoxy group

In further embodiments the invention relates compounds of formula (I) wherein R⁷/R^{7a} is selected from the group consisting of a hydrogen atom and a hydroxy group.

In further embodiments the invention relates compounds of formula (I) wherein R⁷/R^{7a} is a hydroxy group.

In further embodiments the invention relates compounds of formula (I) wherein R⁷/R^{7a} is a methoxy group.

In further embodiments the invention relates compounds of formula (I) wherein R⁸/R^{8a} is selected from the group consisting of a hydrogen atom or a halogen atom.

In further embodiments the invention relates compounds of formula (I) wherein R⁸/R^{8a} is selected from the group consisting of a hydrogen atom or a fluorine atom.

In further embodiments the invention relates compounds of formula (I) wherein R⁸/R^{8a} a hydrogen atom.

In further embodiments the invention relates compounds of formula (I) wherein R⁸/R^{8a} is a fluorine atom.

In further embodiments the invention relates compounds of formula (I) wherein R⁹ is selected from the group consisting of a hydrogen atom or a halogen atom.

In further embodiments the invention relates compounds of formula (I) wherein R⁹ is selected from the group consisting of a hydrogen atom or a fluorine atom.

In further embodiments the invention relates compounds of formula (I) wherein R⁹ is a hydrogen atom.

In further embodiments the invention relates compounds of formula (I) wherein R⁹ is a fluorine atom.

In some embodiments the invention relates to compounds of formula (I) wherein R⁸ is a fluorine atom and R⁹ is a fluorine atom.

In further embodiments the invention relates compounds of formula (I) wherein R¹⁰ is selected from the group consisting of a hydrogen atom or a phenoxy group.

In further embodiments the invention relates compounds of formula (I) wherein R¹⁰ is a hydrogen atom.

In further embodiments the invention relates compounds of formula (I) wherein R¹⁰ is a phenoxy group.

In other embodiments the invention relates to compounds of formula (I) wherein R¹¹ is selected from the group consisting of a hydrogen atom, an C₁-C₃-alkyl group, a C₁-C₃-hydroxyalkyl group, a 3- to 6-membered heterocycloalkyl group which is optionally substituted with a hydroxy group, comprising 1 or 2 heteroatoms independently selected from an oxygen atom and a nitrogen atom, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group.

In other embodiments the invention relates to compounds of formula (I) wherein R¹¹ is selected from the group consisting of a hydrogen atom, a methyl group, a —C(CH₃)₂OH group, an oxetan-3-yl group, a —OCHF₂ group, a trifluoromethyl group, a 1,3,4-oxadiazol-2-yl group, a —OCH₂CH₂OR¹⁷ group, a morpholin-4-yl group, a 3-methyl-1,2,4-oxadiazol-5-yl group, and a 3-hydroxypiperidin-1-yl group.

In other embodiments the invention relates to compounds of formula (I) wherein R¹¹ is selected from the group consisting of a hydrogen atom, a methyl group, a —C(CH₃)₂OH group, a —OCHF₂ group, a trifluoromethyl group, a —OCH₂CH₂OR¹⁷ group, a 3- to 6-membered heterocycloalkyl group, comprising 1 or 2 heteroatoms independently selected from an oxygen atom and a nitrogen atom, and a 5- to 6-membered heteroaryl group.

In other embodiments the invention relates to compounds of formula (I) wherein R¹¹ is a 3- to 6-membered heterocycloalkyl group which is optionally substituted with a hydroxy group, comprising 1 or 2 heteroatoms independently selected from an oxygen atom and a nitrogen atom.

In other embodiments the invention relates to compounds of formula (I) wherein R¹¹ is a 3-membered heterocycloalkyl group which is optionally substituted with a hydroxy group, comprising 1 heteroatom independently selected from an oxygen atom and a nitrogen atom.

In other embodiments the invention relates to compounds of formula (I) wherein R¹¹ is a 4-membered heterocycloalkyl group which is optionally substituted with a hydroxy group, comprising 1 heteroatom independently selected from an oxygen atom and a nitrogen atom.

In other embodiments the invention relates to compounds of formula (I) wherein R¹¹ is a 5-membered heterocycloalkyl group which is optionally substituted with a hydroxy group, comprising 1 or 2 heteroatoms independently selected from an oxygen atom and a nitrogen atom.

In other embodiments the invention relates to compounds of formula (I) wherein R¹¹ is a 6-membered heterocycloalkyl group which is optionally substituted with a hydroxy group, comprising 1 or 2 heteroatoms independently selected from an oxygen atom and a nitrogen atom.

In other embodiments the invention relates to compounds of formula (I) wherein R¹¹ is a 5- to 6-membered heteroaryl group which is optionally substituted with a C₁-C₃-alkyl group.

In other embodiments the invention relates to compounds of formula (I) wherein R¹¹ is a 5- to 6-membered heteroaryl group which is optionally substituted with a methyl group.

In other embodiments the invention relates to compounds of formula (I) wherein R¹¹ is a 5- to 6-membered heteroaryl group which is optionally substituted with a methyl group, comprising 1 or 2 heteroatoms independently selected from an oxygen atom and a nitrogen atom.

In other embodiments the invention relates to compounds of formula (I) wherein R¹¹ is a 5-membered heteroaryl group which is optionally substituted with a methyl group, comprising 1 or 2 heteroatoms independently selected from an oxygen atom and a nitrogen atom.

In other embodiments the invention relates to compounds of formula (I) wherein R¹¹ is a 6-membered heteroaryl group which is optionally substituted with a methyl group, comprising 1 or 2 heteroatoms independently selected from an oxygen atom and a nitrogen atom.

In other embodiments the invention relates to compounds of formula (I) wherein R¹¹ is a C₁-C₃-haloalkyoxy group, particularly a —O—CHF₂ group.

In other embodiments the invention relates to compounds of formula (I) wherein R¹² is a hydrogen atom or a C₁-C₃-alkyl group.

In other embodiments the invention relates to compounds of formula (I) wherein R¹² is a hydrogen atom or a methyl group.

In other embodiments the invention relates to compounds of formula (I) wherein R¹² is a hydrogen atom.

In other embodiments the invention relates to compounds of formula (I) wherein R¹² is a methyl group.

In some other embodiments the invention relates to compounds of formula (I) wherein R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)OH group, and a phenoxy group.

In some other embodiments the invention relates to compounds of formula (I) wherein R¹³ is selected from the group consisting of a hydrogen atom, a chlorine atom, a cyclopropyl group, an oxetan-3-yl group, a trifluoromethyl group, a morpholin-4-yl group, a —OCH₂C(CH₃)₂OH group, and a phenoxy group.

In some other embodiments the invention relates to compounds of formula (I) wherein R¹³ is a hydrogen atom.

In some other embodiments the invention relates to compounds of formula (I) wherein R¹³ is a halogen atom, particularly a chlorine atom.

In some other embodiments the invention relates to compounds of formula (I) wherein R¹³ is a C₃-C₆-cycloalkyl group, particularly a cyclopropyl group.

In other embodiments the invention relates to compounds of formula (I) wherein R¹³ is 4-to 6-membered heterocycloalkyl group comprising one heteroatom selected from oxygen or nitrogen, particularly a morpholine-4-yl or an oxetan-3-yl group.

In other embodiments the invention relates to compounds of formula (I) wherein R¹³ is a C₁-C₃-haloalkyl group, particularly a trifluoromethyl group.

In other embodiments the invention relates to compounds of formula (I) wherein R¹³ is a a —OCH₂C(CH₃)₂OH group, or a phenoxy group.

In other embodiments the invention relates to compounds of formula (I) wherein R¹⁴ is selected from the group consisting of a hydrogen atom and a C₁-C₃-alkyl group.

In other embodiments the invention relates to compounds of formula (I) wherein R¹⁴ is selected from the group consisting of a hydrogen atom and a methyl group.

In other embodiments the invention relates to compounds of formula (I) wherein R¹⁴ is a hydrogen atom.

In other embodiments the invention relates to compounds of formula (I) wherein R¹⁴ is a methyl group.

In further embodiments the invention relates compounds of formula (I) wherein R¹⁵ is selected from the group consisting of a hydrogen atom or a halogen atom.

In further embodiments the invention relates compounds of formula (I) wherein R¹⁵ is selected from the group consisting of a hydrogen atom or a chlorine atom.

In further embodiments the invention relates compounds of formula (I) wherein R¹⁵ is a hydrogen atom.

In further embodiments the invention relates compounds of formula (I) wherein R¹⁵ is a chlorine atom.

In further embodiments the invention relates compounds of formula (I) wherein R¹⁶ is selected from the group consisting of a C₁-C₃-alkyl group and a C₁-C₃-alkoxy group.

In further embodiments the invention relates compounds of formula (I) wherein R¹⁶ is selected from the group consisting of a methyl group and a methoxy group.

In further embodiments the invention relates compounds of formula (I) wherein R¹⁶ is a methyl group.

In further embodiments the invention relates compounds of formula (I) wherein R¹⁶ is a methoxy group.

In further embodiments the invention relates compounds of formula (I) wherein R¹⁷ is selected from the group consisting of a hydrogen atom and a C₁-C₃-alkyl group.

In other embodiments the invention relates to compounds of formula (I) wherein R¹⁷ is selected from the group consisting of a hydrogen atom and a methyl group.

In other embodiments the invention relates to compounds of formula (I) wherein R¹⁷ is a hydrogen atom.

In other embodiments the invention relates to compounds of formula (I) wherein R¹⁷ is a methyl group.

In yet some other embodiments R¹ is

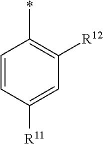

group and R¹¹ is selected from the group consisting of a hydrogen atom, an C₁-C₃-alkyl group, a C₁-C₃-hydroxyalkyl group, a 3- to 6-membered heterocycloalkyl group which is optionally substituted with a hydroxy group, comprising 1 or 2 heteroatoms independently selected from an oxygen atom and a nitrogen atom, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group and R¹² is a hydrogen atom.

In yet some other embodiments R¹ is a

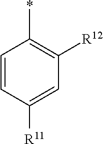

group and R¹¹ is selected from the group consisting of a hydrogen atom, an C₁-C₃-alkyl group, a C₁-C₃-hydroxyalkyl group, a 3- to 6-membered heterocycloalkyl group which is optionally substituted with a hydroxy group, comprising 1 or 2 heteroatoms independently selected from an oxygen atom and a nitrogen atom, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group and R¹² is a methyl group.

In some other embodiments R¹ is a

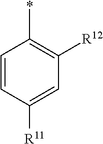

group and R¹¹ is selected from the group consisting of a hydrogen atom, a methyl group, a —C(CH₃)₂OH group, an oxetan-3-yl group, a —OCHF₂ group, a trifluoromethyl group, a 1,3,4-oxadiazol-2-yl group, a —OCH₂CH₂OR¹⁷ group, a morpholin-4-yl group, a 3-methyl-1,2,4-oxadiazol-5-yl group, and a 3-hydroxypiperidin-1-yl group and R¹² is a hydrogen atom.

In some other embodiments R¹ is a

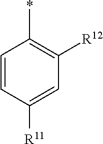

group and R¹¹ is selected from the group consisting of a hydrogen atom, a methyl group, a —C(CH₃)₂OH group, an oxetan-3-yl group, a —OCHF₂ group, a trifluoromethyl group, a 1,3,4-oxadiazol-2-yl group, a —OCH₂CH₂OR¹⁷ group, a morpholin-4-yl group, a 3-methyl-1,2,4-oxadiazol-5-yl group, and a 3-hydroxypiperidin-1-yl group and R¹² is a methyl group.

In some other embodiments R¹ is a

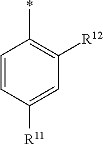

or a

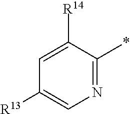

group, R² is a chlorine atom, R³ is a hydrogen atom, R⁴ is a fluorine atom, R⁵═R⁶=R⁷═R^{7a}═R^{8a}=a hydrogen atom, R⁸ is a hydrogen atom or a fluorine atom, R⁹ is a fluorine atom, R¹¹ is selected from —OCHF₂, morpholine-4-yl and —C(OH)(CH₃)₂, R¹³ is morpholine-4-yl and R¹⁴ is a methyl group

In some other embodiments R¹ is a

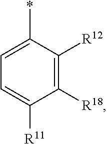

or a

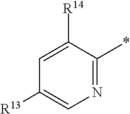

group, R² is selected from the group consisting of a fluorine atom and chlorine atom, R³ is a hydrogen atom, R⁴ is a fluorine atom, R⁵═R⁶=R^{7a}═R⁸═R^{8a}=a hydrogen atom, R⁷ is a methoxy group, R¹¹ is selected from the group consisting of —OCHF₂, and oxetan-3-yl, R¹² is selected from the group consisting of a hydrogen atom and a fluorine atom, R¹³ is selected from the group consisting of a hydrogen atom, a morpholine-4-yl group, R¹⁴ is selected from the group consisting of a hydrogen atom or a methyl group, and R¹⁸ is a hydrogen atom or a fluorine atom.

### General Procedures

### Synthetic Routes:

The compounds according to the invention of general formula (I) can be prepared according to the following routes 1 or 2 reflected in schemes 1, and 2. The schemes and procedures described below illustrate synthetic routes to the compounds of general formula (I) of the invention and are not intended to be limiting. A person skilled in the art relying on the general knowledge knows that the order of transformations as exemplified in schemes 1 and 2 can be modified in various ways. In addition, interconversion of any of the substituents, R¹, R², R³ or R⁴ can be achieved before and/or after the exemplified transformations. These modifications can be such as the introduction of protecting groups, cleavage of protecting groups, reduction or oxidation of functional groups, halogenation, alkylation, acylation, metalation or substitution known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

As outlined in scheme 1 compounds of general formula (I) can be prepared from starting materials of formula (II) by reacting with benzyl amines of the formula (III) under suitable amide coupling conditions to form amides of formula (IV). Amide coupling reagents like T3P or HATU can be used in solvents such as DMSO or DMF.

Route 1:

The general principle of rounte 1 is starting from the central ring moiety bearing a carbocylic acid group and a bromide the benzylamide is formed and after cleavage of the ester the second amide is formed.

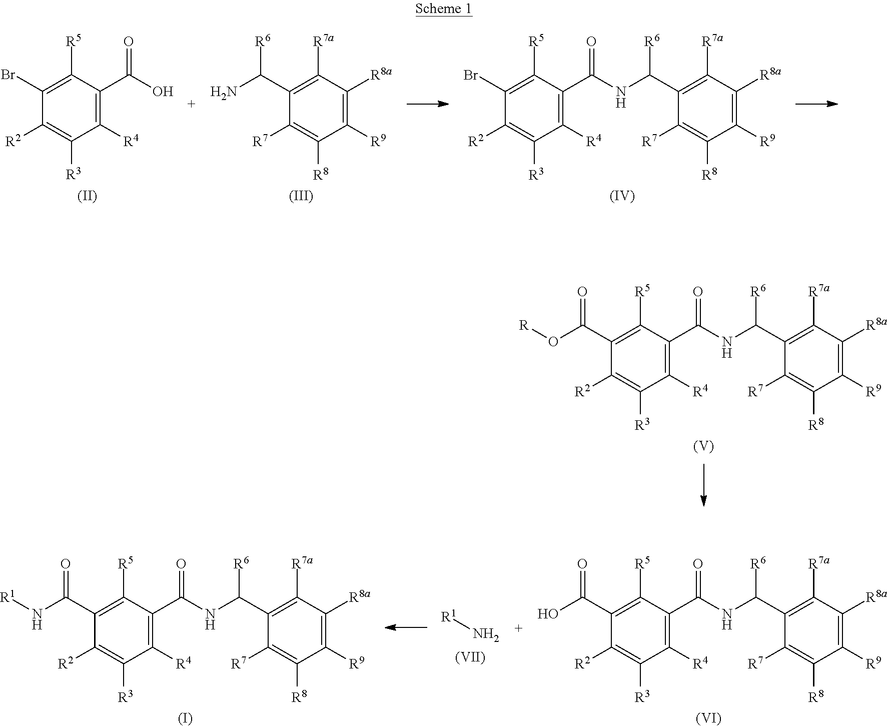

Route 1 for the preparation of compounds of general formula (I), wherein, R¹, R², R³, R⁴, R⁶, R⁷, R^{7a}, R^{8a}, have the meaning as given for general formula (I). R is a methyl or an ethyl group.

Compounds of the general formula (IV) can be reacted with carbon monoxide in a carbonylation reaction, catalyzed by suitable catalysts like [1,1-Bis(diphenylphosphino)ferrocene]dichloro-palladium(II) in solvents such as methanol or ethanol to the corresponding esters of the general formula (V). The ester derivatives of the general formula (V) can be hydrolyzed to the corresponding acids of the general formula (VI) under basic aqueous conditions using bases such as lithium hydroxide or sodium hydroxide. The carboxylic acids of the general formula (VI) can be reacted with amines of the general formula (VII) in an amide formation reaction to the amides of the general formula (I) using amide coupling reagents such as T3P, HATU or DCCI.

An alternative synthetic approach to compounds of the general formula (VII) is outlined in route 2.

Route 2:

An alternative synthetic approach to compounds of the general formula (I) is outlined in Scheme 2.

Starting again from the central ring moiety the first amide is formed reacting the aniline with the carboxylic acid and subsequently the bromide is transformed into an ester moiety which is cleaved and the acid is reacted with the benzylamine and the benzylamide is formed.

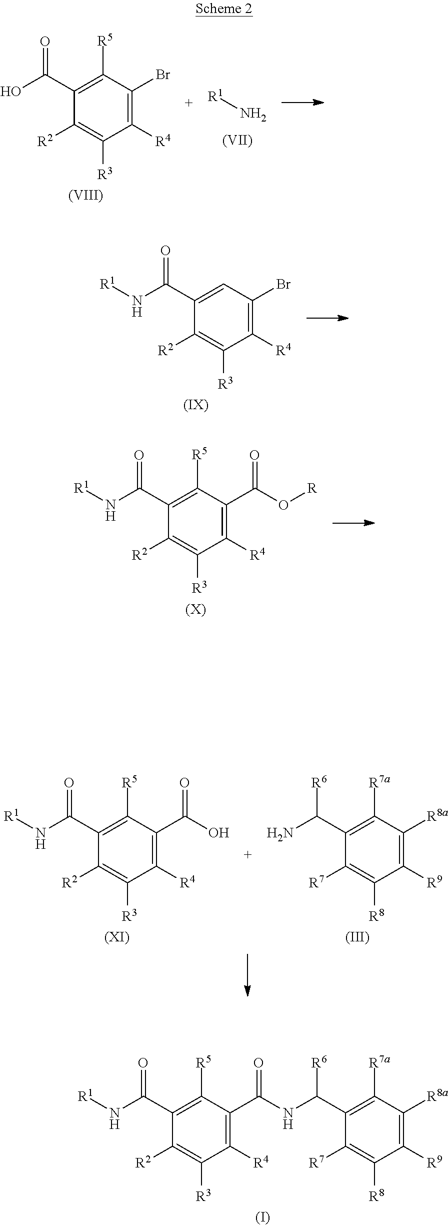

Route for the preparation of compounds of general formula (I), wherein, R¹, R², R^{2a}, R³, R⁴, R^{4a}, R⁵, R^{5a}, R⁶, R⁷ have the meaning as given for general formula (I). R is a methyl or an ethyl group.

Carboxylic acids of the general formula (VII) can be reacted with amines of the general formula (VII) in an amide formation reaction to the amides of the general formula (IX) using amide coupling reagents such as T3P, HATU or DCCI in solvents such as DMF or DMSO. Compounds of the general formula (IX) can be reacted with carbon monoxide in a carbonylation reaction, catalyzed by suitable catalysts like [1,1-Bis(diphenylphosphino)ferrocene]dichloropalladium(II) in solvents such as methanol or ethanol to the corresponding esters of the general formula (X). The ester derivatives of the general formula (X) can be hydrolyzed to the corresponding acids of the general formula (XI) under basic aqueous conditions using bases such as lithium hydroxide or sodium hydroxide. The carboxylic acids of the general formula (XI) can be reacted with amines of the general formula (III) in an amide formation reaction to the amides of the general formula (I) using amide coupling reagents such as T3P, HATU or DCCI.

Methods of Administration

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

In an embodiment, the method of administration may further include a step of administering one or more chemotherapeutic agents by intravesical dosing directly into the bladder via an intraurethral catheter.

Pharmaceutical Composition

In accordance with a further aspect, the present invention includes pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore includes pharmaceutical compositions, in particular medicaments, which comprise at least one compound according to the invention, conventionally together with one or more pharmaceutically suitable excipients, and to their use for the above mentioned purposes.

Furthermore in some embodiments the present invention includes the use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer disorders.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic©),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

### Combinations

In accordance with another aspect, the present invention includes pharmaceutical combinations, in particular medicaments, comprising at least one compound of general formula (I) of the present invention and at least one or more further active ingredients, in particular for the treatment and/or prophylaxis of a hyperproliferative disorder, more specifically a cancer disorder.

Particularly, the present invention includes a pharmaceutical combination, which comprises:
one or more first active ingredients, in particular compounds of general formula (I) as defined supra, and
one or more further active ingredients, in particular chemotherapeutic agents, more particularly anti-cancer agents.

In one embodiment the compound of the present invention are combined with immune checkpoint inhibitors, which are selected from the group consisting of agents targeting PD1, PD-L1, CTLA4, or immune modulators such as Bacillus Calmette-Guérin (BCG) vaccine.

In an embodiment, the one or more chemotherapeutic agents are selected from the group consisting of an alkylating agent, an anti-metabolite, an anti-microtubule agent, and a topoisomerase inhibitor.

In an embodiment, the one or more chemotherapeutic agents are immune checkpoint inhibitors selected from the group consisting of an agents targeting PD1, PD-L1, CTLA4, or other immune modulators.

In an embodiment, the one or more chemotherapeutic agents are receptor tyrosine kinase inhibitors (RTK). RTK inhibitors are selected from the group consisting of an agents targeting FGFR1, FGFR2, FGFR3, EGFR, ERBB2, or ERBB3.

In an embodiment, the one or more chemotherapeutic agents are Mitogen-activated protein kinase (MAPK) pathway inhibitors selected from the group consisting of an agents targeting KRAS, NRAS, HRAS, BRAF, RAF1 (c-RAF), MAP2K1 (MEK1), MAP2K2 (MEK2), MAPK1 (ERK2), or MAPK3 (ERK1).

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also includes such pharmaceutical combinations. For example, the compounds of the present invention can be combined with known anti-cancer.

Examples of anti-cancer agents include:

1311-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, Bacillus Calmette-Guerin (BCG) vaccine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, Ianreotide, Iansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, Ionidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphinesulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

### Kits

Also provided herein are kits that can include the compound of formula (I) in form of a therapeutic composition containing an effective amount of said compound in e.g., a unit dosage form.

In some embodiments, the kit comprises a sterile container which includes a therapeutic or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In particular embodiments, the instructions include at least one of the following: description of the therapeutic agent; dosage schedule and administration for treatment or prevention of malignancy or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

### Utility

Compounds of the present invention have surprisingly been found to be PPARG inverse agonists and it is possible therefore that said compounds be used for the treatment or prophylaxis of diseases, preferably hyperproliferative disorders in humans, especially cancer in humans and animals.

Compounds of the present invention can be utilized to inhibit, block, reduce, decrease, etc., cell proliferation and/or cell division. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the hyperproliferative disorder.

Hyperproliferative disorders include, but are not limited to, for example: psoriasis, keloids, and other hyperplasias affecting the skin, benign prostate hyperplasia (BPH), solid tumours, such as cancers of the brain, breast, digestive tract, eye, head and neck, liver, respiratory tract, reproductive organs, skin, thyroid, parathyroid, urinary tract, and their distant metastases. Those disorders also include leukaemias, lymphomas, and sarcomas.

Examples of breast cancers include, but are not limited to, invasive ductal carcinoma, invasive lobular carcinoma, ductal carcinoma in situ, and lobular carcinoma in situ.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the digestive tract include, but are not limited to, anal, colon, colorectal, oesophageal, gallbladder, gastric, pancreatic, rectal, small-intestine, and salivary gland cancers.

Eye cancers include, but are not limited to, intraocular melanoma and retinoblastoma.

Head-and-neck cancers include, but are not limited to, laryngeal, hypopharyngeal, nasopharyngeal, oropharyngeal cancer, lip and oral cavity cancer and squamous cell.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Examples of cancers of the respiratory tract include, but are not limited to, small-cell and non-small-cell lung carcinoma, as well as bronchial adenoma and pleuropulmonary blastoma.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the urinary tract include, but are not limited to, bladder, penile, kidney, renal pelvis, ureter, urethral and human papillary renal cancers.

Bladder cancer includes luminal and non-luminal bladder cancer, basal bladder cancer, muscle-invasive bladder cancer, or non-muscle-invasive bladder cancer.

Skin cancers include, but are not limited to, squamous cell carcinoma, Kaposi's sarcoma, malignant melanoma, Merkel cell skin cancer, and non-melanoma skin cancer.

Leukemias include, but are not limited to, acute myeloid leukemia, acute lymphoblastic leukemia, chronic lymphocytic leukemia, chronic myelogenous leukemia, and hairy cell leukemia.

The hyperproliferative disorders have been well characterized in humans, but also exist with a similar etiology in other mammals, and can be treated by administering pharmaceutical compositions of the present invention.

The “subject” being treated is a human or non-human mammal (e.g., a bovine, a canine, an equine, a feline, an ovine, a primate, and the like).

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of many indications and stages with or without pre-treatment of the tumour growth.

Thus in some embodiments, the present invention includes a method of inhibiting proliferation of a cell and/or the induction of apoptosis in a cell, comprising contacting the cell with a compound of formula (I) according to any one of claims 1-7 as well as compounds of formula (I) for use in such method.

Particularly in some embodiments, the present invention includes a method of treating a hyperproliferative disorder, more particularly cancer, comprising administering an effective amount of at lest one compound of general formula (I) according to any one of claims 1-7 as well as compounds of formula (I) for use in such methods.

A method of inhibiting PPARG activity in a cancer cell is also provided, wherein the method comprises contacting a cancer cell with a compound of general formula (I). The cancer cell may be in vitro or in vivo.

In accordance with a further aspect, the present invention includes a method of treatment or prophylaxis of diseases, in particular hyperproliferative disorders, particularly cancer disorders, using an effective amount of a compound of general formula (I), as described supra, or stereoisomers, tautomers, N-oxides, hydrates, solvates, and salts thereof, particularly pharmaceutically acceptable salts thereof, or mixtures of same as well as compounds of formula (I) for use in such methods.

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., breast cancer, esophageal cancer, pancreatic cancer, colorectal cancer, hepatocellular cancer, and bladder cancer etc.).

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., bladder cancer and pancreatic cancer).

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., bladder cancer).

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., pancreatic cancer).

Furthermore in some embodiments, the present invention includes a method of treating cancer, particularly breast cancer, esophageal cancer, pancreatic cancer, colorectal cancer, hepatocellular cancer, and bladder cancer comprising administering an effective amount of at least one compound of formula (I) according to any one of claims 1-7.

Furthermore in some embodiments, the present invention includes a method of treating cancer, particularly bladder cancer and pancreatic cancer comprising administering an effective amount of at least one compound of formula (I) according to any one of claims 1-7.

Furthermore in some embodiments, the present invention includes a method of treating cancer, particularly breast cancer, esophageal cancer, pancreatic cancer, colorectal cancer, hepatocellular cancer, and bladder cancer comprising administering an effective amount of at least one compound of formula (I) according to any one of claims 1-7.

Furthermore in some embodiments, the present invention includes a method of treating cancer, particularly bladder cancer and pancreatic cancer comprising administering an effective amount of at least one compound of formula (I) according to any one of claims 1-7.

In accordance with a further aspect, the present invention includes compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyperproliferative disorders, more particularly cancer diseases effecting the following organs: brain, breast, digestive tract, head and neck, liver, respiratory tract, reproductive organs, skin, thyroid, urinary tract, and their distant metastases including leukaemias, lymphomas, and sarcomas.

In some embodiments, the present invention includes a method of using a compound of general formula (I) for the treatment of diseases.

In some embodiments, the present invention includes a compound of general formula (I) for use in a method of inhibiting proliferation of a cell and/or the induction of apoptosis in a cell, comprising contacting the cell with a compound of formula (I) according to any one of claims 1-7.

Particularly in some embodiments, the present invention includes compounds of general formula (I) for use in a method of treating a hyperproliferative disease, more particularly wherein the hyperproliferative disease is cancer, and yet even more particularly wherein the cancer disease is bladder cancer and pancreatic cancer.

In some embodiments the present invention provides for compounds of general formula (I) for use in a method of treating cancer, particularly where the cancer disease is bladder cancer or pancreatic cancer.

In an embodiment, the bladder cancer is luminal and non-luminal bladder cancer, basal bladder cancer, muscle-invasive bladder cancer, or non-muscle-invasive bladder cancer.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of the invention.

### Experimental Section

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| ACN | acetonitrile |
| AcOH | Acetic acid |
| aq. | aqueous |
| BSA | Bovine serum albumin |
| br | broad signal (NMR) |
| d | doublet (NMR) |
| DAD | Diode Array Detector |
| DCM | Dichloromethane |
| dd | doublet of doublet (NMR) |
| DIEA | Diisopropylethylamine |
| DMF | N,N-dimethylformamide |
| DMSO | dimethylsulfoxide |
| DMEM | Dulbecco's Modified Eagle's Medium |
| DTT | Dithiothreitol |
| EDC•HCl | N-(3-Dimethylaminopropyl)-N′-ethylcarbodiimide hydrochloride salt |
| EDTA | Ethylenediamine tetraacetic acid |
| ESI | electrospray (ES) ionization |
| EtOAc | Ethyl acetate |
| EtOH | Ethanol |
| FRET | Fluorescence resonance energy transfer |
| FBS | Fetal bovine serum |
| GI50 | Growth inhibition of 50% to defined baseline |
| h, hr (hrs) | hour(s) |
| HATU | (1-[Bis(dimethylamino)methylene]-1H-1,2,3-triazolo[4,5-b]pyridinium 3- |
| | oxide hexafluorophosphate |
| HCl | hydrogen chloride, hydrochloric acid |
| HEPES | 4-(2-hydroxyethyl)-1-piperazineethanesulfonic acid |
| HPLC | high performance liquid chromatography |
| LBD | Ligand binding domain |
| LC-MS | liquid chromatography-mass spectrometry |
| m | multiplet (NMR) |
| MeCN | Acetonitrile |
| MeOH | Methanol |
| min | minute(s) |
| MS | mass spectrometry |
| MTBE | 2-Methoxy-2-methylpropan |
| NMR | Nuclear Magnetic Resonance spectroscopy: chemical shifts (δ) are |
| | given in ppm. The chemical shifts were corrected by setting the DMSO |
| | signal to 2.50 ppm using unless otherwise stated. |
| PTFE | Polytetrafluoroethylene |
| q | quartet (NMR) |
| Rt or rt | room temperature |
| Rₜ | retention time |
| s | singulet (NMR) |
| RPMI | Roswell Park Memorial Institute |
| sat. | saturated |
| SNP | Single nucleotide polymorphism |
| t | triplet (NMR) |
| td | triplet of doublet (NMR) |
| TEA | triethylamine |
| TFA | Trifluoroacetic acid |
| THF | tetrahydrofuran |
| T3P | propanephosphonic acid anhydride |
| δ | chemical shift |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as gradients of hexane/ethyl acetate or DCM/methanol. In flash column chromatography, unmodified (“regular”) silica gel may be used as well as aminophase functionalized silica gel. If reference is made to flash column chromatography or to flash chromatography in the experimental section without specification of a stationary phase, regular silica gel was used.

In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Analytical LC-MS Methods:

### Method 1

Instrument: Agilent 1290 UPLCMS 6230 TOF; column: BEH C 18 1.7 μm, 50×2.1 mm; Eluent A: water+0.05% formic acid (99%); Eluent B: acetonitrile+0.05% formic acid (99%); gradient: 0-1.7 2-90% B, 1.7-2.0 90% B; flow 1.2 ml/min; Column Temperature: 60° C.; DAD scan: 190-400 nm.

### Method 2:

Column: Kinetex EVO C18 2.1×30 mm, 5 um. MPA: 0.0375% TFA in water. MPB: 0.0188% TFA in acetonitrile. Gradient: 0.0 min 5% B->0.80 min 95% B->1.20 min 95% B->1.21 min 5% B->1.55 min 95% B; flow rate: 1.5 mL/min; Column Temperature: 50° C.; UV detection: 220 nm & 254 nm.

### Method 3:

Column: Kinetex EVO C18 2.1×30 mm, 5 um. MPA: 0.025% NH₃·water in water. MPB: acetonitrile. Gradient: 0.0 min 0% B->1.20 min 60% B->1.60 min 60% B->1.61 min 0% B->2.20 min 0% B; flow rate: 1.0 mL/min; Column Temperature: 40° C.; UV detection: 220 nm & 254 nm.

### Method 4:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; Column Temperature: 60° C.; DAD scan: 210-400 nm.

### Method 5:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; Column Temperature: 60° C.; DAD scan: 210-400 nm.

### Method 6:

Instrument: SHIMADZU LCMS-2020 SingleQuad; Column: Chromolith@Flash RP-18E 25*2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 ml/min; Colmn Temperature: 50° C.; PDA: 220 nm & 254 nm.

### Method 7:

Instrument: SHIMADZU LCMS-2020: Column: Kinetex EVO C18 2.1×30 mm, 5 um. MPA: 0.0375% TFA in water. MPB: 0.0188% TFA in acetonitrile. Gradient: 0.0 min 5% B, 0 min 95% B, 3.6 min 95% B, 3.61 min 5% B, 1.0 min 5% B; flow rate: 0.8 mL/min; Column Temperature: 40° C.; UV detection: 220 nm & 254 nm.

### Method 8:

Column: Kinetex EVO C18 2.1×30 mm, 5 um. MPA: 0.0375% TFA in water. MPB: 0.0188% TFA in acetonitrile. Gradient: 0.0 min 5% B 0.80 min 95% B,1.20 min 95% B, 1.21 min 5% B, 1.55 min 95% B; flow rate: 1.5 mL/min; Column Temperature: 50° C.; UV detection: 220 nm & 254 nm.

### Method 9:

Instrument: SHIMADZU LCMS-2020: Column: Kinetex EVO C18 2.1×30 mm, 5 um. MPA: 0.0375% TFA in water. MPB: 0.0188% TFA in acetonitrile. Gradient: 0.0 min 5% B, 3.0 min 95% B, 3.6 min 95% B, 3.61 min 5% B, 4.0 min 5% B; flow rate: 0.8 mL/min; Column Temperature: 40° C.; UV detection: 220 nm & 254 nm.

### Method 10:

Instrument: Waters Acquity UPLCMS SingleQuad; Column: Acquity UPLC BEH C18 1.7 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm

### NMR Spectra:

The multiplicities of proton signals in ¹H NMR spectra given in the following paragraphs reflect the observed signal form and do not take into account any higher-order signal phenomena. As a rule, the chemical shift data refers to the center of the signal in question. In the case of wide multiplets, a range is specified. Signals hidden by solvent or water were either assigned tentatively or are not listed. Strongly broadened signals—e.g. caused by rapid rotation of molecular moieties or by interchanging protons—have also been assigned tentatively (often referred to as a broad multiplet or broad singlet) or are not shown.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δ₁ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight”<1%.

### Synthesis of Intermediate Compounds

### Intermediate 1

### 4-(difluoromethoxy)-2-methyl-1-nitrobenzene

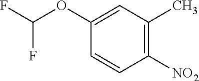

To a solution of 3-methyl-4-nitro-phenol (30 g, 195.91 mmol) and potassium carbonate (32.49 g, 235.09 mmol) in DMF (200 mL) was added (2-chloro-2,2-difluoro-acetyl)oxysodium (59.74 g, 391.81 mmol). The mixture was stirred at 100° C. for 16 hours. The mixture was diluted with H₂O (600 mL) and extracted with TBME (500 mL×6). The combined organic layers were washed with H2O (500 mL×6), dried over Na2SO4, filtered and concentrated under reduced pressure. The residue was purified by column chromatography (SiO2, Petroleum ether/Ethyl acetate=100/1 to 10/1) to give the title compound 4-(difluoromethoxy)-2-methyl-1-nitrobenzene (14 g, 35% yield) as a colorless oil.

1H NMR: (DMSO-d6 400 MHz) δ ppm 8.06 (d, 1H), 7.09 (t, 2H), 6.62 (t, 1H), 2.65 (s, 3H).

19F NMR: (DMSO-d6 376 MHz) −82.20.

### Intermediate 2

### 4-(difluoromethoxy)-2-methylaniline

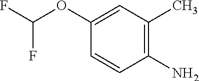

To a solution of 4-(difluoromethoxy)-2-methyl-1-nitro-benzene (13.5 g, 66.46 mmol) in MeOH (150 mL) and NH3·H2O (15 mL) was added Pd/C (1.4 g, 10% purity). The mixture was stirred at 20° C. under H2 for 20 hrs. The reaction mixture was filtered and the liquid was concentrated under reduced pressure to give the title compound 4-(difluoromethoxy)-2-methylaniline (11 g, 95% yield) as orange liquid.

1H NMR: (DMSO-d6 400 MHz) δ ppm 6.79-6.90 (m, 2H) 6.56-6.67 (m, 1H) 3.60 (m, 2H) 2.16 (m, 3H)

19F NMR: (DMSO-d6 376 MHz) −79.82.

### Intermediate 3

### 5-bromo-2-chloro-4-fluorobenzoic acid

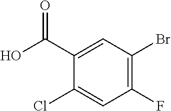 [SMILES: O=C(O)c1cc(Br)c(F)cc1Cl]

To a solution of 2-amino-5-bromo-4-fluoro-benzoic acid (20 g, 85.46 mmol) in 6 M aq. HCl (50 mL) at 0° C. was added dropwise a solution of NaNO2 (7.08 g, 102.55 mmol) in H2O (30 mL). The mixture was stirred at 0° C. for 2 hours, then a solution of CuCl (10.15 g, 102.55 mmol) in HCl (30 mL) was added into the reaction mixture dropwise under stirring while the reaction temperature was kept 0° C. (ice-salt bath). After the addition was complete, the mixture was stirred at 0° C. for another 1 hour. Then the mixture was warmed to 25° C. and stirred for 16 hours. The mixture was filtered and the collected solid was dried in vacuum. The residue was purified by flash silica gel chromatography (ISCO@; 120 g SepaFlash@ Silica Flash Column, Eluent of 0-20% Ethyl acetate/Petroleum ethergradient @ 80 mL/min) to give the title compound 5-bromo-2-chloro-4-fluorobenzoic acid (19.2 g, 88.7% yield) as a white solid.

1H NMR: (DMSO-d6 400 MHz) δ ppm 13.71 (br, 1H), 8.13 (d, 1H), 7.74 (d, 1H).

19F NMR: (DMSO-d6 376 MHz) −101.67.

LC-MS: (method 3) Rₜ=0.20 min, m/z=252.6 (M−H)+

### Intermediate 4

### 5-bromo-2-chloro-N-(4-(difluoromethoxy)-2-methylphenyl)-4-fluorobenzamide

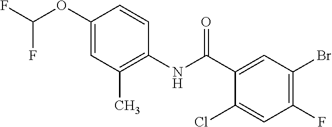 [SMILES: Cc1cc(OC(F)F)ccc1NC(=O)c1cc(Br)c(F)cc1Cl]

To a solution of 5-bromo-2-chloro-4-fluoro-benzoic acid (15 g, 59.18 mmol, Intermediate 3;) and 4-(difluoromethoxy)-2-methyl-aniline (10.97 g, 63.33 mmol) in DMF (150 mL) was added DIEA (15.30 g, 118.37 mmol) and HATU (45.01 g, 118.37 mmol). The mixture was stirred at 25° C. for 16 hours. The mixture was added into water (300 mL) and a white solid was formed. The solid was collected by filtration and dried in vacuo to give the title compound 5-bromo-2-chloro-N-(4-(difluoromethoxy)-2-methylphenyl)-4-fluorobenzamide (18 g, 74% yield) as a white solid.

1H NMR: (DMSO-d6 400 MHz) δ ppm 10.06 (s, 1H), 8.06 (d, 1H), 7.81 (d, 1H), 7.48 (d, 1H), 7.20 (t, 1H), 7.11 (d, 1H), 7.06 (dd, 1H), 2.28 (s, 3H).

LC-MS (method 2): Rₜ=0.973 min, m/z=409.9 (M+H)+.

Intermediate 5

### 4-chloro-5-((4-(difluoromethoxy)-2-methylphenyl)carbamoyl)-2-fluorobenzoate

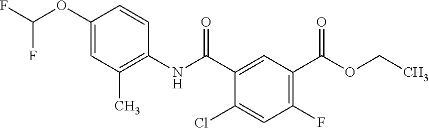 [SMILES: CCOC(=O)c1cc(C(=O)Nc2ccc(OC(F)F)cc2C)c(Cl)cc1F]

To a solution of 5-bromo-2-chloro-N-[4-(difluoromethoxy)-2-methyl-phenyl]-4-fluoro-benzamide (17.9 g, 43.81 mmol, Intermediate 4) and TEA (13.30 g, 131.43 mmol) in EtOH (200 mL) was added Pd(dppf)Cl₂ (3.21 g, 4.38 mmol) under N2 atmosphere. The suspension was degassed and purged with CO for 3 times. The mixture was stirred under CO (50 Psi) at 60° C. for 40 hrs. The mixture was filtered through a celite pad. Then the filtrate was concentrated under reduced pressure. The residue was purified by flash silica gel chromatography (ISCO@; 120 g SepaFlash@ Silica Flash Column, Eluent of 0-40% Ethyl acetate/Petroleum ethergradient @ 60 mL/min) to give the title compound 4-chloro-5-[[4-(difluoromethoxy)-2-methyl-phenyl]carbamoyl]-2-fluoro-benzoate (12 g, 68% yield) as a white solid.

1H NMR: (DMSO-d6 400 MHz) δ ppm: 10.12 (s, 1H), 8.12 (d, 1H), 7.81 (d, 1H), 7.49 (d, 1H), 7.22 (t, 1H), 7.12 (d, 1H), 7.05 (dd, 1H), 4.34-4.39 (m, 2H), 2.33 (t, 3H) 1.34 (t, 3H).

19F NMR: (DMSO-d6 376 MHz) δ [ppm]: −81.70, −106.63.

Intermediate 6

### 4-chloro-5-{[4-(difluoromethoxy)-2-methylphenyl]carbamoyl}-2-fluorobenzoic acid

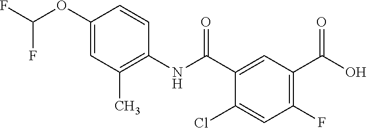 [SMILES: Cc1cc(OC(F)F)ccc1NC(=O)c1cc(C(=O)O)c(F)cc1Cl]

To a solution of ethyl 4-chloro-5-[[4-(difluoromethoxy)-2-methyl-phenyl] carbamoyl]-2-fluoro-benzoate (12 g, 29.87 mmol Intermediate 5;) in water (25 mL) and THF (125 mL) was added LiOH·water (1.25 g, 29.87 mmol). The mixture was stirred at 25° C. for 16 hours. The reaction mixture was diluted with water (100 mL) and the mixture was concentrated under reduced pressure to remove THF. Then the aqueous was washed with TBME (100 mL). To the aqueous phase was added 4M aq. HCl (20 mL) and a white solid was formed. The solid was collected by filtration and dried in vacuo to give the title compound 5-((4-(difluoromethoxy)-2-methylphenyl) carbamoyl)-2-fluorobenzoic acid (8.6 g, 76% yield) as a white solid.

LC-MS (method 2) t=0.789 min, m/z=374.0 (M+H)+.

1H NMR: (DMSO-d6 400 MHz) δ ppm 13.68 (br, 1H), 10.10 (s, 1H), 8.09 (d, 1H), 7.75 (d, 1H), 7.48 (d, 1H), 7.21 (t, 1H), 7.11 (d, 1H), 7.06 (dd, 1H), 2.28 (s, 3H).

19F NMR: (DMSO-d6 376 MHz) δ [ppm]: −81.69, −106.45.

### Intermediate 7

### 5-bromo-4-chloro-N-[(3,4-difluorophenyl)methyl]-2-fluorobenzamide

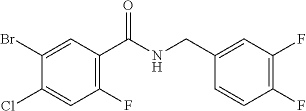 [SMILES: O=C(NCc1ccc(F)c(F)c1)c1cc(Br)c(Cl)cc1F]

To a solution of 5-bromo-4-chloro-2-fluorobenzoic acid (3.50 g, 13.8 mmol) in DCM (35 mL) was added DMF (110 μl). To the mixture was added dropwise oxalyl chloride (1.4 ml, 17 mmol; CAS-RN:[79-37-8]) and stirring continued for 1 h at RT. In a separate flask 1-(3,4-difluorophenyl)methanamine (2.1 ml, 18 mmol) was dissolved in DCM (35 ml) and an aqueous solution of sodium carbonate (35 ml, 2.0 M, 69 mmol) was added. To this solution the previously prepared acid chloride was slowly added and intense stirring continued at RT for 1 h. The biphasic reaction mixture was separated and the aqueous part extracted 2× with DCM. The combined organic phases were washed 1× with hydrochloric acid (1M) and 1× with aqueouse saturated sodium hydrocarbonate solution. After drying with sodium sulfate and removal of the organic solvents the desired product 5.1 g (98% yield) was isolated as a pale brown solid.

LC-MS (Method 4): Rₜ=1.34 min; MS (ESIpos): m/z=378 [M+H]⁺

1H-NMR (400 MHz, DMSO-d6) delta [ppm]: 2.019 (0.41), 2.058 (1.70), 2.073 (1.25), 2.522 (2.95), 2.685 (0.56), 3.158 (1.58), 3.171 (1.74), 3.285 (0.48), 3.302 (1.11), 3.380 (2.21), 3.396 (0.74), 3.412 (0.52), 3.427 (0.40), 3.621 (3.07), 3.915 (0.44), 3.944 (0.74), 4.105 (0.40), 4.119 (0.45), 4.429 (14.82), 4.444 (15.07), 4.759 (0.58), 5.755 (2.09), 7.126 (0.52), 7.164 (2.94), 7.167 (3.05), 7.174 (3.25), 7.180 (3.64), 7.185 (3.73), 7.188 (3.60), 7.194 (3.48), 7.276 (0.72), 7.297 (1.16), 7.302 (0.91), 7.323 (1.15), 7.352 (3.43), 7.357 (3.40), 7.370 (6.27), 7.377 (4.22), 7.381 (4.41), 7.391 (8.47), 7.397 (5.50), 7.401 (4.30), 7.406 (3.85), 7.412 (4.45), 7.418 (7.81), 7.439 (3.61), 7.816 (15.90), 7.841 (15.89), 8.004 (16.00), 8.021 (15.41), 9.058 (2.59), 9.072 (4.71), 9.086 (2.55).

### Intermediate 8

### 2-chloro-5-{[(3,4-difluorophenyl)methyl]carbamoyl}-4-fluorobenzoic acid

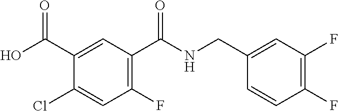 [SMILES: O=C(NCc1ccc(F)c(F)c1)c1cc(C(=O)O)c(Cl)cc1F]

In an autoclave 5-bromo-4-chloro-N-[(3,4-difluorophenyl)methyl]-2-fluorobenzamide (5.10 g, 13.5 mmol; Intermediate 7) was dissolved in DMSO (200 ml), 1,1′-BIS(DIPHENYLPHOSPHINO)-FERROCENE (1.55 g, 97% purity, 2.71 mmol) and triethylamine (1.1 ml, 7.6 mmol; CAS-RN:[121-44-8]) and Palladium(II)acetate (151 mg, 674 μmol; CAS-RN:[3375-31-3]) and potassium acetate (5.29 g, 53.9 mmol; CAS-RN:[127-08-2]) was added and the mixture pressurized with carbon monoxide at 14.6 bar. The mixture was stirred at 80° C. for 28 h. The reaction mixture was stirred for an additional 16 h at RT. The crude reaction mixture was treated with 1 M sodium hydroxide solution until pH 10 and extracted with ethyl acetate. The aqueous layer was treated with 1M hydrochloric acid until pH 3, and extracted 2× with ethyl acetate. The combined organic phases were washed 3× with water and the organic layer was dried with sodium sulfate and the organic solvents were concentrated under reduced pressure. After another acidic/basic extraction procedure 3.46 g (75% yield) of the title compound was prepared and used without further purification.

LC-MS (Method 4): Rₜ=1.04 min; MS (ESIpos): m/z=344 [M+H]⁺

1H-NMR (400 MHz, DMSO-d6) delta [ppm]: 4.442 (15.92), 4.456 (16.00), 4.490 (1.64), 5.756 (0.53), 7.185 (5.31), 7.220 (1.47), 7.355 (3.88), 7.373 (7.54), 7.395 (8.22), 7.400 (8.43), 7.421 (6.42), 7.442 (2.71), 7.478 (0.41), 7.551 (0.53), 7.694 (10.35), 7.719 (10.29), 8.095 (10.50), 8.114 (10.33), 8.231 (1.20), 9.074 (6.18), 13.662 (0.54).

Intermediate 9 3-bromo-4,5-dichloro-N-[(3,4-difluorophenyl)methyl]benzamide

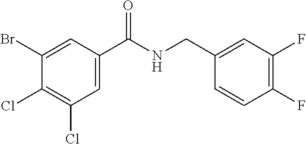 [SMILES: O=C(NCc1ccc(F)c(F)c1)c1cc(Cl)c(Cl)c(Br)c1]

To a solution of 3-bromo-4,5-dichlorobenzoic acid (1.00 g, 3.70 mmol, CAS RN [791137-25-2]), 1-(3,4-difluorophenyl)methanamine (530 μl, 4.4 mmol, CAS RN[72235-53-1]) and HATU (1.69 g, 4.45 mmol; CAS-RN:[148893-10-1]) in DMF (23 ml) was added N,N-diisopropylethylamine (1.7 ml, 9.6 mmol; CAS-RN:[7087-68-5]) The mixture was stirred at rt for 24 h. The reaction mixture was evaporated and then treated with water and extracted with DCM. The combined organic phases were evaporated and the residue was purified by reversed phase preperative.

HPLC (acidic conditions). Fractions containing the product were pooled, ACN was evaporated and the residue freeze dried to yield the desired product 996 mg (95% purity, 65% yield).

LC-MS (Method 1): Rₜ=1.38 min; MS (ESIpos): m/z=396 [M+H]⁺

¹H-NMR (400 MHz, DMSO-d6) δ[ppm]: 2.083 (1.74), 2.518 (2.58), 2.523 (1.82), 4.444 (7.84), 4.458 (7.82), 7.159 (1.50), 7.162 (1.51), 7.164 (1.46), 7.168 (1.59), 7.170 (1.60), 7.175 (1.85), 7.180 (1.96), 7.184 (1.84), 7.186 (1.74), 7.189 (1.68), 7.192 (1.76), 7.195 (1.34), 7.356 (2.82), 7.362 (2.01), 7.368 (2.01), 7.378 (5.16), 7.383 (4.57), 7.388 (2.32), 7.392 (2.25), 7.399 (3.91), 7.404 (4.91), 7.412 (1.99), 7.417 (1.91), 7.426 (2.32), 8.130 (14.97), 8.135 (16.00), 8.228 (15.91), 8.233 (14.68), 9.302 (1.51), 9.316 (2.98), 9.330 (1.45).

### Intermediate 10

### methyl 2,3-dichloro-5-{[(3,4-difluorophenyl)methyl]carbamoyl}benzoate

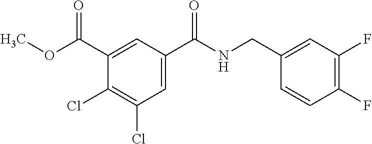 [SMILES: COC(=O)c1cc(C(=O)NCc2ccc(F)c(F)c2)cc(Cl)c1Cl]

In an autoclave 3-bromo-4,5-dichloro-N-[(3,4-difluorophenyl)methyl]benzamide (995 mg, 2.52 mmol; Intermediate 9) was dissolved in 30 ml of a mixture methanol/THF (10:1), Dichloro[1,1′-bis(diphenylphosphino)ferrocene]palladium(II) dichloromethane complex: (206 mg, 252 μmol; CAS-RN:[95464-05-4]) and triethylamine (1.1 ml, 7.6 mmol; CAS-RN: [121-44-8]) were added and the mixture pressurized with carbon monoxide at 14.8 bar. The mixture was stirred at 80° C. for 23 h. The crude reaction mixture was filtered using a PTFE filter (0.2 μm), rinsed with methanol and then the organic solvents were concentrated under reduced pressure. 776 mg (98% purity, 81% yield) of the title compound were prepared after purification by flash chromatography (silica, hexane/EtOAc gradient 0-25%).

LC-MS (Method 1): Rₜ=1.24 min; MS (ESIpos): m/z=374.1 [M+H]⁺

¹H-NMR (400 MHz, DMSO-d6) δ[ppm]: 2.518 (0.95), 2.523 (0.66), 3.908 (16.00), 4.456 (1.98), 4.471 (1.97), 7.174 (0.40), 7.180 (0.46), 7.185 (0.48), 7.188 (0.45), 7.196 (0.44), 7.357 (0.64), 7.367 (0.48), 7.372 (0.49), 7.379 (1.18), 7.385 (1.00), 7.392 (0.55), 7.396 (0.59), 7.400 (0.94), 7.405 (1.18), 7.416 (0.49), 7.421 (0.45), 7.427 (0.56), 8.234 (2.94), 8.239 (3.54), 8.313 (3.47), 8.318 (2.92), 9.354 (0.41), 9.368 (0.80).

### Intermediate 11

### 2,3-dichloro-5-{[(3,4-difluorophenyl)methyl]carbamoyl}benzoic acid

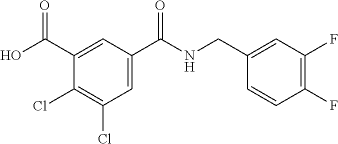 [SMILES: O=C(NCc1ccc(F)c(F)c1)c1cc(Cl)c(Cl)c(C(=O)O)c1]

Methyl 2,3-dichloro-5-{[(3,4-difluorophenyl)methyl]carbamoyl}benzoate (770 mg, 2.06 mmol Intermediate 10;) was dissolved in THF (11 ml) and aqueous sodium hydroxide solution (10 ml, 1.0 M, 10 mmol) was added. The reaction mixture was stirred at RT for 90 min. EtOAc was added, and the organic phase separated. The aqueous phase was treated with 2M aqueous hydrochloric acid until pH was slightly acidic. The solution was extracted 2× with EtOAc and the combined organic phases were filtered and dried through a hydrophobic filter. The organic solvents were concentrated under reduced pressure yielding the title compound (680 mg, 98% purity, 83% yield) which was used without further purification.

LC-MS (Method 1): Rₜ=1.02 min; MS (ESIpos): m/z=360.1 [M+H]⁺

¹H-NMR (400 MHz, DMSO-d6) δ[ppm]: 1.352 (0.41), 2.518 (5.30), 2.523 (3.63), 4.451 (10.23), 4.465 (10.26), 7.161 (2.02), 7.165 (2.00), 7.172 (2.16), 7.177 (2.52), 7.183 (2.59), 7.186 (2.41), 7.193 (2.40), 7.356 (3.41), 7.364 (2.58), 7.369 (2.61), 7.377 (6.32), 7.383 (6.12), 7.388 (3.00), 7.393 (3.10), 7.399 (5.59), 7.404 (6.34), 7.412 (2.59), 7.418 (2.48), 7.425 (2.83), 8.197 (13.38), 8.203 (15.39), 8.249 (16.00), 8.254 (13.88), 9.338 (2.33), 9.354 (4.66), 9.368 (2.27), 13.964 (0.46).

### Intermediate 12

### 3-bromo-4,5-dichloro-N-[(4-fluorophenyl)methyl]benzamide

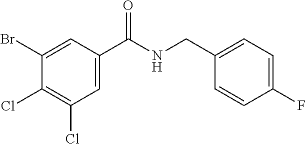 [SMILES: O=C(NCc1ccc(F)cc1)c1cc(Cl)c(Cl)c(Br)c1]

Using an analogous method as described for Intermediate 9, 3-bromo-4,5-dichlorobenzoic acid (1.00 g, 3.70 mmol) and 1-(4-fluorophenyl)methanamine (510 μl, 4.4 mmol) were used as the starting materials. 687 mg (90% purity, 44% yield) of the title compound were prepared.

LC-MS (Method 1): Rₜ=1.35 min; MS (ESIpos): m/z=378 [M+H]⁺

1H-NMR (400 MHz, DMSO-d6) delta [ppm]: 1.154 (0.64), 1.172 (1.03), 1.189 (0.50), 1.987 (1.61), 2.331 (0.97), 2.337 (0.44), 2.518 (5.42), 2.523 (3.83), 2.673 (0.98), 2.678 (0.42), 4.443 (9.64), 4.458 (9.60), 7.127 (0.71), 7.135 (6.91), 7.141 (2.39), 7.151 (2.99), 7.158 (14.52), 7.164 (2.86), 7.174 (2.58), 7.180 (8.99), 7.188 (0.93), 7.337 (0.81), 7.344 (6.83), 7.350 (2.81), 7.358 (7.51), 7.367 (6.41), 7.375 (2.40), 7.381 (5.57), 7.388 (0.56), 8.057 (1.84), 8.063 (2.10), 8.128 (14.32), 8.134 (16.00), 8.147 (2.33), 8.153 (2.04), 8.228 (15.68), 8.233 (14.30), 9.283 (1.76), 9.298 (3.39), 9.312 (1.72).

### Intermediate 13

### methyl 2,3-dichloro-5-{[(4-fluorophenyl)methyl]carbamoyl}benzoate

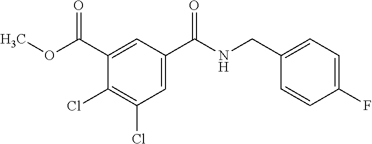 [SMILES: COC(=O)c1cc(C(=O)NCc2ccc(F)cc2)cc(Cl)c1Cl]

Using an analogous method as described for Intermediate 10, 3-bromo-4,5-dichloro-N-[(4-fluorophenyl)methyl]benzamide (680 mg, 90% purity, 1.62 mmol; Intermediate 12) was used as the starting materials. 530 mg (97% purity, 89% yield) of the title compound were prepared.

LC-MS (Method 1): Rₜ=1.21 min; MS (ESIpos): m/z=356.1 [M+H]⁺

¹H-NMR (400 MHz, DMSO-d6) δ[ppm]: 1.987 (0.50), 2.518 (1.07), 2.523 (0.72), 3.884 (0.68), 3.887 (0.63), 3.891 (0.60), 3.906 (16.00), 4.455 (2.40), 4.470 (2.44), 7.136 (1.71), 7.141 (0.64), 7.152 (0.79), 7.158 (3.62), 7.164 (0.79), 7.174 (0.67), 7.180 (2.19), 7.349 (1.69), 7.355 (0.74), 7.363 (1.89), 7.371 (1.61), 7.379 (0.64), 7.385 (1.39), 8.236 (3.03), 8.241 (3.69), 8.309 (3.68), 8.314 (3.01), 9.336 (0.47), 9.350 (0.92), 9.365 (0.45).

### Intermediate 14

### 2,3-dichloro-5-{[(4-fluorophenyl)methyl]carbamoyl}benzoic acid

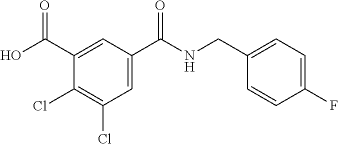 [SMILES: O=C(NCc1ccc(F)cc1)c1cc(Cl)c(Cl)c(C(=O)O)c1]

Methyl 2,3-dichloro-5-{[(4-fluorophenyl)methyl]carbamoyl}benzoate (530 mg, 1.49 mmol; Intermediate 13) was dissolved in THF (7.7 ml) and aqueous sodium hydroxide solution (7.4 ml, 1.0 M, 10 mmol) was added. The reaction mixture was stirred at RT for 90 min. EtOAc was added, and the organic phase separated. The aqueous phase was treated with 2M aqueous hydrochloric acid until the pH was slightly acidic. The solution was extracted 2× with EtOAc and the combined organic phases were filtered and dried through a hydrophobic filter. The organic solvents were concentrated under reduced pressure yielding the title compound 460 mg (98% purity, 89% yield) which was used without further purification.

LC-MS (Method 1): Rₜ=0.98 min; MS (ESIpos): m/z=342.1 [M+H]⁺

¹H-NMR (400 MHz, DMSO-d6) δ[ppm]: 1.154 (1.39), 1.171 (2.63), 1.189 (1.23), 1.352 (1.63), 1.907 (2.35), 1.987 (4.98), 2.518 (5.92), 2.523 (4.42), 3.842 (1.69), 3.999 (0.40), 4.017 (1.14), 4.034 (1.11), 4.446 (9.27), 4.461 (9.47), 7.124 (0.78), 7.131 (7.94), 7.136 (2.63), 7.148 (3.26), 7.154 (16.00), 7.159 (3.31), 7.171 (2.85), 7.176 (9.31), 7.183 (0.96), 7.336 (0.84), 7.344 (7.31), 7.350 (2.94), 7.358 (8.13), 7.366 (6.71), 7.374 (2.60), 7.380 (5.97), 7.388 (0.60), 8.155 (8.12), 8.160 (9.40), 8.205 (10.15), 8.210 (8.19), 9.313 (2.00), 9.328 (3.98), 9.342 (1.92).

### Intermediate 15

### 5-bromo-4-chloro-N-[(3,4-difluorophenyl)methyl]-2-fluoro-3-methylbenzamide

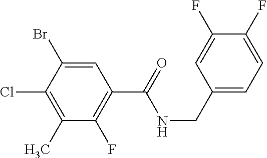 [SMILES: Cc1c(F)c(C(=O)NCc2ccc(F)c(F)c2)cc(Br)c1Cl]

5-bromo-4-chloro-2-fluoro-3-methylbenzoic acid (2.50 g, 9.35 mmol) was dissolved in DCM (21 ml) and DMF (72 μl) was added. Then oxalyl chloride (980 μl, 11 mmol; CAS-RN:[79-37-8]) was added and the mixture was stirred at RT for 1 h. In a second flask 1-(3,4-difluorophenyl)methanamine (1.61 g, 11.2 mmol) were dissolved in DCM (21 ml) and sodium carbonate, 2 M in water (23 ml, 2.0 M, 47 mmol; CAS-RN:[497-19-8]) added. The freshly prepared acid chloride was dropped to this solution and the mixture was stirred for 16 h at RT. The pH was adjusted to pH 7 with 2N hydrochloric acid and the organic layer were separated. The aqueous phase was extracted with DCM and the combined organic layers were dried using a hydrophobic filter. 3.70 g (95% purity, 96% yield) of the title compound were prepared after evaporation of the solvent.

LC-MS (Method 1): Rₜ=1.36 min; MS (ESIpos): m/z=394.1 [M+H]⁺

¹H-NMR (400 MHz, DMSO-d6) δ[ppm]: 2.084 (12.61), 2.331 (0.90), 2.368 (16.00), 2.374 (15.81), 2.518 (5.13), 2.523 (3.53), 2.669 (1.27), 2.673 (0.87), 4.427 (6.21), 4.443 (6.26), 5.759 (0.42), 7.165 (1.16), 7.169 (1.17), 7.176 (1.31), 7.181 (1.46), 7.186 (1.46), 7.189 (1.45), 7.195 (1.40), 7.351 (1.48), 7.357 (1.40), 7.371 (1.68), 7.376 (3.37), 7.381 (1.78), 7.386 (1.60), 7.396 (4.05), 7.402 (3.03), 7.418 (1.88), 7.424 (3.76), 7.445 (1.68), 7.858 (4.53), 7.875 (4.63), 9.056 (1.02), 9.070 (1.92), 9.083 (1.02).

### Intermediate 16

### methyl 2-chloro-5-{[(3,4-difluorophenyl)methyl]carbamoyl}-4-fluoro-3-methylbenzoate

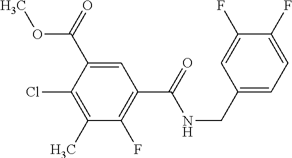 [SMILES: COC(=O)c1cc(C(=O)NCc2ccc(F)c(F)c2)c(F)c(C)c1Cl]

Using an analogous method as described for Intermediate 10, 5-bromo-4-chloro-N-[(3,4-difluorophenyl)methyl]-2-fluoro-3-methylbenzamide (3.68 g, 9.37 mmol; Intermediate 15) was used as the starting material and 1,1′-BIS(DIPHENYLPHOSPHINO)FERROCENE-PALLADIUM(II)DICHLORIDE DICHLOROMETHANE COMPLEX: (1.53 g, 1.87 mmol; CAS-RN:[95464-05-4]) as catalyst. 3.00 g (86% yield) of the title compound were prepared after purification using flash chromatography.

LC-MS (Method 5): Rₜ=1.02 min; MS (ESIpos): m/z=372 [M+H]⁺

### Intermediate 17

### 2-chloro-5-{[(3,4-difluorophenyl)methyl]carbamoyl}-4-fluoro-3-methylbenzoic acid

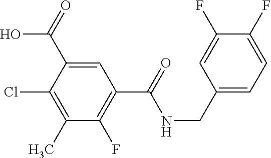 [SMILES: Cc1c(F)c(C(=O)NCc2ccc(F)c(F)c2)cc(C(=O)O)c1Cl]

Using an analogous method as described for Intermediate 14, methyl 2-chloro-5-{[(3,4-difluorophenyl)methyl]carbamoyl}-4-fluoro-3-methylbenzoate (3.00 g, 8.07 mmol; Intermediate 16) was used as the starting material. 2.70 g (97% purity, 91% yield) of the title compound were prepared.

LC-MS (Method 1): Rₜ=1.02 min; MS (ESIpos): m/z=358.2 [M+H]⁺

¹H-NMR (400 MHz, DMSO-d6) δ[ppm]: 1.907 (2.30), 2.318 (16.00), 2.323 (15.25), 2.518 (0.92), 2.523 (0.60), 4.433 (6.48), 4.448 (6.51), 7.161 (1.21), 7.165 (1.22), 7.167 (1.20), 7.171 (1.31), 7.173 (1.33), 7.178 (1.50), 7.181 (1.51), 7.183 (1.61), 7.186 (1.53), 7.188 (1.48), 7.193 (1.50), 7.343 (1.53), 7.348 (1.45), 7.363 (1.64), 7.369 (1.89), 7.372 (3.69), 7.378 (1.69), 7.393 (5.47), 7.399 (3.47), 7.414 (2.03), 7.420 (4.06), 7.442 (1.85), 7.714 (0.40), 7.786 (4.01), 7.805 (4.12), 9.050 (1.14), 9.063 (2.21), 9.077 (1.15).

### Intermediate 18

### methyl 4,5-dichloro-2-fluorobenzoate

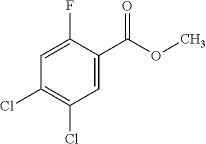

4,5-dichloro-2-fluorobenzoic acid (5.00 g, 23.9 mmol) was dissolved in methanol (50 ml), Conc. sulfuric acid (2.2 ml, 41 mmol; CAS-RN:[7664-93-9]) was added and the mixture was stirred 5 h at 60° C. Stirring continued for 16 h at RT. The reaction mixture was concentrated in vacuo and the residue was dissolved in DCM and washed with aqueous sodium bicarbonate solution (1 M). The organic phase was dried with sodium sulfate. After removal of the solvent 5.10 g (99% purity, 95% yield) of the title compound was obtained as pale yellow solid.

LC-MS (Method 4): Rₜ=1.28 min; MS (ESIpos): m/z=223 [M+H]⁺

¹H-NMR (400 MHz, DMSO-d6) δ[ppm]: 1.227 (0.57), 2.518 (0.42), 3.328 (16.00), 7.896 (4.87), 7.922 (4.80), 8.057 (4.52), 8.074 (4.46).

### Intermediate 19

### methyl 3-bromo-4,5-dichloro-2-fluorobenzoate

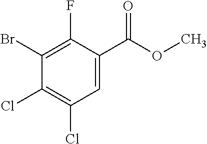

Methyl 4,5-dichloro-2-fluorobenzoate (5.10 g, 22.9 mmol) was dissolved in a mixture of sulfuric acid (30 ml, 560 mmol; CAS-RN:[7664-93-9]) and trifluoroacetic acid (30 ml) and then N-bromosuccinimide (4.88 g, 27.4 mmol; CAS-RN:[128-08-5]) was added. The mixture was stirred at 45° C. for 6 h. After cooling the mixture was added to ice water and extracted with EtOAc. The combined organic phases were washed with aqueous sodium bicarbonate solution (1M) and then the organic phase dried with sodium sulfate. After removal of the solvent the residue was purified by flash chromatography on silica gel (hexane/EtOAc 0-50%). 6.63 g (95% purity, 91% yield) of the title compound was obtained as yellow solid.

¹H-NMR (400 MHz, CHLOROFORM-d) δ [ppm]: 3.950 (1.36), 3.969 (16.00), 4.003 (2.87), 8.040 (3.01), 8.057 (3.02).

### Intermediate 20

### 3-bromo-4,5-dichloro-2-fluorobenzoic acid

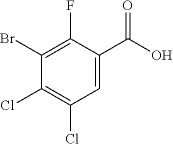 [SMILES: O=C(O)c1cc(Cl)c(Cl)c(Br)c1F]

Using an analogous method as described for Intermediate 14, methyl 3-bromo-4,5-dichloro-2-fluorobenzoate (6.63 g, 22.0 mmol; Intermediate 19) was used as the starting material in DMF. 10.7 g (59% purity, 100% yield) of the title compound were prepared.

LC-MS (Method 4): Rₜ=1.14 min; MS (ESIpos): m/z=287.2 [M−H]⁺

### Intermediate 21

### N-benzyl-3-bromo-4,5-dichloro-2-fluorobenzamide

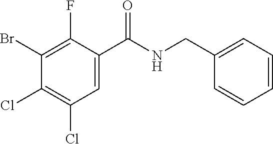 [SMILES: O=C(NCc1ccccc1)c1cc(Cl)c(Cl)c(Br)c1F]

Using an analogous method as described for Intermediate 9, 3-bromo-4,5-dichloro-2-fluorobenzoic acid (10.7 g, 59% purity, 21.9 mmol; Intermediate 20) and 1-phenylmethanamine (4.70 g, 43.9 mmol) were used as the starting material. 8.30 g (100% yield) of the title compound were prepared.

LC-MS (Method 4): Rₜ=1.36 min; MS (ESIpos): m/z=378 [M+H]⁺

¹H-NMR (400 MHz, DMSO-d6) δ[ppm]: 2.686 (16.00), 2.729 (0.73), 2.888 (0.87), 4.460 (1.47), 4.475 (1.47), 7.264 (0.42), 7.332 (0.66), 7.339 (4.65), 7.346 (1.59), 7.355 (1.38), 7.361 (0.80), 7.926 (1.51), 7.942 (1.56).

### Intermediate 22

### methyl 3-(benzylcarbamoyl)-5,6-dichloro-2-fluorobenzoate

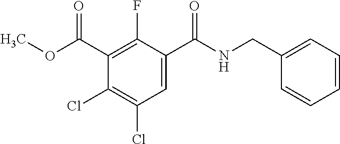 [SMILES: COC(=O)c1c(F)c(C(=O)NCc2ccccc2)cc(Cl)c1Cl]

Using an analogous method as described for Intermediate 10, N-benzyl-3-bromo-4,5-dichloro-2-fluorobenzamide (5.50 g, 14.6 mmol; Intermediate 21) was used as the starting material and Dichloro[1,1′-bis(diphenylphosphino)ferrocene]palladium(II) complex with dichloromethane (1:1), (2.38 g, 2.92 mmol; CAS-RN:[95464-05-4]) as catalyst. 663 mg (95% purity, 12% yield) of the title compound was prepared after purification using flash chromatography.

LC-MS (Method 1): Rₜ=1.24 min; MS (ESIpos): m/z=356 [M+H]⁺

1H-NMR (400 MHz, DMSO-d6) δ[ppm]: 1.154 (0.44), 1.172 (0.97), 1.190 (0.50), 1.988 (1.61), 3.961 (16.00), 4.455 (2.71), 4.469 (2.74), 7.253 (0.48), 7.258 (0.43), 7.261 (0.77), 7.268 (0.71), 7.277 (0.66), 7.283 (0.59), 7.320 (0.70), 7.324 (0.49), 7.335 (8.40), 7.341 (2.81), 7.351 (2.49), 7.357 (0.75), 7.370 (0.43), 8.042 (2.85), 8.059 (2.68), 9.184 (0.74).

### Intermediate 23

### 3-(benzylcarbamoyl)-5,6-dichloro-2-fluorobenzoic acid

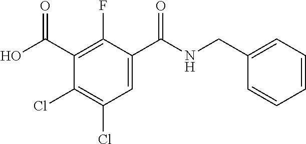 [SMILES: O=C(NCc1ccccc1)c1cc(Cl)c(Cl)c(C(=O)O)c1F]

Using an analogous method as described for Intermediate 14, methyl 3-(benzylcarbamoyl)-5,6-dichloro-2-fluorobenzoate (663 mg, 1.86 mmol; Intermediate 22) was used as the starting material. 520 mg (95% purity, 78% yield) of the title compound was prepared.

LC-MS (Method 4): Rₜ=0.90 min; MS (ESIpos): m/z=342 [M+H]⁺

¹H-NMR (400 MHz, DMSO-d6) δ[ppm]: 1.167 (0.60), 2.518 (1.79), 2.523 (1.19), 3.711 (1.04), 3.867 (0.52), 3.912 (0.82), 4.451 (5.32), 4.465 (5.27), 7.238 (0.44), 7.245 (0.75), 7.252 (1.04), 7.256 (0.89), 7.260 (1.68), 7.267 (1.56), 7.274 (1.24), 7.276 (1.36), 7.281 (1.24), 7.319 (1.49), 7.324 (1.02), 7.335 (16.00), 7.340 (5.93), 7.351 (5.21), 7.356 (1.95), 7.365 (0.83), 7.370 (0.93), 7.372 (0.71), 7.846 (0.46), 7.921 (2.65), 7.938 (2.61), 9.141 (0.79), 9.156 (1.50), 9.171 (0.75).

### Intermediate 24

### 5-bromo-2-chloro-4-fluoro-N-(4-methylphenyl)benzamide

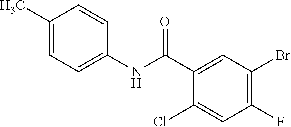 [SMILES: Cc1ccc(NC(=O)c2cc(Br)c(F)cc2Cl)cc1]

Using an analogous method as described for Intermediate 9, 5-bromo-2-chloro-4-fluorobenzoic acid (400 mg, 1.58 mmol; Intermediate 3) and 4-methylaniline (203 mg, 1.89 mmol) were used as the starting material. 420 mg (100% purity, 78% yield) of the title compound was prepared.

LC-MS (Method 4): Rₜ=1.28 min; MS (ESIpos): m/z=344 [M+H]⁺

¹H-NMR (400 MHz, CHLOROFORM-d) δ [ppm]: 1.253 (0.80), 2.351 (12.82), 7.179 (2.94), 7.199 (3.32), 7.238 (3.30), 7.260 (16.00), 7.482 (4.24), 7.503 (3.60), 7.804 (0.79), 8.013 (3.16), 8.031 (3.25).

### Intermediate 25

### methyl 4-chloro-2-fluoro-5-[(4-methylphenyl)carbamoyl]benzoate

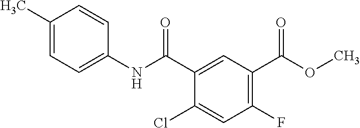

Using an analogous method as described for Intermediate 10, 5-bromo-2-chloro-4-fluoro-N-(4-methylphenyl)benzamide (420 mg, 1.23 mmol; Intermediate 24) was used as the starting material and 1,1′-BIS(DIPHENYLPHOSPHINO)FERROCENE-PALLADIUM(II)DICHLORIDE DICHLOROMETHANE COMPLEX (100 mg, 123 μmol; CAS-RN: [95464-05-4]) as catalyst. 295 mg (95% purity, 71% yield) of the title compound were prepared after purification using flash chromatography.

LC-MS (Method 4): Rt=1.17 min; MS (ESIpos): m/z=322 [M+H]⁺

¹H-NMR (400 MHz, CHLOROFORM-d) δ [ppm]: 1.219 (0.62), 1.225 (0.57), 2.011 (0.43), 2.319 (9.97), 3.860 (0.44), 3.915 (16.00), 3.948 (0.44), 7.148 (2.39), 7.169 (2.69), 7.227 (12.88), 7.245 (2.25), 7.463 (3.36), 7.484 (2.90), 7.707 (0.81), 8.321 (2.19), 8.340 (2.20).

### Intermediate 26

### 4-chloro-2-fluoro-5-[(4-methylphenyl)carbamoyl]benzoic acid

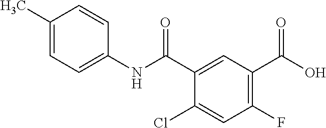 [SMILES: Cc1ccc(NC(=O)c2cc(C(=O)O)c(F)cc2Cl)cc1]

Using an analogous method as described for Intermediate 14, methyl 4-chloro-2-fluoro-5-[(4-methylphenyl)carbamoyl]benzoate (295 mg, 917 μmol; Intermediate 25) was used as the starting material. 272 mg (96% yield) of the title compound were prepared.

LC-MS (Method 4): Rₜ=1.03 min; MS (ESIpos): m/z=308 [M+H]⁺

### Intermediate 27

### 5-bromo-2-chloro-N-[4-(difluoromethoxy)phenyl]-4-fluorobenzamide

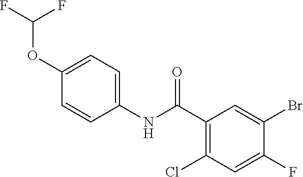 [SMILES: O=C(Nc1ccc(OC(F)F)cc1)c1cc(Br)c(F)cc1Cl]

Using an analogous method as described for Intermediate 9, 5-bromo-2-chloro-4-fluorobenzoic acid (502 mg, 1.98 mmol; Intermediate 3) and 4-(difluoromethoxy)aniline (630 mg, 3.96 mmol) were used as the starting material. 730 mg (99% purity, 92% yield) of the title compound were prepared.

LC-MS (Method 4): Rₜ=1.28 min; MS (ESIpos): m/z=396 [M+H]⁺

¹H-NMR (400 MHz, DMSO-d6) δ[ppm]: 1.232 (1.35), 2.518 (1.28), 2.523 (0.92), 6.991 (6.27), 7.176 (14.29), 7.181 (12.25), 7.186 (3.69), 7.199 (3.80), 7.204 (12.55), 7.212 (1.21), 7.362 (6.21), 7.701 (1.46), 7.710 (16.00), 7.715 (4.37), 7.727 (4.34), 7.732 (14.13), 7.740 (1.29), 7.802 (12.21), 7.823 (12.49), 8.058 (11.67), 8.076 (12.07), 10.652 (7.86).

### Intermediate 28

### methyl 4-chloro-5-{[4-(difluoromethoxy)phenyl]carbamoyl}-2-fluorobenzoate

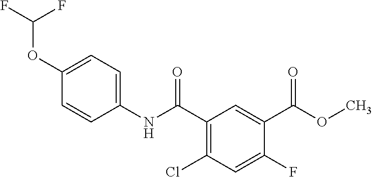 [SMILES: COC(=O)c1cc(C(=O)Nc2ccc(OC(F)F)cc2)c(Cl)cc1F]

Using an analogous method as described for Intermediate 10, 5-bromo-2-chloro-N-[4-(difluoromethoxy)phenyl]-4-fluorobenzamide (730 mg, 1.85 mmol; Intermediate 27) was used as the starting material and 1,1′-BIS(DIPHENYLPHOSPHINO)FERROCENE-PALLADIUM(II)DICHLORIDE DICHLOROMETHANE COMPLEX:(151 mg, 185 μmol; CAS-RN:[95464-05-4]) as catalyst. 533 mg (77% yield) of the title compound were prepared after purification using flash chromatography.

LC-MS (Method 4): Rₜ=1.22 min; MS (ESIpos): m/z=374 [M+H]⁺

### Intermediate 29

### 4-chloro-5-{[4-(difluoromethoxy)phenyl]carbamoyl}-2-fluorobenzoic acid

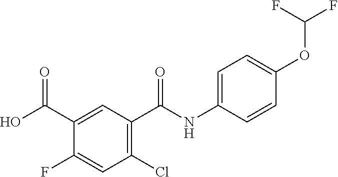 [SMILES: O=C(O)c1cc(C(=O)Nc2ccc(OC(F)F)cc2)c(Cl)cc1F]

Using an analogous method as described for Intermediate 14, methyl methyl 4-chloro-5-{[4-(difluoromethoxy)phenyl]carbamoyl}-2-fluorobenzoate (533 mg, 1.43 mmol; Intermediate 28) was used as the starting material. 355 mg (86% purity, 60% yield) of the title compound were prepared.

LC-MS (Method 4): Rt=1.03 min; MS (ESIpos): m/z=360 [M+H]⁺

¹H-NMR (400 MHz, DMSO-d6) δ[ppm]: 0.851 (0.54), 1.154 (1.64), 1.172 (3.62), 1.190 (1.89), 1.231 (2.01), 1.907 (6.80), 1.987 (7.18), 2.332 (0.61), 2.518 (2.30), 2.523 (1.53), 3.999 (0.47), 4.017 (1.48), 4.034 (1.44), 4.052 (0.47), 6.981 (1.10), 6.993 (6.93), 7.167 (3.50), 7.179 (16.00), 7.183 (13.54), 7.200 (4.16), 7.205 (12.62), 7.213 (1.30), 7.352 (0.93), 7.364 (5.95), 7.587 (0.41), 7.674 (0.89), 7.685 (2.16), 7.691 (0.93), 7.696 (1.06), 7.701 (1.87), 7.707 (3.30), 7.715 (15.98), 7.720 (4.77), 7.732 (4.69), 7.737 (14.44), 7.746 (10.66), 7.772 (8.99), 8.000 (1.01), 8.018 (1.05), 8.037 (9.30), 8.056 (9.15), 8.376 (0.42), 8.394 (0.41), 10.539 (0.49), 10.577 (0.42), 10.679 (9.61), 13.703 (0.45).

### Intermediate 30

### 5-bromo-2-chloro-4-fluoro-N-(2-methylphenyl)benzamide

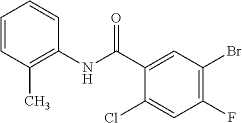 [SMILES: Cc1ccccc1NC(=O)c1cc(Br)c(F)cc1Cl]

Using an analogous method as described for Intermediate 9, 5-bromo-2-chloro-4-fluorobenzoic acid (400 mg, 1.58 mmol; Intermediate 3) and 2-methylaniline (203 mg, 1.89 mmol) were used as the starting material. 280 mg (77% purity, 40% yield) of the title compound were prepared.

LC-MS (Method 4): Rt=1.26 min; MS (ESIpos): m/z=344 [M+H]⁺

¹H-NMR (400 MHz, CHLOROFORM-d) δ [ppm]: 1.254 (0.84), 2.169 (0.56), 2.346 (14.92), 2.799 (2.10), 2.885 (0.67), 3.124 (0.65), 7.135 (0.57), 7.156 (1.43), 7.173 (1.09), 7.239 (1.71), 7.260 (16.00), 7.279 (3.54), 7.297 (0.57), 7.800 (0.63), 7.928 (1.44), 7.948 (1.32), 8.092 (2.23), 8.111 (2.24).

### Intermediate 31

### methyl 4-chloro-2-fluoro-5-[(2-methylphenyl)carbamoyl]benzoate

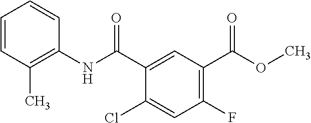 [SMILES: COC(=O)c1cc(C(=O)Nc2ccccc2C)c(Cl)cc1F]

Using an analogous method as described for Intermediate 10, 5-bromo-2-chloro-4-fluoro-N-(2-methylphenyl)benzamide (280 mg, 817 μmol: Intermediate 30) was used as the starting material and 1,1′-BIS(DIPHENYLPHOSPHINO)FERROCENE-PALLADIUM(II)DICHLORIDE DICHLOROMETHANE COMPLEX (66.7 mg, 81.7 μmol; CAS-RN:[95464-05-4]) as catalyst. 380 mg (75% purity, 108% yield) of the title compound were prepared after purification using flash chromatography.

LC-MS (Method 4): Rt=1.11 min; MS (ESIpos): m/z=322 [M+H]+

¹H-NMR (400 MHz, CHLOROFORM-d) δ [ppm]: 0.889 (0.52), 1.262 (1.10), 1.268 (0.73), 1.275 (0.64), 1.317 (0.51), 2.207 (0.42), 2.211 (0.50), 2.330 (0.51), 2.360 (13.41), 2.884 (0.97), 3.151 (0.99), 3.929 (2.01), 3.940 (1.15), 3.955 (0.43), 3.968 (16.00), 3.993 (0.52), 3.997 (0.61), 4.024 (0.48), 4.086 (0.62), 7.150 (0.53), 7.169 (1.33), 7.187 (1.02), 7.241 (0.41), 7.253 (1.60), 7.293 (1.23), 7.312 (2.57), 7.337 (2.06), 7.709 (0.73), 7.946 (1.49), 7.967 (1.30), 8.446 (1.96), 8.465 (1.96).

### Intermediate 32

### 4-chloro-2-fluoro-5-[(2-methylphenyl)carbamoyl]benzoic acid

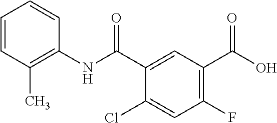 [SMILES: Cc1ccccc1NC(=O)c1cc(C(=O)O)c(F)cc1Cl]

Using an analogous method as described for Intermediate 14, methyl 4-chloro-2-fluoro-5-[(2-methylphenyl)carbamoyl]benzoate (380 mg, 1.18 mmol; Intermediate 31) was used as the starting material. 355 mg (98% yield) of the title compound were prepared.

LC-MS (Method 4): Rt=0.95 min; MS (ESIpos): m/z=308 [M+H]+

### Intermediate 33

### 5-bromo-2-chloro-4-fluoro-N-phenylbenzamide

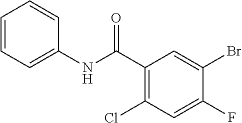 [SMILES: O=C(Nc1ccccc1)c1cc(Br)c(F)cc1Cl]

Using an analogous method as described for Intermediate 9, 5-bromo-2-chloro-4-fluorobenzoic acid 350 mg, 1.38 mmol; CAS-RN: [1204219-98-6]; Intermediate 3) and aniline (150 μl, 1.7 mmol; CAS-RN:[62-53-3]) were used as the starting material. 423 mg (100% purity, 93% yield) of the title compound were prepared.

LC-MS (Method 4): Rₜ=1.22 min; MS (ESIpos): m/z=328 [M+H]⁺

### Intermediate 34

### methyl 4-chloro-2-fluoro-5-(phenylcarbamoyl)benzoate

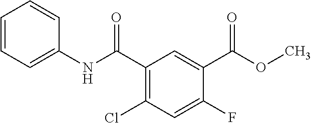 [SMILES: COC(=O)c1cc(C(=O)Nc2ccccc2)c(Cl)cc1F]

Using an analogous method as described for Intermediate 10, 5-bromo-2-chloro-4-fluoro-N-phenylbenzamide (Int. 33; 420 mg, 1.28 mmol) was used as the starting material and 1,1′-BIS(DIPHENYLPHOSPHINO)FERROCENE-PALLADIUM(II)DICHLORIDE DICHLOROMETHANE COMPLEX: (104 mg, 128 μmol; CAS-RN: [95464-05-4]) as catalyst. 264 mg (67% yield) of the title compound were prepared after purification using flash chromatography.

LC-MS (Method 4): Rₜ=1.11 min; MS (ESIpos): m/z=308 [M+H]⁺

### Intermediate 35

### 4-chloro-2-fluoro-5-(phenylcarbamoyl)benzoic acid

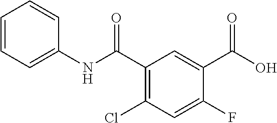 [SMILES: O=C(O)c1cc(C(=O)Nc2ccccc2)c(Cl)cc1F]

Using an analogous method as described for Intermediate 14, methyl 4-chloro-2-fluoro-5-(phenylcarbamoyl)benzoate (263 mg, 855 μmol; Intermediate 34) was used as the starting material. 79.3 mg (100% purity, 32% yield) of the title compound were prepared.

LC-MS (Method 4): Rₜ=0.96 min; MS (ESIpos): m/z=293 [M+H]⁺

### Intermediate 36

### 5-bromo-2-chloro-4-fluoro-N-[2-methyl-4-(trifluoromethyl)phenyl]benzamide

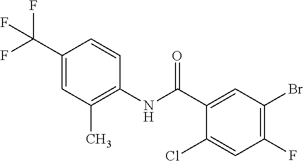 [SMILES: Cc1cc(C(F)(F)F)ccc1NC(=O)c1cc(Br)c(F)cc1Cl]

Using an analogous method as described for Intermediate 9, 2-methyl-4-(trifluoromethyl)aniline (350 mg, 2.00 mmol) and 5-bromo-2-chloro-4-fluorobenzoic acid (608 mg, 2.40 mmol; Intermediate 3) were used as the starting material. 563 mg (69% yield) of the title compound were prepared.

LC-MS (Method 4): Rt=1.40 min; MS (ESIpos): m/z=410 [M+H]+

¹H-NMR (400 MHz, DMSO-d6) δ[ppm]: 1.232 (0.61), 2.074 (2.04), 2.327 (0.63), 2.364 (16.00), 2.518 (2.79), 2.523 (2.00), 2.669 (0.45), 7.596 (1.18), 7.618 (1.42), 7.655 (2.82), 7.767 (1.86), 7.788 (1.49), 7.815 (2.75), 7.837 (2.78), 8.115 (2.96), 8.133 (3.00), 10.268 (2.81).

### Intermediate 37

### methyl 4-chloro-2-fluoro-5-{[2-methyl-4-(trifluoromethyl)phenyl]carbamoyl}benzoate

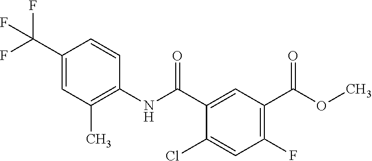 [SMILES: COC(=O)c1cc(C(=O)Nc2ccc(C(F)(F)F)cc2C)c(Cl)cc1F]

Using an analogous method as described for Intermediate 10, 5-bromo-2-chloro-4-fluoro-N-[2-methyl-4-(trifluoromethyl)phenyl]benzamide (12.5 g, 30.4 mmol; Intermediate 36) was used as the starting material and 1,1′-BIS(DIPHENYLPHOSPHINO)FERROCENE-PALLADIUM(II)DICHLORIDE DICHLOROMETHANE COMPLEX:(4.97 g, 6.09 mmol; CAS-RN: [95464-05-4]) as catalyst. 3.94 g (90% purity, 30% yield) of the title compound were prepared after purification using flash chromatography.

LC-MS (Method 4): Rₜ=1.31 min; MS (ESIpos): m/z=390 [M+H]⁺

### Intermediate 38

### 4-chloro-2-fluoro-5-{[2-methyl-4-(trifluoromethyl)phenyl]carbamoyl}benzoic acid

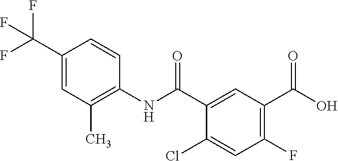 [SMILES: Cc1cc(C(F)(F)F)ccc1NC(=O)c1cc(C(=O)O)c(F)cc1Cl]

Using an analogous method as described for Intermediate 14, methyl 4-chloro-2-fluoro-5-{[2-methyl-4-(trifluoromethyl)phenyl]carbamoyl}benzoate (1.59 g, 4.08 mmol; Intermediate 37) was used as the starting material. 1.35 g (90% purity, 80% yield) of the title compound were prepared.

LC-MS (Method 4): Rₜ=1.17 min; MS (ESIpos): m/z=376 [M+H]⁺

¹H-NMR (400 MHz, DMSO-d6) δ[ppm]: 1.352 (1.72), 2.327 (0.51), 2.332 (0.43), 2.337 (0.68), 2.344 (2.70), 2.364 (16.00), 2.401 (0.75), 2.518 (1.40), 2.523 (0.94), 3.919 (0.42), 4.076 (0.45), 7.593 (1.34), 7.614 (1.51), 7.660 (2.82), 7.750 (2.32), 7.776 (3.36), 7.794 (1.33), 8.109 (2.54), 8.128 (2.40), 10.305 (3.65).

### Intermediate 39

### 5-phenoxypyrazin-2-amine

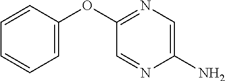 [SMILES: Nc1cnc(Oc2ccccc2)cn1]

Phenol (1.78 g, 18.97 μmol, 1.67 mL, 1.1 eq), 5-bromopyrazin-2-amine (3 g, 17.24 mmol, 1 eq), N,N-dimethylglycine (177.79 mg, 1.72 mmol, 0.1 eq), and Cesium Carbonate (7.86 g, 24.14 mmol, 1.4 eq) were suspended in 1,4-dioxane (25 mL). The resulting suspension was degassed in vacuo and purged with N₂ 3×. Copper iodide (328.36 mg, 1.72 mmol, 0.1 eq) was added in one portion, and the resulting mixture was stirred at 115° C. for 4 h. Water (30 mL) was added to quench the reaction, and the resulting mixture was extracted 4× with ethyl acetate (60 mL). The combined organic layers were washed 4× with brine (20 mL), dried over sodium sulfate, filtered, and the solvent removed in vacuo. The resulting residue was purified on a normal phase column (SiO2, isocratic Petroleum ether/Ethyl acetate 10/1) to provide 2.24 g (11.97 mmol, 69.4% yield) of the title compound as an orange solid.

LCMS (method 7): Rt=0.478 min; MS (ESIpos): m/z=188.2 [M+H]⁺

1H NMR (400 MHz, DMSO-d6) delta [ppm]: 6.235, 6.954, 6.974, 7.089, 7.319, 7.339, 7.358, 7.563, 7.566, 7.826, 7.828

### Intermediate 40

### tert-butyl (3-methyl-5-(oxetan-3-yl)pyridin-2-yl)carbamate

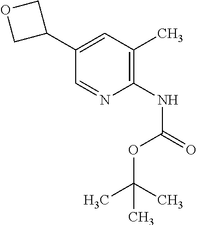 [SMILES: Cc1cc(C2COC2)cnc1NC(=O)OC(C)(C)C]

To a 40 mL vial equipped with a magnetic stir bar was added tert-butyl N-(5-bromo-3-methyl-2-pyridyl)carbamate (689.2 mg, 2.4 mmol, 1 eq), 3-bromooxetane (493.1 mg, 3.60 mmol, 1.5 eq), Ir[dF(CF3)ppy]2(dtbpy)(PF6) (26.9 mg, 24.0 μmol, 0.01 eq), NiCl2-dtbbpy (14.3 mg, 36.0 μmol, 0.015 eq), TTMSS (596.8 mg, 740.4 μL, 2.40 mmol, 1 eq), and sodium carbonate (508.8 mg, 4.80 mmol, 2 eq). The reaction was suspended in dimethoxyethane (24 mL), sealed, and evacuated and backfilled with N2 3×. The reaction was stirred in a cooling water bath set to 25° C. while under irradiation from a 50×4 W blue LED lamp placed 3 cm away for 14 h. Water (40 mL) was added, and the mixture was extracted 3× with ethyl acetate (50 mL). The combined organic layers were washed 2× with brine (50 mL), dried over sodium sulfate, filtered, and concentrated in vacuo to give a residue. The resulting residue was purified by flash normal phase chromatography on SiO2 (Teledyne Isco 25 g SepaFlash silica flash column, gradient Petroleum ether—Ethyl acetate 0-100% at 80 mL/min) to provide 2.2 g (8.32 mmol, 86.7% yield) of the title compound as a white solid.

1H NMR (400 MHz, DMSO-d6) δ[ppm]: 1.440, 4.202, 4.224, 4.243, 4.260, 4.280, 4.597, 4.613, 4.629, 4.910, 4.925, 4.931, 4.946, 7.759, 7.763, 9.072

### Intermediate 41

### 3-methyl-5-(oxetan-3-yl)pyridin-2-amine

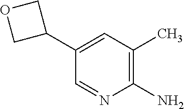

To a solution of Intermediate 40 (tert-butyl N-[3-methyl-5-(oxetan-3-yl)-2-pyridyl]carbamate, (1 g, 3.78 mmol, 1 eq) in DCM (10 mL) was added trifluoroacetic acid (3.08 g, 2 mL, 27.01 mmol, 7.14 eq). The resulting mixture was stirred at 25° C. for 12 h, at which point the solution was concentrated in vacuo. To the resulting residue was added NH3-water (1.5 mL), and the resulting solution was extracted 3× with ethyl acetate (5 mL). The combined organic layers were washed 3× with brine (5 mL), dried over sodium sulfate, filtered, and concentrated in vacuo. The resulting residue was purified by normal phase column chromatography on SiO2 (isocratic, DCM:MeOH 10:1) to provide 320 mg (1.95 mmol, 51.5% yield) of the title compound as a yellow oil.

LCMS (method 7, 5-95CD_1 min): Rt=0.694 min; MS (ESIpos): m/z=165.2 [M+H]⁺

1H NMR (400 MHz, DMSO-d6) delta [ppm]: 1.986, 3.343, 4.105, 4.126, 4.143, 4.161, 4.164, 4.546, 4.561, 4.577, 4.842, 4.857, 4.862, 4.877, 7.168, 7.775, 7.847

### Intermediate 42

### methyl 4-chloro-2-fluoro-5-((3-methyl-5-(oxetan-3-yl)pyridin-2-yl)carbamoyl)benzoate

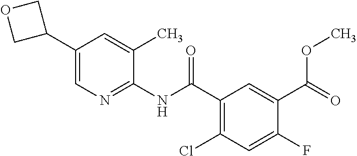 [SMILES: COC(=O)c1cc(C(=O)Nc2ncc(C3COC3)cc2C)c(Cl)cc1F]

To a solution of methyl 4-chloro-5-chlorocarbonyl-2-fluoro-benzoate (250 mg, 995.9 μmol, 1 eq, Intermediate 88), in DCM (1.5 mL) was added 3-methyl-5-(oxetan-3-yl)pyridine-2-amine (245.3 mg, 1.49 mmol, 1.5 eq) and Et₃N (503.9 mg, 693.1 μL, 4.98 mmol, 5 eq). The resulting mixture was stirred at 25° C. for 16 h. The resulting yellow suspension was poured into water (10 mL), and extracted 3× with ethyl acetate (10 mL). The combined organic layers were washed 3× with brine (10 mL), dried over sodium sulfate, and concentrated in vacuo. The resulting residue was purified by flash column chromatography (SiO₂, gradient DCM—MeOH 100:1-97:3) to provide 60 mg (120.4 μmol, 12.1% yield, 76% purity) as a yellow solid.

LCMS (method 7): Rt=0.421 min; MS (ESIpos): m/z=378.8 [M+]+

### Intermediate 43

### 4-chloro-2-fluoro-5-((3-methyl-5-(oxetan-3-yl)pyridin-2-yl)carbamoyl)benzoic acid

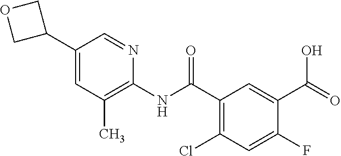 [SMILES: Cc1cc(C2COC2)cnc1NC(=O)c1cc(C(=O)O)c(F)cc1Cl]

To a solution of methyl 4-chloro-2-fluoro-5-[[3-methyl-5-(oxetan-3-yl)-2-pyridyl]carbamoyl]-benzoate (60 mg, 158.4 μmol, 1 eq; Intermediate 42) in a mixture of MeOH (1 mL) and THF (1 mL), then a solution of LiOH-water (13.29 mg, 316.8 μmol, 2 eq) in water (1 mL) was added. The resulting mixture was stirred at 25° C. for 1 h. The resulting yellow solution was adjusted to pH 3 by the addition of 3 M aq HCl, and extracted 3× with ethyl acetate (10 mL). The combined organic layers were dried over sodium sulfate, filtered, and concentrate in vacuo. The resulting residue was used without further purification, representing 40 mg (109.7 μmol) of the title compound as a yellow solid.

LCMS (method 7): Rt=0.369 min; MS (ESIneg): m/z=364.8 [M−]⁻

1H NMR (400 MHz, DMSO-d6) δ 10.71 (s, 1H), 8.24 (br s, 1H), 8.02 (d, J=7.6 Hz, 1H), 7.88 (d, J=2.0 Hz, 1H), 7.76-7.69 (m, 1H), 4.95 (dd, J=6.0, 8.4 Hz, 2H), 4.65 (t, J=6.4 Hz, 2H), 4.34-4.25 (m, 1H), 2.32 (s, 3H)

### Intermediate 44

### methyl 3,4-dichloro-5-((5-morpholinopyridin-2-yl)carbamoyl)benzoate

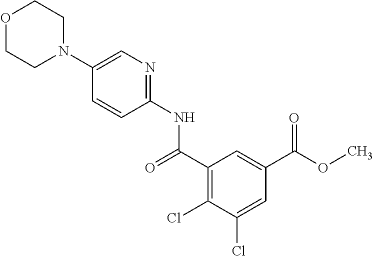 [SMILES: COC(=O)c1cc(Cl)c(Cl)c(C(=O)Nc2ccc(N3CCOCC3)cn2)c1]

To a solution of 2,3-dichloro-5-methoxycarbonyl-benzoic acid (500 mg, 2.01 mmol, 1 eq), 5-morpholinopyridin-2-amine (431.8 mg, 2.41 mmol, 1.2 eq) in pyridine (1 mL) was added a 50% solution of T3P (3.83 g, 3.58 mL, 6.02 mmol, 3 eq) in DMF. The resulting mixture was stirred at 20° C. for 4 h, at which time the reaction mixture was diluted with water (15 mL), and subsequently extracted 3× with ethyl acetate (20 mL). The combined organic layers were washed 3× with brine (12 mL), dried over sodium sulfate, filtered, and concentrated in vacuo. The resulting residue was purified by flash column chromatography (SiO2, gradient Petroleum ether—ethyl acetate 2:1-1:1) to provide 700 mg (1.71 mmol, 84.99% yield) of the title compound as a white solid.

LCMS (method 7): Rₜ=0.536 min; MS (ESIpos): m/z=412.1 [M+H]⁺

¹H NMR (400 MHz, CDCl₃) δ 3.159, 3.171, 3.175, 3.183, 3.887, 3.899, 3.911, 3.957, 7.475, 7.482, 7.498, 7.505, 7.750, 7.757, 8.214, 8.219, 8.239, 8.244, 8.418, 8.441, 9.753

### Intermediate 45

### 3,4-dichloro-5-((5-morpholinopyridin-2-yl)carbamoyl)benzoic acid

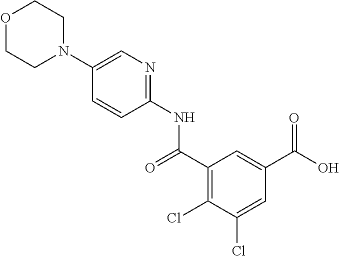 [SMILES: O=C(O)c1cc(Cl)c(Cl)c(C(=O)Nc2ccc(N3CCOCC3)cn2)c1]

To a solution of methyl 3,4-dichloro-5-[(5-morpholino-2-pyridyl)carbamoyl]benzoate (300 mg, 731.3 μmol, 1 eq; Intermediate 44) in a 5:1 mixture of THF (3 mL) and water (0.6 mL) was added LiOH-water (61.4 mg, 1.46 mmol, 3 eq). The mixture was stirred 30° C. for 2 h, at which point the reaction mixture was diluted with water (5 mL) and adjusted to pH 3-4 by the addition of a 2 M aq HCl solution. The resulting suspension was filtered, and the filtrate concentrated in vacuo to provide 250 mg (630.9 μmol, 86.3% yield) of the title compound as a white solid.

LCMS (method 7): Rₜ=0.457 min; MS (ESIneg): m/z=396.0 [M−]⁻

### Intermediate 46

### methyl 3,4-dichloro-5-vinylbenzoate

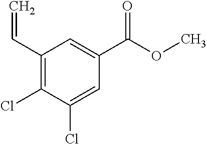

To a solution of methyl 3-bromo-4,5-dichloro-benzoate (5 g, 17.6 mmol, 1 eq), potassium vinyl trifluoro-boronate (3.54 g, 26.4 mmol, 1.5 eq) and potassium carbonate (7.30 g, 52.8 mmol, 3 eq) in 1,4-dioxane (50 mL) and water (10 mL) was added [1,1′-Bis-(diphenylphosphino)-ferrocen]-dichloro-palladium(II) (1.29 g, 1.76 mmol, 0.1 eq) under N2. The resulting mixture was stirred at 80° C. for 12 h, at which point saturated aq ammonium chloride (80 mL) was added, and the mixture extracted 3× with ethyl acetate (100 mL). The combined organic layers were washed 3× with brine (80 mL), dried over sodium sulfate, filtered, and concentrated in vacuo.

The resulting residue was purified by flash column chromatography (SiO2, gradient petroleum ether/ethyl acetate 1/0-100/1) to provide 2.7 g (11.68 mmol, 66.3% yield) of the title compound as a white solid.

¹H NMR (400 MHz, CDCl3) delta [ppm]: 1.575, 3.947, 5.503, 5.531, 5.836, 5.880, 7.071, 7.098, 7.114, 7.142, 8.030, 8.035, 8.122, 8.127

### Intermediate 47

### methyl 3,4-dichloro-5-formylbenzoate

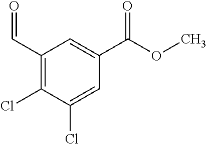

To a solution of methyl 3,4-dichloro-5-vinyl-benzoate (1 g, 4.33 mmol, 1 eq; Intermediate 46) in THF (40 mL) and water (13 mL) was added sodium periodate (1.85 g, 8.66 mmol, 479.6 μL, 2 eq) and osmium tetroxide (55.01 mg, 11.23 μL, 216.4 μmol, 0.05 eq) at 0° C. under N2. The mixture was stirred at 25° C. for 2 h, at which point the reaction mixture was quenched by the addition of an aqueous saturated solution of sodium sulfite (30 mL), and the resulting mixture extracted 3× with ethyl acetate (30 mL). The combined organic layers were washed 4× with brine, dried over sodium sulfate, filtered, and concentrated in vacuo. The residue was used without further purification, providing 1 g (3.00 mmol, 69.4% yield, 70% purity) of the title compound as a white solid.

¹H NMR (400 MHz, CDCl3) delta [ppm]: 1.776, 3.972, 7.270, 8.359, 8.364, 8.466, 10.497

### Intermediate 48

### 2,3-dichloro-5-(methoxycarbonyl)benzoic acid

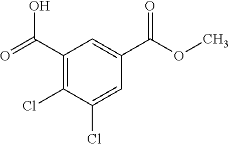

To a solution of methyl 3,4-dichloro-5-formyl-benzoate (1 g, 3.00 mmol, 70% purity, 1 eq; Intermediate 47) in acetone (30 mL) was added potassium permanganate (1.90 g, 12.0 mmol, 4 eq). The mixture was stirred at 25° C. for 3 h. The resulting mixture was diluted with water (20 mL) and saturated aqueous sodium thiosulfate (10 mL). After the resulting mixture was adjusted to pH 2-3 by the addition of sulfuric acid. The mixture was extracted 3× with ethyl acetate (30 mL), the combined organic layers were washed 3× with saturated sodium thiosulfate (20 mL) and 3× with brine (20 mL), dried over sodium sulfate, filtered, and concentrated in vacuo. The resulting compound was used without further purification, providing 800 mg of the title compound as a yellow solid.

¹H NMR (400 MHz, DMSO-d6) delta [ppm]: 3.333, 3.897, 3.912, 8.196, 8.201, 8.221, 8.226, 13.987, 13.996, 14.012, 14.019, 14.031, 14.039, 14.043, 14.056

### Intermediate 49

### 2-(m-tolyl)benzo[d]oxazol-5-amine

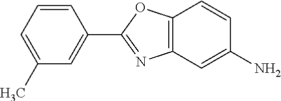 [SMILES: Cc1cccc(-c2nc3cc(N)ccc3o2)c1]

To a solution of 2-(m-tolyl)-5-nitro-1,3-benzoxazole (500 mg, 1.97 mmol, 1 eq) in a mixture of water (3 mL) and ethanol (10 mL) was added iron (549.1 mg, 9.83 mmol, 5 eq) and ammonium chloride (841.6 mg, 15.7 mmol, 8 eq). The resulting mixture was stirred at 80° C. for 12 hr. The resulting solution was filtered, and the filtrate adjusted to pH 7-8 by the addition of saturated aqueous sodium bicarbonate. The resulting solution was extracted 3× with ethyl acetate (10 mL), and the combined organic layers washed 2× with brine (10 mL), dried over sodium sulfate, filtered, and concentrated in vacuo. The resulting residue was purified by flash chromatography (SiO2, isocratic, petroleum ether/ethyl acetate 5/1) to provide 375 mg (1.67 mmol, 85% yield) or the title compound as a white solid.

¹H NMR (400 MHz, CDCl3) delta [ppm]: 1.589, 2.382, 3.658, 6.632, 6.654, 6.976, 6.981, 7.931, 7.950, 7.991

### Intermediate 50

### 4-bromo-N1-(3,4-difluorobenzyl)-6-fluoro-N3-phenylisophthalamide

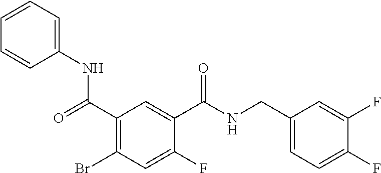 [SMILES: O=C(NCc1ccc(F)c(F)c1)c1cc(C(=O)Nc2ccccc2)c(Br)cc1F]

To a solution of 2-bromo-5-[(3,4-difluorophenyl)methylcarbamoyl]-4-fluoro-benzoic acid (200 mg, 515.3 μmol, 1 eq, Intermediate 97) and aniline (72.0 mg, 70.6 μL, 772.9 μmol, 1.5 eq) in pyridine (2 mL) was added a 50% solution of T3P (983.7 mg, 919.4 μL, 1.55 mmol, 3 eq) in DMF. The resulting mixture was stirred at 25° C. for 2 h, at which point saturated aqueous ammonium chloride (5 mL) was added, and the solution extracted 3× with Ethyl acetate (5 mL). The combined organic layers were washed 3× with brine (5 mL), dried over sodium sulfate, filtered, and concentrated in vacuo to provide a residue. The residue was purified by flash column chromatography (SiO2, Gradient: Petroleum ether/ethyl acetate 10:1-1:1) to provide 140 mg (272.0 μmol, 53% yield, 90% purity) of the title compound as a red solid.

LCMS (method 7): Rₜ=0.633 min; MS (ESIpos): m/z=464.9 [M+H]⁺

### Intermediate 51

### N1-(3,4-difluorobenzyl)-6-fluoro-N3-phenyl-4-((trimethylsilyl)ethynyl)isophthalamide

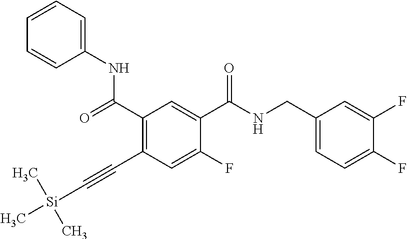 [SMILES: C[Si](C)(C)C#Cc1cc(F)c(C(=O)NCc2ccc(F)c(F)c2)cc1C(=O)Nc1ccccc1]

To a solution of 4-bromo-N1-[(3,4-difluorophenyl)methyl]-6-fluoro-N3-phenyl-benzene-1,3-dicarboxamide (130 mg, 280.6 μmol, 1 eq; Intermediate 50) and ethynyl(trimethyl)silane (137.8 mg, 194.4 μL, 1.40 mmol, 5 eq) in DMF (2 mL) was added Copper iodide (5.34 mg, 28.1 μmol, 0.1 eq) and Pd(PPh3)₂Cl2 (19.70 mg, 28.1 μmol, 0.1 eq), and Et3N (1.45 g, 2 mL, 14.4 mmol, 51.2 eq). The resulting mixture was stirred at 90° C. for 2 h, at which point a saturated aqueous solution of ammonium chloride (5 mL) was added, and the solution extracted 3× with ethyl acetate (5 mL). The combined organic layers were washed 3× with brine (5 mL), dried over sodium sulfate, filtered, and concentrated in vacuo. The resulting residue was used without further purification, providing 65 mg of a crude mixture of the title compound as a brown solid.

### Intermediate 52

### 5-bromo-3-methylpyridin-2-amine

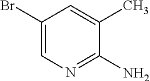

To a solution of 5-bromo-3-methyl-2-nitro-pyridine (3.00 g, 13.8 mmol, 1 eq) in a mixture of ethanol (27 mL) and water (9 mL) was added iron (3.86 g, 69.1 mmol, 5 eq) and ammonium chloride (7.39 g, 138.2 mmol, 10 eq). The resulting mixture was stirred at 70° C. for 12 h, at which time the mixture was diluted with water (30 mL) and extracted 3× with ethyl acetate (30 mL). The combined organic layers were washed with water (90 mL) and 3× with brine (30 mL), dried over sodium sulfate, filtered, and concentrated in vacuo. The resulting residue was purified by flash chromatography (ISCO 40 g SepaFlash Silica Flash Column, Gradient: Petroleum ether-ethyl acetate, 0%-30% @ 50 mL/min), to provide 2.3 g (11.7 mmol, 84.5% yield, 95% purity) of the title compound as a yellow solid.

LCMS (method 7): Rₜ=0.195 min; MS (ESIpos): m/z=186.9 [M+]⁺

¹H NMR (400 MHz, DMSO-d6) delta [ppm]: 1.153, 1.171, 1.189, 3.999, 4.017, 4.034, 4.052, 5.921, 7.403, 7.406, 7.821, 7.827

### Intermediate 53

### 3-methyl-5-morpholinopyridin-2-amine

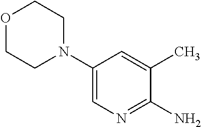

To a solution of 5-bromo-3-methyl-pyridin-2-amine (1.1 g, 5.88 mmol, 1 eq; Intermediate 52) and 1 M LiHMDS solution in THF (14.7 mL, 14.7 mmol, 2.5 eq) in THF (5 mL) was added morpholine (2.56 g, 2.59 mL, 29.4 mmol, 5 eq). The suspension was evacuated and backfilled with N2 3×, and Pd2(dba)3 (538.6 mg, 588.1 μmol, 0.1 eq) and XPhos (560.7 mg, 1.18 mmol, 0.2 eq) were added in one portion. The resulting mixture was stirred at 40° C. for 2 h, at which point saturated aqueous ammonium chloride was added, followed by dilution with water (15 mL). The resulting mixture was extracted 3× with ethyl acetate (15 mL). The combined organic layers were washed 3× with brine (15 mL), dried over sodium sulfate, and concentrated in vacuo. The resulting residue was purified by flash column chromatography (SiO2, ISCO 40 g SepaFlash Silica Flash Column, Gradient: Petroleum ether—ethyl acetate, 0%-100% at 100 mL/min) to provide 560 mg (2.90 mmol, 49.3% yield) of the title compound as a red solid.

LCMS (method 7): Rₜ=0.598 min; MS (ESIpos): m/z=235.2 [M+MeCN]⁺

### Intermediate 54

### methyl 3,4-dichloro-5-(pyrazin-2-ylcarbamoyl)benzoate

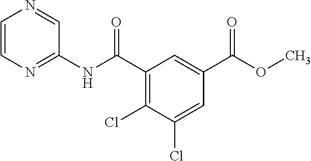 [SMILES: COC(=O)c1cc(Cl)c(Cl)c(C(=O)Nc2cnccn2)c1]

To a solution of 2,3-dichloro-5-methoxycarbonyl-benzoic acid (800 mg, 3.21 mmol, 1 eq; Intermediate 53) and pyrazin-2-amine (458.2 mg, 4.82 mmol, 1.5 eq) in pyridine (8 mL) was added a 50% solution of T3P in DMF (6.13 g, 5.73 mL, 9.64 mmol, 3 eq). The resulting mixture was stirred at 25° C. for 12 h, at which point the mixture was diluted with water (20 mL) and extracted 3× with ethyl acetate (20 mL). The combined organic layers were washed 4× with brine (15 mL), dried over sodium sulfate, filtered, and concentrated in vacuo. The resulting residue was purified by flash column chromatography (SiO2, Gradient: petroleum ether—ethyl acetate 3:1-2:1) to provide 550 mg (1.69 mmol, 5.25% yield) of the title compound as a white solid.

LCMS (method 7): Rₜ=0.596 min; MS (ESIpos): m/z=325.9 [M+]⁺

¹H NMR (400 MHz, DMSO-d6) delta [ppm]: 3.902, 8.116, 8.214, 8.219, 8.466, 9.420, 11.535

### Intermediate 55

### 3,4-dichloro-5-(pyrazin-2-ylcarbamoyl)benzoic acid

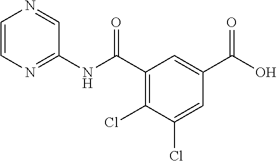 [SMILES: O=C(O)c1cc(Cl)c(Cl)c(C(=O)Nc2cnccn2)c1]

To a solution of methyl 3,4-dichloro-5-(pyrazin-2-ylcarbamoyl)benzoate (300 mg, 919.9 μmol, 1 eq; Intermediate 54) in a mixture of THF (3 mL) and water (0.6 mL) was added LiOH·water (77.2 mg, 1.84 mmol, 2 eq). The mixture was stirred at 25° C. for 1 h, at which point the reaction was diluted with water (5 mL) and the pH adjusted to 3-4 by the addition of an aqueous 2.5 M solution of HCl. The mixture was extracted 3× with ethyl acetate (10 mL), and the combined organic layers were washed 3× with brine (8 mL), dried over sodium sulfate, filtered, and concentrated in vacuo. The residue was used without further purification to provide 300 mg of crude title compound as a white solid.

LCMS (method 7): Rₜ=0.455 min; MS (ESIneg): m/z=311.8 [M−]⁻

### Intermediate 56

### methyl 4-amino-3-bromo-5-chlorobenzoate

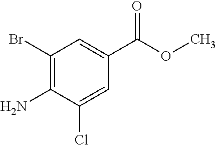

To a stirred solution of methyl 4-amino-3-chlorobenzoate (2.80 g, 15.1 mmol) in tetrahydrofuran (30 ml) was added N-bromosuccinimide (2.95 g, 16.6 mmol) in portions at 25° C. The reaction mixture was stirred at room temperature for 1 hour. The mixture was poured into ethyl acetate, and the organic layer was washed with 10% sodium thiosulfate, followed by 10% sodium carbonate, and brine. The organic layer was dried over anhydrous sodium sulfate and concentrated to dryness under vacuum. The residue was purified by flash chromatography (80 g, eluting with 8% ethyl acetate in petroleum ether) to afford methyl 4-amino-3-bromo-5-chlorobenzoate (3.80 g, 95% yield) as a light yellow solid.

¹H NMR (400 MHz, DMSO-d₆): δ [ppm]=7.89 (d, J=1.6 Hz, 1H), 7.77 (d, J=1.6 Hz, 1H), 6.32 (s, 2H), 3.79 (s, 3H).

### Intermediate 57

### 3-bromo-4-[(tert-butoxycarbonyl)amino]-5-chlorobenzoic acid

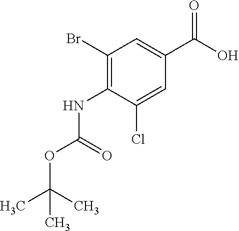 [SMILES: CC(C)(C)OC(=O)Nc1c(Cl)cc(C(=O)O)cc1Br]

To a mixture of methyl 4-amino-3-bromo-5-chlorobenzoate (2.00 g, 7.56 mmol, Intermediate 56 in tetrahydrofuran (30 ml) was added lithium bis(trimethylsilyl)amide (9.8 ml, 1.0 M in tetrahydrofuran, 9.8 mmol) dropwisely at −78° C. under nitrogen. Then the mixture was stirred at 0° C. for 30 minutes. The mixture was cooled to −78° C. and dropwise added a solution of di-tert-butyl dicarbonate (2.1 ml, 9.1 mmol) in tetrahydrofuran (10 ml) to the reaction mixture, the mixture was stirred at 25° C. for 2 hours. The mixture was poured into saturated ammonium chloride solution, the mixture was extracted with ethyl acetate. The organic phase was washed with brine, dried over anhydrous sodium sulfate, filtered and concentrated to give a residue. To the residue in tetrahydrofuran (20 ml) was added a solution of lithium hydroxide monohydrate in water ((19 ml, 2.0 M, 38 mmol) at 25° C. The mixture was stirred at 25° C. for 16 hours. The mixture was adjusted pH=5 with hydrochloric acid in water (1N), the mixture was extracted with ethyl acetate. The organic phase was dried over anhydrous sodium sulfate, filtered and concentrated to give 3-bromo-4-[(tert-butoxycarbonyl)amino]-5-chlorobenzoic acid (2.30 g, 87% yield) as yellow solid.

### Intermediate 58

### tert-butyl [4-(benzylcarbamoyl)-2-bromo-6-chlorophenyl]carbamate

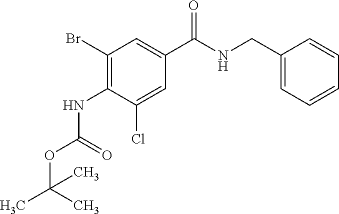 [SMILES: CC(C)(C)OC(=O)Nc1c(Cl)cc(C(=O)NCc2ccccc2)cc1Br]

To a solution of 3-bromo-4-[(tert-butoxycarbonyl)amino]-5-chlorobenzoic acid (2.30 g, 6.56 mmol, Intermediate 57) and 1-phenylmethanamine (844 mg, 7.87 mmol) in N,N-dimethylformamide (26 ml) were added N,N-diisopropylethylamine (2.3 ml, 13 mmol) and 1-[Bis(dimethylamino)methylene]-1H-1,2,3-triazolo[4,5-b]pyridinium 3-oxid hexafluorophosphate (3.24 g, 8.53 mmol) at room temperature. The reaction mixture was stirred at room temperature for 16 hours. The mixture was poured into water and extracted with ethyl acetate. The organic phase was washed with brine, dried over anhydrous sodium sulfate, filtered and concentrated to give a residue. The residue was triturated with ethyl acetate (20 ml) and dried over vacuum to afford tert-butyl [4-(benzylcarbamoyl)-2-bromo-6-chlorophenyl]carbamate (2.30 g, 80% yield) as white solid.

### Intermediate 59

### methyl 5-(benzylcarbamoyl)-2-[(tert-butoxycarbonyl)amino]-3-chlorobenzoate

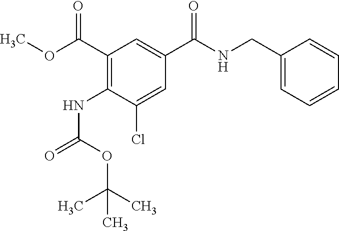 [SMILES: COC(=O)c1cc(C(=O)NCc2ccccc2)cc(Cl)c1NC(=O)OC(C)(C)C]

To a solution of tert-butyl [4-(benzylcarbamoyl)-2-bromo-6-chlorophenyl]carbamate (2.30 g, 5.23 mmol, Intermediate 58) in methanol (50 ml) were added [1,1-Bis(diphenylphosphino)ferrocene]dichloropalladium(II) (383 mg, 0.52 mmol) and trimethylamine (2.2 ml, 16 mmol) in one portion. The reaction mixture was stirred at 80° C. for 16 hours under carbon monoxide atmosphere. The mixture was poured into water and extracted with ethyl acetate. The organic phase was dried over anhydrous sodium sulfate, filtered and concentrated to give a residue. The residue was purified by silica gel chromatography (100-200 mesh silica gel, Petroleum ether/Ethyl acetate=100/1, 20/1, 10/1, 5/1, 3/1) to give methyl 5-(benzylcarbamoyl)-2-[(tert-butoxycarbonyl)amino]-3-chlorobenzoate (500 mg, 23% yield) as a yellow solid.

LC-MS (Method C): Rt=0.853 min; MS (ESIpos): m/z=419.2 [M+H]⁺.

¹H NMR (400 MHz, DMSO-d₆): δ [ppm]=9.28-9.24 (m, 2H), 8.17 (dd, J=7.6, 2.0 Hz, 2H), 7.35-7.30 (m, 5H), 7.27-7.22 (m, 1H), 4.47 (d, J=6.0 Hz, 2H), 3.79 (s, 3H), 1.43 (s, 9H).

### Intermediate 60

### methyl 2-amino-5-(benzylcarbamoyl)-3-chlorobenzoate

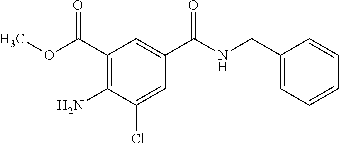 [SMILES: COC(=O)c1cc(C(=O)NCc2ccccc2)cc(Cl)c1N]

To a solution of methyl 5-(benzylcarbamoyl)-2-[(tert-butoxycarbonyl)amino]-3-chlorobenzoate (500 mg, 1.19 mmol, Intermediate 59) in dioxane (5 ml) was added hydrochloric acid in dioxane (5 ml, 4 M) at 20° C., the mixture was stirred at room temperature for 2 hours. The mixture was concentrated in reduced pressure to give methyl 2-amino-5-(benzylcarbamoyl)-3-chlorobenzoate (370 mg, 97% yield) as a gray solid.

LC-MS (Method C): Rt=0.921 min; MS (ESIpos): m/z=319.3 [M+H]⁺.

### Intermediate 61

### methyl 5-(benzylcarbamoyl)-2-bromo-3-chlorobenzoate

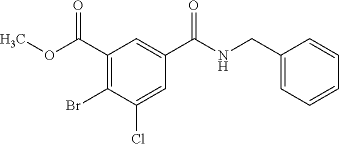 [SMILES: COC(=O)c1cc(C(=O)NCc2ccccc2)cc(Cl)c1Br]

To a solution of copper(II) bromide (252 mg, 1.13 mmol) in acetonitrile (6.0 ml) was added tert-butyl nitrite (0.15 ml, 1.2 mmol) dropwise at 0° C., then methyl 2-amino-5-(benzylcarbamoyl)-3-chlorobenzoate (300 mg, 0.94 mmol, Intermediate 60) was added the mixture at 0° C., the mixture was stirred at room temperature for 2 hours. The mixture was concentrated in reduced pressure to give a residue. The residue was purified by flash column chromatography (20 g, ethyl acetate in petroleum ether was 0%-50%) to give methyl 5-(benzylcarbamoyl)-2-bromo-3-chlorobenzoate (200 mg) as a yellow solid.

LC-MS (Method C): Rt=0.957 min; MS (ESIpos): m/z=384.2 [M+H]⁺.

### Intermediate 62

### 5-(benzylcarbamoyl)-2-bromo-3-chlorobenzoic acid

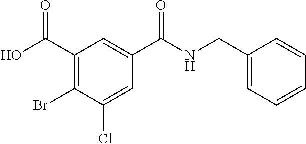 [SMILES: O=C(NCc1ccccc1)c1cc(Cl)c(Br)c(C(=O)O)c1]

A mixture of methyl 5-(benzylcarbamoyl)-2-bromo-3-chlorobenzoate (200 mg, 0.52 mmol, Intermediate 61 Intermediate 61) in tetrahydrofuran (4.0 ml) was added lithium hydroxide monohydrate in water (1.3 ml, 2.0 M, 2.6 mmol) at 20° C. The mixture was stirred at room temperature for 16 hours. The mixture was adjusted pH=5 with hydrochloric acid in water (1N), the mixture was extracted with ethyl acetate. The organic phase was dried over anhydrous sodium sulfate, filtered and concentrated to give 5-bromo-4-[(tert-butoxycarbonyl)amino]-2-chlorobenzoic acid (160 mg, 83% yield) as a white solid.

LC-MS (Method C): Rt=0.874 min; MS (ESIpos): m/z=370.2 [M+H]⁺.

### Intermediate 63

### 5-bromo-2,3-dichloro-N-phenylbenzamide

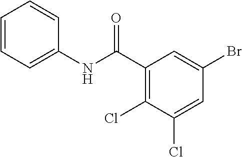 [SMILES: O=C(Nc1ccccc1)c1cc(Br)cc(Cl)c1Cl]

5-bromo-2,3-dichlorobenzoic acid (300 mg, 1.11 mmol) was dissolved in 4.3 ml DMF. Aniline (120 μl, 1.3 mmol; CAS-RN:[62-53-3])], HATU (507 mg, 1.33 mmol; CAS-RN:[148893-10-1])] and N,N-diisopropylethylamine (500 μl, 2.9 mmol; CAS-RN:[7087-68-5]) was added and reaction mixture was stirred at rt for 72 h. The reaction mixture was extracted with ethyl acetate. The combined organic layers were dried over sodium sulfate and concentrated. Crude product was purified by flash chromatography to give 384 mg (98% purity, 98% yield) of the desired product.

LC-MS (Method 4): Rₜ=1.31 min; MS (ESIpos): m/z=345 [M+H]⁺

### Intermediate 64

### 5-bromo-4-chloro-2-fluoro-N-(4-fluorobenzyl)benzamide

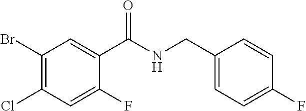 [SMILES: O=C(NCc1ccc(F)cc1)c1cc(Br)c(Cl)cc1F]

To a solution of 5-bromo-4-chloro-2-fluoro-benzoic acid (5 g, 19.73 mmol; Intermediate 3) and (4-fluorophenyl)methanamine (4.94 g, 39.46 mmol) in DMF (50 mL) was added DIEA (5.10 g, 39.46 mmol) and HATU (15.00 g, 39.46 mmol). The mixture was stirred at 25° C. for 16 hours. Then the mixture was added into H2O (200 mL) and a white solid was formed. The solid was collected by filtration and dried in vacuum to give compound 5-bromo-4-chloro-2-fluoro-N-(4-fluorobenzyl)benzamide (7 g, 98% yield) as a white solid.

1H NMR (DMSO-d6 400 MHz) 9.03 (s, 1H), 7.98 (d, 1H), 7.79 (d, 2H), 7.34-7.38 (m, 2H), 7.13-7.17 (m, 2H), 4.44 (d, 2H).

19F NMR(DMSO-d6 376 MHz) −113.02, −115.95.

LC-MS (method 8) Rₜ=0.957 min, m/z=361.7 (M+H)+

### Intermediate 65

### ethyl-2-chloro-4-fluoro-5-[(4-fluorophenyl)methylcarbamoyl]benzoate

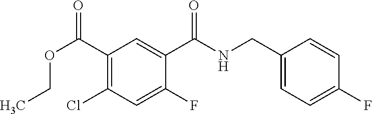 [SMILES: CCOC(=O)c1cc(C(=O)NCc2ccc(F)cc2)c(F)cc1Cl]

To a solution of 5-bromo-4-chloro-2-fluoro-N-[(4-fluorophenyl)methyl]benzamide (20 g, 55.47 mmol; Intermediate 64) and TEA (16.84 g, 166.40 mmol) in EtOH (200 mL) was added Pd(dppf)Cl2 (4.06 g, 5.55 mmol) under N2 atmosphere. The suspension was degassed and purged with CO for 3 times. The mixture was stirred under CO (50 Psi) at 60° C. for 40 hrs. The mixture was filtered through a celite pad, then the solvent was removed by evaporation. The residue was purified by flash silica gel chromatography (ISCO@; 120 g SepaFlash@ Silica Flash Column, Eluent of 0-40% Ethyl acetate/Petroleum ethergradient @ 60 mL/min) to give compound 2-chloro-4-fluoro-5-((4-fluorobenzyl)carbamoyl)benzoate (18 g, crude).

### Intermediate 66

### 2-chloro-4-fluoro-5-((4-fluorobenzyl)carbamoyl)benzoic acid

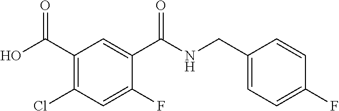 [SMILES: O=C(NCc1ccc(F)cc1)c1cc(C(=O)O)c(Cl)cc1F]

To a solution of ethyl-2-chloro-4-fluoro-5-[(4-fluorophenyl)methylcarbamoyl]benzoate (17.50 g, Intermediate 65) in H₂O (20 mL) and THF (100 mL) was added LiOH·H₂O (1.14 g, 27.14 mmol). The mixture was stirred at 25° C. for 16 hours. The reaction mixture was diluted with H₂O (100 mL) and concentrated under reduced pressure to remove THF. Then the aqueous phase was washed with TBME (100 mL). To the aqueous phase was added 4M aq. HCl (20 mL) and a white solid was formed. The solid was collected by filtration and dried in vacuum to give compound 2-chloro-4-fluoro-5-((4-fluorobenzyl)carbamoyl)benzoic acid (5.4 g) as a white solid.

1H NMR (DMSO-d6 400 MHz) 13.64 (br, 1H), 9.03 (t, 1H), 8.09 (d, 1H), 7.70 (d, 1H), 7.35-7.38 (m, 2H), 7.14-7.19 (m, 2H), 4.45 (d, 2H).

19F NMR (DMSO-d6 400 MHz) −107.50, −116.00.

LC-MS (method 8) Rₜ=0.839 min, m/z=325.7 (M+H)+

### Intermediate 67

### tert-butyl 5-(benzylcarbamoyl)-2-chlorobenzoate

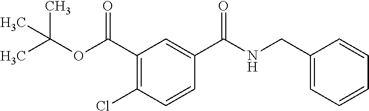 [SMILES: CC(C)(C)OC(=O)c1cc(C(=O)NCc2ccccc2)ccc1Cl]

To a solution of 3-(tert-butoxycarbonyl)-4-chlorobenzoic acid (2.00 g, 7.79 mmol, CAS 862112-39-8), phenylmethanamine (1.0 ml, 9.4 mmol; CAS-RN: [100-46-9]) and N,N′-diisopropylethylamine (3.5 ml, 20 mmol; CAS-RN: [7087-68-5]) in DMF (22 mL) was added HATU (3.56 g, 9.35 mmol; CAS-RN: [148893-10-1]). The mixture was stirred at RT for 16 h, at which time the reaction was diluted with sat. aqueous sodium hydrogencarbonate solution and extracted 3× with ethyl acetate. The combined organic layers were dried over sodium sulfate, filtered, and concentrated in vacuo to yield 2.70 g (94% purity, 94% yield) of the desired product.

LC-MS (Method 4): Rₜ=1.31 min; MS (ESIpos): m/z=345 [M+H]⁺

### Intermediate 68

### 5-(benzylcarbamoyl)-2-chlorobenzoic acid

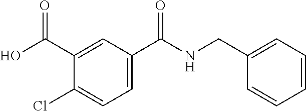 [SMILES: O=C(NCc1ccccc1)c1ccc(Cl)c(C(=O)O)c1]

Trifluoroacetic acid (22 ml, 290 mmol; CAS-RN:[76-05-1]) was dissolved in DCM (210 ml) and then tert-butyl 5-(benzylcarbamoyl)-2-chlorobenzoate (2.51 g, 7.27 mmol; Intermediate 67) was added to the mixture and stirred at rt for 16 h. The reaction mixture evaporated, and reevaporated twice with toluene. The residue was suspended in MTBE, the solid was filtered off and washed with 2 ml DCM to obtain 1.80 g (85% yield) of the desired product.

LC-MS (Method 4): Rₜ=0.89 min; MS (ESIpos): m/z=290 [M+H]⁺

### Intermediate 69

### tert-butyl 4-chloro-3-(phenylcarbamoyl)benzoate

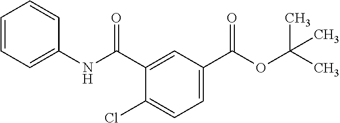 [SMILES: CC(C)(C)OC(=O)c1ccc(Cl)c(C(=O)Nc2ccccc2)c1]

To a solution of 5-(tert-butoxycarbonyl)-2-chlorobenzoic acid (3.71 g, 14.5 mmol; Intermediate 68), aniline (1.6 ml, 17 mmol) and triethylamine (5.2 ml, 38 mmol; CAS-RN: [121-44-8]) in DMF (56 mL) was added HATU (6.59 g, 17.3 mmol; CAS-RN: [148893-10-1]). The mixture was stirred at RT for 21 h. The reaction mixture was poured into ice water and stirred for 30 min. Solid was filtered off, dried under vacuum and purified by flash chromatography to give 3.14 g (65% yield) of the desired product.

LC-MS (Method 4): Rt=1.34 min; MS (ESIpos): m/z=332 [M+H]⁺

¹H-NMR (400 MHz, DMSO-d6) δ[ppm]: 1.154 (0.46), 1.172 (0.90), 1.189 (0.43), 1.557 (16.00), 1.987 (1.63), 2.518 (0.44), 7.129 (0.63), 7.346 (0.73), 7.367 (1.03), 7.385 (0.70), 7.697 (0.98), 7.699 (1.18), 7.708 (0.98), 7.718 (1.00), 7.721 (0.80), 7.730 (1.02), 7.971 (0.64), 7.976 (0.88), 7.992 (0.46), 7.997 (1.14), 8.004 (1.31), 8.008 (0.76), 10.597 (0.76).

### Intermediate 70

### 4-chloro-3-(phenylcarbamoyl)benzoic acid

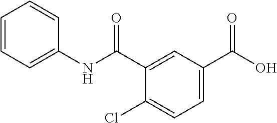 [SMILES: O=C(O)c1ccc(Cl)c(C(=O)Nc2ccccc2)c1]

[Tert-butyl 4-chloro-3-(phenylcarbamoyl)benzoate (3.13 g, 9.43 mmol; Intermediate 69) was dissolved in 200 ml DCM. Trifluoroacetic acid (18 ml, 240 mmol; CAS-RN: [76-05-1]) was slowly added and stirred at rt for 20 h. The reaction mixture was concentrated and then poured into ice water. Solid was filtered off, dried under vacuum to give 2.12 g (82% yield) of the desired product.

LC-MS (Method 4): Rt=0.95 min; MS (ESIpos): m/z=276 [M+H]⁺

¹H-NMR (400 MHz, DMSO-d6) δ[ppm]: 2.518 (3.59), 2.523 (2.43), 5.758 (2.40), 7.106 (2.08), 7.108 (3.62), 7.112 (2.25), 7.127 (7.76), 7.137 (1.43), 7.143 (3.03), 7.146 (4.78), 7.148 (2.89), 7.156 (0.71), 7.344 (9.06), 7.348 (3.97), 7.365 (13.08), 7.379 (3.16), 7.384 (8.78), 7.679 (1.73), 7.697 (12.83), 7.698 (14.83), 7.707 (12.49), 7.717 (12.62), 7.721 (10.03), 7.728 (12.98), 8.009 (7.98), 8.014 (10.00), 8.022 (2.68), 8.030 (4.75), 8.035 (11.63), 8.043 (16.00), 8.047 (9.73), 8.296 (2.17), 8.301 (2.00), 10.608 (10.13), 10.666 (1.24), 13.424 (0.94).

### Intermediate 71

### 5-bromo-4-chloro-N-(3,4-difluorobenzyl)-2-fluorobenzamide

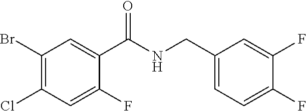 [SMILES: O=C(NCc1ccc(F)c(F)c1)c1cc(Br)c(Cl)cc1F]

To a mixture of 5-bromo-4-chloro-2-fluoro-benzoic acid (2 g, 7.89 mmol, 1 eq, CAS-RN: [289038-22-8]), HATU (4.50 g, 11.84 mmol, 1.5 eq, CAS-RN: [148893-10-1]) and DIEA (3.06 g, 23.67 mmol, 4.12 mL, 3 eq, CAS-RN: [7087-68-5]) in DMF (20 mL) was added 3,4-difluorobenzylamine (1.13 g, 7.89 mmol, 933.45 uL, 1 eq, CAS-RN: [72235-53-1]). The mixture was stirred at 25° C. for 1 hr. The reaction mixture was diluted with water (50 mL) and extracted with EA (20 mL*3), the combined organic phase was washed with brine (20 mL*2) and dried over anhydrous Na₂SO₄, then concentrated to afford the crude product. The crude product was purified by flash silica gel chromatography (ISCO@; 40 g SepaFlash@ Silica Flash Column, Eluent of 0-40% Ethylacetate/Petroleum ethergradient @ 50 mL/min), the eluent was concentrated to afford 5-bromo-4-chloro-N-[(3,4-difluorophenyl)methyl]-2-fluoro-benzamide (2.6 g, 6.70 mmol, 84.91% yield) as white solid.

LC-MS (Method 9): Rₜ 0.912 min; MS (ESIpos) m/z: =379.8 [M+H]⁺

¹H NMR (400 MHz, DMSO-d₆) δ [ppm]: 9.07-9.04 (m, 1H), 8.02 (d, J=6.8 Hz, 1H), 7.83 (d, J=10.0 Hz, 1H), 7.41-7.36 (m, 2H), 7.18-7.16 (m, 1H), 4.44 (d, J=6.0 Hz, 2H) ppm.

### Intermediate 72

### 2-chloro-5-((3,4-difluorobenzyl)carbamoyl)-4-fluorobenzoic acid

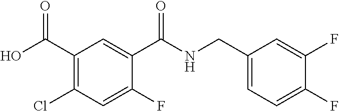 [SMILES: O=C(NCc1ccc(F)c(F)c1)c1cc(C(=O)O)c(Cl)cc1F]

To a solution of 5-bromo-4-chloro-N-[(3,4-difluorophenyl)methyl]-2-fluoro-benzamide (Intermediate 71, 500 mg, 1.32 mmol, 1 eq) in DMSO (5 mL) and H2O (5 mL) was added Pd(OAc)₂ (14.83 mg, 66.04 umol, 0.1 eq) dicyclohexyl(3-dicyclohexylphosphaniumylpropyl)-phosphonium; ditetrafluoroborate (80.86 mg, 132.08 umol, 0.1 eq) and potassium carbonate (365.07 mg, 2.64 mmol, 2 eq), then the mixture was stirred under CO atmosphere(15 psi) at 100° C. for 12 hrs. The mixture was poured into water (5 mL) and extracted by EtOAc (20 mL), then the aqueous phase was adjusted pH to 6-7 with 1 M HCl (10 mL) and extracted by EtOAc (20 mL*3), the organic layer was separated and evaporated to give crude product. The crude product was purified by column chromatography (SiO2, Petroleum ether/Ethyl acetate=1/100 to 100/1), TLC (PE: EA=1:1, Rt=0.4), the eluent was concentrated to give 2-chloro-5-[(3,4-difluorophenyl)methylcarbamoyl]-4-fluoro-benzoic acid (105 mg, 305.51 umol, 23.61% yield) as white solid.

LC-MS (Method 9): Rt 0.717 min; MS (ESIpos): m/z=344.0 [M+H]⁺

### Intermediate 73

### methyl 3,4-dichloro-5-(pyrazin-2-ylcarbamoyl)benzoate

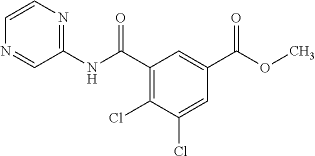 [SMILES: COC(=O)c1cc(Cl)c(Cl)c(C(=O)Nc2cnccn2)c1]

To a solution of 2,3-dichloro-5-methoxycarbonyl-benzoic acid (800 mg, 3.21 mmol, 1 eq) and pyrazin-2-amine (458.24 mg, 4.82 mmol, 1.5 eq, CAS-RN: [5049-61-6]) in pyridine (8 mL) was 10 added T3P (6.13 g, 9.64 mmol, 5.73 mL, 50% purity, 3 eq, CAS-RN: [68957-94-8]). The mixture was stirred at 25° C. for 12 h. The reaction mixture was diluted with water (20 mL) and extracted with EtOAc (20 mL×3). The combined organic layers were washed with brine (15 mL×4), dried over Na2SO4, filtered, and the solvent removed in vacuo. The resulting residue was purified by flash column chromatography (SiO2, Gradient of petroleum ether/ethyl acetate=3/1 to 2/1). The fraction containing the title compound were concentrated to provide 550 mg (1.69 mmol, 52.50% yield) of the title compound as a white solid confirmed by HNMR.

¹H-NMR (400 MHz, DMSO-d6) δ[ppm]: 3.902, 8.116, 8.214, 8.219, 8.466, 9.420, 11.535

LCMS (Method 6): Rₜ=0.596 min, MS (ESIpos): m/z=325.9 (M+)

### Intermediate 74

### 3,4-dichloro-5-(pyrazin-2-ylcarbamoyl)benzoic acid

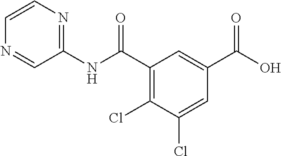 [SMILES: O=C(O)c1cc(Cl)c(Cl)c(C(=O)Nc2cnccn2)c1]

To a solution of methyl 3,4-dichloro-5-(pyrazin-2-ylcarbamoyl)benzoate (300 mg, 919.86 umol, 1 eq) in THF (3 mL) and water (0.6 mL) was added LiOH·H2O (77.20 mg, 1.84 mmol, 2 eq, CAS-RN: [1310-66-3]). The mixture was stirred at 25° C. for 1 h. The reaction mixture was diluted with water (5 mL), the pH was adjusted to 4-3 by the addition 2.5 M HCl. Then the mixture was extracted with EtOAc (10 mL×3). The combined organic layers were washed with brine (8 mL×3), dried over Na2SO4, filtered, and concentrated in vacuo evaporated to provide 300 mg of the title compound as a crude white solid that was used without purification.

LCMS (method 6): Rₜ=0.455; MS (ESIneg): m/z=311.8 (M−)

### Intermediate 75

### 5-phenoxypyridin-2-amine

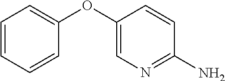 [SMILES: Nc1ccc(Oc2ccccc2)cn1]

To a solution of 2-nitro-5-phenoxy-pyridine (300 mg, 1.39 mmol, 1 eq, CAS-RN: [779345-38-9]) in H2O (0.3 mL) and EtOH (1 mL) was added iron fillings (387.47 mg, 6.94 mmol, 5 eq, CAS-RN: [7439-89-6]) and ammonium chloride (593.82 mg, 11.10 mmol, 8 eq, CAS-RN: [12125-02-9]), the mixture was stirred at 80° C. for 2 hr. Additional ammonium chloride (148.45 mg, 2.78 mmol, 2 eq, CAS-RN: [12125-02-9]) was added and kept on stirred for 2 hr. The reaction mixture was filtered and diluted with MeOH (5 mL), extracted with EtOAc (4 mL×3). The combined organic layers were washed with brine (4 mL×3), dried over sodium sulfate, filtered, and concentrated in vacuo. The residue was purified by column chromatography (SiO₂, Petroleum diethylether/Ethyl acetate=10/1 to 1/1), and fractions containing the desired product were concentrated to provide 227 mg (1.22 mmol, 87.85% yield) of the title compound as a white solid.

LCMS (Method 6): Rₜ=0.311, MS (ESIpos): m/z=187.1 (M+H)+

### Intermediate 76

### methyl 4-chloro-5-{[4-(difluoromethoxy)-3-fluorophenyl]carbamoyl}-2-fluorobenzoate

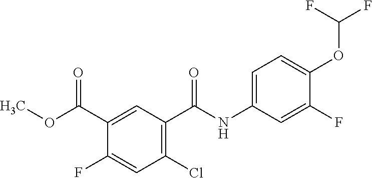 [SMILES: COC(=O)c1cc(C(=O)Nc2ccc(OC(F)F)c(F)c2)c(Cl)cc1F]

Analogous to the reaction conditions described for the synthesis of Intermediate 59, 410 mg (64% yield) of the desired product were obtained starting from 5-bromo-2-chloro-N-[4-(difluoromethoxy)-3-fluorophenyl]-4-fluorobenzamide (670 mg, 1.62 mmol).

LC-MS (method 4): Rₜ=1.21 min; MS (ESipos): m/z=392 [M+H]⁺

### Intermediate 77

### 4-chloro-5-{[4-(difluoromethoxy)-3-fluorophenyl]carbamoyl}-2-fluorobenzoic acid

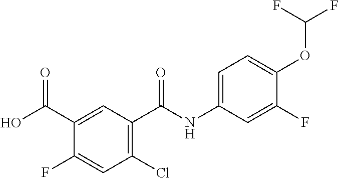 [SMILES: O=C(O)c1cc(C(=O)Nc2ccc(OC(F)F)c(F)c2)c(Cl)cc1F]

To a solution of methyl 4-chloro-5-{[4-(difluoromethoxy)-3-fluorophenyl]carbamoyl}-2-fluorobenzoate (410 mg, 1.05 mmol, Intermediate 76) in THF (6.0 ml) was added sodium hydroxide (5.2 ml, 1.0 M aqueous solution, 5.2 mmol; CAS-RN:[1310-73-2]) and the mixture was stirred at rt for 8 h. The reaction mixture was diluted with water (10 mL), extracted with EtOAc (12 mL×3). The aqueous phase was acidified with 1M HCl and extracted with EtOAc. The combined organic layers were washed with brine (8 mL×3), dried over Na2SO4, filtered, and concentrated in vacuo to give 213 mg (99% purity, 53% yield) of the title compound.

LC-MS (method 4): Rt=1.08 min; MS (ESIpos): m/z=378 [M+H]+

¹H-NMR (400 MHz, DMSO-d6) δ[ppm]: 0.851 (0.82), 1.154 (2.58), 1.172 (5.62), 1.190 (2.85), 1.232 (3.25), 1.907 (10.70), 1.987 (9.89), 2.336 (0.54), 2.518 (5.88), 2.523 (4.21), 2.678 (0.56), 3.999 (0.67), 4.017 (2.01), 4.034 (1.93), 4.052 (0.60), 7.010 (7.94), 7.193 (16.00), 7.364 (3.44), 7.376 (7.74), 7.386 (8.14), 7.408 (6.09), 7.437 (6.53), 7.441 (6.40), 7.444 (5.66), 7.459 (3.11), 7.463 (3.05), 7.466 (3.00), 7.760 (12.71), 7.786 (12.46), 7.798 (6.44), 7.804 (6.30), 7.830 (6.14), 7.836 (5.91), 8.063 (13.07), 8.082 (13.28), 10.872 (12.40), 13.704 (0.43).

### Intermediate 78

### methyl 5-(benzylcarbamoyl)-2-nitrobenzoate

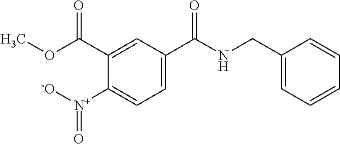

3-(methoxycarbonyl)-4-nitrobenzoic acid (2.00 g, 8.88 mmol, CAS 64152-09-6) was dissolved in DCM (60 ml) and DMF (68 μl) was added then ethanedioyl dichloride (930 μl, 11 mmol) was added portionwise. The suspension was stirred for 1 h. (solution A). 1-phenylmethanamine (1.3 ml, 12 mmol) was dissolved in DCM and 2 M sodium carbonate (22 ml, 2.0 M, 44 mmol; CAS-RN:[497-19-8]) was added. Then solution A was added portionwise. The resulting mixture was stirred at RT for 1 h. The reaction mixture was diluted with water and extracted with DCM. The organic layer were combined and extracted with 1 M HCl and washed with saturated NaHCO3 solution. The organic layer was dried over a coated filter and evaporated. 2.80 g (90% purity, 90% yield) of the crude desired product was obtained.

LC-MS (method 4): Rₜ=1.02 min; MS (ESIpos): m/z=315 [M+H]⁺

¹H-NMR (400 MHz, DMSO-d6) δ[ppm]: 2.518 (0.56), 2.523 (0.40), 3.878 (16.00), 4.502 (2.64), 4.517 (2.60), 5.758 (2.31), 7.247 (0.52), 7.250 (0.40), 7.258 (0.94), 7.268 (0.84), 7.271 (0.77), 7.280 (0.63), 7.334 (10.30), 7.345 (6.56), 8.169 (1.85), 8.190 (2.86), 8.258 (1.86), 8.262 (1.77), 8.279 (1.14), 8.284 (1.31), 8.343 (2.47), 8.348 (2.26), 9.467 (0.72).

### Intermediate 79

### methyl 2-amino-5-(benzylcarbamoyl)benzoate

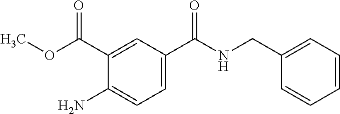 [SMILES: COC(=O)c1cc(C(=O)NCc2ccccc2)ccc1N]

Methyl 5-(benzylcarbamoyl)-2-nitrobenzoate (2.82 g, 8.97 mmol, Intermediate 78) was dissolved in EtOAc and Pd/C (955 mg, 10% purity, 897 μmol; CAS-RN:[7440-05-3]) was added. The reaction mixture was shaked under hydrogen atmosphere for 5 h. The solution was filtered and stored over the weekend. For restart the cat was added again (new) and the mixture was shaked again for 45 minutes. The suspension was filtered and the filtrate was evaporated to give 3.43 g (90% purity, 121% yield) of the crude desired product.

LC-MS (method 4): Rₜ=0.89 min; MS (ESIpos): m/z=285 [M+H]⁺

1H-NMR (400 MHz, DMSO-d6) delta [ppm]: 1.154 (2.53), 1.171 (4.83), 1.190 (2.34), 1.987 (9.76), 2.518 (0.49), 3.816 (16.00), 3.999 (0.71), 4.017 (2.08), 4.034 (2.04), 4.052 (0.65), 4.421 (2.21), 4.437 (2.21), 6.781 (2.17), 6.803 (2.20), 7.087 (2.18), 7.222 (0.93), 7.226 (0.44), 7.229 (0.44), 7.232 (0.43), 7.239 (0.63), 7.244 (0.54), 7.276 (0.85), 7.280 (0.50), 7.291 (3.93), 7.297 (5.26), 7.304 (0.42), 7.314 (2.08), 7.318 (0.85), 7.333 (0.56), 7.784 (1.15), 7.790 (1.12), 7.806 (1.04), 7.811 (1.08), 8.346 (2.16), 8.351 (2.07), 8.787 (0.42), 8.802 (0.83).

### Intermediate 80

### methyl 5-(benzylcarbamoyl)-2-bromobenzoate

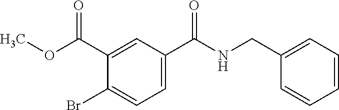 [SMILES: COC(=O)c1cc(C(=O)NCc2ccccc2)ccc1Br]

Methyl 2-amino-5-(benzylcarbamoyl)benzoate (2.50 g, 8.79 mmol, Intermediate 79) was suspended in 10 ml water, hydrogen bromide (16 ml, 32% purity, 88 mmol; CAS-RN:[10035-10-6]) was added and the mixture was cooled to 0° C. sodium nitrite (673 mg, 9.76 mmol; CAS-RN:[7632-00-0]) was dissolved in 7 ml water and added to the mixture and stirring continued at 0° C. for 1 h. A suspension of copper(I)bromide (3.78 g, 26.4 mmol; CAS-RN:[7787-70-4]) in 15eq hydrogen bromide was added dropwise and the dark solution was allowed warm to RT and was stirred for an additional 90 min. Water was added and the mixture was extracted with ethyl acetate. The combined organic layers were extracted with Na2S2O3-solution (green->yellow). After removal of the solvent 3.00 g (90% purity, 88% yield) of the crude desired product was obtained.

LC-MS (method 4): Rₜ=1.08 min; MS (ESIpos): m/z=348,350 [M+H]⁺

¹H-NMR (400 MHz, DMSO-d6) δ[ppm]: 1.886 (9.30), 2.074 (0.64), 3.896 (0.43), 4.473 (3.78), 4.489 (3.85), 7.231 (0.53), 7.236 (0.52), 7.240 (0.62), 7.245 (1.02), 7.250 (0.80), 7.252 (0.67), 7.254 (0.68), 7.258 (0.87), 7.260 (0.94), 7.266 (0.77), 7.305 (0.87), 7.309 (0.71), 7.318 (16.00), 7.326 (4.42), 7.333 (3.88), 7.338 (0.74), 7.352 (0.46), 7.875 (2.70), 7.896 (4.46), 7.952 (2.90), 7.958 (2.88), 7.973 (1.68), 7.979 (1.82), 8.269 (3.49), 8.274 (3.46), 9.273 (0.55), 9.288 (1.10), 9.302 (0.55).

### Intermediate 81

### 5-(benzylcarbamoyl)-2-bromobenzoic acid

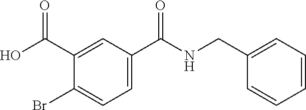 [SMILES: O=C(NCc1ccccc1)c1ccc(Br)c(C(=O)O)c1]

Analogous to the reaction conditions described for the synthesis of Intermediate 77, 2.28 g (95% purity, 75% yield) of the desired product were obtained starting from methyl 5-(benzylcarbamoyl)-2-bromobenzoate (3.00 g, 8.62 mmol, Intermediate 80).

LC-MS (method 4): Rₜ=0.84 min; MS (ESIpos): m/z=334 [M+H]⁺

¹H-NMR (400 MHz, DMSO-d6) δ[ppm]: 1.352 (0.42), 2.518 (1.66), 2.523 (1.17), 4.468 (4.06), 4.482 (4.14), 7.230 (0.60), 7.235 (0.63), 7.240 (0.69), 7.244 (1.16), 7.250 (1.15), 7.258 (0.93), 7.260 (1.08), 7.266 (0.87), 7.303 (1.08), 7.307 (0.73), 7.317 (16.00), 7.324 (4.91), 7.333 (4.19), 7.338 (0.88), 7.351 (0.61), 7.354 (0.48), 7.832 (2.98), 7.853 (5.08), 7.903 (3.07), 7.909 (3.01), 7.924 (1.70), 7.930 (1.82), 8.248 (3.85), 8.254 (3.86), 9.252 (0.69), 9.266 (1.38), 9.281 (0.67).

### Intermediate 82

### 5-bromo-4-chloro-2-fluoro-N-(2-methoxybenzyl)benzamide

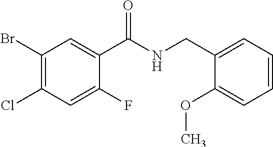 [SMILES: COc1ccccc1CNC(=O)c1cc(Br)c(Cl)cc1F]

To a mixture of 5-bromo-4-chloro-2-fluoro-benzoic acid (2 g, 7.89 mmol, 1 eq, Intermediate 3), HATU (4.50 g, 11.84 mmol, 1.5 eq) and DIEA (3.06 g, 23.67 mmol, 4.12 mL, 3 eq) in DMF (20 mL) was added (2-methoxyphenyl)methanamine (1.08 g, 7.89 mmol, 933.45 uL, 1 eq). The mixture was stirred at 25° C. for 1 hr. The reaction mixture was diluted with water (50 mL) and extracted with EA (20 mL*3), the combined organic phase was washed with brine (20 mL*2) and dried over anhydrous Na2SO4, then concentrated to afford the crude product. The crude product was purified by flash silica gel chromatography (ISCO@; 40 g SepaFlash@ Silica Flash Column, Eluent of 0-40% Ethylacetate/Petroleum ethergradient @ 50 mL/min), the eluent was concentrated to afford 5-bromo-4-chloro-2-fluoro-N-(2-methoxybenzyl)benzamide (2.5 g, 6.71 mmol, 85.02% yield) as white solid.

LC-MS (Method 8): Rt 0.955 min; MS (ESIpos) m/z: =374.0 [M+H]+

1H NMR (400 MHz, DMSO-d6) δ=8.86-8.83 (m, 1H), 7.98 (d, J=7.2 Hz, 1H), 7.82 (d, J=10.0 Hz, 1H), 7.36-7.18 (m, 2H), 7.09-6.88 (m, 2H), 4.42 (d, J=6.0 Hz, 2H), 3.83 (s, 3H).

### Intermediate 83

### 2-chloro-4-fluoro-5-((2-methoxybenzyl)carbamoyl)benzoic acid

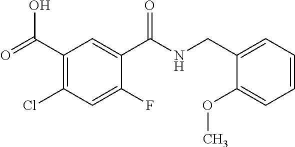 [SMILES: COc1ccccc1CNC(=O)c1cc(C(=O)O)c(Cl)cc1F]

To a solution of 5-bromo-4-chloro-2-fluoro-N-(2-methoxybenzyl)benzamide (Intermediate 82, 500 mg, 1.32 mmol, 1 eq) in DMSO (5 mL) and H2O (5 mL) was added Pd(OAc)2 (14.83 mg, 66.04 umol, 0.1 eq) dicyclohexyl(3-dicyclohexylphosphaniumylpropyl)-phosphonium; ditetrafluoroborate (80.86 mg, 132.08 umol, 0.1 eq) and K₂CO₃ (365.07 mg, 2.64 mmol, 2 eq), then the mixture was stirred under CO atmosphere(15 psi) at 100° C. for 12 hrs. The mixture was poured into water (5 mL) and extracted by EA (20 mL), then the aqueous phase was adjusted pH to 6-7 with 1M HCl (10 mL) and extracted by EA (20 mL*3), the organic layer was separated and evaporated to give crude product. The crude product was purified by column chromatography (SiO2, Petroleum ether/Ethyl acetate=1/100 to 100/1), TLC (PE: EA=1:1, Rt=0.4), the eluent was concentrated to give 2-chloro-4-fluoro-5-((2-methoxybenzyl)carbamoyl)benzoic acid (120 mg, 355.51 umol, 26.48% yield) as white solid.

LC-MS (Method 8): Rt 0.707 min; MS (ESIpos): m/z=338.0 [M+H]⁺

### Intermediate 84

### 2-fluoro-4-(oxetan-3-yl)aniline

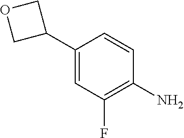 [SMILES: Nc1ccc(C2COC2)cc1F]

A solution of 3-iodooxetane (100 mg, 543.55 umol) in 2-PROPANOL (2 mL) were added 2-fluoro-4-(4,4,5,5-tetramethyl-1,3,2-dioxaborolan-2-yl)aniline (257.73 mg, 1.09 mmol), diiodonickel (16.99 mg, 54.36 umol, 2.91 uL), NaHMDS (1 M, 1.09 mL) and (1R,2R)-2-aminocyclohexanol (6.26 mg, 54.36 umol). The reaction mixture was quenched by addition sat·aq·NH4Cl 20 mL and extracted with EA (10 mL*3). The combined organic layers were washed with brine 20 mL, dried over anhydrous Na2SO4, filtered and concentrated under reduced pressure to give a residue. The residue was purified by flash silica gel chromatography (ISCO@; 12 g SepaFlash@Silica Flash Column, Eluent of 0-40% Ethylacetate/Petroleum ethergradient @ 25 mL/min) and concentrated under reduced pressure to get Intermediate 4 (90 mg, 413.64 umol, 76.10% yield, 76.835% purity) as red oil.

1H NMR (400 MHz, DMSO-d6) δ=7.11-7.07 (m, 1H), 6.99-6.97 (m, 1H), 6.81-6.77 (m, 1H), 5.07-5.03 (m, 2H), 4.76-4.67 (m, 2H), 4.14-4.10 (m, 1H), 3.72 (s, 2H)

### Intermediate 85

### Methyl-4-Chloro-3-fluoro-5-vinyl-benzoate

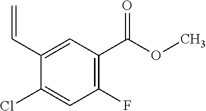

To a solution of methyl 5-bromo-4-chloro-2-fluoro-benzoate (36.8 g, 137.58 mmol, 1 eq) and potassium trifluoro(vinyl)boronate (27.64 g, 206.37 mmol, 1.5 eq) in a mixture of 1,4-dioxane (368 mL) and H₂O (75 mL) was

added Pd(dppf)Cl2 (5.03 g, 6.88 mmol, 0.05 eq) and K₂CO₃ (57.04 g, 412.74 mmol, 3 eq) under N2. The mixture was stirred at 60° C. for 12 hr. The reaction solution was stirred at 80° C. for 12 hrto give a black solution. TLC (PE:EA=10:1) indicated full consumption of the starting material.

The reaction mixture was quenched by addition sat. NH₄Cl (250 mL), and extracted with EtOAc (500 mL×3). The combined organic layers were washed with brine (300 mL×3), dried over Na₂SO₄, filtered, and concentrated in vacuo to give a residue. The residue was purified by normal phase column chromatography (SiO₂, Petroleum ether:Ethyl acetate=10:1), and the fractions containing the desired compound were dried under reduced pressure to provide 21 g (97.85 mmol, 71.12% yield) of the title compound as a white solid.

1H NMR (400 MHz, CHLOROFORM-d) δ ppm 4.06 (s, 3H) 5.56 (d, J=12 Hz, 1H) 5.90 (d, J=18 Hz, 1H) 7.05-7.17 (m, 1H) 7.31 (s, 1H) 8.25 (d, J=7.6 Hz, 1H)

### Intermediate 86

### 4-Chloro-2-fluoro-5-formyl-methylbenzoate

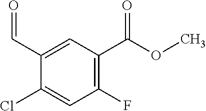

To a solution of methyl 4-chloro-2-fluoro-5-vinyl-benzoate (21 g, 97.85 mmol, 1 eq, Intermediate 85) in a mixture of THF (120 mL) and H₂O (30 mL) was added K₂OsO₄-2H₂O (2.52 g, 6.85 mmol, 0.07 eq) and NalO₄ (104.64 g, 489.24 mmol, 27.11 mL, 5 eq). The mixture was stirred at 25° C. for 24 hr to give a black suspension. TLC (PE:EA=10:1) indicated full consumption of the starting material. The reaction mixture was quenched by the addition of 200 mL of H₂O, and the resulting solution extracted with EtOAc (300 mL*3). The combined organic layers were washed with brine (300 mL*3), dried over Na₂SO₄, filtered, and concentrated in vacuo to give a residue. The residue was purified by normal phase column chromatography (SiO2, Petroleum ether:Ethyl acetate=10:1), the fractions containing the desired compound were dried in vacuo to provide 23 g of the title compound as a yellow solid.

1H NMR (400 MHz, DMSO-d6) δ ppm 3.89 (s, 3H) 7.85 (d, J=12.0 Hz, 1H) 8.33 (d, J=7.6 Hz, 1H) 10.24 (s, 1H)

### Intermediate 87

### 2-Chloro-4-fluoro-5-methoxycarbonyl-benzoic acid

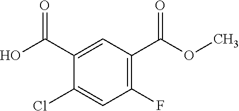

To a solution of methyl 4-chloro-2-fluoro-5-formyl-benzoate (21 g, 96.96 mmol, 1 eq, Intermediate 86) and sodium dihydrogen phosphate (3.49 g, 29.09 mmol, 0.3 eq) in MeCN (120 mL) and H₂O (60 mL) was added H₂O₂(32.98 g, 290.87 mmol, 27.95 mL, 30% purity, 3 eq) under N₂ at 0° C. Then a solution of sodium chlorite (17.54 g, 193.91 mmol, 2 eq) in H₂O (60 mL) was added, and the mixture was stirred at 20° C. for 2 h to give yellow suspension.

TLC (PE:EA=10:1) showed complete consumption of the starting material. The reaction mixture was quenched with water (75 mL) and sat. Na₂S2O₃ (225 mL), and the pH was adjusted to 3-2 by the addition 2 M HCl. Then the mixture was extracted with EtOAc (300 mL×3). The combined organic layers were washed with sat. Na₂S₂O₃ (225 mL×3) and brine (225 mL×3), the dried over Na₂SO₄, filtered, and concentrated in vacuo to give 8.9 g of the title compound as a white solid.

1H NMR (400 MHz, DMSO-d6) δ ppm 3.87 (s, 3H) 7.74 (d, J=12.0 Hz, 1H) 8.34 (d, J=7.6 Hz,

1H) 13.64-13.80 (m, 1H)

### Intermediate 88

### methyl 4-chloro-5-chlorocarbonyl-2-fluoro-benzoate

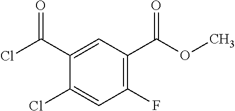

To a solution of 2-chloro-4-fluoro-5-methoxycarbonyl-benzoic acid (200 mg, 859.87 umol, 1 eq, Intermediate 87) in DCM (1 mL) was added DMF (3.14 mg, 42.99 umol, 3.31 uL, 0.05 eq). Then a solution of oxalyl chloride (163.71 mg, 1.29 mmol, 112.90 uL, 1.5 eq) in DCM (1 mL) was added dropwise at 0° C., and the resulting mixture was stirred at 0° C. for 30 min. The solution was allowed to warm to 25° C. and stirred for a further 2 hr to give a pink solution. TLC (PE:EA=10:1) showed consumption of the starting material. The reaction mixture was concentrated in vacuo to give 200 mg of the title compound as a yellow solid, which was used without further purification.

### Intermediate 89

### methyl 4-amino-2-fluoro-5-iodobenzoate

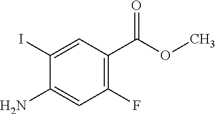

To a solution of methyl 4-amino-2-fluoro-benzoate (24 g, 141.88 mmol, 1 eq) and N-iodo-succinimide (35.11 g, 156.07 mmol, 1.1 eq) in acetic acid (240 mL) and DMF (48 mL) was stirred at 25° C. for 1 h. TLC (PE/EA=10/1) showed complete consumption of the starting material. The reaction mixture was diluted with water (50 mL), and the pH of the aqueous was adjusted to 7-8 by the addition sat. Na₂CO₃. Then the mixture was extracted with EtOAc (250 mL×3). The combined organic layers were washed with sat. Na₂SO₃ (200 mL×2) and brine (200 mL×3), dried over Na₂SO₄, filtered, and concentrated in vacuo. The residue was purified by MPLC (normal phase column chromatography: SiO₂, Petroleum ether/Ethyl acetate=0/1 to 20/1). 13 g (44.06 mmol, 31.05% yield, 99% pure) of the title compound was obtained as a white solid, while 15 g (25.42 mmol, 17.92% yield, 50% purity) was obtained as a white solid.

1H NMR (CDCl3) d 8.25 (d, J=7.6 Hz, 1H), 6.44 (d, J=12 Hz, 1H), 4.60 (bs, 2H), 3.88 (s, 3H)

### Intermediate 90

### methyl4-((tert-butoxycarbonyl)amino)-2-fluoro-5-iodobenzoate

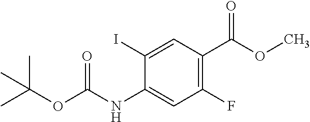 [SMILES: COC(=O)c1cc(I)c(NC(=O)OC(C)(C)C)cc1F]

To a solution of methyl 4-amino-2-fluoro-5-iodo-benzoate (8.5 g, 28.81 mmol, 1 eq, Intermediate 89) in DCM (85 mL) was added Et₃N (8.75 g, 86.43 mmol, 12.03 mL, 3 eq), DMAP (351.96 mg, 2.88 mmol, 0.1 eq), and (Boc)2O (9.43 g, 43.21 mmol, 9.93 mL, 1.5 eq). The mixture was stirred at 40° C. for 12 h. TLC (PE/EA=5/1) showed full consumption of the starting material. The reaction mixture was diluted with water (150 mL) and extracted with DCM (150 mL×3). The combined organic layers were washed with brine (100 mL×3), dried over Na₂SO₄, filtered, and concentrated in vacuo. The residue was purified by normal phase column chromatography (SiO₂, Petroleum ether/Ethyl acetate=50/1 to 10/1), and the fractions containing the title compound were combined and concentrated in vacuo to provide 3.2 g (8.10 mmol, 28.11% yield) of the title compound as a white solid.

1H NMR (CDCl3) d 8.45 (d, J=8 Hz, 1H), 8.09 (d, J=13.6 Hz, 1H), 7.08 (bs, 1H), 3.91 (s, 3H), 1.56 (s, 9H)

### Intermediate 91

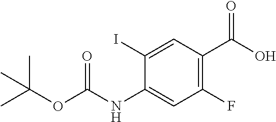 [SMILES: CC(C)(C)OC(=O)Nc1cc(F)c(C(=O)O)cc1I]

Cleavage of the ester of Intermediate 90 can be achieved under basic conditions known to the person of ordinary skill making sure that the protecting group of the amino group remains untouched which is cleaved later under acidic conditions (see Intermediate 96).

### Intermediate 92

### tert-butyl-N-[4-[(3,4-difluorophenyl)methylcarbamoyl]-5-fluoro-2-iodo-phenyl]carbamate

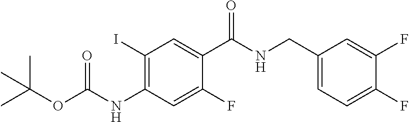 [SMILES: CC(C)(C)OC(=O)Nc1cc(F)c(C(=O)NCc2ccc(F)c(F)c2)cc1I]

To a solution of 4-(tert-butoxycarbonylamino)-2-fluoro-5-iodo-benzoic acid (5.8 g, 15.22 mmol, 1 eq, Intermediate 91) in Pyridine (45 mL) was added T3P (29.05 g, 45.65 mmol, 27.15 mL, 50% purity, 3 eq) and (3,4-difluoro)benzylamine (3.27 g, 22.83 mmol, 2.70 mL, 1.5 eq). The resulting mixture was stirred at 25° C. for 17 hr. TLC (PE:EA=1:1) showed starting material remained, therefore the mixture was stirred at 30° C. for 15 hr. TLC (PE/EA=1:1) showed starting material remained, therefore Pyridine (10 mL) and T3P (9.68 g, 15.22 mmol, 9.05 mL, 50% purity, 1 eq) were added and the resulting mixture was stirred at 30° C. for 2 hr. TLC (PE:EA=1:1) showed full consumption of the starting material, and the solution was concentrated in vacuum. The resulting residue was purified by column chromatography (SiO₂, Petroleum ether/Ethyl acetate=10:1), and fractions containing the desired product were concentrated in vacuo to provide 6.60 g (13.03 mmol, 86% yield) of the title compound as a white solid.

1H NMR (CDCl3): d 8.51 (d, J=8.4 Hz, 1H), 8.066 (d, J=15.2 Hz, 1H), 7.164-7.067 (m, 5H), 4.73 (d, J=5.6 Hz, 2H), 1.54 (s, 9H)

### Intermediate 93

### tert-butyl-N-[4-[(3,4-difluorophenyl)methylcarbamoyl]-5-fluoro-2-vinyl-phenyl]carbamate

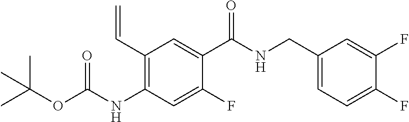 [SMILES: C=Cc1cc(C(=O)NCc2ccc(F)c(F)c2)c(F)cc1NC(=O)OC(C)(C)C]

To a solution of tert-butyl-N-[4-[(3,4-difluorophenyl)methylcarbamoyl]-5-fluoro-2-iodo-phenyl]carbamate (6.6 g, 13.04 mmol, 1 eq, Intermediate 92), potassium trifluoro(vinyl)boranate (1.75 g, 13.04 mmol, 1 eq) and Pd(dppf)Cl2 (953.91 mg, 1.30 mmol, 0.1 eq) in a mixture of 1,4-dioxane (60 mL) and H2O (12 mL) was added K2CO3 (5.41 g, 39.11 mmol, 3 eq) under N2. The resulting mixture was stirred at 80° C. for 12 h. TLC (Petroleum ether/Ethyl acetate=3:1) indicated full consumption of the starting material. The reaction mixture was quenched by the addition of sat. NH4CI (30 mL) and extracted with EtOAc (60 mL×3). The combined organic layers were washed with brine (40 mL×3), dried over Na2SO4, filtered, and concentrated in vacuo. The resulting residue was purified by column chromatography (SiO2, Petroleum ether/Ethyl acetate=10:1), and fractions containing the desired product were combined and concentrated in vacuo to provide 3.5 g (8.61 mmol, 66% yield) of the title compound as a white solid.

### Intermediate 94

### tert-butyl-N-[4-[(3,4-difluorophenyl)methylcarbamoyl]-5-fluoro-2-formyl-phenyl]carbamate

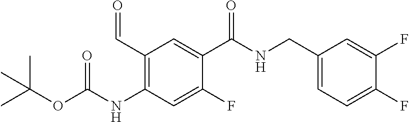 [SMILES: CC(C)(C)OC(=O)Nc1cc(F)c(C(=O)NCc2ccc(F)c(F)c2)cc1C=O]

To a solution of tert-butyl-N-[4-[(3,4-difluorophenyl)methylcarbamoyl]-5-fluoro-2-vinyl-phenyl]carbamate (2.98 g, 7.33 mmol, 1 eq, Intermediate 93) in H₂O (10 mL) and THF (30 mL) was added NalO₄ (4.71 g, 22.00 mmol, 1.22 mL, 3 eq) and OsO₄ (93.21 mg, 366.64 umol, 19.02 uL, 0.05 eq) at 0° C. under N₂. The mixture was stirred at 0° C. for 2 h. The reaction mixture was quenched by the addition of sat. aq Na₂SO₃ (30 mL) and extracted with EtOAc (70 mL×3). The combined organic layers were washed with sat. aq Na₂SO₃ (50 mL×4) and brine (50 mL×3), dried over Na₂SO₄, filtered, and concentrated in vacuo to provide 2.6 g of the title compound as a white solid which was used without further purification.

1H NMR (DMSO-d6): d 10.60 (s, 1H), 9.61 (s, 1H), 8.91 (d, J=2.4 Hz, 1H), 8.32 (d, J=8.4 Hz, 1H), 8.05 (d, J=14 Hz, 1H), 7.41-7.36 (m, 2H), 7.19 (bs, 1H), 4.46 (d, J=6 Hz, 2H), 1.48 (s, 9H)

### Intermediate 95

### 2-((tert-butoxycarbonyl)amino)-5-[(3,4-difluorophenyl)methylcarbamoyl]-4-fluorobenzoic acid

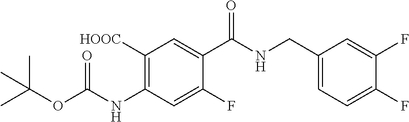 [SMILES: CC(C)(C)OC(=O)Nc1cc(F)c(C(=O)NCc2ccc(F)c(F)c2)cc1C(=O)O]

To a solution of tert-butyl-N-[4-[(3,4-difluorophenyl)methylcarbamoyl]-5-fluoro-2-formyl-phenyl]carbamate (2.59 g, 6.34 mmol, 1 eq, Intermediate 94), sodium; dihydrogen phosphate (228.10 mg, 1.90 mmol, 0.3 eq) and H₂O₂(1.49 g, 13.18 mmol, 1.27 mL, 30% purity, 2.08 eq) in MeCN (25 mL) and H₂O (5 mL) was added a solution of sodium chlorite (802.43 mg, 8.87 mmol, 1.4 eq) in H₂O (2 mL) dropwise at 0° C., the reaction mixture was stirred at 25° C. for 20 hr. The reaction mixture was diluted with sat. Na₂SO₃ (10 mL), the pH was adjusted to 3-2 by the addition of HCl. Then the mixture was extracted with EtOAc (60 mL×3). The combined organic layers were washed with brine (20 mL×3), dried over Na₂SO₄, filtered, and concentrated in vacuo. The resulting residue was purified by column chromatography (SiO₂, PE/EA=0:1), and fractions containing the desired compound were concentrated in vacuo to provide 2.2 g (5.18 mmol, 82% yield) of the title compound as a white solid.

1H NMR (DMSO-d6): d 13.41 (s, 1H), 8.70 (d, J=3.2 Hz, 1H), 8.37 (d, J=9.2 Hz, 1H), 7.98 (d, J=14 Hz, 1H), 7.40-7.34 (m, 2H), 7.172 (bs, 1H), 4.42 (d, J=6 Hz, 2H), 1.47 (s, 9H)

### Intermediate 96

### 2-amino-5-[(3,4-difluorophenyl)methylcarbamoyl]-4-fluorobenzoic acid

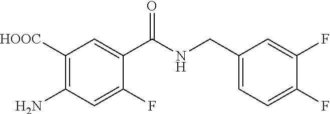 [SMILES: Nc1cc(F)c(C(=O)NCc2ccc(F)c(F)c2)cc1C(=O)O]

To a solution of 2-(tert-butoxycarbonylamino)-5-[(3,4-difluorophenyl)methylcarbamoyl]-4-fluoro-benzoic acid (1.9 g, 4.48 mmol, 1 eq, Intermediate 95) in TFA (4 mL) and CH₂Cl₂ (20 mL) and the resulting mixture was stirred at 25° C. for 2 hr. The reaction mixture was concentrated in vacuo to give 1.3 g of the title compound as a red oil, which was used without purification.

### Intermediate 97

### 2-bromo-5-[(3,4-difluorophenyl)methylcarbamoyl]-4-fluoro-benzoic acid

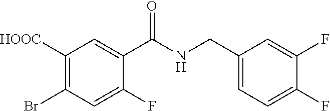 [SMILES: O=C(NCc1ccc(F)c(F)c1)c1cc(C(=O)O)c(Br)cc1F]

To a solution of 2-amino-5-[(3,4-difluorophenyl)methylcarbamoyl]-4-fluorobenzoic acid (1.3 g, 4.01 mmol, 1 eq, Intermediate 96) in MeCN (10 mL) was added to CuBr (1.67 g, 11.63 mmol, 354.11 uL, 2.9 eq) and tert-butyl nitrite (1.20 g, 11.63 mmol, 1.38 mL, 2.9 eq), and the resulting mixture was stirred at 25° C. for 12 hr. The reaction mixture was quenched by the addition of sat. aq. NH₄Cl (10 mL), and then extracted with EtOAc (10 mL*3). The combined organic layers were washed with brine (10 mL*3), dried over Na₂SO₄, filtered, and concentrated in vacuo to give a residue. The resulting residue was purified by column chromatography (SiO₂, Petroleum ether/Ethyl acetate=5/1 to 0/1). Fractions containing the desired compound were combined and concentrated in vacuo to provide 0.9 g (1.67 mmol, 42% yield, 72% purity) of the title compound as a red solid.

### SYNTHESIS OF EXAMPLE COMPOUNDS

### Example 1

### 4-chloro-N³-[4-(difluoromethoxy)-2-methylphenyl]-6-fluoro-N′-[(2-hydroxyphenyl)methyl]benzene-1,3-dicarboxamide

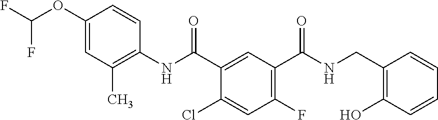 [SMILES: Cc1cc(OC(F)F)ccc1NC(=O)c1cc(C(=O)NCc2ccccc2O)c(F)cc1Cl]

To a solution of 4-chloro-5-{[4-(difluoromethoxy)-2-methylphenyl]carbamoyl}-2-fluorobenzoic acid (300 mg, 803 μmol, Intermediate 6 Intermediate 6) in DMSO (5 ml) was added 2-(aminomethyl)phenol (109 mg, 883 μmol), HATU (366 mg, 963 μmol; CAS-RN:[148893-10-1]) and then N,N-diisopropylethylamine (350 μl, 2.0 mmol; CAS-RN:[7087-68-5]). The mixture was stirred at rt for 90 min. The reaction mixture was treated with water and the resulting precipitate was filtered through a PTFE-filter. The filter cake was dissolved in DMSO and purified by reversed phase preparative. HPLC (acidic conditions). The product rich fractions were pooled, ACN was evaporated and the residue freeze dried to yield the desired product (273.1 mg, 99% purity, 70% yield).

LC-MS (method 1): Rₜ=1.13 min; MS (ESIpos): m/z=479.1 [M+H]⁺

1H-NMR (400 MHz, DMSO-d6) delta [ppm]: 2.073 (2.67), 2.287 (16.00), 2.518 (2.05), 2.522 (1.34), 3.322 (0.78), 4.418 (3.81), 4.433 (3.78), 6.758 (1.22), 6.761 (1.35), 6.777 (2.47), 6.779 (2.80), 6.795 (1.46), 6.798 (1.61), 6.812 (2.45), 6.815 (2.40), 6.832 (2.99), 6.834 (2.58), 7.030 (3.32), 7.039 (1.49), 7.053 (1.32), 7.064 (1.95), 7.068 (1.50), 7.084 (1.75), 7.087 (1.84), 7.102 (1.25), 7.109 (3.11), 7.116 (2.38), 7.171 (1.82), 7.174 (1.76), 7.190 (1.68), 7.216 (5.21), 7.401 (2.30), 7.444 (3.47), 7.466 (3.12), 7.715 (3.80), 7.740 (3.70), 7.886 (3.61), 7.905 (3.48), 8.856 (0.69), 8.868 (1.30), 8.883 (0.67), 9.598 (6.82), 10.103 (4.22).

The following examples listed in table 2 below were prepared analogous to the preparation of example 1 starting from Intermediate 6, Intermediate 29, Intermediate 38 by reacting with the corresponding amines.

Table 2

TABLE 2

| | Structure | |
|---|---|---|
| Example | Name | Analytical Data |
| Example 2 | 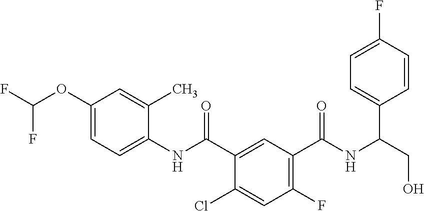 [SMILES: Cc1cc(OC(F)F)ccc1NC(=O)c1cc(C(=O)NC(CO)c2ccc(F)cc2)c(F)cc1Cl] | 1H-NMR (400 MHz, DMSO-d6) delta [ppm]: 2.084 (16.00), 2.287 (10.27), 2.518 (1.41), 2.522 (0.90), 3.616 (0.92), 3.629 (1.60), 3.644 (1.47), 3.659 (0.77), 5.000 (0.98), 5.014 (2.68), 5.028 (1.34), 5.049 (0.73), 7.030 (1.91), 7.039 (0.96), 7.054 (0.84), 7.061 (1.05), 7.109 (1.87), 7.116 (1.54), 7.145 (1.89), 7.150 (0.66), 7.162 (0.80), 7.167 (3.85), 7.185 (0.68), 7.190 (2.13), 7.216 (2.89), 7.400 (1.55), 7.410 (1.76), 7.415 (0.83), 7.424 (1.96), 7.431 (1.81), 7.446 (3.57), 7.468 (1.98), 7.709 (2.30), 7.733 (2.22), 7.844 (2.18), 7.863 (2.15), |
| | | 8.892 (0.98), 8.912 (0.96), 10.100 (2.75). |
| | 4-chloro-N³-[4-(difluoromethoxy)-2- | LC-MS (method 1): Rt = 1.11 min; MS |
| | methylphenyl]-6-fluoro-N¹-[1-(4- | (ESIpos): m/z = 511.1 [M + H]+ |
| | fluorophenyl)-2- | |
| | hydroxyethyl]benzene-1,3- | |
| | dicarboxamide | |
| Example 3; synthesis analogous example 58 | 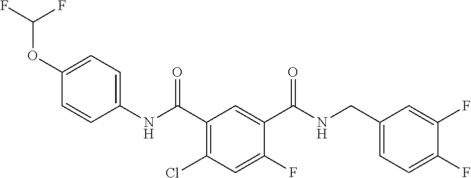 [SMILES: O=C(NCc1ccc(F)c(F)c1)c1cc(C(=O)Nc2ccc(OC(F)F)cc2)c(Cl)cc1F] | ¹H-NMR (400 MHz, DMSO-d6) δ [ppm]: 0.852 (0.76), 1.232 (2.75), 2.006 (0.41), 2.332 (0.78), 2.518 (3.54), 2.523 (2.35), 4.449 (7.67), 4.464 (7.65), 6.991 (5.81), 7.177 (16.00), 7.180 (12.89), 7.185 (5.23), 7.197 (5.37), 7.202 (12.54), 7.362 (6.35), 7.374 (3.02), 7.379 (2.25), 7.384 (2.27), 7.388 (2.40), 7.395 (6.03), 7.401 (3.04), 7.408 (2.18), 7.414 (2.29), 7.416 (2.83), 7.423 (4.82), 7.444 (2.16), 7.708 (1.44), 7.717 (13.65), 7.721 (4.11), 7.739 (14.67), |
| | | 7.747 (1.33), 7.762 (8.04), 7.870 (7.80), |
| | 4-chloro-N³-[4- | 7.888 (7.81), 9.085 (1.56), 9.100 (2.99), |
| | (difluoromethoxy)phenyl]-N¹-[(3,4- | 9.114 (1.52), 10.666 (8.53). |
| | difluorophenyl)methyl]-6- | LC-MS (§HTpost1_acid): Rt = 1.24 min; MS |
| | fluorobenzene-1,3-dicarboxamide | (ESIpos): m/z = 485 [M + H]+ |
| Example 4 | 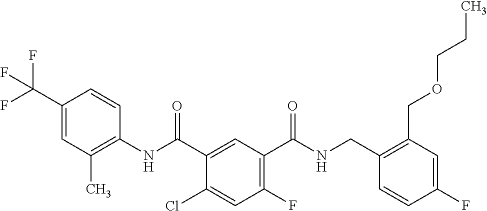 [SMILES: CCCOCc1cc(F)ccc1CNC(=O)c1cc(C(=O)Nc2ccc(C(F)(F)F)cc2C)c(Cl)cc1F] | ¹H-NMR (400 MHz, DMSO-d6) δ [ppm]: 0.867 (0.79), 0.877 (6.67), 0.886 (1.92), 0.896 (15.72), 0.904 (1.22), 0.908 (1.03), 0.914 (7.68), 1.532 (0.57), 1.551 (2.04), 1.567 (4.12), 1.577 (0.77), 1.586 (3.99), 1.602 (1.94), 1.620 (0.50), 2.241 (0.50), 2.323 (0.44), 2.327 (0.60), 2.332 (0.47), 2.365 (16.00), 2.518 (2.44), 2.523 (1.59), 2.665 (0.40), 2.669 (0.56), 2.673 (0.40), 3.423 (0.61), 3.430 (4.57), 3.439 (1.34), 3.446 (9.44), 3.456 (0.83), 3.463 (4.35), 4.477 (3.90), 4.490 (3.69), 4.573 (1.59), 4.582 (10.77), 7.104 (0.84), 7.111 (0.96), |
| | | 7.125 (1.77), 7.132 (2.00), 7.147 (1.03), |
| | 4-chloro-6-fluoro-N¹-{[4-fluoro-2- | 7.154 (1.14), 7.183 (1.89), 7.190 (1.54), |
| | (propoxymethyl)phenyl]methyl}-N³- | 7.207 (1.92), 7.214 (1.49), 7.367 (1.59), |
| | [2-methyl-4- | 7.381 (1.83), 7.388 (1.55), 7.402 (1.43), |
| | (trifluoromethyl)phenyl]benzene-1,3- | 7.591 (1.07), 7.612 (1.34), 7.660 (2.88), |
| | dicarboxamide | 7.737 (2.55), 7.750 (2.19), 7.763 (2.69), |
| | | 7.772 (1.65), 7.900 (2.84), 7.919 (2.72), |
| | | 8.957 (0.81), 8.970 (1.54), 8.984 (0.79), |
| | | 10.303 (3.79). |
| | | LC-MS (Method 4): Rt = 1.48 min; MS |
| | | (ESIpos): m/z = 555 [M + H]+ |

### Example 5

### 4-chloro-N′-[(3,4-difluorophenyl)methyl]-6-fluoro-N³-{4-[3-hydroxypiperidin-1-yl]-2-methylphenyl}benzene-1,3-dicarboxamide (stereoisomer 1)

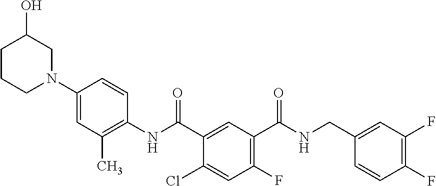 [SMILES: Cc1cc(N2CCCC(O)C2)ccc1NC(=O)c1cc(C(=O)NCc2ccc(F)c(F)c2)c(F)cc1Cl]

For the preparation of the racemic title compound see Example 1. Separation of 59 mg racemate into the enantiomers by preparative chiral HPLC (method I) gave 25 mg of the title compound (Rₜ=6.8-10.4 min).

Analytical chiral HPLC (method 4): Rₜ=2.53 min.

Optical rotation:[α]_{D}=2.9°+/−0.64° (c=1.0 g/100 ml DMSO)

1H-NMR (400 MHz, DMSO-d6) delta [ppm]: 1.239 (0.93), 1.249 (0.70), 1.263 (0.66), 1.272 (0.79), 1.504 (0.55), 1.537 (0.64), 1.718 (0.84), 1.727 (0.64), 1.742 (0.48), 1.751 (0.61), 1.761 (0.42), 1.866 (0.62), 1.898 (0.62), 2.210 (14.62), 2.336 (1.24), 2.444 (1.12), 2.470 (3.24), 2.518 (16.00), 2.522 (10.60), 2.585 (0.52), 2.592 (0.67), 2.615 (1.03), 2.621 (0.95), 2.644 (0.65), 2.651 (0.52), 2.660 (1.31), 3.450 (0.86), 3.480 (0.80), 3.546 (0.45), 3.569 (1.74), 3.591 (1.42), 4.458 (3.30), 4.473 (3.25), 4.806 (3.51), 4.817 (3.24), 6.736 (1.06), 6.743 (1.34), 6.758 (1.08), 6.765 (1.55), 6.790 (2.66), 6.796 (2.00), 7.167 (3.13), 7.188 (3.46), 7.362 (0.86), 7.367 (0.80), 7.381 (1.89), 7.386 (0.95), 7.392 (0.99), 7.397 (0.96), 7.402 (2.17), 7.408 (1.49), 7.416 (0.92), 7.423 (1.08), 7.430 (2.00), 7.450 (0.97), 7.706 (3.85), 7.731 (3.74), 7.837 (3.61), 7.856 (3.63), 9.085 (0.67), 9.099 (1.27), 9.113 (0.65), 9.811 (4.06).

LC-MS (method 1): Rₜ=0.96 min; MS (ESIpos): m/z=532.2 [M+H]⁺

### Example 6

### 4-chloro-N′-[(3,4-difluorophenyl)methyl]-6-fluoro-N³-{4-[3-hydroxypiperidin-1-yl]-2-methylphenyl}benzene-1,3-dicarboxamide (stereoisomer 2)

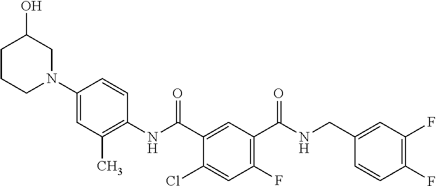 [SMILES: Cc1cc(N2CCCC(O)C2)ccc1NC(=O)c1cc(C(=O)NCc2ccc(F)c(F)c2)c(F)cc1Cl]

For the preparation of the racemic title compound see Example 1. Separation of 59 mg racemate into the enantiomers by preparative chiral HPLC (method I) gave 21 mg of the title compound (Rₜ=14.2-20 min).

Analytical chiral HPLC (method 4): Rₜ=4.2 min.

Optical rotation:[α]_{D}=−6.5°+/−0.94° (c=1.0 g/100 ml DMSO)

LC-MS (method 1): Rₜ=0.96 min; MS (ESIpos): m/z=532.2 [M+H]⁺

The following examples listed in table 3 below, were prepared analogous to the preparation of example 1 starting from Intermediate 38, Intermediate 11, Intermediate 8, Intermediate 55 by reacting with the corresponding amines Intermediate 2,

TABLE 3

| | Structure | |
|---|---|---|
| Example | Name | Analytical Data |
| Example 7 | 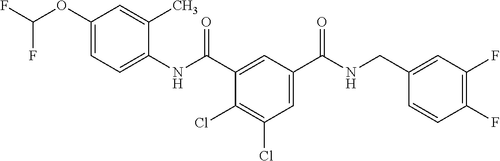 [SMILES: Cc1cc(OC(F)F)ccc1NC(=O)c1cc(C(=O)NCc2ccc(F)c(F)c2)cc(Cl)c1Cl] | ¹H-NMR (400 MHz, DMSO-d6) δ [ppm]: 2.075 (0.98), 2.297 (16.00), 2.518 (3.18), 2.523 (2.38), 4.476 (3.35), 4.490 (3.35), 7.035 (2.77), 7.048 (1.21), 7.055 (1.41), 7.069 (1.24), 7.076 (1.60), 7.122 (2.79), 7.129 (2.29), 7.171 (0.69), 7.177 (0.69), 7.182 (0.74), 7.188 (0.86), 7.193 (0.92), 7.197 (0.86), 7.204 (0.82), 7.221 (5.75), 7.369 (2.08), 7.375 (0.89), 7.390 (2.97), 7.397 (2.04), 7.405 (3.19), 7.412 (1.25), |
| | | 7.418 (2.84), 7.425 (0.91), 7.439 (0.98), |
| | 4,5-dichloro-N³-[4-(difluoromethoxy)- | 7.469 (3.64), 7.490 (3.27), 8.064 (5.32), |
| | 2-methylphenyl]-N¹-[(3,4- | 8.069 (5.49), 8.233 (5.73), 8.239 (5.18), |
| | difluorophenyl)methyl]benzene-1,3- | 9.361 (0.85), 9.375 (1.76), 9.390 (0.85), |
| | dicarboxamide | 10.179 (4.18). |
| | | LC-MS (method 1): Rt = 1.30 min; MS |
| | | (ESIpos): m/z = 515.2 [M + H]+ |
| Example 8 | 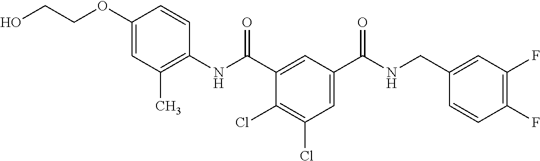 [SMILES: Cc1cc(OCCO)ccc1NC(=O)c1cc(C(=O)NCc2ccc(F)c(F)c2)cc(Cl)c1Cl] | 1H-NMR (400 MHz, DMSO-d6) delta [ppm]: 2.255 (16.00), 2.518 (2.79), 2.523 (2.17), 3.688 (1.48), 3.702 (3.52), 3.714 (4.06), 3.727 (1.84), 3.961 (3.50), 3.974 (5.05), 3.986 (2.87), 4.475 (3.35), 4.489 (3.33), 4.852 (2.60), 4.866 (5.81), 4.880 (2.35), 6.788 (1.33), 6.795 (1.61), 6.809 (1.34), 6.816 (1.79), 6.853 (3.05), 6.860 (2.34), 7.171 (0.71), 7.176 (0.70), 7.180 (0.74), 7.187 (0.88), 7.192 (0.92), 7.196 (0.86), |
| | | 7.203 (0.83), 7.276 (3.57), 7.298 (3.23), |
| | 4,5-dichloro-N¹-[(3,4- | 7.369 (2.24), 7.374 (0.92), 7.390 (2.92), |
| | difluorophenyl)methyl]-N³-[4-(2- | 7.396 (2.04), 7.404 (0.98), 7.411 (1.21), |
| | hydroxyethoxy)-2- | 7.418 (3.14), 7.423 (0.92), 7.438 (1.05), |
| | methylphenyl]benzene-1,3- | 8.041 (5.15), 8.047 (5.57), 8.218 (6.38), |
| | dicarboxamide | 8.223 (5.70), 9.357 (0.91), 9.372 (1.86), |
| | | 9.387 (0.86), 9.984 (4.35). |
| | | LC-MS (method 1): Rt = 1.09 min; MS |
| | | (ESIpos): m/z = 509.2 [M + H]+ |
| Example 9 | 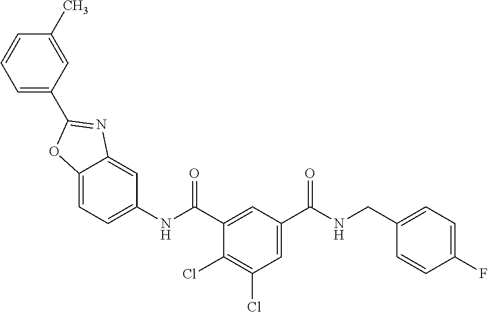 [SMILES: Cc1cccc(-c2nc3cc(NC(=O)c4cc(C(=O)NCc5ccc(F)cc5)cc(Cl)c4Cl)ccc3o2)c1] | ¹H-NMR (400 MHz, DMSO-d6) δ [ppm]: 2.518 (1.31), 2.523 (0.91), 2.763 (16.00), 4.471 (3.32), 4.486 (3.33), 7.143 (3.04), 7.149 (1.00), 7.160 (1.21), 7.166 (6.07), 7.172 (1.19), 7.183 (1.15), 7.188 (3.85), 7.196 (0.41), 7.360 (2.75), 7.365 (1.12), 7.373 (3.03), 7.381 (2.64), 7.390 (0.97), 7.395 (2.36), 7.418 (0.75), 7.421 (0.82), 7.440 (1.72), 7.458 (2.43), 7.474 (2.61), 7.504 (1.93), 7.508 (1.98), 7.523 (1.75), 7.526 (1.59), 7.541 (0.72), 7.545 (0.69), 7.640 (2.26), 7.645 (2.21), 7.662 (2.65), 7.667 (2.83), 7.794 (4.24), 7.815 (3.11), 8.112 (5.23), 8.117 (5.58), 8.129 (2.04), 8.132 (2.01), 8.148 (1.96), 8.151 (1.74), 8.246 (5.64), 8.251 (5.50), 8.265 (3.74), 8.270 (3.81), 9.335 (0.84), 9.349 (1.77), 9.364 (0.84), 10.880 (4.27). LC-MS (method 1): Rt = 1.46 min; MS |
| | | (ESIpos): m/z = 548.2 [M + H]+ |
| | 4,5-dichloro-N¹-[(4- | |
| | fluorophenyl)methyl]-N³-[2-(3- | |
| | methylphenyl)-1,3-benzoxazol-5- | |
| | yl]benzene-1,3-dicarboxamide | |
| Example 10 | 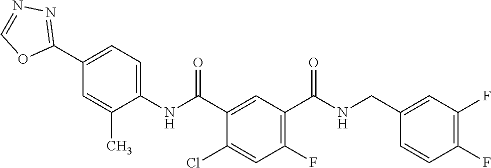 [SMILES: Cc1cc(-c2nnco2)ccc1NC(=O)c1cc(C(=O)NCc2ccc(F)c(F)c2)c(F)cc1Cl] | 1H-NMR (400 MHz, DMSO-d6) delta [ppm]: 1.137 (0.67), 2.084 (5.33), 2.390 (16.00), 2.409 (1.09), 2.518 (5.73), 2.522 (3.67), 4.465 (3.73), 4.480 (3.72), 7.201 (1.07), 7.207 (1.09), 7.235 (0.51), 7.368 (1.02), 7.373 (1.03), 7.384 (1.65), 7.393 (1.38), 7.397 (1.33), 7.405 (2.82), 7.411 (1.71), 7.417 (1.19), 7.422 (1.20), 7.426 (1.49), 7.432 (2.34), 7.453 (1.16), 7.752 (2.18), 7.778 (2.38), 7.785 (1.63), 7.807 (2.07), |
| | | 7.894 (1.73), 7.916 (1.25), 7.939 (2.67), |
| | 4-chloro-N¹-[(3,4- | 7.948 (3.91), 8.044 (0.49), 9.104 (0.74), |
| | difluorophenyl)methyl]-6-fluoro-N³- | 9.119 (1.37), 9.133 (0.73), 9.337 (0.95), |
| | [2-methyl-4-(1,3,4-oxadiazol-2- | 9.345 (10.90), 10.280 (4.00). |
| | yl)phenyl]benzene-1,3- | LC-MS (Method 4): Rt = 1.13 min; MS |
| | dicarboxamide | (ESIpos): m/z = 501 [M + H]+ |
| Example 11 | 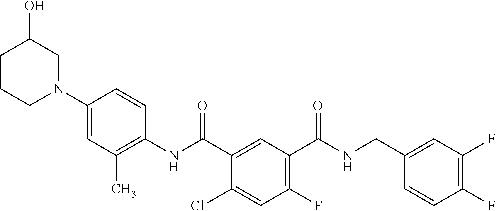 [SMILES: Cc1cc(N2CCCC(O)C2)ccc1NC(=O)c1cc(C(=O)NCc2ccc(F)c(F)c2)c(F)cc1Cl] | ¹H-NMR (400 MHz, DMSO-d6) δ [ppm]: 1.138 (1.65), 1.270 (0.62), 1.295 (0.70), 1.523 (0.64), 1.555 (0.69), 1.744 (0.77), 1.872 (0.88), 1.895 (0.84), 2.084 (0.43), 2.116 (0.74), 2.219 (16.00), 2.327 (0.52), 2.522 (2.03), 2.534 (1.93), 2.548 (2.95), 2.562 (1.47), 2.665 (0.88), 2.669 (1.03), 2.687 (0.69), 3.459 (1.60), 3.490 (1.38), 3.575 (1.66), 3.599 (2.17), 4.459 (4.55), 4.474 (4.57), 5.026 (0.41), 5.758 (6.59), 6.838 (0.89), 7.200 (2.32), 7.212 (1.88), 7.362 (1.08), 7.367 (1.06), 7.381 (2.37), 7.387 (1.40), 7.392 (1.43), 7.397 (1.41), |
| 11 | | 7.402 (2.70), 7.408 (1.96), 7.416 (1.23), |
| | 4-chloro-N¹-[(3,4- | 7.423 (1.38), 7.429 (2.44), 7.450 (1.07), |
| | difluorophenyl)methyl]-6-fluoro-N³- | 7.709 (4.06), 7.734 (3.99), 7.843 (3.81), |
| | {4-[3-hydroxypiperidin-1-yl]-2- | 7.862 (3.77), 7.977 (0.45), 8.245 (0.46), |
| | methylphenyl}benzene-1,3- | 9.086 (0.95), 9.099 (1.83), 9.113 (0.96), |
| | dicarboxamide | 9.840 (1.84). |
| | | LC-MS (Method 4): Rt = 1.05 min; MS |
| | | (ESIpos): m/z = 532 [M + H]+ |
| Example 12 | 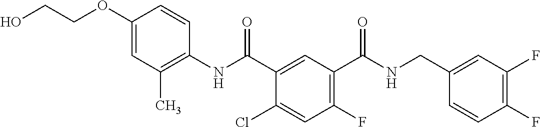 [SMILES: Cc1cc(OCCO)ccc1NC(=O)c1cc(C(=O)NCc2ccc(F)c(F)c2)c(F)cc1Cl] | ¹H-NMR (400 MHz, DMSO-d6) δ [ppm]: 2.239 (16.00), 2.331 (0.50), 2.518 (3.01), 2.523 (2.06), 2.673 (0.50), 3.686 (1.46), 3.699 (3.58), 3.712 (4.06), 3.725 (1.80), 3.957 (3.40), 3.970 (4.97), 3.982 (2.79), 4.460 (3.68), 4.475 (3.63), 4.854 (2.23), 4.868 (5.20), 4.882 (2.16), 6.773 (1.36), 6.780 (1.62), 6.795 (1.39), 6.802 (1.79), |
| | | 6.842 (3.12), 6.849 (2.46), 7.181 (0.77), |
| | 4-chloro-N¹-[(3,4- | 7.185 (0.77), 7.192 (0.82), 7.197 (0.95), |
| | difluorophenyl)methyl]-6-fluoro-N³- | 7.202 (0.96), 7.206 (0.93), 7.212 (0.89), |
| | [4-(2-hydroxyethoxy)-2- | 7.252 (3.40), 7.274 (3.10), 7.363 (0.90), |
| | methylphenyl]benzene-1,3- | 7.369 (0.87), 7.382 (1.95), 7.388 (1.08), |
| | dicarboxamide | 7.393 (1.12), 7.398 (1.10), 7.403 (2.30), |
| | | 7.408 (1.50), 7.412 (1.11), 7.417 (0.96), |
| | | 7.424 (1.13), 7.430 (2.09), 7.451 (0.97), |
| | | 7.715 (3.86), 7.740 (3.79), 7.861 (3.73), |
| | | 7.880 (3.62), 9.089 (0.75), 9.104 (1.44), |
| | | 9.118 (0.72), 9.904 (4.36). |
| | | LC-MS (Method 4): Rt = 1.09 min; MS |
| | | (ESIpos): m/z = 493 [M + H]+ |

### Example 13

### 4,5-dichloro-N′-[(3,4-difluorophenyl)methyl]-N³-[5-(trifluoromethyl)pyridin-2-yl]benzene-1,3-dicarboxamide

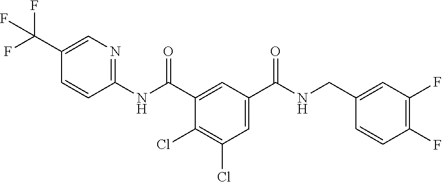 [SMILES: O=C(NCc1ccc(F)c(F)c1)c1cc(Cl)c(Cl)c(C(=O)Nc2ccc(C(F)(F)F)cn2)c1]

To a solution of 2,3-dichloro-5-{[(3,4-difluorophenyl)methyl]carbamoyl}benzoic acid (40.0 mg, 111 μmol, Intermediate 11) in dichloromethane (1.4 ml) was added 5-(trifluoromethyl)pyridin-2-amine (27.0 mg, 167 μmol; CAS-RN:[74784-70-6]), then pyridine (45 μl) followed by 1-chloro-N,N,2-trimethylprop-1-en-1-amine (44 μl, 330 μmol) and the mixture was stirred at rt for 16 h. The reaction mixture was treated with 3 drops of water and the reactions mixture was extracted 2× with DCM. The combined organic phases were evaporated and the residue purified by preparative HPLC (acidic conditions) to give 22.8 mg (99% purity, 40% yield) of the title compound.

LC-MS (method 1): Rt=1.36 min; MS (ESIpos): m/z=504.2 [M+H]⁺

1H-NMR (400 MHz, DMSO-d6) delta [ppm]: 2.336 (0.90), 2.518 (11.94), 2.523 (8.89), 2.679 (0.87), 4.462 (9.07), 4.476 (9.05), 7.167 (1.83), 7.170 (1.78), 7.178 (1.97), 7.183 (2.31), 7.188 (2.40), 7.192 (2.22), 7.197 (2.19), 7.362 (4.31), 7.369 (2.38), 7.383 (7.74), 7.389 (5.82), 7.393 (2.97), 7.399 (2.61), 7.404 (3.13), 7.410 (6.65), 7.418 (2.35), 7.431 (2.71), 8.077 (13.61), 8.082 (14.43), 8.231 (16.00), 8.236 (15.60), 8.276 (2.93), 8.281 (3.03), 8.298 (4.58), 8.304 (4.57), 8.375 (3.46), 8.397 (2.28), 8.786 (5.50), 9.312 (2.24), 9.327 (4.72), 9.342 (2.25), 11.679 (6.20).

The following examples listed in table 4 below were prepared analogous to the preparation of example 1 starting from Intermediate 11 by reacting with the corresponding amines.

TABLE 4

| | Structure | |
|---|---|---|
| Example | Name | Analytical Data |
| Example 14 | 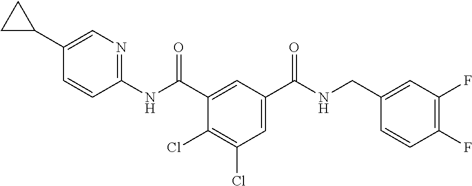 [SMILES: O=C(NCc1ccc(F)c(F)c1)c1cc(Cl)c(Cl)c(C(=O)Nc2ccc(C3CC3)cn2)c1] | ¹H-NMR (400 MHz, DMSO-d6) δ [ppm]: 0.700 (1.87), 0.711 (6.46), 0.716 (5.84), 0.724 (6.24), 0.729 (6.10), 0.739 (2.14), 0.955 (2.33), 0.965 (5.78), 0.971 (5.82), 0.976 (3.11), 0.982 (3.03), 0.986 (6.13), 0.991 (5.58), 1.003 (2.04), 1.231 (0.48), 1.913 (0.76), 1.926 (1.52), 1.934 (1.70), 1.947 (2.80), 1.959 (1.58), 1.967 (1.41), 1.980 (0.67), 2.318 (0.72), 2.518 (8.84), 2.523 (6.77), 2.679 (0.70), 4.456 (7.82), 4.471 (7.80), 7.162 (1.59), 7.166 (1.58), 7.172 (1.70), 7.178 (2.03), 7.183 (2.12), |
| | | 7.187 (2.01), 7.193 (1.90), 7.359 (4.95), |
| | 4,5-dichloro-N³-(5- | 7.364 (2.10), 7.380 (6.49), 7.386 (4.73), |
| | cyclopropylpyridin-2-yl)-N¹-[(3,4- | 7.393 (2.25), 7.402 (2.82), 7.407 (7.46), |
| | difluorophenyl)methyl]benzene-1,3- | 7.413 (2.15), 7.429 (2.32), 7.497 (2.96), |
| | dicarboxamide | 7.503 (2.99), 7.519 (3.14), 7.525 (3.18), |
| | | 8.024 (6.67), 8.029 (6.88), 8.052 (4.18), |
| | | 8.073 (3.78), 8.193 (16.00), 8.198 (15.14), |
| | | 9.304 (1.92), 9.318 (3.98), 9.333 (1.89), |
| | | 11.099 (7.64). |
| | | LC-MS (method 1): Rt = 1.30 min; MS |
| | | (ESIpos): m/z = 476.2 [M + H]+ |

### Example 15

### 4,5-dichloro-N-[(2-methoxyphenyl)methyl]-N³-phenylbenzene-_,3-dicarboxamide

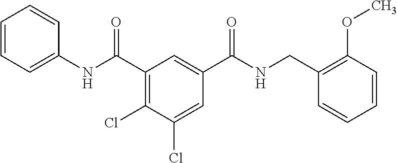 [SMILES: COc1ccccc1CNC(=O)c1cc(Cl)c(Cl)c(C(=O)Nc2ccccc2)c1]

In an autoclave 5-bromo-2,3-dichloro-N-phenylbenzamide (50.0 mg, 145 μmol, Intermediate 63 Intermediate 63) was dissolved in THF (2 ml), 1-(2-methoxyphenyl)methanamine (110 μl, 870 μmol) and triethylamine (61 μl, 430 μmol) 1,1′-Bis(diphenylphosphino)ferrocene-palladium(II)dichloride dichlormethane complex: (23.7 mg, 29.0 μmol; CAS-RN: [95464-05-4]) was added. The reaction mixture was purged 2× with carbon monoxide then the reaction vessel pressurized with carbon monoxide at 16.1 bar. The mixture was stirred at RT for 30 min and then the temperature was increased to 100° C. and stirring continued for 20 h. The crude reaction mixture was filtered, the residue washed with methanol and the organic solvents were removed under reduced pressure. After purification by reversed phase HPLC 29.4 mg (100% purity, 47% yield) of the title compound were obtained.

LC-MS (Method 4): Rt=1.26 min; MS (ESIpos): m/z=429 [M+H]+

1H-NMR (400 MHz, DMSO-d6) delta [ppm]: 2.075 (3.44), 2.518 (3.16), 2.523 (2.22), 3.805 (0.44), 3.817 (16.00), 4.448 (2.26), 4.463 (2.28), 6.891 (0.72), 6.893 (0.78), 6.909 (1.55), 6.911 (1.66), 6.927 (0.86), 6.930 (0.91), 6.991 (1.34), 7.009 (1.60), 7.011 (1.50), 7.115 (0.42), 7.117 (0.73), 7.121 (0.44), 7.136 (1.55), 7.152 (0.56), 7.155 (0.98), 7.157 (0.55), 7.191 (1.00), 7.195 (1.20), 7.210 (0.94), 7.214 (1.02), 7.231 (0.82), 7.235 (0.69), 7.252 (1.07), 7.270 (0.61), 7.275 (0.52), 7.352 (1.84), 7.357 (0.68), 7.373 (2.50), 7.387 (0.57), 7.392 (1.72), 7.686 (2.26), 7.688 (2.65), 7.702 (0.67), 7.706 (2.35), 7.710 (1.90), 8.090 (3.13), 8.095 (3.30), 8.243 (3.47), 8.248 (3.18), 9.119 (0.51), 9.133 (1.03), 9.148 (0.48), 10.676 (2.23).

The following examples listed in table 5 below were prepared analogous to the preparation of example 1 starting from Intermediate 63 Intermediate 63 by reacting with the corresponding benzyl amines.

TABLE 5

| | Structure | |
|---|---|---|
| Example | Name | Analytical Data |
| Example 16 | 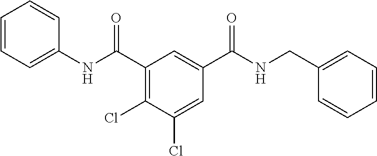 [SMILES: O=C(NCc1ccccc1)c1cc(Cl)c(Cl)c(C(=O)Nc2ccccc2)c1] | 1H-NMR (400 MHz, DMSO-d6) delta [ppm]: 2.074 (1.27), 2.084 (7.32), 2.322 (1.99), 2.326 (2.71), 2.331 (1.99), 2.522 (12.38), 2.664 (1.99), 2.669 (2.67), 4.484 (3.93), 4.499 (3.93), 7.117 (0.95), 7.136 (2.12), 7.155 (1.27), 7.248 (0.81), 7.253 (1.13), 7.266 (1.04), 7.274 (0.86), 7.313 (1.04), 7.325 (16.00), 7.334 (4.84), 7.339 (5.06), 7.351 (2.76), 7.372 (3.75), 7.391 (2.35), 7.683 (3.75), 7.702 (3.39), 8.071 (3.66), |
| | | 8.076 (3.84), 8.231 (3.98), 8.236 (3.75), |
| | N1-benzyl-4,5-dichloro-N³- | 9.310 (0.77), 9.325 (1.54), 9.339 (0.77), |
| | phenylbenzene-1,3-dicarboxamide | 10.673 (3.21). |
| | | LC-MS (OA01b02): Rₜ = 1.30 min; MS |
| | | (ESIpos): m/z = 397 [M-H]⁺ |
| Example 17 | 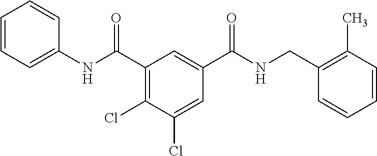 [SMILES: Cc1ccccc1CNC(=O)c1cc(Cl)c(Cl)c(C(=O)Nc2ccccc2)c1] | 1H-NMR (400 MHz, DMSO-d6) delta [ppm]: 2.316 (16.00), 2.327 (0.78), 2.332 (0.50), 2.518 (2.07), 2.523 (1.39), 2.669 (0.44), 4.468 (3.39), 4.482 (3.40), 7.114 (0.59), 7.117 (0.97), 7.119 (0.63), 7.135 (2.23), 7.144 (0.58), 7.151 (1.60), 7.154 (1.58), 7.159 (1.78), 7.168 (3.12), 7.174 (8.97), 7.182 (2.10), 7.189 (0.60), 7.245 (1.09), 7.254 (1.68), 7.264 (0.89), 7.350 (2.44), 7.371 (3.42), 7.385 (0.81), 7.390 (2.31), |
| | | 7.682 (3.09), 7.685 (3.59), 7.704 (3.23), |
| | 4,5-dichloro-N¹-[(2- | 7.706 (2.50), 8.086 (3.88), 8.091 (4.06), |
| | methylphenyl)methyl]-N³- | 8.243 (4.15), 8.248 (4.02), 9.150 (0.68), |
| | phenylbenzene-1,3-dicarboxamide | 9.164 (1.39), 9.178 (0.68), 10.674 (3.01). |
| | | LC-MS (Method 4): Rₜ = 1.29 min; MS |
| | | (ESIpos): m/z = 413 [M + H]⁺ |

The following examples listed in table 6 below were prepared analogous to the preparation of example 1 starting from Intermediate 17 by reacting with the corresponding amines.

TABLE 6

| | | |
|---|---|---|
| Example 18 | 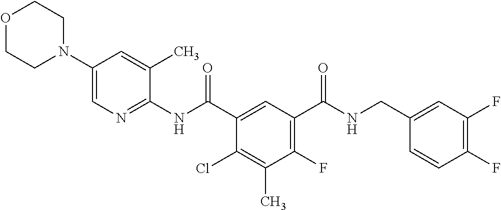 [SMILES: Cc1cc(N2CCOCC2)cnc1NC(=O)c1cc(C(=O)NCc2ccc(F)c(F)c2)c(F)c(C)c1Cl] | ¹H-NMR (400 MHz, DMSO-d6) δ [ppm]: 2.254 (16.00), 2.323 (0.77), 2.327 (0.87), 2.357 (4.84), 2.518 (1.68), 2.523 (1.11), 2.669 (0.49), 2.771 (0.84), 3.001 (0.80), 3.160 (4.15), 3.714 (0.66), 3.739 (3.77), 3.751 (4.87), 3.762 (3.47), 4.455 (3.16), 4.470 (3.13), 7.199 (0.95), 7.306 (1.62), 7.359 (0.76), 7.363 (0.76), 7.383 (2.15), 7.393 (0.91), 7.405 (2.75), 7.410 (2.01), 7.426 (1.22), 7.432 (2.38), 7.453 (1.11), 7.637 (0.79), 7.653 (0.76), 7.990 (0.98), 9.090 (1.00), 10.376 (1.64). LC-MS (method 1): Rₜ = 1.04 min; MS |
| | | (ESIpos): m/z = 533.3 [M + H]⁺ |
| | 4-chloro-N¹-[(3,4- | |
| | difluorophenyl)methyl]-6-fluoro-5- | |
| | methyl-N³-[3-methyl-5-(morpholin-4- | |
| | yl)pyridin-2-yl]benzene-1,3- | |
| | dicarboxamide | |

The following examples listed in table 7 below were prepared analogous to the preparation of example 1 starting from Intermediate 17 Intermediate 17 by reacting with the corresponding amines.

TABLE 7

| | Structure | |
|---|---|---|
| Example | Name | Analytical Data |
| Example 19 | 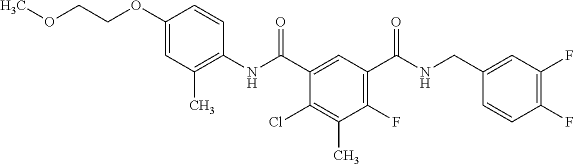 [SMILES: COCCOc1ccc(NC(=O)c2cc(C(=O)NCc3ccc(F)c(F)c3)c(F)c(C)c2Cl)c(C)c1] | ¹H-NMR (400 MHz, DMSO-d6) δ [ppm]: 2.238 (2.09), 2.362 (1.14), 2.368 (1.09), 3.308 (5.66), 3.330 (16.00), 3.639 (0.44), 3.650 (0.48), 3.661 (0.50), 4.066 (0.47), 4.078 (0.47), 4.088 (0.41), 4.457 (0.51), 4.471 (0.50), 6.847 (0.40), 7.257 (0.46), 7.279 (0.41), 9.870 (0.55). LC-MS (method 1): Rₜ = 1.20 min; MS (ESIpos): m/z = 521.3 [M + H]⁺ |
| | 4-chloro-N¹-[(3,4- | |
| | difluorophenyl)methyl]-6-fluoro-N³- | |
| | [4-(2-methoxyethoxy)-2- | |
| | methylphenyl]-5-methylbenzene-1,3- | |
| | dicarboxamide | |
| Example 20 | 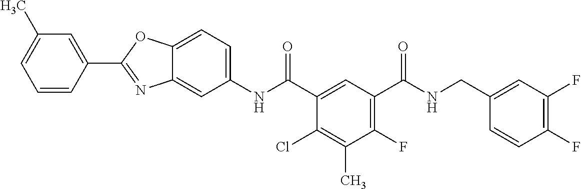 [SMILES: Cc1cccc(-c2nc3cc(NC(=O)c4cc(C(=O)NCc5ccc(F)c(F)c5)c(F)c(C)c4Cl)ccc3o2)c1] | ¹H-NMR (400 MHz, DMSO-d6) δ [ppm]: 1.230 (0.51), 2.085 (0.50), 2.379 (7.46), 2.385 (7.59), 2.518 (3.48), 2.522 (2.23), 2.686 (1.30), 2.712 (2.24), 2.744 (0.57), 2.761 (16.00), 3.732 (0.76), 4.456 (3.36), 4.471 (3.36), 6.691 (0.41), 6.886 (0.50), 6.892 (0.48), 7.185 (0.70), 7.188 (0.71), 7.200 (0.87), 7.206 (0.90), 7.209 (0.88), 7.215 (0.86), 7.367 (0.92), 7.373 (0.92), 7.381 (1.29), 7.387 (1.14), 7.392 (1.15), 7.397 (1.15), 7.402 (3.57), 7.408 (1.70), 7.417 (1.70), 7.423 (2.60), 7.430 (2.50), |
| | | 7.438 (1.91), 7.457 (2.54), 7.473 (2.59), |
| | 4-chloro-N¹-[(3,4- | 7.502 (1.94), 7.506 (1.98), 7.521 (1.77), |
| | difluorophenyl)methyl]-6-fluoro-5- | 7.524 (1.64), 7.539 (0.65), 7.543 (0.65), |
| | methyl-N³-[2-(3-methylphenyl)-1,3- | 7.647 (1.95), 7.652 (1.98), 7.669 (2.42), |
| | benzoxazol-5-yl]benzene-1,3- | 7.674 (2.65), 7.729 (2.31), 7.747 (2.40), |
| | dicarboxamide | 7.776 (4.08), 7.799 (2.96), 8.126 (1.96), |
| | | 8.130 (2.00), 8.146 (1.98), 8.262 (3.73), |
| | | 8.267 (3.78), 9.103 (0.73), 9.118 (1.45), |
| | | 9.133 (0.72), 10.759 (4.23). |
| | | LC-MS (method 1): Rₜ = 1.45 min; MS |
| | | (ESlpos): m/z = 564.3 [M + H]⁺ |

The following examples listed in table 8 below were prepared analogous to the preparation of example 1 starting from Intermediate 66 by reacting with the corresponding amines.

TABLE 8

| | Structure | |
|---|---|---|
| Example | Name | Analytical Data |
| Example 21 | 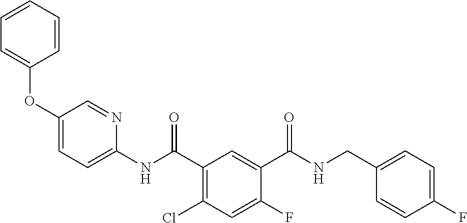 [SMILES: O=C(NCc1ccc(F)cc1)c1cc(C(=O)Nc2ccc(Oc3ccccc3)cn2)c(Cl)cc1F] | ¹H-NMR (400 MHz, DMSO-d6) δ [ppm]: 2.518 (5.51), 2.523 (3.66), 4.448 (9.85), 4.464 (9.77), 5.759 (1.66), 7.036 (1.29), 7.042 (9.63), 7.044 (11.05), 7.048 (6.06), 7.058 (3.60), 7.064 (12.99), 7.067 (10.46), 7.140 (2.87), 7.143 (4.23), 7.148 (8.11), 7.153 (3.10), 7.161 (9.20), 7.164 (6.60), 7.170 (16.00), 7.177 (5.69), 7.179 (5.85), 7.182 (3.18), 7.187 (3.14), 7.193 (9.51), 7.200 (1.04), 7.361 (7.35), 7.367 (3.28), 7.375 (8.29), 7.383 (7.87), 7.390 (12.54), 7.396 (7.91), 7.403 (2.40), 7.408 (11.17), 7.412 (11.53), 7.418 (1.57), 7.425 (3.00), 7.431 (7.46), 7.436 (1.01), 7.576 (5.86), |
| | | 7.584 (5.60), 7.599 (5.67), 7.606 (5.92), |
| | 4-chloro-6-fluoro-N¹-[(4- | 7.691 (9.56), 7.716 (9.38), 7.845 (8.06), |
| | fluorophenyl)methyl]-N³-(5- | 7.864 (7.77), 8.187 (8.03), 8.194 (9.41), |
| | phenoxypyridin-2-yl)benzene-1,3- | 8.218 (2.76), 9.022 (1.95), 9.037 (3.76), |
| | dicarboxamide | 9.052 (1.87), 11.182 (9.95). |
| | | LC-MS (method 1): Rt = 1.31 min; MS |
| | | (ESlpos): m/z = 494.2 [M + H]+ |

The following examples listed in table 9 below were prepared analogous to the preparation of example 1 starting from Intermediate 66, Intermediate 26, Intermediate 32, Intermediate 38,

Intermediate 35 by reacting with the corresponding amines.

TABLE 9

| | | |
|---|---|---|
| Example 22 | 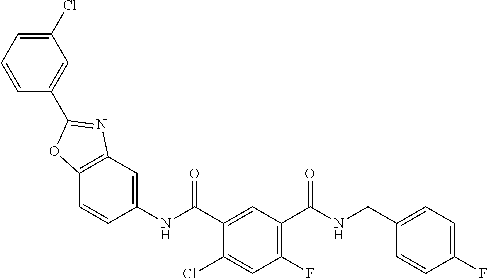 [SMILES: O=C(NCc1ccc(F)cc1)c1cc(C(=O)Nc2ccc3oc(-c4cccc(Cl)c4)nc3c2)c(Cl)cc1F] | ¹H-NMR (400 MHz, DMSO-d6) δ [ppm]: 1.230 (0.83), 2.336 (1.29), 2.518 (16.00), 2.523 (11.86), 2.679 (1.26), 4.454 (8.03), 4.468 (7.92), 7.144 (0.76), 7.152 (6.74), 7.157 (2.10), 7.168 (2.92), 7.174 (13.68), 7.180 (2.53), 7.191 (2.53), 7.197 (7.89), 7.204 (0.82), 7.367 (6.24), 7.373 (2.52), 7.381 (6.82), 7.389 (5.65), 7.398 (2.19), 7.403 (4.89), 7.648 (2.99), 7.668 (7.51), 7.673 (4.87), 7.678 (4.48), 7.687 (5.86), 7.695 (5.70), 7.700 (5.70), 7.719 (4.75), 7.721 (5.24), 7.723 (4.78), 7.727 (4.69), 7.739 (2.74), 7.742 (2.80), 7.744 (3.42), 7.747 (3.58), 7.750 (8.26), 7.775 (8.03), 7.803 (9.40), 7.825 (6.58), 7.896 (7.88), 7.915 (7.64), 8.156 (3.31), 8.160 (5.23), 8.163 (3.96), 8.175 (2.54), 8.179 (5.74), |
| | | 8.182 (4.77), 8.186 (6.44), 8.190 (7.92), |
| | 4-chloro-N³-[2-(3-chlorophenyl)-1,3- | 8.194 (3.96), 8.263 (8.31), 8.268 (8.29), |
| | benzoxazol-5-yl]-6-fluoro-N¹-[(4- | 9.078 (1.64), 9.093 (3.19), 9.108 (1.59), |
| | fluorophenyl)methyl]benzene-1,3- | 10.810 (9.16). |
| | dicarboxamide | LC-MS (method 1): Rₜ = 1.42 min; MS |
| | | (ESIpos): m/z = 552.2 [M + H]⁺ |
| Example 23 | 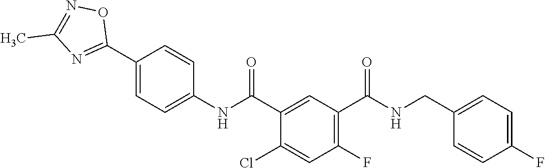 [SMILES: Cc1noc(-c2ccc(NC(=O)c3cc(C(=O)NCc4ccc(F)cc4)c(F)cc3Cl)cc2)n1] | ¹H-NMR (400 MHz, DMSO-d6) δ [ppm]: 2.408 (16.00), 4.449 (3.29), 4.463 (3.29), 7.148 (2.12), 7.171 (4.51), 7.193 (2.64), 7.362 (2.24), 7.376 (2.58), 7.383 (2.29), 7.398 (1.82), 7.755 (2.61), 7.780 (2.59), 7.902 (2.68), 7.920 (3.32), 7.926 (3.27), 7.948 (3.79), 8.092 (4.91), 8.114 (3.68), 9.068 (0.70), 9.083 (1.36), 9.097 (0.70), 11.012 (3.39). LC-MS (method 1): Rₜ = 1.18 min; MS |
| | | (ESIpos): m/z = 483.2 [M + H]⁺ |
| | 4-chloro-6-fluoro-N¹-[(4- | |
| | fluorophenyl)methyl]-N³-[4-(3- | |
| | methyl-1,2,4-oxadiazol-5- | |
| | yl)phenyl]benzene-1,3- | |
| | dicarboxamide | |
| Example 24 | 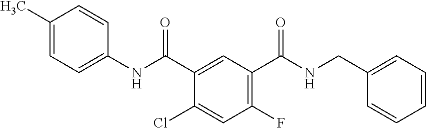 [SMILES: Cc1ccc(NC(=O)c2cc(C(=O)NCc3ccccc3)c(F)cc2Cl)cc1] | ¹H-NMR (400 MHz, CHLOROFORM-d) δ [ppm]: 0.889 (0.84), 1.262 (2.59), 1.319 (0.76), 2.227 (0.41), 2.358 (16.00), 2.623 (0.67), 4.678 (4.36), 4.692 (4.36), 6.911 (0.65), 6.924 (0.68), 6.938 (0.65), 7.185 (4.10), 7.206 (4.54), 7.296 (3.96), 7.305 (0.82), 7.311 (0.83), 7.321 (1.98), 7.328 (1.35), 7.338 (1.76), 7.341 (2.30), 7.346 |
| | | (2.01), 7.362 (10.14), 7.365 (11.60), 7.379 |
| | N¹-benzyl-4-chloro-6-fluoro-N³-(4- | (3.41), 7.382 (3.48), 7.386 (1.53), 7.396 |
| | methylphenyl)benzene-1,3- | (0.51), 7.400 (0.95), 7.404 (0.62), 7.509 |
| | dicarboxamide | (5.66), 7.530 (4.97), 7.720 (1.90), 8.436 |
| | | (3.77), 8.456 (3.76). |
| | | LC-MS (Method 4): Rₜ = 1.22 min; MS |
| | | (ESIpos): m/z = 397 [M + H]⁺ |
| Example 25 | 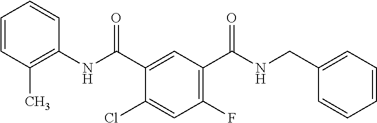 [SMILES: Cc1ccccc1NC(=O)c1cc(C(=O)NCc2ccccc2)c(F)cc1Cl] | ¹H-NMR (400 MHz, CHLOROFORM-d) δ [ppm]: 1.164 (1.72), 1.496 (1.17), 2.251 (8.00), 4.590 (2.25), 4.603 (2.72), 6.838 (0.49), 7.050 (0.44), 7.069 (1.12), 7.087 (0.96), 7.148 (1.46), 7.170 (16.00), 7.190 (3.17), 7.207 (1.40), 7.218 (2.16), 7.239 (1.23), 7.244 (1.25), 7.283 (3.46), 7.474 (1.00), 7.806 (1.06), 7.827 (1.11), 8.414 |
| | | (1.26), 8.434 (1.37). |
| | N1-benzyl-4-chloro-6-fluoro-N³-(2- | LC-MS (Method 4): Rt = 1.14 min; MS |
| | methylphenyl)benzene-1,3- | (ESIpos): m/z = 397 [M + H]+ |
| | dicarboxamide | |
| Example 26 | 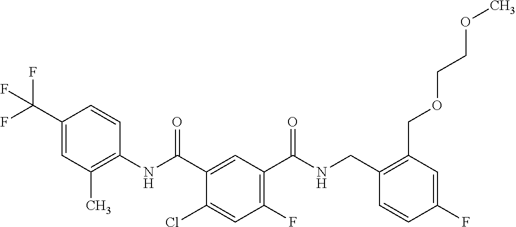 [SMILES: COCCOCc1cc(F)ccc1CNC(=O)c1cc(C(=O)Nc2ccc(C(F)(F)F)cc2C)c(Cl)cc1F] | ¹H-NMR (400 MHz, DMSO-d6) δ [ppm]: 2.240 (0.91), 2.364 (8.33), 2.518 (2.68), 2.523 (1.64), 3.216 (0.62), 3.223 (0.52), 3.235 (16.00), 3.245 (2.31), 3.293 (0.42), 3.492 (1.56), 3.502 (2.38), 3.507 (1.81), 3.514 (2.69), 3.602 (2.41), 3.610 (1.73), 3.615 (2.39), 3.626 (1.84), 4.472 (1.96), 4.486 (1.96), 4.619 (5.45), 7.110 (0.41), 7.116 (0.51), 7.131 (0.93), 7.138 (1.11), 7.152 (0.59), 7.160 (0.69), 7.198 (0.94), 7.205 (0.84), 7.222 (0.98), 7.229 (0.80), 7.374 (0.87), 7.388 (0.90), 7.395 (0.80), 7.410 (0.81), 7.591 (0.65), 7.612 (0.81), 7.660 (1.68), 7.737 (1.31), 7.749 (1.11), |
| | | 7.762 (1.38), 7.897 (1.28), 7.915 (1.24), |
| | 4-chloro-6-fluoro-N¹-({4-fluoro-2-[(2- | 8.915 (0.41), 8.928 (0.78), 8.942 (0.41), |
| | methoxyethoxy)methyl]phenyl}methyl)- | 10.300 (2.14). |
| | N³-[2-methyl-4- | LC-MS (Method 4): Rₜ = 1.38 min; MS |
| | (trifluoromethyl)phenyl]benzene-1,3- | (ESlpos): m/z = 571 [M + H]⁺ |
| | dicarboxamide | |
| Example 27 | 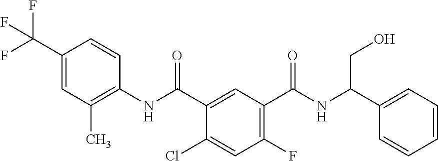 [SMILES: Cc1cc(C(F)(F)F)ccc1NC(=O)c1cc(C(=O)NC(CO)c2ccccc2)c(F)cc1Cl] | ¹H-NMR (400 MHz, DMSO-d6) δ [ppm]: 1.232 (0.43), 2.084 (0.42), 2.246 (1.18), 2.327 (0.65), 2.331 (0.55), 2.372 (16.00), 2.665 (0.46), 2.669 (0.60), 3.643 (2.39), 5.014 (0.71), 5.032 (1.62), 5.048 (1.86), 5.065 (1.58), 7.234 (0.86), 7.252 (2.43), 7.270 (1.97), 7.318 (2.56), 7.338 (5.34), 7.356 (3.52), 7.384 (5.57), 7.402 (3.22), 7.592 (1.28), 7.612 (1.55), 7.660 (3.20), 7.719 (2.39), 7.745 (2.70), 7.755 (2.26), |
| | | 7.776 (1.55), 7.901 (2.54), 7.919 (2.54), |
| | 4-chloro-6-fluoro-N¹-[(1R)-2- | 8.951 (1.70), 8.972 (1.65), 10.343 (3.80). |
| | hydroxy-1-phenylethyl]-N³-[2-methyl- | LC-MS (Method 4): Rₜ = 1.24 min; MS |
| | 4-(trifluoromethyl)phenyl]benzene- | (ESIpos): m/z = 495 [M + H]⁺ |
| | 1,3-dicarboxamide | |
| Example 28 | 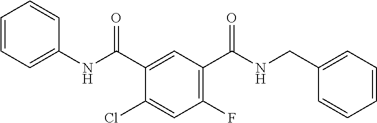 [SMILES: O=C(NCc1ccccc1)c1cc(C(=O)Nc2ccccc2)c(Cl)cc1F] | 1H-NMR (600 MHz, DMSO_3mm) delta [ppm]: 0.000 (1.23), 2.496 (4.60), 2.498 (10.36), 2.502 (14.54), 2.505 (10.31), 2.508 (4.54), 3.336 (11.52), 4.478 (4.52), 4.488 (4.39), 7.109 (1.00), 7.122 (2.31), 7.134 (1.27), 7.242 (0.41), 7.249 (1.00), 7.256 (1.85), 7.263 (1.61), 7.270 (0.84), 7.334 (0.54), 7.341 (16.00), 7.345 (4.26), 7.348 |
| | | (13.70), 7.358 (3.71), 7.359 (3.81), 7.369 |
| | N¹-benzyl-4-chloro-6-fluoro-N³- | (0.94), 7.372 (2.46), 7.693 (3.33), 7.695 |
| | phenylbenzene-1,3-dicarboxamide | (3.64), 7.707 (3.52), 7.709 (3.01), 7.713 |
| | | (3.04), 7.730 (2.72), 7.846 (2.73), 7.858 |
| | | (2.64), 8.314 (0.66), 9.056 (0.61), 9.066 |
| | | (1.14), 9.075 (0.60), 10.584 (2.45). |
| | | LC-MS (Method 4): Rₜ = 1.17 min; MS |
| | | (ESIpos): m/z = 383 [M + H]⁺ |

### Example 29

### 4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 1)

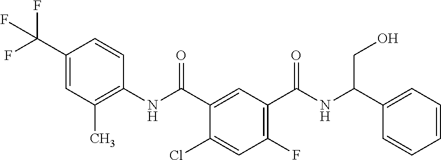 [SMILES: Cc1cc(C(F)(F)F)ccc1NC(=O)c1cc(C(=O)NC(CO)c2ccccc2)c(F)cc1Cl]

For the preparation of the racemic title compound see Example 27 Example 27. Separation of 33 mg racemate into the enantiomers by preparative chiral HPLC (method II) gave 15 mg of the title compound (Rₜ=7.0-9.0 min).

Analytical chiral HPLC (method 5): Rₜ=1.89 min.

Optical rotation:[α]_{D}=−15,61°+/−0.76° (c=1.0 g/100 ml DMSO)

¹H-NMR (400 MHz, DMSO-d6) δ[ppm]: 1.228 (1.04), 1.246 (0.45), 2.085 (0.99), 2.336 (0.52), 2.344 (0.73), 2.370 (16.00), 2.518 (5.09), 2.523 (3.53), 2.540 (3.81), 2.679 (0.44), 3.631 (1.59), 3.645 (2.53), 3.659 (1.43), 3.667 (1.30), 4.995 (1.13), 5.010 (2.65), 5.017 (1.00), 5.024 (1.36), 5.036 (1.41), 5.052 (1.19), 5.070 (0.63), 7.232 (0.47), 7.235 (0.88), 7.239 (0.56), 7.247 (0.70), 7.253 (2.41), 7.259 (0.95), 7.268 (1.14), 7.271 (1.99), 7.275 (1.09), 7.319 (2.48), 7.335 (2.20), 7.338 (5.52), 7.352 (1.08), 7.357 (3.42), 7.383 (5.15), 7.401 (2.81), 7.405 (1.96), 7.592 (1.09), 7.613 (1.33), 7.660 (2.72), 7.724 (2.39), 7.749 (2.80), 7.756 (1.95), 7.777 (1.30), 7.890 (2.64), 7.908 (2.60), 8.898 (1.46), 8.919 (1.41), 10.308 (3.79).

LC-MS (method 1): Rₜ=1.21 min; MS (ESIpos): m/z=495.2 [M+H]⁺

### Example 30

### 4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 2)

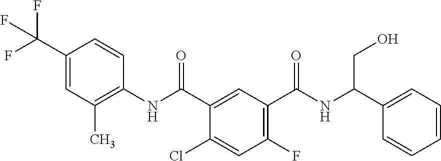 [SMILES: Cc1cc(C(F)(F)F)ccc1NC(=O)c1cc(C(=O)NC(CO)c2ccccc2)c(F)cc1Cl]

For the preparation of the racemic title compound see Example 27. Separation of 33 mg racemate into the enantiomers by preparative chiral HPLC (method II) gave 15 mg of the title compound (Rₜ=16.0-26.0 min).

Analytical chiral HPLC (method 5): Rₜ=5.41 min.

Optical rotation:[α]_{D}=13.81°+/−0.84° (c=1.0 g/100 ml DMSO)

LC-MS (method 1): Rₜ=1.21 min; MS (ESipos): m/z=495.2 [M+H]⁺

The following examples listed in table 10 below were prepared analogous to the preparation of example 1 starting from Intermediate 23 by reacting with the corresponding amines.

TABLE 10

| | | |
|---|---|---|
| Example 31 | 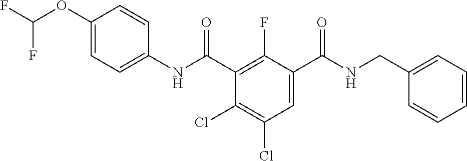 [SMILES: O=C(NCc1ccccc1)c1cc(Cl)c(Cl)c(C(=O)Nc2ccc(OC(F)F)cc2)c1F] | ¹H-NMR (400 MHz, DMSO-d6) δ [ppm]: 2.518 (1.48), 2.523 (0.95), 4.464 (3.26), 4.480 (3.21), 7.003 (2.09), 7.188 (3.83), 7.207 (3.54), 7.212 (1.10), 7.224 (1.19), 7.229 (3.71), 7.237 (0.60), 7.244 (0.52), 7.249 (0.47), 7.253 (0.66), 7.258 (0.93), 7.262 (0.73), 7.265 (0.59), 7.267 (0.61), 7.271 (0.83), 7.273 (0.85), 7.280 (0.69), 7.324 (0.73), 7.338 (16.00), 7.346 (4.10), 7.350 (4.10), 7.357 (0.56), 7.373 (2.20), |
| | | 7.684 (0.45), 7.693 (4.74), 7.698 (1.33), |
| | N¹-benzyl-4,5-dichloro-N³-[4- | 7.710 (1.33), 7.715 (4.16), 8.005 (3.10), |
| | (difluoromethoxy)phenyl]-2- | 8.021 (3.06), 9.196 (0.56), 9.210 (1.12), |
| | fluorobenzene-1,3-dicarboxamide | 9.225 (0.54), 11.001 (2.90). |
| | | LC-MS (method 1): Rₜ = 1.28 min; MS |
| | | (ESIpos): m/z = 483.1 [M + H]⁺ |
| Example 32 | 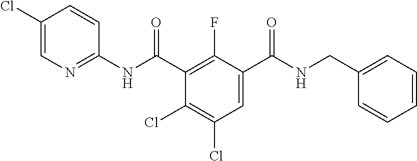 [SMILES: O=C(NCc1ccccc1)c1cc(Cl)c(Cl)c(C(=O)Nc2ccc(Cl)cn2)c1F] | 1H-NMR (400 MHz, DMSO-d6) delta [ppm]: 1.233 (1.19), 2.337 (0.72), 2.518 (8.80), 2.523 (5.79), 2.678 (0.73), 3.674 (0.52), 4.460 (3.54), 4.475 (3.50), 7.241 (0.52), 7.250 (0.70), 7.255 (1.03), 7.259 (0.82), 7.270 (0.97), 7.276 (0.78), 7.320 (0.81), 7.333 (16.00), 7.341 (4.35), 7.347 (4.45), 7.366 (0.48), 7.987 (2.89), 8.004 (4.30), 8.011 (1.79), 8.027 (1.90), 8.033 (2.11), 8.204 (2.90), 8.225 (2.30), 8.449 (2.47), |
| | N¹-benzyl-4,5-dichloro-N³-(5- | 8.451 (2.63), 8.456 (2.63), 9.171 (0.60), |
| | chloropyridin-2-yl)-2-fluorobenzene- | 9.185 (1.19), 9.200 (0.60), 11.654 (2.10). |
| | 1,3-dicarboxamide | LC-MS (method 1): Rₜ = 1.30 min; MS |
| | | (ESIpos): m/z = 452 [M + H]⁺ |
| Example 33 | 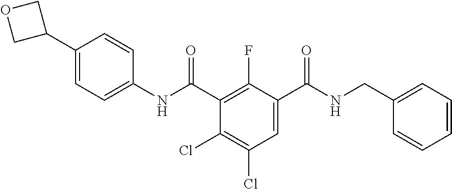 [SMILES: O=C(NCc1ccccc1)c1cc(Cl)c(Cl)c(C(=O)Nc2ccc(C3COC3)cc2)c1F] | 1H-NMR (400 MHz, DMSO-d6) delta [ppm]: 2.074 (1.45), 2.518 (2.80), 2.523(1.79), 4.213 (0.57), 4.234 (1.13), 4.251 (0.67), 4.464 (3.33), 4.480 (3.28), 4.586 (3.44), 4.601 (4.19), 4.603 (3.99), 4.617 (3.46), 4.917 (3.74), 4.932 (3.84), 4.938 (3.73), 4.953 (3.18), 7.244 (0.45), 7.247 (0.45), 7.252 (0.67), 7.257 (0.92), 7.261 (0.78), 7.264 (0.62), 7.270 (0.83), 7.271 (0.84), 7.279 (0.68), 7.324 (0.70), 7.337 (16.00), 7.346 (4.19), 7.350 (4.45), 7.366 (0.43), 7.412 (3.29), 7.434 (4.00), 7.644 (0.53), |
| | | 7.650 (4.66), 7.655 (1.34), 7.667 (1.18), |
| | N¹-benzyl-4,5-dichloro-2-fluoro-N3- | 7.672 (3.71), 7.996 (2.98), 8.013 (3.03), |
| | [4-(oxetan-3-yl)phenyl]benzene-1,3- | 9.193 (0.55), 9.208 (1.12), 9.223 (0.55), |
| | dicarboxamide | 10.913 (2.90). |
| | | LC-MS (method 1): Rₜ = 1.15 min; MS |
| | | (ESIpos): m/z = 473.1 [M + H]⁺ |

### Example 34

### 4-chloro-N¹-(3,4-difluorobenzyl)-6-fluoro-N³-(pyridin-2-yl)isophthalamide

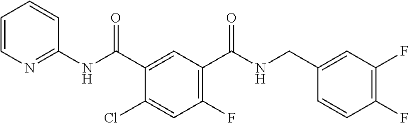 [SMILES: O=C(NCc1ccc(F)c(F)c1)c1cc(C(=O)Nc2ccccn2)c(Cl)cc1F]

To a solution of Intermediate 8 (2-chloro-5-[(3,4-difluorophenyl)methylcarbamoyl]-4-fluoro-benzoic acid, 70 mg, 203.6 μmol, 1 eq; Intermediate 8) and pyridine-2-amine (28.75 mg, 305.5 μmol, 1.5 eq) in Pyridine (1 mL) was added a 50% solution of T3P in DMF (388.8 mg, 611.0 μmol, 363.4 μL, 3 eq). The mixture was stirred at 20° C. for 2 h, at which time the mixture was concentrated in vacuo and the residue purified by preparative HPLC (column: Phenomenex Luna C18 150*25 mm*10 um; gradient: water (modified with 0.225% formic acid)—MeCN] 35%-65% over 10 min) followed by lyophilization to provide 60 mg (140.5 μmol, 69% yield, 98.3% purity) of the title compound as a white solid.

LCMS (method 9): Rt=1.923 min; MS (ESIpos): m/z=420.0 [M+H]⁺

1H NMR (400 MHz, MeOD) delta [ppm]: 4.545, 7.179, 7.192, 7.211, 7.217, 7.237, 7.258, 7.276, 7.280, 7.500, 7.525, 7.969, 7.985, 8.206, 8.217, 8.334

### Example 35

### 4-chloro-N¹-(3,4-difluorobenzyl)-6-fluoro-N³-(4-morpholinophenyl)isophthalamide

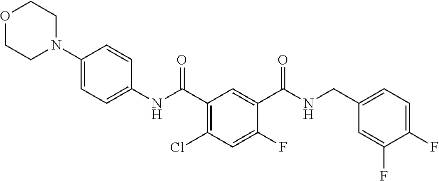 [SMILES: O=C(NCc1ccc(F)c(F)c1)c1cc(C(=O)Nc2ccc(N3CCOCC3)cc2)c(Cl)cc1F]

To a solution of Intermediate 8 (2-chloro-5-[(3,4-difluorophenyl)methylcarbamoyl]-4-fluoro-benzoic acid, 70 mg, 203.7 μmol, 1 eq; Intermediate 8) and 4-morpholinoaniline (54.45 mg, 305.5 μmol, 1.5 eq) in pyridine (1 mL) was added a 50% solution of T3P in DMF (388.8 mg, 363.4 μL, 611.0 μmol). The resulting mixture was stirred at 20° C. for 5 h, at which point the solution was concentrated in vacuo and the resulting residue purified by preparative HPLC (column: Phenomenex Luna C18 150 mm*25 mm*10 μm; gradient: water (modified with 0.225% formic acid)—MeCN) 37%-67% over 11.5 min) followed by lyophilization to provide 54.24 mg (51.54% yield, 97.5% purity) of the title compound as a white solid.

LCMS (method 9: Rt=1.629 min; MS (ESIpos): m/z=504.0 [M+]+

1H NMR (400 MHz, DMSO-d6) delta [ppm]: 3.051, 3.063, 3.075, 3.725, 3.737, 3.749, 4.448, 4.463, 6.926, 6.948, 7.391, 7.397, 7.412, 7.418, 7.543, 7.566, 7.701, 7.726, 7.824, 7.843, 9.061, 9.075, 9.089, 10.347

### Example 36

### 4-chloro-N¹-(3,4-difluorobenzyl)-6-fluoro-N³-(5-phenoxypyrazin-2-yl)isophthalamide

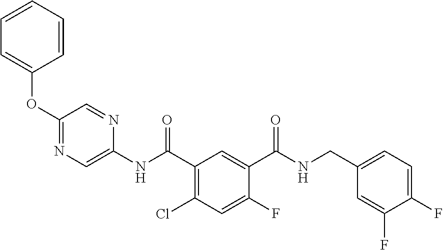 [SMILES: O=C(NCc1ccc(F)c(F)c1)c1cc(C(=O)Nc2cnc(Oc3ccccc3)cn2)c(Cl)cc1F]

To a solution of (2-chloro-5-[(3,4-difluorophenyl)methylcarbamoyl]-4-fluoro-benzoic acid, 70 mg, 203.6 μmol, 1 eq, Intermediate 8 Intermediate 8) and Intermediate 39 Intermediate 39(5-phenoxypyrazin-2-amine, 57.19 mg, 305.5 μmol, 1.5 eq) in pyridine (1 mL) was added a 50% solution of T3P in DMF (388.8 mg, 363.4 μL, 611.0 μmol, 3 eq). The resulting mixture was stirred at 25° C. for 12 h, at which point the solution was concentrated in vacuo, and the resulting residue was purified by preparative HPLC (column Phenomenex Luna C18 150 mm*25 mm*10 μm; Gradient: water (modified with 0.225% formic acid)—MeCN; 49%-79% over 11.5 min) followed by lyophilization to provide 40.25 mg (77.72 μmol, 38.16% yield, 99% purity) of the title compound as a white solid.

LCMS (method 9): Rt=2.072 min; MS (ESIpos): m/z=513.2 [M+H]⁺

¹H NMR (400 MHz, DMSO-d6) delta [ppm]: 4.448, 4.463, 7.182, 7.202, 7.237, 7.387, 7.414, 7.420, 7.439, 7.712, 7.737, 7.897, 7.916, 8.350, 8.352, 8.935, 9.041, 9.055, 9.070, 11.380

### Example 37

### 4-chloro-N¹-(3,4-difluorobenzyl)-6-fluoro-N³-(3-methyl-5-(oxetan-3-yl)pyridin-2-yl)isophthalamide

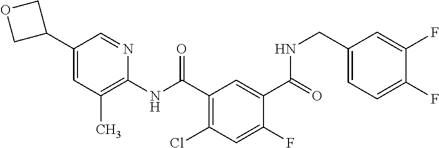 [SMILES: Cc1cc(C2COC2)cnc1NC(=O)c1cc(C(=O)NCc2ccc(F)c(F)c2)c(F)cc1Cl]

To a solution of 4-chloro-2-fluoro-5-[[3-methyl-5-(oxetan-3-yl)-2-pyridyl]carbamoyl]benzoic acid (30 mg, 82.25 μmol, 1 eq, Intermediate 8) and (3,4-difluorophenyl)methanamine (17.7 mg, 14.6 μL, 123.4 μmol, 1.5 eq) in pyridine (1 mL) was added a 50% solution of T3P (130.9 mg, 122.3 μL, 411.24 μmol, 5 eq) in DMF. The resulting mixture was stirred at 25° C. for 2 h. The resulting yellow solution was concentrated in vacuo, and the resulting residue was purified by preparative HPLC (column: Welch Xtimate C18 150 mm*25 mm*5 μm, gradient: water (modified with NH3-_{water})—MeCN 25%-55% over 8 min), followed by lyophilization to provide 11.97 mg (24.43 μmol, 29.7% yield, 100% purity) of the title compound as a yellow solid.

LCMS (method 9): Rₜ=1.733 min; MS (ESIpos): m/z=490.1 [M+H]⁺

1H NMR (400 MHz, METHANOL-d4) δ 8.28 (br s, 1H), 8.05 (d, J=7.6 Hz, 1H), 7.97 (d, J=2.0 Hz, 1H), 7.52 (d, J=10.4 Hz, 1H), 7.32-7.16 (m, 3H), 5.12 (dd, J=6.0, 8.4 Hz, 2H), 4.76 (t, J=6.4 Hz, 2H), 4.56 (s, 2H), 4.39-4.29 (m, 1H), 2.43 (s, 3H)

### Example 38

### 4,5-dichloro-N¹-(2-methoxybenzyl)-N³-(5-morpholinopyridin-2-yl)isophthalamide

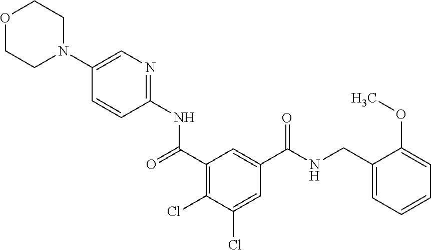 [SMILES: COc1ccccc1CNC(=O)c1cc(Cl)c(Cl)c(C(=O)Nc2ccc(N3CCOCC3)cn2)c1]

To a solution of 3,4-dichloro-5-[(5-morpholino-2-pyridyl)carbamoyl]benzoic acid (70 mg, 176.7 μmol, 1 eq, Intermediate 45 Intermediate 45), (2-methoxyphenyl)methanamine (36.4 mg, 34.30 μL, 265.0 μmol, 1.5 eq) in pyridine (1 mL) was added a 50% solution of T3P (337.3 mg, 315.2 μL, 530.0 μmol, 3 eq). The resulting mixture was stirred at 20° C. for 12 h, at which point the reaction was concentrated in vacuo. The residue was purified by preparative HPLC (column: Phenomenex Luna C18 150 mm*25 mm*10 um; Gradient: water (modified with 0.225% formic acid)—MeCN; 40%-70% over 11.5 min) followed by lyophilization to provide 64 mg (116.7 μmol, 66% yield, 94% purity) of the title compound as a brown solid.

LCMS (method 9): Rₜ=1.689 min; MS (ESIpos): m/z=515.0 [M+]+

¹H NMR (400 MHz, DMSO-d6) delta [ppm]: 3.125, 3.137, 3.148, 3.742, 3.753, 3.765, 3.818, 4.445, 4.459, 6.908, 6.927, 6.988, 7.008, 7.192, 7.210, 7.250, 8.033, 8.049, 8.054, 8.063, 8.070, 8.205, 8.210, 9.079, 9.093, 9.106, 10.946

### Example 39

### 4-chloro-N¹-(3,4-difluorobenzyl)-6-fluoro-N³-(2-(m-tolyl)benzo[d]oxazol-5-yl)isophthalamide

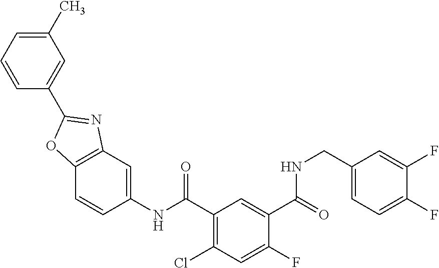 [SMILES: Cc1cccc(-c2nc3cc(NC(=O)c4cc(C(=O)NCc5ccc(F)c(F)c5)c(F)cc4Cl)ccc3o2)c1]

To a solution of 80% pure 2-(m-tolyl)-1,3-benzoxazol-5-amine (176.2 mg, 628.5 μmol, 1.2 eq; Intermediate 8) and 2-chloro-5-[(3,4-difluorophenyl)methylcarbamoyl]-4-fluoro-benzoic acid (180 mg, 523.7 μmol, 1 eq, Intermediate 8 Intermediate 8) in pyridine (2 mL) was added a 50% solution of T3P (999.9 mg, 934.5 μL, 1.57 mmol, 3 eq) in DMF. The resulting mixture was stirred at 25° C. for 12 h, at which time the solution was concentrated in vacuo. The resulting residue was purified by preparative HPLC (column: Phenomenex Synergi Polar RP 100 mm*25 mm*4 μm; Gradient: water (modified with 0.225% formic acid)—MeCN, 60%-90% over 8 min) followed by lyophilization to provide 55.6 mg (101.2 μmol, 19.3% yield, 100% purity) of the title compound as a yellow solid.

LCMS (method 9): Rₜ=2.130 min; MS (ESIpos): m/z=550.2 [M+]⁺

¹H NMR (400 MHz, DMSO-d6) delta [ppm]: 2.442, 4.459, 4.473, 7.205, 7.213, 7.396, 7.421, 7.473, 7.495, 7.515, 7.671, 7.751, 7.771, 7.776, 7.792, 7.918, 7.937, 7.996, 8.016, 8.043, 8.228, 8.230, 9.092, 9.105, 9.121, 10.769

### Example 40

### N¹-(3,4-difluorobenzyl)-4-ethynyl-6-fluoro-N³-phenylisophthalamide

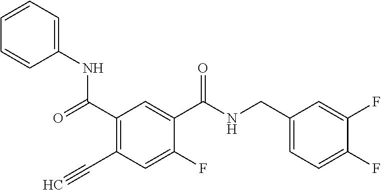 [SMILES: C#Cc1cc(F)c(C(=O)NCc2ccc(F)c(F)c2)cc1C(=O)Nc1ccccc1]

To a solution of N³-[(3,4-difluorophenyl)methyl]-4-fluoro-N¹-phenyl-6-(2-trimethylsilyl-ethynyl)benzene-1,3-dicarboxamide (65 mg, 67.6 μmol, 1 eq, Intermediate 51 Intermediate 51) in THF (2 mL) was added a 1 M solution of TBAF9322 (169.1 μL, 169.1 μmol, 2.5 eq) in THF. The resulting mixture was stirred at 25° C. for 2 h, at which point a saturated aqueous solution of ammonium chloride (5 mL) was added, and the resulting solution extracted 3× with ethyl acetate (5 mL), dried over sodium sulfate, filtered, and concentrated in vacuo to provide a residue. The residue was purified by preparative HPLC (column: Phenomenex Luna C18 150 mm*25 mm*10 μm; Gradient: water (modified with 0.225% formic acid)—MeCN; 44%-74% over 11.5 min), followed by lyophilization to provide 1.5 mg (3.42 μmol, 5.05% yield, 93% purity) of the title compound as a white solid.

LCMS (method 9): Rₜ=1.872 min; MS (ESIpos): m/z=409.2 [M+H]⁺

¹H NMR (400 MHz, DMSO-d6) delta [ppm]: 4.480, 4.495, 4.845, 4.850, 5.683, 5.688, 7.222, 7.03, 7.415, 7.421, 7.441, 7.485, 7.558, 7.578, 7.596, 8.048, 8.063, 8.156, 8.181, 9.129, 9.137, 9.149, 9.161

### Example 41

### 4-chloro-N¹-(3,4-difluorobenzyl)-6-fluoro-N³-(3-methyl-5-morpholinopyridin-2-yl)isophthalamide

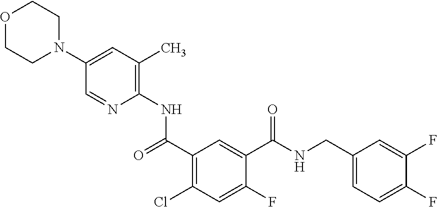 [SMILES: Cc1cc(N2CCOCC2)cnc1NC(=O)c1cc(C(=O)NCc2ccc(F)c(F)c2)c(F)cc1Cl]

To a solution of 2-chloro-5-[(3,4-difluorophenyl)methylcarbamoyl]-4-fluoro-benzoic acid (405 mg, 1.18 mmol, 1 eq, Intermediate 8 Intermediate 8) and 3-methyl-5-morpholino-pyridin-2-amine (341.6 mg, 1.77 mmol, 1.5 eq) in pyridine (10 mL) was added a 50% solution of T3P in DMF (2.25 g, 2.10 mL, 3.54 mmol, 3 eq). The resulting mixture was stirred at 25° C. for 12 h, at which time the reaction mixture was concentrated in vacuo, and the resulting residue was purified by flash column chromatography (ISCO, 20 g SepaFlash Silica Flash Column, Gradient: petroleum ether—ethyl acetate, 0%-100% at 100 mL/min) to provide 561.3 mg (1.06 mmol, 89.9% yield, 98% purity) as a grey solid.

LCMS(method 7) methyl 3,4-dichloro-5-(pyrazin-2-ylcarbamoyl)benzoate

Rₜ=0.475 min; MS (ESIpos): m/z=519.0 [M+]⁺

¹H NMR (400 MHz, DMSO-d6) delta [ppm]: 2.250, 3.160, 3.750, 4.459, 4.473, 7.199, 7.305, 7.364, 7.378, 7.389, 7.398, 7.405, 7.420, 7.425, 7.830, 7.843, 7.985, 9.082, 10.401

### Example 42

### 4,5-dichloro-N¹-(3,4-difluorobenzyl)-N³-(pyrazin-2-yl)isophthalamide

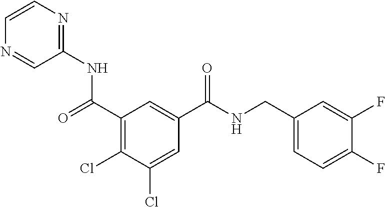 [SMILES: O=C(NCc1ccc(F)c(F)c1)c1cc(Cl)c(Cl)c(C(=O)Nc2cnccn2)c1]

To a solution of 3,4-dichloro-5-(pyrazin-2-ylcarbamoyl)benzoic acid (150 mg, 480.6 μmol, 1 eq, Intermediate 55), (3,4-difluorophenyl)methanamine (103.2 mg, 85.28 μL, 720.9 μmol, 1.5 eq) and N,N′-diisopropylethylamine (186.3 mg, 251.1 μL, 1.44 mmol, 3 eq) in DMF (1.5 mL) was added HATU (219.3 mg, 576.7 μmol, 1.2 eq). The mixture was stirred at 30° C. for 12 h, at which time the reaction was diluted with water (5 mL), extracted 3× with ethyl acetate (8 mL). The combined organic layers were washed 3× with brine (5 mL), dried over sodium sulfate, filtered, and concentrated in vacuo. The resulting residue was purified by preparative HPLC (column: Phenomenex Luna C18 150 mm*25 mm*10 um; Gradient: water (modified with 0.225% formic acid)—MeCN, 44%-74% over 10 min) followed by lyophilization to provide 85 mg (191.9 μmol, 39.9% yield, 98.7% purity) as a white solid.

LCMS (method 9): Rₜ=1.759 min; MS (ESIpos): m/z=437.0 [M+]⁺

1H NMR (400 MHz, MeOD) delta [ppm]: 4.533, 7.166, 7.185, 7.205, 7.211, 7.231, 7.241, 7.266, 7.269, 8.020, 8.025, 8.176, 8.181, 8.390, 8.396, 8.422, 9.503

### Example 43

### N¹-benzyl-4-chloro-N³-phenylbenzene-1,3-dicarboxamide

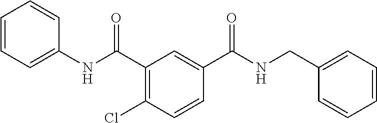 [SMILES: O=C(NCc1ccccc1)c1ccc(Cl)c(C(=O)Nc2ccccc2)c1]

Using an analogous method as described for Intermediate 71, 5-(benzylcarbamoyl)-2-chlorobenzoic acid (1.00 g, 3.45 mmol, Intermediate 68 Intermediate 68) and aniline (410 μl, 4.5 mmol; CAS-RN: [62-53-3]) were used as the starting materials. 754 mg (100% purity, 60% yield) of the title compound were prepared.

LC-MS (Method 4): Rₜ=1.14 min; MS (ESIpos): m/z=365 [M+H]⁺

1H-NMR (400 MHz, DMSO-d6) delta [ppm]: 2.518 (1.76), 2.522 (1.07), 4.483 (3.57), 4.498 (3.55), 7.103 (0.90), 7.122 (1.99), 7.141 (1.21), 7.231 (0.51), 7.236 (0.49), 7.240 (0.69), 7.245 (1.02), 7.249 (0.81), 7.252 (0.70), 7.260 (0.93), 7.267 (0.78), 7.308 (0.85), 7.320 (16.00), 7.329 (4.45), 7.334 (4.61), 7.342 (2.64), 7.363 (3.35), 7.377 (0.79), 7.382 (2.19), 7.688 (3.32), 7.701 (3.16), 7.704 (3.58), 7.709 (4.46), 7.722 (3.19), 7.725 (2.53), 8.000 (1.83), 8.006 (2.04), 8.021 (1.59), 8.026 (1.77), 8.119 (3.28), 8.125 (3.09), 9.226 (0.67), 9.241 (1.37), 9.256 (0.67), 10.595 (2.81).

The following examples listed in table 11 below were prepared analogous to the preparation of example 1 starting from Intermediate 70, or Intermediate 81 by reacting with the corresponding amines.

TABLE 11

| | Structure | |
|---|---|---|
| Example | Name | Analytical Data |
| Example 44 | 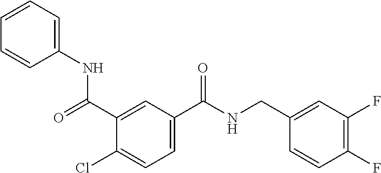 [SMILES: O=C(NCc1ccc(F)c(F)c1)c1ccc(Cl)c(C(=O)Nc2ccccc2)c1] | LC-MS (Method 10): Rₜ = 1.14 min; MS (ESIpos): m/z = 401 [M + H]⁺ |
| | 4-chloro-N¹-[(3,4- | |
| | difluorophenyl)methyl]-N³- | |
| | phenylbenzene-1,3-dicarboxamide | |
| Example 45 | 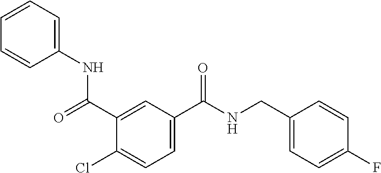 [SMILES: O=C(NCc1ccc(F)cc1)c1ccc(Cl)c(C(=O)Nc2ccccc2)c1] | LC-MS (Method 10): Rₜ = 1.11 min; MS (ESIpos): m/z = 383 [M + H]⁺ |
| | 4-chloro-N¹-[(4-fluorophenyl)methyl]- | |
| | N³-phenylbenzene-1,3- | |
| | dicarboxamide | |
| Example 46 | 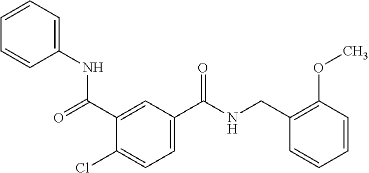 [SMILES: COc1ccccc1CNC(=O)c1ccc(Cl)c(C(=O)Nc2ccccc2)c1] | LC-MS (Method 10): Rₜ = 1.12 min; MS (ESIpos): m/z = 395 [M + H]⁺ |
| | 4-chloro-N¹-[(2- | |
| | methoxyphenyl)methyl]-N³- | |
| | phenylbenzene-1,3-dicarboxamide | |
| Example 47 | 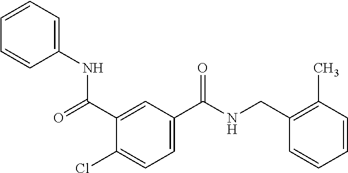 [SMILES: Cc1ccccc1CNC(=O)c1ccc(Cl)c(C(=O)Nc2ccccc2)c1] | LC-MS (Method 10): Rₜ = 1.15 min; MS (ESIpos): m/z = 379 [M + H]⁺ |
| | 4-chloro-N¹-[(2- | |
| | methylphenyl)methyl]-N³- | |
| | phenylbenzene-1,3-dicarboxamide | |
| Example 48 | 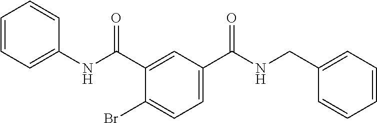 [SMILES: O=C(NCc1ccccc1)c1ccc(Br)c(C(=O)Nc2ccccc2)c1] | 1H-NMR (600 MHz, DMSO_3mm) delta [ppm]: 2.496 (4.80), 2.499 (11.36), 2.503 (16.00), 2.506 (11.20), 2.509 (4.77), 3.349 (10.00), 4.484 (1.57), 4.494 (1.58), 7.111 (0.46), 7.123 (1.01), 7.125 (0.47), 7.136 (0.50), 7.248 (0.50), 7.258 (0.44), 7.312 (0.41), 7.323 (4.68), 7.326 (1.64), 7.334 (1.85), 7.337 (0.42), 7.347 (0.41), 7.351 |
| | | (1.08), 7.363 (1.36), 7.365 (1.44), 7.377 |
| | N¹-benzyl-4-bromo-N³- | (1.05), 7.707 (1.33), 7.709 (1.54), 7.722 |
| | phenylbenzene-1,3-dicarboxamide | (1.40), 7.724 (1.12), 7.847 (1.32), 7.861 |
| | | (1.88), 7.915 (1.13), 7.919 (1.08), 7.929 |
| | | (0.73), 7.933 (0.75), 8.081 (1.59), 8.085 |
| | | (1.49), 9.260 (0.56), 10.591 (1.17). |
| | | LC-MS (method 1): Rₜ = 1.09 min; MS |
| | | (ESIpos): m/z = 408.7 [M + H]⁺ |

### Example 49

### N¹-benzyl-4-bromo-5-chloro-N³-phenylbenzene-1,3-dicarboxamide

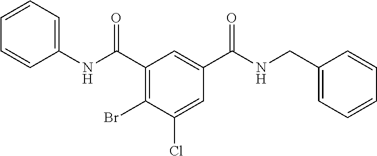 [SMILES: O=C(NCc1ccccc1)c1cc(Cl)c(Br)c(C(=O)Nc2ccccc2)c1]

To a solution of 5-(benzylcarbamoyl)-2-bromo-3-chlorobenzoic acid (70.0 mg, 0.19 mmol, Intermediate 62 Intermediate 62) and aniline (23.0 mg, 0.25 mmol) in N,N-dimethylformamide (2.0 ml) were added N,N-diisopropylethylamine (0.066 ml, 0.38 mmol) and 1-[Bis(dimethylamino)methylene]-1H-1,2,3-triazolo[4,5-b]pyridinium 3-oxid hexafluorophosphate (108 mg, 0.29 mmol) at room temperature. The reaction mixture was stirred at room temperature for 2 hours. The mixture was purified by preparative HPLC [Instrument:GX-Q; Column: Shim-pack C18 150*25*10 um; eluent A: water (0.225% formic acid in water), eluent B: acetonitrile; gradient: 0-10 min 53-73% B; flow 25 ml/min; temperature: RT; Detector: UV 220/254 nm.] to give N¹-benzyl-4-bromo-5-chloro-N³-phenylisophthalamide (64.6 mg, 96% purity, 74% yield) as white solid.

LC-MS (Method 6): Rₜ=0.857 min; MS (ESIpos): m/z=444.9 [M+H]⁺.

¹H NMR (400 MHz, DMSO-d₆): δ [ppm]=10.64 (s, 1H), 9.32 (t, J=6.0 Hz, 1H), 8.20 (d, J=2.0 Hz, 1H), 8.01 (d, J=2.0 Hz, 1H), 7.72-7.68 (m, 2H), 7.40-7.31 (m, 6H), 7.28-7.23 (m, 1H), 7.14 (t, J=7.6 Hz, 1H), 4.50 (d, J=5.6 Hz, 2H).

### Example 50

### N¹-benzyl-4-bromo-5-chloro-N³-(2-methylphenyl)benzene-1,3-dicarboxamide

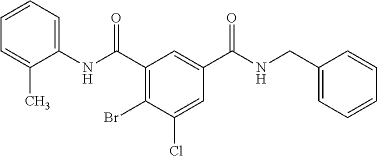 [SMILES: Cc1ccccc1NC(=O)c1cc(C(=O)NCc2ccccc2)cc(Cl)c1Br]

To a solution of 5-(benzylcarbamoyl)-2-bromo-3-chlorobenzoic acid (70.0 mg, 0.19 mmol, Intermediate 62 and 2-methylaniline (26.5 mg, 0.25 mmol) in N,N-dimethylformamide (2.0 ml) were added N,N-diisopropylethylamine (0.066 ml, 0.38 mmol) and 1-[Bis(dimethylamino)methylene]-1H-1,2,3-triazolo[4,5-b]pyridinium 3-oxid hexafluorophosphate (108 mg, 0.29 mmol) at room temperature. The reaction mixture was stirred at room temperature for 2 hours. The mixture was purified by preparative HPLC [Instrument:GX-Q; Column: Shim-pack C18 150*25*10 um; eluent A: water (0.225% formic acid in water), eluent B: acetonitrile; gradient: 0-11 min 51-73% B; flow 25 ml/min; temperature: RT; Detector: UV 220/254 nm.] to give N¹-benzyl-4-bromo-5-chloro-N³-(2-methylphenyl)isophthalamide (55.3 mg, 90% purity, 57% yield) as white solid.

LC-MS (Method 6): Rₜ=0.857 min; MS (ESIpos): m/z=459.0 [M+H]⁺.

¹H NMR (400 MHz, DMSO-d6): δ [ppm]=10.11 (s, 1H), 9.36 (t, J=6.0 Hz, 1H), 8.20 (d, J=2.0 Hz, 1H), 8.01 (d, J=2.0 Hz, 1H), 7.48-7.45 (m, 2H), 7.37-7.32 (m, 4H), 7.29-7.22 (m, 3H), 7.20-7.15 (m, 1H), 4.51 (d, J=5.6 Hz, 2H), 2.31 (s, 3H).

### Example 51

### 4-chloro-N¹-[(3,4-difluorophenyl)methyl]-6-fluoro-N³-[4-(2-hydroxypropan-2-yl)phenyl]benzene-1,3-dicarboxamide

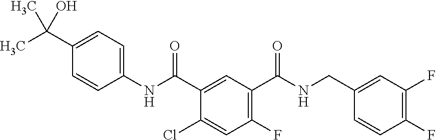 [SMILES: CC(C)(O)c1ccc(NC(=O)c2cc(C(=O)NCc3ccc(F)c(F)c3)c(F)cc2Cl)cc1]

In a round bottom flask, to a solution of 2-chloro-5-{[(3,4-difluorophenyl)methyl]carbamoyl}-4-fluorobenzoic acid (50.0 mg, 145 μmol, Intermediate 8) in DCM (1.5 ml) was added Ghosez reagent 1-chloro-N,N,2-trimethylprop-1-en-1-amine (58 μl, 440 μmol). The mixture was stirred for 30 minutes before 2-(4-aminophenyl)propan-2-ol (33.0 mg, 218 μmol; CAS-RN: [23243-04-1]) in DCM (0.75 ml) and pyridine (56 μl) was added. The reaction mixture was stirred at ambient temperature for 2 hours. The solution was diluted with water and extracted with DCM. The organic phase was isolated, evaporated, dissolved in DMSO and purified by prep. HPLC (RP, acidic conditions). The product rich fractions were pooled, ACN evaporated and freeze dried to yield the desired product (33.2 mg, 95% purity, 45% yield).

LC-MS (method 1): Rₜ=1.08 min; MS (ESIpos): m/z=477.1 [M+H]⁺

1H-NMR (400 MHz, DMSO-d6) delta [ppm]: 1.411 (16.00), 2.518 (1.83), 2.523 (1.18), 4.449 (1.64), 4.464 (1.65), 4.978 (4.75), 7.190 (0.42), 7.195 (0.44), 7.199 (0.42), 7.359 (0.42), 7.364 (0.41), 7.374 (0.67), 7.378 (0.49), 7.384 (0.50), 7.388 (0.53), 7.395 (1.31), 7.402 (0.69), 7.408 (0.54), 7.413 (0.77), 7.419 (2.60), 7.423 (1.79), 7.436 (0.92), 7.441 (2.95), 7.579 (0.43), 7.585 (3.08), 7.590 (0.92), 7.602 (0.80), 7.607 (2.23), 7.722 (1.79), 7.747 (1.76), 7.836 (1.71), 7.855 (1.71), 9.098 (0.65), 10.506 (1.89).

The following examples listed In table 12 below were prepared analogous to the preparation of Example 51, Example 51 starting from Intermediate 17, or Intermediate 6 by reacting with the corresponding amines.

TABLE 12

| | Structure | |
|---|---|---|
| Example | Name | Analytical Data |
| Example 52 | 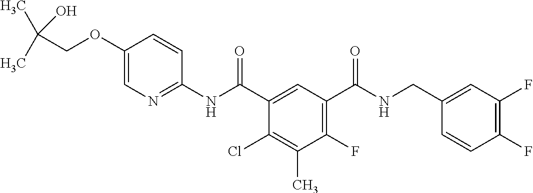 [SMILES: Cc1c(F)c(C(=O)NCc2ccc(F)c(F)c2)cc(C(=O)Nc2ccc(OCC(C)(C)O)cn2)c1Cl] | ¹H-NMR (400 MHz, DMSO-d6) δ [ppm]: 1.206 (16.00), 2.344 (2.93), 2.349 (2.96), 2.518 (0.93), 2.523 (0.63), 3.717 (0.40), 3.788 (3.93), 4.444 (1.42), 4.458 (1.42), 4.677 (4.03), 7.374 (0.83), 7.379 (0.42), 7.384 (0.44), 7.395 (0.95), 7.401 (0.68), 7.416 (0.48), 7.422 (0.88), 7.443 (0.42), 7.481 (0.56), 7.489 (0.56), 7.504 (0.58), 7.512 (0.59), 7.651 (0.75), 7.669 (0.76), 8.075 (0.96), 8.082 (1.04), 8.088 (0.89), 8.111 (0.68), 9.052 (0.57), 10.923 (1.40). LC-MS (method 1): Rt = 1.12 min; MS |
| | | (ESIpos): m/z = 522.3 [M + H]+ |
| | 4-chloro-N¹-[(3,4- | |
| | difluorophenyl)methyl]-6-fluoro-N³- | |
| | [5-(2-hydroxy-2- | |
| | methylpropoxy)pyridin-2-yl]-5- | |
| | methylbenzene-1,3-dicarboxamide | |
| Example 53 | 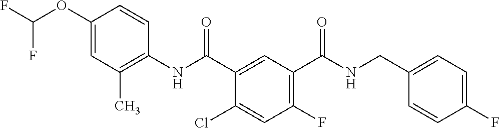 [SMILES: Cc1cc(OC(F)F)ccc1NC(=O)c1cc(C(=O)NCc2ccc(F)cc2)c(F)cc1Cl] | ¹H-NMR (400 MHz, DMSO-d6) δ [ppm]: 2.283 (16.00), 2.331 (0.56), 2.518 (2.45), 2.523 (1.66), 2.673 (0.53), 4.457 (3.69), 4.471 (3.68), 7.030 (3.54), 7.039 (1.49), 7.053 (1.31), 7.060 (1.60), 7.108 (2.86), 7.115 (2.35), 7.155 (3.21), 7.160 (1.11), 7.171 (1.41), 7.177 (6.69), 7.183 (1.34), 7.194 (1.19), 7.199 (4.00), 7.207 (0.53), |
| | | 7.216 (5.16), 7.366 (2.79), 7.372 (1.24), |
| | 4-chloro-N³-[4-(difluoromethoxy)-2- | 7.380 (3.14), 7.388 (2.68), 7.402 (4.42), |
| | methylphenyl]-6-fluoro-N¹-[(4- | 7.441 (3.43), 7.462 (3.04), 7.722 (3.82), |
| | fluorophenyl)methyl]benzene-1,3- | 7.747 (3.71), 7.873 (3.51), 7.892 (3.57), |
| | dicarboxamide | 9.069 (0.73), 9.083 (1.39), 9.097 (0.70), |
| | | 10.098 (4.16). |
| | | LC-MS (method 1): Rt = 1.21 min; MS |
| | | (ESIpos): m/z = 481.2 [M + H]+ |

General procedure A with HATU.

To a solution of 2-chloro-5-[(3,4-difluorophenyl)methylcarbamoyl]-4-fluoro-benzoic acid Intermediate 72 Intermediate 72, 50 mg, 145.48 umol, 1 eq) and amine (174.58 umol, 1.2 eq) in DMF (0.5 mL) was added HATU (82.98 mg, 218.22 umol, 1.5 eq) and DIEA (75.22 mg, 615.27 umol, 101.37 uL, 4 eq), then the mixture was stirred at 25° C. for 1 hr. The mixture was washed with water and then extracted with EtOAc (1 ml*2), the organic layer was separated and concentrated to give crude product and then purified by Prep-HPLC(FA or NH₄HCO₃) and the eluent was lyophilized to give product.

### Example 54

### 4-chloro-N¹-[(3,4-difluorophenyl)methyl]-6-fluoro-N³-(3-methyl-2-pyridyl)benzene-1,3-dicarboxamide

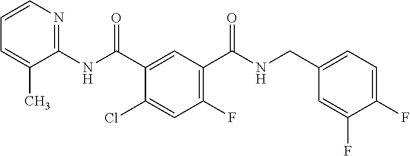 [SMILES: Cc1cccnc1NC(=O)c1cc(C(=O)NCc2ccc(F)c(F)c2)c(F)cc1Cl]

The reaction was performed according to the general procedure A on a 60 mg (174.58 umol) scale. The crude mixture was purified by Prep-HPLC (column: Phenomenex luna C18 150*25 mm*10 um; mobile phase: [water (0.05% ammonia hydroxide v/v)-ACN]; B %: 25%-55%,8 min), the eluent was lyophilized to give 4-chloro-N¹-[(3,4-difluorophenyl)methyl]-6-fluoro-N³-(3-methyl-2-pyridyl)benzene-1,3-dicarboxamide (9 mg, 20.12 umol, 11.53% yield, 97% purity) as a yellow solid

LC-MS (Method 9): Rₜ=0.804 min; MS (ESIpos): m/z=434.1 [M+H]⁺

1H NMR (400 MHz, DMSO-d6) δ[ppm]: 10.69 (br s, 1H), 9.09 (br t, J=4.8 Hz, 1H), 8.33-8.23 (m, 1H), 7.86 (d, J=7.6 Hz, 1H), 7.77-7.67 (m, 2H), 7.48-7.32 (m, 2H), 7.31-7.16 (m, 2H), 4.47 (d, J=6.0 Hz, 2H), 2.29 (s, 3H)

### Example 55

### 4-chloro-N¹-(3,4-difluorobenzyl)-6-fluoro-N³-(naphthalen-2-yl)isophthalamide

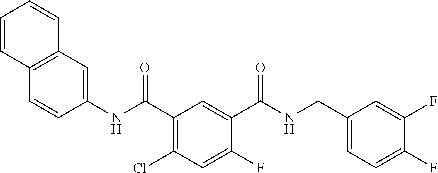 [SMILES: O=C(NCc1ccc(F)c(F)c1)c1cc(C(=O)Nc2ccc3ccccc3c2)c(Cl)cc1F]

The reaction was performed according to the general procedure A on a 60 mg (174.58 umol, 1 eq) scale. The residue was purified by prep-HPLC (column: Phenomenex Gemini-NX C18 75*30 mm*3 um; mobile phase: [water(10 mM NH4HCO3)-ACN]; B %: 42%-72%,8 min) to give crude product, then the crude product was purified by prep-HPLC (column:Welch Ultimate XB-CN 250*70*10 um; mobile phase: [Hexane-EtOH]; B %: %-%,15 min) and lyophilizated to give 4-chloro-N¹-[(3,4-difluorophenyl)methyl]-6-fluoro-N³-(2-naphthyl)benzene-1,3-dicarboxamide (9.57 mg, 20.17 umol, 11.56% yield) was off-white.

LC-MS (Method 9): Rt=0.966 min; MS (ESIpos): m/z=469.3 [M+H]⁺

1H NMR (400 MHz, DMSO-d6) δ[ppm]: 10.79 (s, 1H), 9.12-9.08 (m, 1H), 8.42 (d, J=1.6 Hz, 1H), 7.96-7.85 (m, 4H), 7.76 (d, J=10.0 Hz, 1H), 7.69-7.66 (m, 1H), 7.53-7.48 (m, 1H), 7.47-7.36 (m, 3H), 7.24-7.17 (m, 1H), 4.47 (d, J=6.0 Hz, 2H) ppm.

### Example 56

### 4,5-dichloro-N¹-[(2-methoxyphenyl)methyl]-N³-pyrazin-2-yl-benzene-1,3-dicarboxamide

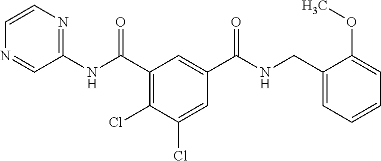 [SMILES: COc1ccccc1CNC(=O)c1cc(Cl)c(Cl)c(C(=O)Nc2cnccn2)c1]

To a solution of 3,4-dichloro-5-(pyrazin-2-ylcarbamoyl)benzoic acid (Intermediate 74 Intermediate 74, 150 mg, 480.60 umol, 1 eq), 2-methoxybenzylamine (98.89 mg, 720.90 umol, 93.29 uL, 1.5 eq, CAS-RN: [6850-57-3]) and DIEA (186.34 mg, 1.44 mmol, 251.13 uL, 3 eq, CAS-RN: [7087-68-5]) in DMF (1.5 mL) was added HATU (219.29 mg, 576.72 umol, 1.2 eq, CAS-RN: [148893-10-1]). The mixture was stirred at 30° C. for 12 h. The reaction mixture was diluted with water (10 mL), extracted with EtOAc (12 mL×3). The combined organic layers were washed with brine (8 mL×3), dried over Na2SO4, filtered, and concentrated in vacuo. The resulting residue was purified by prep-HPLC (column: Phenomenex luna C18 150*25 mm*10 um; mobile phase: [water modified with 0.225% FA-ACN]; Gradient B %: 42%-72% over 10 min). The fractions containing the title compound were lyophilized to provide 96.56 mg (221.75 umol, 46.14% yield, 99.04% purity) of the title compound as a yellow solid.

¹H NMR (400 MHz, MeOD-d4) δ [ppm]: 3.884, 4.588, 6.906, 6.925, 6.944, 6.981, 7.002, 7.257, 7.275, 7.293, 8.031, 8.036, 8.181, 8.186, 8.404, 8.410, 8.435

LCMS (Method 9): Rₜ=1.722, MS (ESIpos): m/z=431.0 (M+)

### Example 57

### 4-chloro-N¹-[(3,4-difluorophenyl)methyl]-6-fluoro-N³-(5-phenoxy-2-pyridyl)benzene-1,3-dicarboxamide

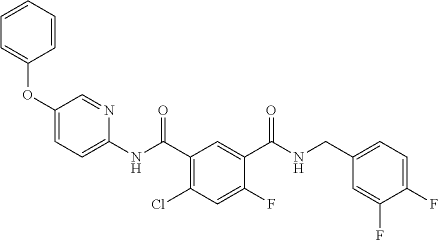 [SMILES: O=C(NCc1ccc(F)c(F)c1)c1cc(C(=O)Nc2ccc(Oc3ccccc3)cn2)c(Cl)cc1F]

To a solution of 5-phenoxypyridin-2-amine (Intermediate 75, 81.27 mg, 436.45 umol, 1.5 eq) and 2-chloro-5-[(3,4-difluorophenyl)methylcarbamoyl]-4-fluoro-benzoic acid (Intermediate 72, 100 mg, 290.96 umol, 1 eq) in pyridine (1 mL) was added T3P (555.48 mg, 872.89 umol, 519.14 uL, 50% purity, 3 eq), the mixture was stirred at 25° C. for 12 hr. The mixture was concentrated under reduced pressure to give a residue, which was purified by prep-HPLC (column: Phenomenex luna C18 150*25 mm*10 um; mobile phase: [water modified with 0.225% FA-ACN]; Gradient B %: 53%-83% over 11.5 min). Fractions containing the title compound were concentrated by lyophilization to provide 50 mg (97.68 umol, 33.57% yield, 100% purity) of the title compound as an off-white solid.

¹H NMR (400 MHz, MeOD-d4) δ [ppm]: 4.551, 7.037, 7.056, 7.161, 7.180, 7.197, 7.375, 7.396, 7.415, 7.499, 7.525, 7.960, 7.979, 8.094, 8.101, 8.205

LCMS (Method 9): Rₜ=2.068 min, MS (ESIpos): m/z=512.1 (M+H)+

### Example 58

### 4-chloro-N³-[4-(difluoromethoxy)-3-fluorophenyl]-6-fluoro-N¹-[(2-methoxyphenyl)methyl]benzene-1,3-dicarboxamide

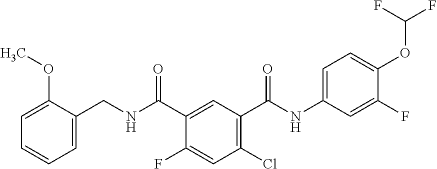 [SMILES: COc1ccccc1CNC(=O)c1cc(C(=O)Nc2ccc(OC(F)F)c(F)c2)c(Cl)cc1F]

To a solution of 4-chloro-5-{[4-(difluoromethoxy)-3-fluorophenyl]carbamoyl}-2-fluorobenzoic acid (43.5 mg, 115 μmol, Intermediate 77 Intermediate 77) and 1-(2-methoxyphenyl)methanamine (31.6 mg, 230 μmol) and 4-(Dimethylamino)pyridin (56.3 mg, 461 μmol; CAS-RN:[1122-58-3]) in DMF (2.0 ml) was added 1-(3-Dimethylaminopropyl)-3-ethylcarbodiimidhydrochlorid (44.2 mg, 230 μmol; CAS-RN:[25952-53-8]) and the mixture was stirred at rt for 16 h. The crude reaction mixture was purified by preparative reversed phase HPLC to give 9.00 mg (95% purity, 15% yield) of the title compound.

LC-MS (method 10): Rₜ=1.27 min; MS (ESIpos): m/z=497 [M+H]⁺

¹H-NMR (400 MHz, DMSO-d6) δ[ppm]: 1.232 (0.65), 2.518 (1.07), 2.523 (0.70), 2.784 (0.71), 3.820 (16.00), 4.429 (2.24), 4.444 (2.22), 6.909 (0.69), 6.911 (0.77), 6.927 (1.35), 6.930 (1.57), 6.946 (0.80), 6.948 (0.84), 6.993 (1.29), 7.008 (1.63), 7.012 (1.49), 7.192 (2.57), 7.237 (2.12), 7.256 (2.34), 7.275 (0.67), 7.280 (0.48), 7.362 (0.57), 7.374 (1.21), 7.385 (1.28), 7.407 (0.90), 7.445 (0.97), 7.448 (0.97), 7.451 (0.85), 7.467 (0.51), 7.471 (0.52), 7.473 (0.50), 7.741 (2.07), 7.766 (2.03), 7.805 (1.01), 7.811 (0.98), 7.837 (0.95), 7.843 (0.96), 7.869 (2.13), 7.888 (2.09), 8.858 (0.40), 8.872 (0.78), 8.886 (0.41), 10.868 (1.98).

### Example 59

### 4-chloro-6-fluoro-N³-[2-fluoro-4-(oxetan-3-yl)phenyl]-N¹-[(2-methoxyphenyl)methyl]benzene-1,3-dicarboxamide

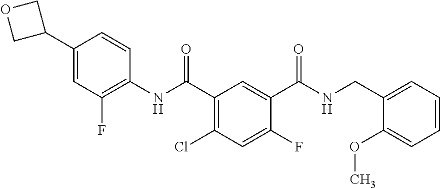 [SMILES: COc1ccccc1CNC(=O)c1cc(C(=O)Nc2ccc(C3COC3)cc2F)c(Cl)cc1F]

To a solution of 2-chloro-4-fluoro-5-[(2-methoxyphenyl)methylcarbamoyl]benzoic acid (50 mg, 148.05 umol, Intermediate 79) in DMF (1 mL) was added HATU (84.44 mg, 222.07 umol) and DIEA (57.40 mg, 444.14 umol, 77.36 uL). The reaction mixture was stirred at 25° C. for 0.5 h. Then 2-fluoro-4-(oxetan-3-yl)aniline (37.13 mg, 222.07 umol, Intermediate 84) was added. The reaction solution was stirred at 25° C. for another 2 hrs. The reaction mixture was diluted with 3 mL of H2O and extracted with EA (3 mL*3). The combined organic layers were washed with 5 mL of brine, dried over anhydrous Na2SO4, filtered and concentrated under reduced pressure to give a residue. The residue was purified by prep-HPLC (column: Phenomenex Gemini-NX C18 75*30 mm*3 um; mobile phase: [water (0.05% ammonia hydroxide v/v)-ACN]; B %: 27%-57%,11.5 min) and concentrated under reduced pressure to remove MeCN, then lyophilized to afford 13.77 mg (28.05 mmol, 99.193% yield) of the title compound as an off-white solid.

LC-MS (Method A): Rt=0.934 min; MS (ESIpos): m/z=487.1 [M+H]+

1H NMR (400 MHz, DMSO-d6) δ=10.41 (s, 1H), 8.86-8.83 (m, 1H), 7.85 (d, J=7.6 Hz, 1H), 7.80-7.77 (m, 1H), 7.72 (d, J=0.0 Hz, 1H), 7.38-7.35 (m, 1H), 7.30-7.21 (m, 3H), 7.01 (d, J=8.0 Hz, 1H), 6.97-6.90 (m, 1H), 4.95-4.92 (m, 2H), 4.63-0.60 (m, 2H), 4.44 (d, J=5.6 Hz, 2H), 4.33-4.22 (m, 1H), 3.83 (s, 3H).

19FNMR (376 MHz, DMSO-d6) 5-109.711, −122.218.

### Example 60

### 4-chloro-6-fluoro-N¹-[(2-methoxyphenyl)methyl]-N³-5-(morpholin-4-yl)pyridin-2-yl]benzene-1,3-dicarboxamide

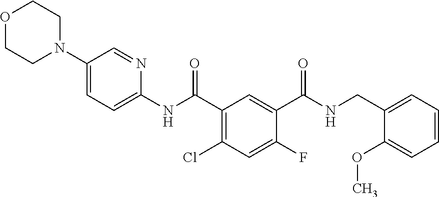 [SMILES: COc1ccccc1CNC(=O)c1cc(C(=O)Nc2ccc(N3CCOCC3)cn2)c(Cl)cc1F]

To a solution of 2-chloro-4-fluoro-5-((2-methoxybenzyl)carbamoyl)benzoic acid (Intermediate 83, 60 mg, 177.66 umol, 1 eq) and 5-morpholinopyridin-2-amine (38.2 mg, 213.13 umol, 1.2 eq, CAS 571189-78-1) in pyridine (0.5 mL) was added EDCl (51 mg, 266.49 umol, 1.5 eq), then the mixture was stirred at 80° C. for 12 hrs. The mixture was washed with water (1 ml) and then extracted with EA (1 ml*2), the organic layer was separated and concentrated to give crude product. The residue was purified by prep-HPLC (column: Shim-pack C18 150*25*10 um; mobile phase: [water(0.225% FA)-ACN]; B %:39%-59%,10 min) and lyophilized to give 30.74 mg (60.73 umol, 34.19% yield) of the title compound as a white solid.

LC-MS (Method 8): Rt=0.894 min; MS (ESIpos): m/z=499.1 [M+H]+

¹H NMR (400 MHz, DMSO-d6) δ=10.86 (s, 1H), 8.82-8.78 (m, 1H), 8.11-7.99 (m, 2H), 7.83 (d, J=7.6 Hz, 1H), 7.68 (d, J=10.4 Hz, 1H), 7.49-7.46 (m, 1H), 7.30-7.22 (m, 2H), 7.01 (d, J=8.0 Hz, 1H), 6.97-6.89 (m, 1H), 4.44 (d, J=6.0 Hz, 2H), 3.83 (s, 3H), 3.79-3.72 (m, 4H), 3.17-3.11 (m, 4H) ppm.

### Experimental Section—Biological Assays

The pharmacological activity of the compounds according to the invention can be assessed using in vitro- and/or in vivo-assays, as known to the person skilled in the art. The following examples describe the biological activity of the compounds according to the invention.

Example compounds according to the invention were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batches.

The in vitro activity of the compounds of the present invention can be demonstrated in the following assays:

RT-112-FABP4-NLuc Reporter Gene Assay

The identification and quantification of inverse agonism on the PPARG receptors was carried out using the bladder cancer cell line RT-112/84. The NanoLuc Luciferase gene was inserted into the 3′ untranslated region (UTR) of the canonical PPARG target gene, FABP4, in RT-112/84 cells using CRISPR/Cas9-guided homology-directed repair genome engineering leading to PPARG dependent expression of the NanoLuc@ Luciferase (Goldstein, J. T.; Berger, A. C.; Shih, J.; Duke, F. F.; Furst, L.; Kwiatkowski, D. J.; Cherniack, A. D.; Meyerson, M.; Strathdee, C. A., Genomic Activation of PPARG Reveals a Candidate Therapeutic Axis in Bladder Cancer. Cancer Res 2017, 77 (24), 6987-6998). Single cell clones were isolated and the clone with the widest dynamic range in signal upon treatment with agonist, rosiglitazone, was selected for use. Inverse agonists reduce the basal expression of the Luciferase resulting in a decrease of the Luciferase signal after addition of substrate and cell lysis. To exclude toxic effects of the compounds a parallel measurement of cell viability was performed based on the CellTiter Glo Assay (Promega).

Test Procedure:

5000 cells per well were plated out in 30 μl culture medium (DMEM/F12 (PAN Biotech), 10% FBS (Sigma), 2 mM glutamine (Gibco), 10 mM HEPES (Gibco)) in 384-well microtiter plates and kept in a cell incubator (96% humidity, 5% v/v CO2, 37° C.). On the next day 10 μl of test compounds dissolved in DMEM/F12 2% FBS and 0,01% BSA are placed in the wells of the microtiter plates. Final concentrations ranged from 50 μM to 0.03 nM. Plates were incubated over night in a cell incubator (96% humidity, 5% v/v CO2, 37° C.). On the next day the NanoLuc Luciferase activity was measured by addition of 40 μl of NanoLuc substrate and lysis buffer (Promega, Madison, Wisconsin) diluted in 2 mM Ca-tyrode buffer (20 mM HEPES, 130 mM NaCl, 5 mM KCl, 5 mM NaHCO₃, 2 mM MgCl2, pH 7,4). The resulting light signal was measured for 60 seconds in a luminometer. Every sample was tested in quadruples and mean values were used to calculate the IC50 values and efficacies of the compounds.

TABLE 1

| | PPARG_RT- | PPARG_RT- |
|---|---|---|
| | 112_FABP4_NLuc_inverse | 112_FABP4_NLuc_inverse |
| Example | agonist | agonist |
| No. | IC50 - [mol/l] | Efficacy - [%] |
| 1 | 3.80E−9 | 83.50 |
| 2 | 3.30E−8 | 110.00 |
| 3 | 3.15E−9 | 90.50 |
| 4 | 8.35E−9 | 87.00 |
| 5 | 1.27E−9 | 116.50 |
| 6 | 4.10E−9 | 134.50 |
| 7 | 1.40E−9 | 102.00 |
| 8 | 9.00E−10 | 96.00 |
| 9 | 3.55E−10 | 105.00 |
| 10 | 4.80E−9 | 108.00 |
| 11 | 4.20E−9 | 103.00 |
| 12 | 1.40E−8 | 117.00 |
| 13 | 4.00E−10 | 77.50 |
| 14 | 4.20E−10 | 82.50 |
| 15 | 5.20E−10 | 70.67 |
| 16 | 1.10E−9 | 67.80 |
| 17 | 1.30E−9 | 71.00 |
| 18 | 1.35E−9 | 95.00 |
| 19 | 1.22E−9 | 144.00 |
| 20 | 4.55E−10 | 114.00 |
| 21 | 1.48E−9 | 141.50 |
| 22 | 1.30E−9 | 106.00 |
| 23 | 8.15E−10 | 148.00 |
| 24 | 7.65E−9 | 92.50 |
| 25 | 4.00E−8 | 99.33 |
| 26 | 7.35E−9 | 94.00 |
| 27 | 2.10E−8 | 81.00 |
| 28 | 8.90E−8 | 73.67 |
| 29 | 1.30E−7 | 81.00 |
| 30 | 1.75E−8 | 105.50 |
| 31 | 3.00E−10 | 86.75 |
| 32 | 1.30E−9 | 74.00 |
| 33 | 1.05E−9 | 94.00 |
| 34 | 5.45E−8 | 95.00 |
| 35 | 2.15E−8 | 102.50 |
| 36 | 1.26E−9 | 97.50 |
| 37 | 5.00E−9 | 135.00 |
| 38 | 7.30E−10 | 98.50 |
| 39 | 6.55E−9 | 95.50 |
| 40 | 1.60E−6 | 114.00 |
| 41 | 9.20E−9 | 108.40 |
| 42 | 4.70E−9 | 102.00 |
| 43 | 7.20E−7 | 67.44 |
| 44 | 7.40E−6 | 51.50 |
| 45 | 5.25E−6 | 92.00 |
| 46 | 2.15E−6 | 104.75 |
| 47 | 8.95E−7 | 92.00 |
| 48 | 1.40E−7 | 78.50 |
| 49 | 9.90E−10 | 72.50 |
| 50 | 4.50E−10 | 108.00 |
| 51 | 1.50E−8 | 93.71 |
| 52 | 1.50E−9 | 96.00 |
| 53 | 3.75E−9 | 100.50 |

Proliferation UMUC9 H2B-GFP assay

Instrument: Incucyte® (Sartorius).

Description: Incucyte® Proliferation Assay measures cell proliferation based on nuclei count using live-cell time-lapse imaging. UMUC9 cells (Sigma—ECaCC 08090505) transduced with pTagGFP2-H2B (Evrogen) were seeded in 384-well plates (Corning #353962), at a density of 500 cells/well, in 40 μl/well MEM-alpha phenol-red free medium (Gibco Cat #41061-029) supplemented with 10% fetal calf serum. Next day cells were treated with compounds in a dose-response format with max 0.25% DMSO using a D300 digital dispenser instrument (HP). Measurements were performed on the day of treatment (Day 1 reading) and after 7 days of treatment (Day 8 reading). Green fluorescence in cell nuclei is measured using the instrument (Image Channels: Phase, Green; Acquisition time: 300 ms; Magnification: 4×).

Analysis: Selected metrics for analysis: Phase Object Confluence (Percent), Nuclei Count (1/Well) and Total Nuclei Area (μm²/Well). Day 1 measurements were subtracted from each Day 8 measurement and expressed as percentage of the vehicle (DMSO) control. Percentage proliferation values were then plotted against compound concentration using GraphPad PRISM software. The Sigmoidal 4PL X is log (concentration) model of linear regression was used. Relative IC50 values and % efficacy (span) is reported below.

TABLE 2

| Example | UM-UC-9 GFP proliferation | UM-UC-9 GFP proliferation |
|---|---|---|
| No. | IC50 - [mol/l] | Efficacy - [%] |
| 1 | 2.89E−9 | 51.41 |
| 2 | 2.04E−7 | 72.71 |
| 3 | >6.00E−6 | |
| 4 | 3.22E−9 | 138.19 |
| 5 | 5.38E−9 | 78.67 |
| 6 | 2.17E−8 | 79.55 |
| 7 | 9.09E−9 | 64.88 |
| 8 | 3.22E−8 | 71.51 |
| 9 | 4.51E−9 | 58.09 |
| 10 | 6.50E−8 | 86.72 |
| 11 | 8.23E−9 | 104.23 |
| 12 | 6.27E−8 | 108.79 |
| 13 | 4.80E−6 | 77.03 |
| 14 | 1.57E−6 | 85.00 |
| 15 | 7.96E−9 | 60.63 |
| 16 | 2.47E−8 | 24.81 |
| 17 | 1.52E−9 | 28.30 |
| 18 | 3.10E−8 | 59.55 |
| 19 | 1.16E−8 | 65.88 |
| 20 | 5.91E−9 | 69.62 |
| 21 | 1.06E−8 | 56.12 |
| 22 | 2.16E−8 | 86.60 |
| 23 | 3.77E−9 | 53.95 |
| 24 | 2.51E−8 | 24.39 |
| 25 | >6.00E−6 | n.d. |
| 26 | 2.75E−8 | 73.39 |
| 27 | 8.41E−8 | 47.53 |
| 28 | 1.35E−7 | 31.23 |
| 29 | n.d. | n.d. |
| 30 | 6.61E−8 | 49.62 |
| 31 | >6.00E−6 | n.d. |
| 32 | n.d. | n.d. |
| 33 | 4.91E−9 | 59.81 |
| 34 | 4.83E−6 | 100.15 |
| 35 | 1.82E−7 | 92.27 |
| 36 | 5.98E−9 | 65.97 |
| 37 | 3.09E−8 | 88.40 |
| 38 | 4.23E−9 | 49.49 |
| 39 | 3.42E−8 | 88.69 |
| 40 | 3.91E−7 | 72.07 |
| 41 | 9.83E−8 | 98.90 |
| 42 | 1.94E−9 | 68.63 |
| 43 | 3.20E−6 | 27.10 |
| 44 | 4.72E−6 | 102.60 |
| 45 | 2.68E−6 | 57.60 |
| 46 | 2.63E−6 | 70.60 |
| 47 | 2.24E−6 | 66.90 |
| 48 | 1.38E−7 | 43.13 |
| 49 | 3.17E−6 | 34.49 |
| 50 | 5.06E−9 | 50.83 |
| 51 | 3.85E−8 | 69.17 |
| 52 | 7.46E−7 | 28.64 |
| 53 | 1.84E−8 | 54.97 |

n.d. = not determined

Biochemical assay for determining inverse agonist activity of compounds A biochemical interaction assay measuring the ligand-dependent changes in interactions between the PPARG-LBD and a fluorescent peptide from the corepressor, NCOR2 (Smrt 102, ThermoFisher), was performed to evaluate inverse agonist activity.

Test procedure:

This assay was performed according to the manufacturers protocol (LanthaScreen TR-FRET PPAR gamma Corepressor Assay, ThermoFisher). PPARG-LBD, corepressor peptide NCOR2, LanthaScreen™ Tb-anti-GST antibody and compounds in 10 concentrations ranging from 50 μM to 0.03 nM were incubated in 15 μl per well of a 384 small volume black non-binding microtiter plate (Greiner) in Tris buffer (50 mM Tris, 50 mM NaCl, 20 mM MgCl2, 1 mM EDTA, 0,01% Casein (Sigma), 0,0003% Tween 20 (Sigma), 1 mM DTT (Sigma), pH 7,5) for 2 hours at room temperature. After incubation the FRET signal was measured using an PHERAstar Plus Plate Reader (BMG). Every sample was tested in duplicate and mean values were used to calculate the EC50 values and efficacies of the compounds. A model of nonlinear regression curve fit was used for calculation

Proliferation Assay for Determining Antiproliferative Effects Across a Panel of Cell Lines

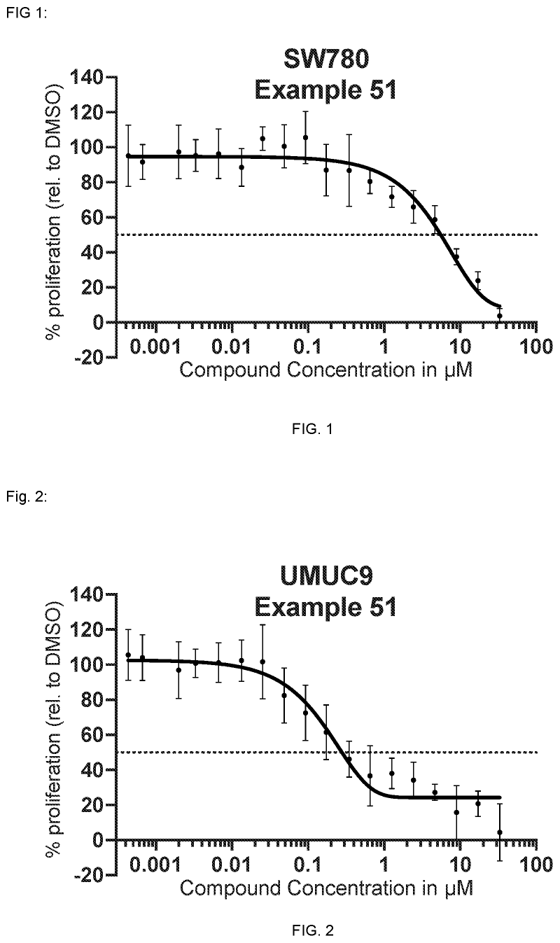

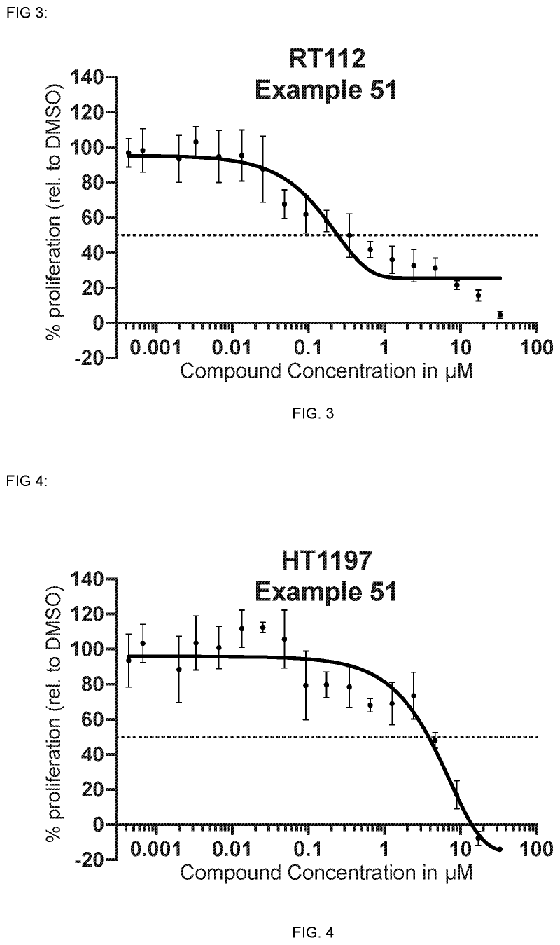

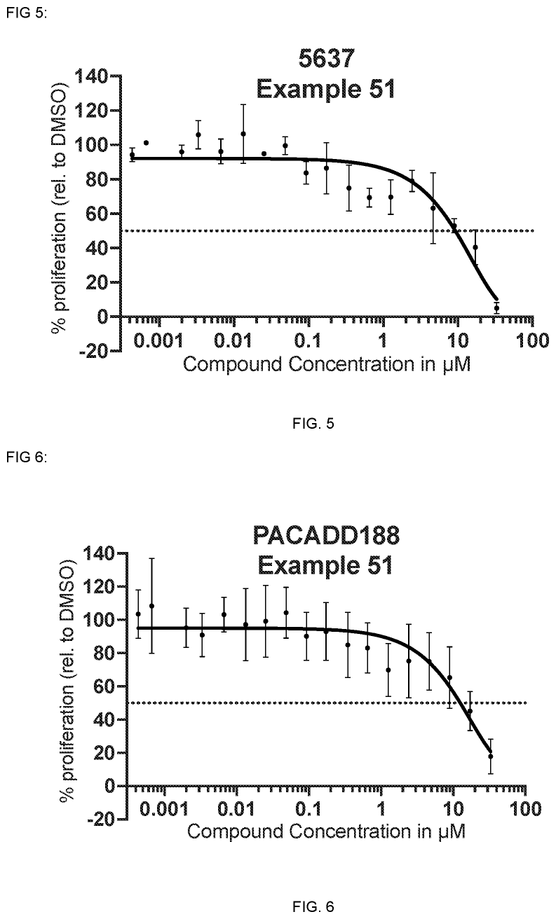

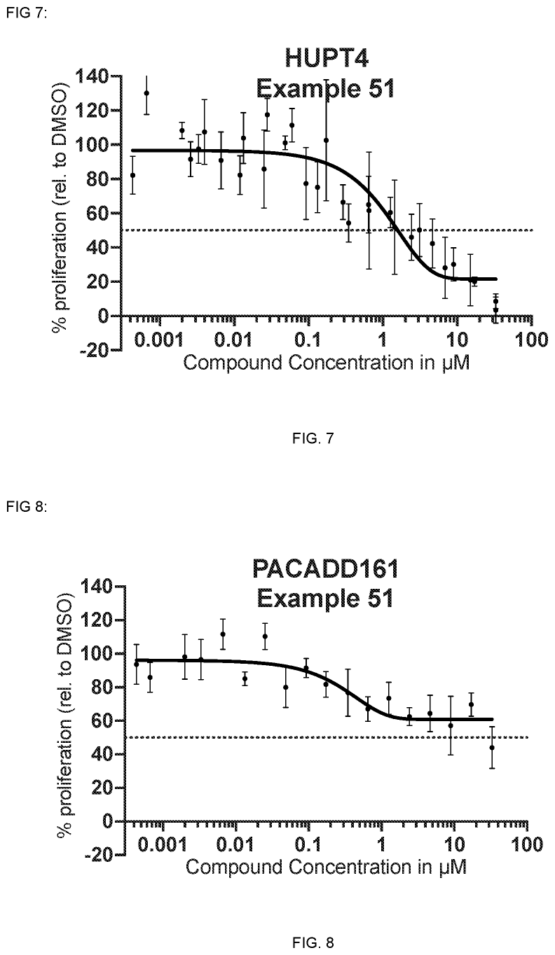

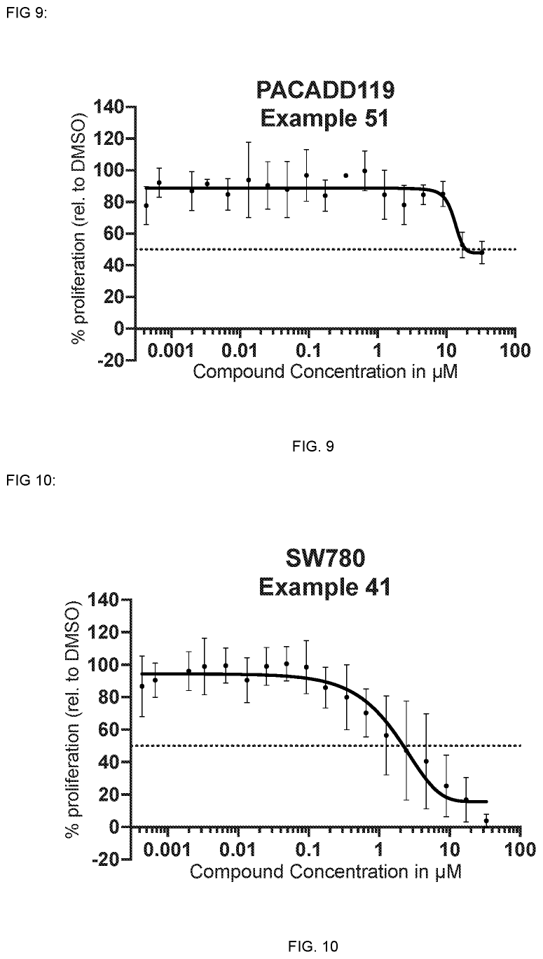

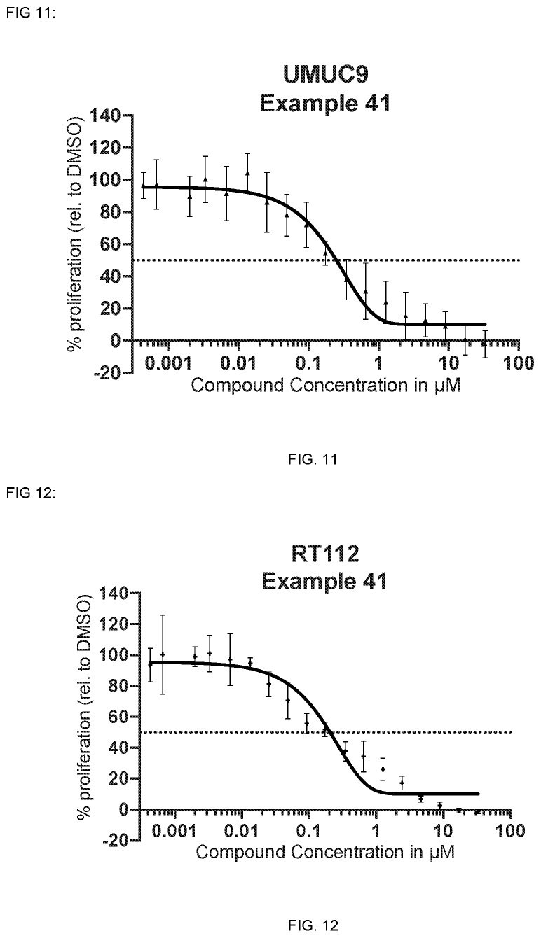

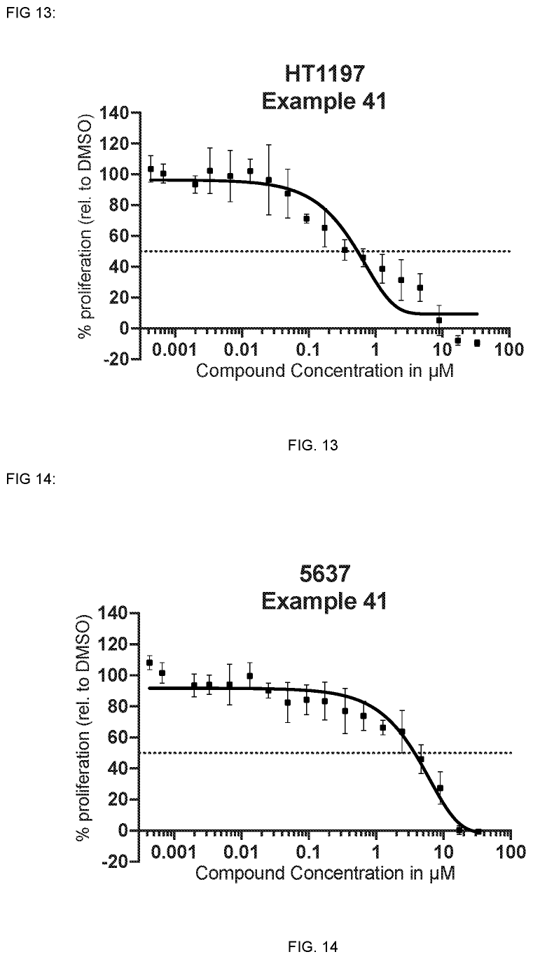

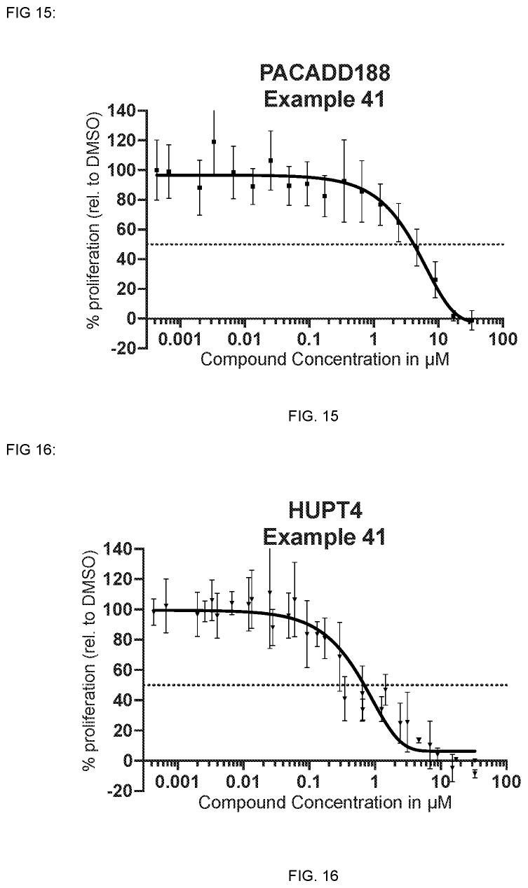

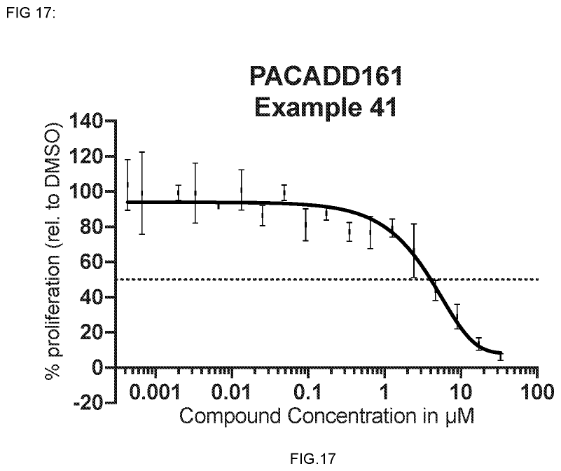

This assay protocol is followed in order to obtain the data underlying the figures FIG. 1 to FIG. 17 using a broader variety of cell lines supporting bladder cancer and pancreatic cancer. The UMUC9 cell line was purchased from Sigma-Aldrich; RT112, HUPT4, CAL29, PaCaDD188, PaCaDD161, and PaCaDD119 were obtained from DSMZ; 5637, HT1197, and SW780 were obtained from ATCC. All cell lines were cultured in the media recommended by the supplier, except SW780 which was cultured in RPMI-1640 with 10% fetal bovine serum.

Proliferation of a panel of bladder and pancreatic cancer cell lines was evaluated by CyQuant staining. Cells were seeded at the following counts per well in black clear-bottom 384-well plates: UMUC9 300-400, RT112 100, HUPT4 200-300, CAL29 400, PaCaDD188 600-800, PaCaDD161 200, PaCaDD119 400, 5637 100, HT1197 200, and SW780 200-400. The following day, cells were treated in triplicate with a 3.30×10-5 M to 4.32×10-10 M dose-range (18 points) of the respective compounds. To determine cell growth during the assay time, baseline signals were obtained at the start of treatment by freezing and staining a separate plate. After 7 days, the assay was stopped by discarding the supernatant and freezing the plates at −80° C. overnight. Next the cells were stained using CyQuant dye (Life Technologies, C7026) according to manufacturer's instructions and signals read on a PHERAstar FSX (BMG Labtech) using the fluorescence signal (485±10 nm/530±10 nm) measured in a spiral average over a 3 mm diameter per well. Using in-house software (SNAP-DRC) proliferation was calculated, determined as the growth that occurred between the start and end point measurement, with DMSO treated cells serving as control. G150 values were calculated with Graphpad Prism software using a four-parameter logistic regression as the concentration at which proliferation was 50% relative to DMSO-treated cells.

1. A compound of formula (I) 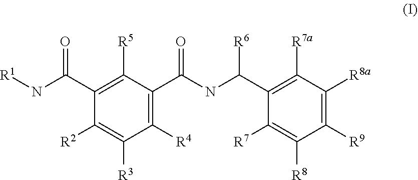
wherein, independently from each occurrence
R¹ is selected from 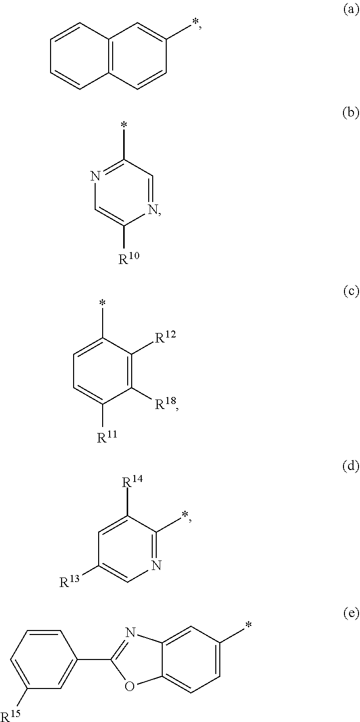
and * is the point of attachment to the nitrogen atom;
R² is selected from the group consisting of a halogen atom, and a —C═CH group;
R³ is selected from the group consisting of a hydrogen atom, an C₁-C₃-alkyl group, and a halogen atom;
R⁴ is selected from the group consisting of a hydrogen atom and a halogen atom;
R⁵ is selected from the group consisting of a hydrogen atom and a halogen atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, a (C₁-C₃)alkoxy group, and a —CH₂OCH₂CH₂R¹⁶ group;
R, R^{8a} is independently selected from the group consisting of a hydrogen atom and a halogen atom;
R⁹ is selected from the group consisting of a hydrogen atom and a halogen atom,
R¹⁰ is selected from the group consisting of a hydrogen atom and a phenoxy group;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from oxygen and nitrogen which is optionally substituted with a hydroxy group, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a halogen atom;
R¹⁶ is selected from the group consisting of a C₁-C₃-alkyl group and a C₁-C₃-alkoxy group;
R¹⁷ is selected from the group consisting of a hydrogen atom and a C₁-C₃-alkyl group;
R¹⁸ is selected from the group consisting of a hydrogen atom and a halogen atom;
or a stereoisomer, a tautomer, an N-oxide, a hydrate, a solvate, or a salt thereof, or a mixture of same.

2. The compound of claim 1,
wherein, independently from each occurrence
R¹ is selected from 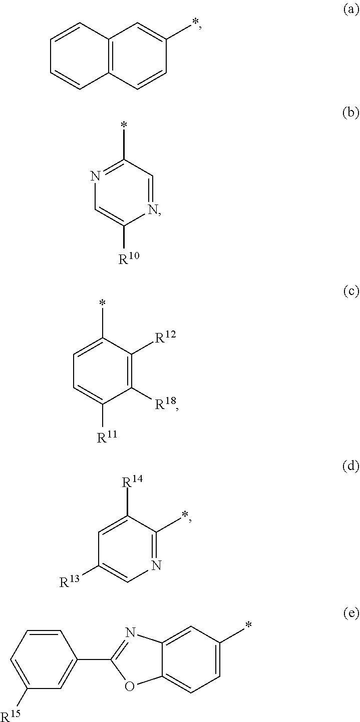
and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom,
R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3-6-membered heterocycloalkyl group is optionally substituted with a hydroxy group, a C₁-C₃-hyloalkyoxy group, a C₁-C₃-haloalkyl group, a 5- to 6-membered heteroaryl group which is optionally substituted with a C₁-C₃-alkyl group, and a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a chlorine atom;
R¹⁷ is selected from the group consisting of a hydrogen atom and a C₁-C₃-alkyl group;
R¹⁸ is selected from the group consisting of a hydrogen atom and a fluorine atom;
or a stereoisomer, a tautomer, an N-oxide, a hydrate, a solvate, or a salt thereof, or a mixture of same.

3. The compound according to claim 1, wherein independently from each occurrence R¹ is selected from 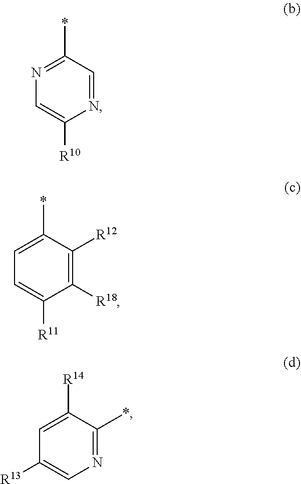
and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom,
R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a hydrogen atom and a fluorine atom;
or a stereoisomer, a tautomer, an N-oxide, a hydrate, a solvate, or a salt thereof, or a mixture of same.

4. The compound according to claim 1, wherein independently from each occurrence
R¹ is selected from 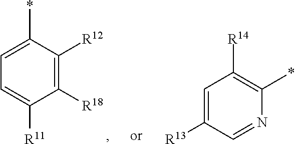
and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom,
R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹³ is selected from the group consisting of a hydrogen atom, a chlorine atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁸ is selected from the group consisting of a hydrogen atom and a fluorine atom; or a stereoisomer, a tautomer, an N-oxide, a hydrate, a solvate, or a salt thereof, or a mixture of same.

5. The compound of claim 1, wherein
R¹ is 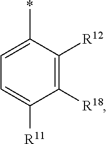
and * is the point of attachment to the nitrogen atom;
R² is a chlorine atom;
R³ is selected from the group consisting of a hydrogen atom, an C₁-C₃-alkyl group, and a fluorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, and a (C₁-C₃)alkoxy group;
R⁸, R^{8a} is independently selected from the group consisting of a hydrogen atom and a fluorine atom;
R⁹ is selected from the group consisting of a hydrogen atom and a fluorine atom,
R¹⁰ is a hydrogen atom;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from nitrogen and oxygen and said 3- to 6-membered heterocycloalkyl group is optionally substituted with a hydroxy group, a C₁-C₃-haloalkyoxy group, a C₁-C₃-haloalkyl group, and a 5- to 6-membered heteroaryl group which is optionally substituted with a C₁-C₃-alkyl group;
R¹² is selected from the group consisting of a hydrogen atom and a C₁-C₃-alkyl group;
R¹⁸ is selected from the group consisting of a hydrogen atom and a fluorine atom; or a stereoisomer, a tautomer, an N-oxide, a hydrate, a solvate, or a salt thereof, or a mixture of same.

6. The compound according to claim 1,
wherein, independently from each occurrence
R¹ is selected from 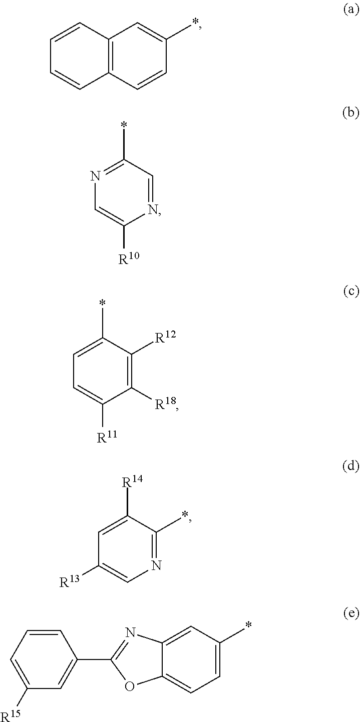
and * is the point of attachment to the nitrogen atom;
R² is selected from the group consisting of a chlorine atom, and a bromine atom,
R³ is selected from the group consisting of a hydrogen atom, a methyl group, and a chlorine atom;
R⁴ is selected from the group consisting of a hydrogen atom and a fluorine atom;
R⁵ is selected from the group consisting of a hydrogen atom and a fluorine atom;
R⁶ is selected from the group consisting of a hydrogen atom and a hydroxymethyl group;
R⁷, R^{7a} is independently selected from the group consisting of a hydrogen atom, a methyl group, a hydroxy group, a methoxy group, and a —CH₂OCH₂CH₂R¹⁶ group;
R⁸, R^{8a} is independently selected from the group consisting of a hydrogen atom and a fluorine atom;
R⁹ is selected from the group consisting of a hydrogen atom and a fluorine atom;
R¹⁰ is a hydrogen atom;
R¹¹ is selected from the group consisting of a hydrogen atom, a methyl group, a —C(CH₃)₂OH group, a trifluoromethyl group, a —OCHF₂ group, an oxetan-3-yl group, a morpholin-4-yl group, a 3-hydroxypiperidin-1-yl group, a 1,3,4-oxadiazol-2-yl group, a —OCH₂CH₂OR¹⁷ group, and a 3-methyl-1,2,4-oxadiazol-5-yl group;
R¹² is selected from the group consisting of a hydrogen atom and a methyl group;
R¹³ is selected from the group consisting of a hydrogen atom, a chlorine atom, a trifluoromethyl group, a cyclopropyl group, a oxetan-3-yl group, a morpholin-4-yl group, a —OCH₂C(CH₃)₂OH group, and a phenoxy group;
R¹⁴ is selected from the group consisting of a hydrogen atom and a methyl group;
R¹⁵ is selected from the group consisting of a methyl group and a chlorine atom;
R¹⁶ is selected from the group consisting of a methyl group and a methoxy group;
R¹⁷ is selected from the group consisting of a hydrogen atom and a methyl group;
R¹⁸ is selected from the group consisting of a hydrogen atom and a fluorine atom;
or a stereoisomer, a tautomer, an N-oxide, a hydrate, a solvate, or a salt thereof, or a mixture of same.

7. The compound according to claim 1 selected from the group consisting of
4-chloro-N3-[4-(difluoromethoxy)-2-methylphenyl]-6-fluoro-N1-[(2-hydroxyphenyl)methyl]benzene-1,3-dicarboxamide,
4-chloro-N3-[4-(difluoromethoxy)-2-methylphenyl]-6-fluoro-N1-[1-(4-fluorophenyl)-2-hydroxyethyl]benzene-1,3-dicarboxamide,
4-chloro-N3-[4-(difluoromethoxy)phenyl]-N1-[(3,4-difluorophenyl)methyl]-6-fluorobenzene-1,3-dicarboxamide,
4-chloro-6-fluoro-N1-{[4-fluoro-2-(propoxymethyl)phenyl]methyl}-N3-[2-methyl-4-(trifluoromethyl)phenyl]benzene-1,3-dicarboxamide,
4-chloro-N¹-[(3,4-difluorophenyl)methyl]-6-fluoro-N³-{4-[3-hydroxypiperidin-1-yl]-2-methylphenyl}benzene-1,3-dicarboxamide (stereoisomer 1),
4-chloro-N¹-[(3,4-difluorophenyl)methyl]-6-fluoro-N³-{4-[3-hydroxypiperidin-1-yl]-2-methylphenyl}benzene-1,3-dicarboxamide (stereoisomer 2),
4,5-dichloro-N3-[4-(difluoromethoxy)-2-methylphenyl]-N1-[(3,4-difluorophenyl)methyl]benzene-1,3-dicarboxamide,
4,5-dichloro-N1-[(3,4-difluorophenyl)methyl]-N3-[4-(2-hydroxyethoxy)-2-methylphenyl]benzene-1,3-dicarboxamide,
4,5-dichloro-N1-[(4-fluorophenyl)methyl]-N3-[2-(3-methylphenyl)-1,3-benzoxazol-5-yl]benzene-1,3-dicarboxamide,
4-chloro-N1-[(3,4-difluorophenyl)methyl]-6-fluoro-N3-[2-methyl-4-(1,3,4-oxadiazol-2-yl)phenyl]benzene-1,3-dicarboxamide,
4-chloro-N1-[(3,4-difluorophenyl)methyl]-6-fluoro-N3-{4-[3-hydroxypiperidin-1-yl]-2-methylphenyl}benzene-1,3-dicarboxamide,
4-chloro-N1-[(3,4-difluorophenyl)methyl]-6-fluoro-N3-[4-(2-hydroxyethoxy)-2-methylphenyl]benzene-1,3-dicarboxamide,
4,5-dichloro-N¹-[(3,4-difluorophenyl)methyl]-N³-[5-(trifluoromethyl)pyridin-2-yl]benzene-1,3-dicarboxamide,
4,5-dichloro-N3-(5-cyclopropylpyridin-2-yl)-N1-[(3,4-difluorophenyl)methyl]benzene-1,3-dicarboxamide,
4,5-dichloro-N¹-[(2-methoxyphenyl)methyl]-N³-phenylbenzene-1,3-dicarboxamide,
N¹-benzyl-4,5-dichloro-N³-phenylbenzene-1,3-dicarboxamide,
4,5-dichloro-N1-[(2-methylphenyl)methyl]-N3-phenylbenzene-1,3-dicarboxamide,
4-chloro-N1-[(3,4-difluorophenyl)methyl]-6-fluoro-5-methyl-N3-[3-methyl-5-(morpholin-4-yl)pyridin-2-yl]benzene-1,3-dicarboxamide,
4-chloro-N1-[(3,4-difluorophenyl)methyl]-6-fluoro-N3-[4-(2-methoxyethoxy)-2-methylphenyl]-5-methylbenzene-1,3-dicarboxamide,
4-chloro-N1-[(3,4-difluorophenyl)methyl]-6-fluoro-5-methyl-N3-[2-(3-methylphenyl)-1,3-benzoxazol-5-yl]benzene-1,3-dicarboxamide,
4-chloro-6-fluoro-N1-[(4-fluorophenyl)methyl]-N3-(5-phenoxypyridin-2-yl)benzene-1,3-dicarboxamide,
4-chloro-N3-[2-(3-chlorophenyl)-1,3-benzoxazol-5-yl]-6-fluoro-N1-[(4-fluorophenyl)methyl]benzene-1,3-dicarboxamide,
4-chloro-6-fluoro-N¹-[(4-fluorophenyl)methyl]-N³-[4-(3-methyl-1,2,4-oxadiazol-5-yl)phenyl]benzene-1,3-dicarboxamide,
N¹-benzyl-4-chloro-6-fluoro-N³-(4-methylphenyl)benzene-1,3-dicarboxamide,
N¹-benzyl-4-chloro-6-fluoro-N³-(2-methylphenyl)benzene-1,3-dicarboxamide,
4-chloro-6-fluoro-N¹-({4-fluoro-2-[(2-methoxyethoxy)methyl]phenyl}methyl)-N³-[2-methyl-4-(trifluoromethyl)phenyl]benzene-1,3-dicarboxamide,
4-chloro-6-fluoro-N¹-[(1R)-2-hydroxy-1-phenylethyl]-N³-[2-methyl-4-(trifluoromethyl)phenyl]benzene-1,3-dicarboxamide,
N^{l}-benzyl-4-chloro-6-fluoro-N³-phenylbenzene-1,3-dicarboxamide,
4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 1),
4-chloro-6-fluoro-N¹-[(1R*)-2-hydroxy-1-phenylethyl]-N³-[2-methyl-4-(trifluoromethyl)phenyl]benzene-1,3-dicarboxamide (steroisomer 2),
N¹-benzyl-4,5-dichloro-N³-[4-(difluoromethoxy)phenyl]-2-fluorobenzene-1,3-dicarboxamide,
N¹-benzyl-4,5-dichloro-N³-(5-chloropyridin-2-yl)-2-fluorobenzene-1,3-dicarboxamide,
N¹-benzyl-4,5-dichloro-2-fluoro-N3-[4-(oxetan-3-yl)phenyl]benzene-1,3-dicarboxamide,
4-chloro-N¹-(3,4-difluorobenzyl)-6-fluoro-N³-(pyridin-2-yl)isophthalamide,
4-chloro-N¹-(3,4-difluorobenzyl)-6-fluoro-N³-(4-morpholinophenyl)isophthalamide,
4-chloro-N¹-(3,4-difluorobenzyl)-6-fluoro-N³-(5-phenoxypyrazin-2-yl)isophthalamide,
4-chloro-N¹-(3,4-difluorobenzyl)-6-fluoro-N³-(3-methyl-5-(oxetan-3-yl)pyridin-2-yl)isophthalamide,
4,5-dichloro-N¹-(2-methoxybenzyl)-N³-(5-morpholinopyridin-2-yl)isophthalamide,
4-chloro-N¹-(3,4-difluorobenzyl)-6-fluoro-N³-(2-(m-tolyl)benzo[d]oxazol-5-yl)isophthalamide
N¹-(3,4-difluorobenzyl)-4-ethynyl-6-fluoro-N³-phenylisophthalamide,
4-chloro-N¹-(3,4-difluorobenzyl)-6-fluoro-N³-(3-methyl-5-morpholinopyridin-2-yl)isophthalamide,
,5-dichloro-N¹-(3,4-difluorobenzyl)-N³-(pyrazin-2-yl)isophthalamide,
N¹-benzyl-4-chloro-N³-phenylbenzene-1,3-dicarboxamide,
4-chloro-N¹-[(3,4-difluorophenyl)methyl]-N³-phenylbenzene-1,3-dicarboxamide,
4-chloro-N¹-[(4-fluorophenyl)methyl]-N³-phenylbenzene-1,3-dicarboxamide,
4-chloro-N¹-[(2-methoxyphenyl)methyl]-N³-phenylbenzene-1,3-dicarboxamide,
4-chloro-N¹-[(2-methylphenyl)methyl]-N³-phenylbenzene-1,3-dicarboxamide,
N¹-benzyl-4-bromo-N³-phenylbenzene-1,3-dicarboxamide
N¹-benzyl-4-bromo-5-chloro-N³-phenylbenzene-1,3-dicarboxamide,
N¹-benzyl-4-bromo-5-chloro-N³-(2-methylphenyl)benzene-1,3-dicarboxamide,
4-chloro-N¹-[(3,4-difluorophenyl)methyl]-6-fluoro-N³-[4-(2-hydroxypropan-2-yl)phenyl]benzene-1,3-dicarboxamide,
4-chloro-N¹-[(3,4-difluorophenyl)methyl]-6-fluoro-N³-[5-(2-hydroxy-2-methylpropoxy)pyridin-2-yl]-5-methylbenzene-1,3-dicarboxamide, and
4-chloro-N³-[4-(difluoromethoxy)-2-methylphenyl]-6-fluoro-N¹-[(4-fluorophenyl)methyl]benzene-1,3-dicarboxamide,
4-chloro-N¹-[(3,4-difluorophenyl)methyl]-6-fluoro-N³-(3-methyl-2-pyridyl)benzene-1,3-dicarboxamide
4-chloro-N¹-(3,4-difluorobenzyl)-6-fluoro-N³-(naphthalen-2-yl)isophthalamide
4,5-dichloro-N¹-[(2-methoxyphenyl)methyl]-N³-pyrazin-2-yl-benzene-1,3-dicarboxamide
4-chloro-N¹-[(3,4-difluorophenyl)methyl]-6-fluoro-N³-(5-phenoxy-2-pyridyl)benzene-1,3-dicarboxamide
4-chloro-N³-[4-(difluoromethoxy)-3-fluorophenyl]-6-fluoro-N¹-[(2-methoxyphenyl)methyl]benzene-1,3-dicarboxamide
4-chloro-6-fluoro-N³-[2-fluoro-4-(oxetan-3-yl)phenyl]-N¹-[(2-methoxyphenyl)methyl]benzene-1,3-dicarboxamide
4-chloro-6-fluoro-N¹-[(2-methoxyphenyl)methyl]-N³-5-(morpholin-4-yl)pyridin-2-yl]benzene-1,3-dicarboxamide
or a stereoisomer, a tautomer, an N-oxide, a hydrate, a solvate, or a salt thereof, or a mixture of same.

8. A method of preparing a compound of general formula (I) according to claim 1, said method comprising reacting a compound of formula (VI) 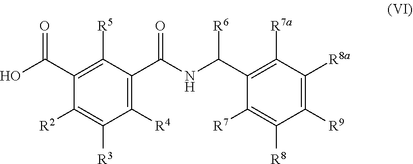
wherein
R² is selected from the group consisting of a halogen atom, and a —C═CH group;
R³ is selected from the group consisting of a hydrogen atom, an C₁-C₃-alkyl group, and a halogen atom;
R⁴ is selected from the group consisting of a hydrogen atom and a halogen atom;
R⁵ is selected from the group consisting of a hydrogen atom and a halogen atom;
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, a (C₁-C₃)alkoxy group, and a —CH₂OCH₂CH₂R¹⁶ group;
R⁸, R^{8a} is independently selected from the group consisting of a hydrogen atom and a halogen atom;
R⁹ is selected from the group consisting of a hydrogen atom and a halogen atom,
and an amine of formula (VII) 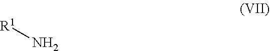
wherein
R¹ is selected from 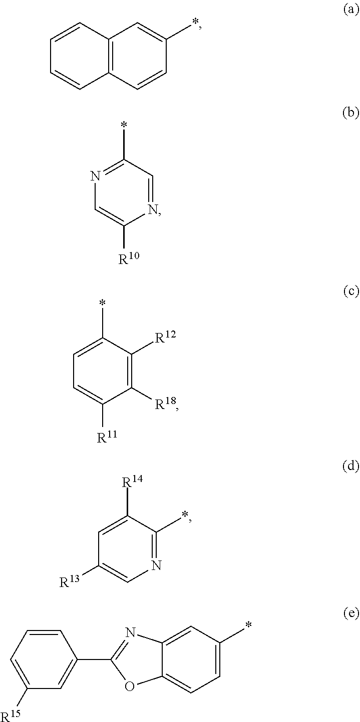
and * is the point of attachment to the nitrogen atom;
whereby
R¹⁰ is selected from the group consisting of a hydrogen atom and a phenoxy group;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from oxygen and nitrogen which is optionally substituted with a hydroxy group, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a halogen atom;
R¹⁶ is selected from the group consisting of a C₁-C₃-alkyl group and a C₁-C₃-alkoxy group;
R¹⁷ is selected from the group consisting of a hydrogen atom and a C₁-C₃-alkyl group;
R¹⁸ is selected from the group consisting of a hydrogen atom and a halogen atom;
under amide coupling conditions in order to obtain the compound of formula (I)

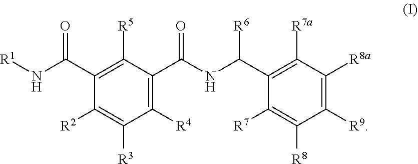

9. A method of preparing a compound of general formula (I) according to claim 1, said method comprising reacting a compound of formula (XI) 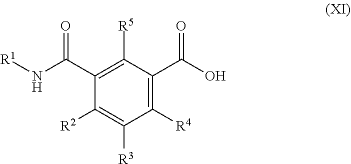
wherein
R¹ is selected from 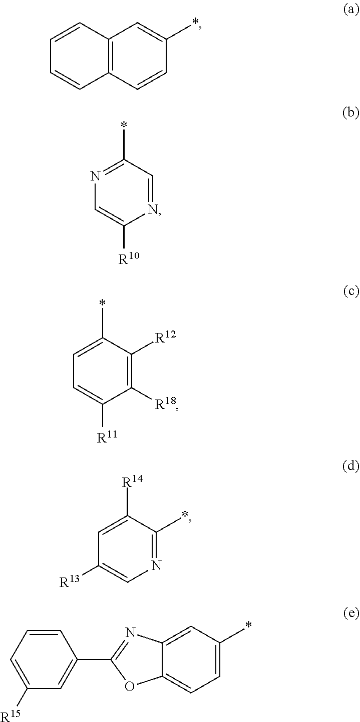
and * is the point of attachment to the nitrogen atom;
R² is selected from the group consisting of a halogen atom, and a —C═CH group;
R³ is selected from the group consisting of a hydrogen atom, an C₁-C₃-alkyl group, and a halogen atom;
R⁴ is selected from the group consisting of a hydrogen atom and a halogen atom;
R⁵ is selected from the group consisting of a hydrogen atom and a halogen atom;
R¹⁰ is selected from the group consisting of a hydrogen atom and a phenoxy group;
R¹¹ is selected from the group consisting of a hydrogen atom, an C₁-C₃-alkyl group, a C₁-C₃-hydroxyalkyl group, a 3- to 6-membered heterocycloalkyl group comprising 1 or 2 heteroatoms independently selected from oxygen and nitrogen which is optionally substituted with a hydroxy group, a C₁-C₃-haloalkyoxy group, a C₁-C₃-haloalkyl group, a 5- to 6-membered heteroaryl group which is optionally substituted with a C₁-C₃-alkyl group, a —O—(C₁-C₃)-alkylen-OR¹⁷ group;
R¹² is selected from the group consisting of a hydrogen atom and a C₁-C₃-alkyl group;
R¹³ is selected from the group consisting of a hydrogen atom, a halogen atom, a C₃-C₆-cycloalkyl group, a 4- to 6-membered heterocycloalkyl group comprising one heteroatom selected from oxygen or nitrogen, a C₁-C₃-haloalkyl group, a —O—(C₃-C₆-alkylen)-OH group, and a phenoxy group;
R¹⁴ is selected from the group consisting of a hydrogen atom and a C₁-C₃-alkyl group;
R¹⁵ is selected from the group consisting of a C₁-C₃-alkyl group and a halogen atom;
R¹⁷ is selected from the group consisting of a hydrogen atom and a C₁-C₃-alkyl group;
R¹⁸ is selected from the group consisting of a hydrogen atom and a halogen atom;
under amide coupling conditions with a compound of formula (III) 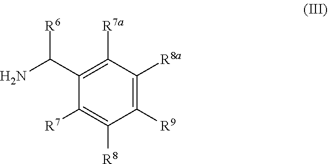
wherein
R⁶ is selected from the group consisting of a hydrogen atom and a hydroxy(C₁-C₃)-alkyl group;
R⁷, R^{7a} is independently selected from the group consisting of a hydrogen atom, an C₁-C₃-alkyl group, a hydroxy group, a (C₁-C₃)alkoxy group, and a —CH₂OCH₂CH₂R¹⁶ group;
R⁸, R^{8a} is independently selected from the group consisting of a hydrogen atom and a halogen atom;
R⁹ is selected from the group consisting of a hydrogen atom and a halogen atom,
R¹⁶ is selected from the group consisting of a C₁-C₃-alkyl group and a C₁-C₃-alkoxy group;
in order to obtain a compound of formula (I)

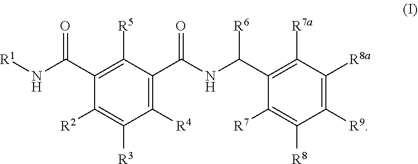

10. A method according to claim 8 where the amide coupling conditions comprise addition of a coupling reagent selected from T3P, HATU, DCCI and Ghosez reagent.

11. A pharmaceutical composition comprising a compound of general formula (I) according to claim 1 and one or more pharmaceutically acceptable excipients.

12. A pharmaceutical combination comprising:
one or more first active ingredients, in particular compounds of general formula (I) according to claim 1, and
one or more further active ingredients.

13. A method for the preparation of a medicament for the treatment or prophylaxis of a disease comprising combining the compound of general formula (I) according to claim 1 with one or more pharmaceutically suitable excipients.

14. A method for the treatment or prophylaxis of a disease in a subject in need thereof comprising administering the compound of general formula (I) according to claim 1 to the subject.

15. The method according to claim 14, wherein the disease is a hyperproliferative disease.

16. The method according to claim 15, wherein the hyperproliferative disease is a cancer disease.

17. The method according to claim 16, wherein the cancer disease is selected from a cancer appearing in an organ selected from the anus, the brain, the breast, the bones, the central and peripheral nervous system, the colon, the eye, the kidney, the endocrine glands, the endometrium, the esophagus, the gastrointestinal tract, the germ cells, the head and the neck, the kidney, the liver, the larynx and hypopharynx, the lung, the mesothelioma, the pancreas, the prostate, the rectum, the reproductive organs, the respiratory tract, the small intestine, the skin, the soft tissue, the stomach, the testis, the thyroid gland, the parathyroid gland, ureter, the urogenital tract, vagina and vulva and the connective tissue and metastases of these tumors.

18. The method according to claim 16, wherein the cancer disease is selected from breast cancer, esophageal cancer, pancreatic cancer, colorectal cancer, hepatocellular cancer, bladder cancer.

19. The method according to claim 16, wherein the cancer disease is selected from bladder cancer and pancreatic cancer.

20. A method of treating cancer in a subject, the method comprising administering to the subject a compound according to claim 1, thereby treating the cancer.
